(19) United States
(12) Patent Application Publication
(10) Pub. No.: US 2025/0228835 A1
(43) Pub. Date: Jul. 17, 2025

(54) PYRIDINYLOXYPYRIDINES AND RELATED COMPOUNDS AND THEIR USE IN TREATING MEDICAL CONDITIONS

(71) Applicant: Rectify Pharmaceuticals, Inc., Boston, MA (US)

(72) Inventors: Nathan Oliver Fuller, Arlington, MA (US); Robert Owen Hughes, Newtown, CT (US); Guy Bemis, San Diego, CA (US); Todd Bosanac, Burlington, CT (US); Pui Yee Ng, Lexington, MA (US)

(21) Appl. No.: 19/016,300

(22) Filed: Jan. 10, 2025

Related U.S. Application Data
(60) Provisional application No. 63/620,311, filed on Jan. 12, 2024.

(51) Int. Cl. A61K 31/444; A61K 31/4418; A61K 31/4995; C07D 213/76; C07D 401/04; C07D 487/08

(52) U.S. Cl. CPC A61K 31/444; A61K 31/4418; A61K 31/4995; C07D 213/76; C07D 401/04; C07D 487/08

(57) ABSTRACT

The invention provides pyridinyloxypyridines and related compounds, pharmaceutical compositions, and their use in treating medical conditions.

### CROSS-REFERENCE TO RELATED APPLICATIONS

This application claims the benefit of and priority to U.S. Provisional Patent Application Ser. No. 63/620,311, filed Jan. 12, 2024, the contents of which are hereby incorporated by reference in their entirety.

### FIELD OF THE INVENTION

The invention provides pyridinyloxypyridines and related compounds, pharmaceutical compositions, and their use in treating medical conditions.

### BACKGROUND

ATP-binding cassette (ABC) transporters are a large, phylogenetically conserved gene family with broad physiological and pathological relevance.[1,2] They are expressed throughout the body and transport a diverse range of substrates across lipid membranes. ABC transporters are transmembrane, ATP-binding proteins that use the energy released during ATP hydrolysis to move substrates from one side of a lipid membrane to the other.[2,3]

At least 21 ABC transporters underly rare monogenic disorders with even more implicated in the predisposition to and symptomology of common and complex diseases. Such broad (patho)physiological relevance places this class of proteins at the intersection of disease causation and therapeutic potential, underlining them as promising targets for drug discovery. Based on a handful of characterized disorders, including cystic fibrosis (CF), progressive familial intrahepatic cholestasis 2 (PFIC2), and Stargardt disease (STGD), there is a mechanistic commonality to pathogenic ABC transporter missense mutations, principally their impact on protein folding leading to endoplasmic reticulum (ER) degradation (trafficking defects), or protein function, leading to decreased substrate transport (transport defects). Importantly, small molecule compounds may enable treatment of disease symptoms not directly caused by mutations in ABC transporter genes.

There is a need for small molecule compounds that can positively modulate ABC transporter function and therefore be used for the treatment of diseases in which such enhancement is predicted to be of therapeutic value, either in modifying disease or in treating disease symptomology. For example, there is a need for modulators that can address mutations in ABCB11, ABCB4, ABCC6, ABCA4, ABCD1, ABCA1 and ABCA7 for the treatment of PFIC2, PFIC3, PXE, GACI, STGD, X-ALD and Alzheimer's disease, respectively.

Accordingly, the need exists for new therapeutic methods and compounds for treating ABC transporter dysfunction. The present invention addresses the foregoing needs and provides other related advantages.

### SUMMARY

The invention provides pyridinyloxypyridines and related compounds, pharmaceutical compositions, and their use in treating medical conditions. In particular, one aspect of the invention provides a collection of pyridinyloxypyridine compounds, such as a compound represented by Formula I.

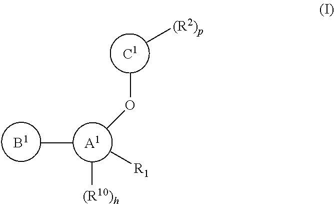

or a pharmaceutically acceptable salt thereof, where the variables are as defined in the detailed description. Further description of additional collections of pyridinyloxypyridine compounds are described in the detailed description. The compounds may be part of a pharmaceutical composition comprising a pharmaceutically acceptable carrier.

Another aspect of the invention provides a method of treating ABC transporter dysfunction. The method comprises administering to a subject in need thereof a therapeutically effective amount of a compound described herein, such as a compound of Formula I, to treat the ABC transporter dysfunction, as further described in the detailed description.

Another aspect of the invention provides a method of modulating the function of a protein selected from ABCB11, ABCB11 E297G, ABCC6, ABCB4, ABCA4 P1380L, ABCD1, and ABCD2 in a subject. The method comprises administering to a subject in need thereof an effective amount of a compound described herein, such as a compound of Formula I, to thereby modulate the function of said protein, as further described in the detailed description.

### DETAILED DESCRIPTION

The invention provides pyridinyloxypyridines and related compounds, pharmaceutical compositions, and their use in treating medical conditions. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In certain embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In certain embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In certain embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In certain embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

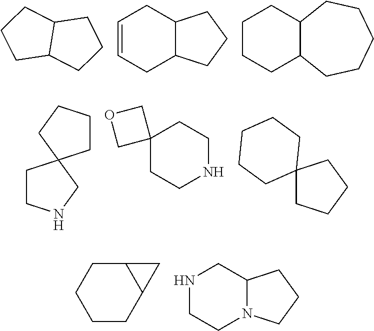

Exemplary bridged bicyclics include:

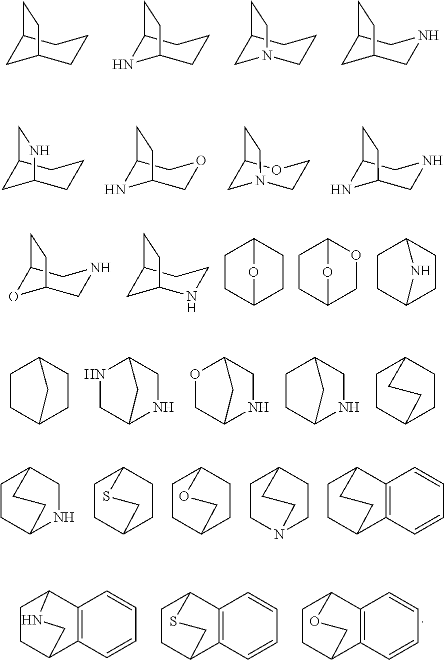

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)-group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The terms “halo” and “halogen” mean F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it. For example, “phenylene” is a bivalent phenyl group when it has two groups attached to it (e.g.,

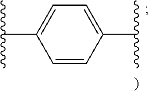 [SMILES: *c1ccc(*)cc1]

“phenylene” is a trivalent phenyl group when it has three groups attached to it (e.g.,

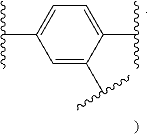 [SMILES: *c1ccc(*)c(*)c1]

The term “arylene” refers to a bivalent aryl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “heteroarylene” refers to a multivalent heteroaryl group having the appropriate number of open valences to account for groups attached to it. For example, “heteroarylene” is a bivalent heteroaryl group when it has two groups attached to it; “heteroarylene” is a trivalent heteroaryl group when it has three groups attached to it.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by one or more oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it. The term “oxo-heterocyclylene” refers to a multivalent oxo-heterocyclyl group having the appropriate number of open valences to account for groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

The term “partially aromatic bicyclic ring” refers to a bicyclic ring in which one ring is aromatic and the other ring is not aromatic.

As used herein, the term “pyridinylene-N-oxide” refers

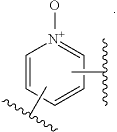

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R*, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}.

Each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, =NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, RT is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66 (1) 1-19; P. Gould. International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. The invention includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as an atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The terms “alkenyl” and “alkynyl” are art-recognized and refer to unsaturated aliphatic groups analogous in length and possible substitution to the alkyls described above, but that contain at least one double or triple bond respectively.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “=O” substituent. For example, a cyclopentane substituted with an oxo group is cyclopentanone.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

The term “EC₅₀” is art recognized and refers to the concentration of a compound that is required to achieve a response that is 50% of the maximum target effect relative to the baseline.

The term “Eₘₐₓ” is art recognized and refers to the concentration of a compound that is required to achieve maximal target effect.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Pyridinyloxypyridine Compounds

### I.1 Pyridinyloxypyridine Compounds of Formula (I)

One aspect of the invention provides pyridinyloxypyridine compounds. The compounds may be used in the pharmaceutical compositions and therapeutic methods described herein. Exemplary compounds are described in the following sections, along with exemplary procedures for making the compounds. One aspect of the invention provides a compound represented by Formula I.

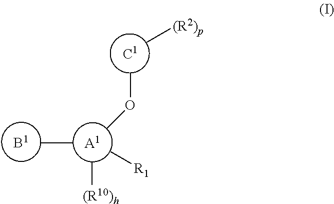

or a pharmaceutically acceptable salt thereof, wherein:
Ring A¹ is (i) a 6-membered heteroarylene having 1 or 2 heteroatoms selected from nitrogen; (ii) phenylene; or (iii) a 5-7 membered saturated or partially unsaturated monocyclic oxo-heterocyclyl having 1-2 heteroatoms independently selected from nitrogen, oxygen, and sulfur;
Ring B¹ is a 5-6 membered monocyclic heteroaryl having 1-3 heteroatoms independently selected from nitrogen, oxygen, and sulfur, a 3-7 membered monocyclic heterocyclyl having 1-2 heteroatoms independently selected from nitrogen, oxygen, and sulfur, a 3-7 membered saturated or partially unsaturated monocyclic oxo-heterocyclyl having 1-2 heteroatoms independently selected from nitrogen, oxygen, and sulfur, a 7-12 membered bridged or spirocyclic heterocyclyl having 1-2 heteroatoms independently selected from nitrogen, oxygen, and sulfur, or a partially aromatic 9-10 membered bicyclic ring containing 2, 3, or 4 heteroatoms independently selected from nitrogen, oxygen, and sulfur, wherein each ring is substituted with j instances of R³; or Ring B¹ is phenyl substituted with k instances of R³;
Ring C¹ is phenylene, a 5 or 6-membered heteroarylene containing 1 or 2 heteroatoms selected from nitrogen, oxygen and sulfur, or pyridinylene-N-oxide;
R¹ is —N(R⁹)—SO₂—R⁶, —SO₂—N(R⁹)—R⁶, —SO₂—N(R⁷)₂, —S(NR⁹)(O)—R⁶, —N═S(O)(R⁹)₂, or —S(O₂)—R⁶, or

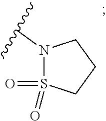 [SMILES: *N1CCCS1(=O)=O]

R² represents independently for each occurrence hydroxyl, C₁₋₃ alkyl, halo, C₁₋₃ haloalkyl, C₁₋₃ hydroxyalkyl, cyano, C₃₋₄ cycloalkyl, —O—R¹¹, a 3-5 membered heterocyclyl having 1-2 heteroatoms independently selected from nitrogen, oxygen, and sulfur, or a 7-12 membered bridged or spirocyclic heterocyclyl having 1-2 heteroatoms independently selected from nitrogen, oxygen, and sulfur; or two vicinal occurrences of R² are taken together with the atoms to which they are attached to form a 3-6 membered ring having 0, 1, or 2 heteroatoms independently selected from nitrogen, oxygen, and sulfur;
R³ represents independently for each occurrence halo, cyano, C₁₋₆ alkyl, C₂₋₃ alkynyl, C₁₋₆ haloalkyl, hydroxyl, (C₀₋₂ alkylene)-C₁₋₆ alkoxyl, C₁₋₃ hydroxyalkyl, —CON(R⁴)₂, —C(O)—R⁵, —C(O)—OR⁵, —S(O)₂—R⁵, —N(R⁴)—C(O)—R⁵, —S(NR⁴)(O)—R⁵, C₁₋₆ haloalkoxyl, C₃₋₄ cycloalkyl, or a 3-6 membered heterocyclyl having 1-2 heteroatoms independently selected from nitrogen, oxygen, and sulfur, wherein each cycloalkyl and heterocyclyl is substituted with r instances of R¹³;
R⁴ represents independently for each occurrence hydrogen, C₁₋₄ alkyl, or cyclopropyl;
R⁵ and R⁶ each represent independently for each occurrence C₁₋₄ alkyl, C₃₋₅ cycloalkyl, —(C₀₋₂ alkylene)-phenyl, —(C₀₋₂ alkylene)-(3-6 membered monocyclic heterocyclyl having 1-2 heteroatoms independently selected from nitrogen, oxygen, and sulfur), or —(C₀₋₂ alkylene)-(5-10 membered heteroaryl having 1-3 heteroatoms independently selected from nitrogen, oxygen, and sulfur), wherein the cycloalkyl, phenyl, heterocyclyl, and heteroaryl are substituted with m instances of R⁸, and wherein each alkylene is substituted with n instances of R⁸;
R⁷ is hydrogen, or two R⁷ groups attached to the same nitrogen atom are taken together with the nitrogen to which they are attached to form a 3-6 membered saturated monocyclic ring having in addition to said nitrogen atom 0, 1, or 2 heteroatoms independently selected from nitrogen, oxygen, and sulfur;
R⁸ represents independently for each occurrence C₁₋₄ alkyl, halo, C₁₋₃ haloalkyl, hydroxyl, C₁₋₄ alkoxy, cyano, or cyclopropyl;
R⁹ represents independently for each occurrence hydrogen, C₁₋₃ alkyl, or cyclopropyl;
R¹⁰ is C₁₋₃ alkyl, C₁₋₃ haloalkyl, halo, hydroxyl, or cyclopropyl;
R¹¹ is C₁₋₄ alkyl, C₁₋₄ haloalkyl, cyclopropyl, or a 3-5 membered saturated heterocyclyl having 1-2 heteroatoms independently selected from nitrogen, oxygen, and sulfur;
R¹² is C₁₋₃ alkyl, halo, cyano, or —SO₂—(C₁₋₃ alkyl);
R¹³ is halo, C₁₋₃ alkyl, or C₁₋₃ alkoxyl; h is 0, 1, or 2;
j is 0, 1, or 2;
k is 1 or 2;
m is 0, 1, or 2;
n is 0, 1, 2, or 3;
p is 1, 2, 3, or 4
q is 0, 1, or 2; and
r is 0, 1, or 2.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

As defined generally above, Ring A¹ is (i) a 6-membered heteroarylene having 1 or 2 heteroatoms selected from nitrogen; (ii) phenylene; or (iii) a 5-7 membered saturated or partially unsaturated monocyclic oxo-heterocyclyl having 1-2 heteroatoms independently selected from nitrogen, oxygen, and sulfur. In certain embodiments, Ring A¹ is phenylene. In certain embodiments, Ring A¹ is a 6-membered monocyclic heteroaromatic ring containing 1 or 2 heteroatoms selected from nitrogen. In certain embodiments, Ring A¹ is a a 5-7 membered saturated or partially unsaturated monocyclic oxo-heterocyclyl having 1-2 heteroatoms independently selected from nitrogen, oxygen, and sulfur. In certain embodiments, Ring A¹ is pyridinylene or pyrimidinylene. In certain embodiments, Ring A¹ is pyridinylene. In certain embodiments, Ring A¹ is pyrimidinylene. In certain embodiments, Ring A¹ is selected from the groups depicted in the compounds in Table 1, below.

As defined generally above, Ring B¹ is a 5-6 membered monocyclic heteroaryl having 1-3 heteroatoms independently selected from nitrogen, oxygen, and sulfur, a 3-7 membered monocyclic heterocyclyl having 1-2 heteroatoms independently selected from nitrogen, oxygen, and sulfur, a 3-7 membered saturated or partially unsaturated monocyclic oxo-heterocyclyl having 1-2 heteroatoms independently selected from nitrogen, oxygen, and sulfur, a 7-12 membered bridged or spirocyclic heterocyclyl having 1-2 heteroatoms independently selected from nitrogen, oxygen, and sulfur, or a partially aromatic 9-10 membered bicyclic ring containing 2, 3, or 4 heteroatoms independently selected from nitrogen, oxygen, and sulfur, wherein each ring is substituted with j instances of R³; or Ring B¹ is phenyl substituted with k instances of R.

In certain embodiments, Ring B¹ is a 3-7 membered monocyclic heterocyclyl having 1-2 heteroatoms independently selected from nitrogen, oxygen, and sulfur, a 5-6 membered monocyclic heteroaryl having 1-3 heteroatoms independently selected from nitrogen, oxygen, and sulfur, or a 7-12 membered bridged heterocyclyl having 1-2 heteroatoms independently selected from nitrogen, oxygen, and sulfur, wherein each ring is substituted with j instances of R³; or Ring B¹ is phenylene substituted with k instances of R³.

In certain embodiments, Ring B¹ is a 3-7 membered monocyclic heterocyclyl having 1-2 heteroatoms independently selected from nitrogen, oxygen, and sulfur, a 5-6 membered monocyclic heteroaryl having 1-3 heteroatoms independently selected from nitrogen, oxygen, and sulfur, or a 7-12 membered bridged heterocyclyl having 1-2 heteroatoms independently selected from nitrogen, oxygen, and sulfur, wherein each ring is substituted with j instances of R³. In certain embodiments, Ring B¹ is a 3-7 membered monocyclic heterocyclyl having 1-2 heteroatoms independently selected from nitrogen, oxygen, and sulfur, a 5-6 membered monocyclic heteroaryl having 1-3 heteroatoms independently selected from nitrogen, oxygen, and sulfur, or a 7-12 membered bridged heterocyclyl having 1-2 heteroatoms independently selected from nitrogen, oxygen, and sulfur.

In certain embodiments, Ring B¹ is a 3-7 membered monocyclic heterocyclyl having 1-2 heteroatoms independently selected from nitrogen, oxygen, and sulfur, substituted with j instances of R³. In certain embodiments, Ring B¹ is a 5-6 membered monocyclic heteroaryl having 1-3 heteroatoms independently selected from nitrogen, oxygen, and sulfur, substituted with j instances of R³. In certain embodiments, Ring B¹ is a 7-12 membered bridged heterocyclyl having 1-2 heteroatoms independently selected from nitrogen, oxygen, and sulfur, substituted with j instances of R³.

In certain embodiments, Ring B¹ is a 3-7 membered monocyclic oxo-heterocyclyl having 1-2 heteroatoms independently selected from nitrogen, oxygen, and sulfur, substituted with j instances of R³.

In certain embodiments, Ring B¹ is a 3-7 membered monocyclic heterocyclyl having 1-2 heteroatoms independently selected from nitrogen, oxygen, and sulfur. In certain embodiments, Ring B¹ is a 5-6 membered monocyclic heteroaryl having 1-3 heteroatoms independently selected from nitrogen, oxygen, and sulfur. In certain embodiments, Ring B¹ is a 7-12 membered bridged heterocyclyl having 1-2 heteroatoms independently selected from nitrogen, oxygen, and sulfur.

In certain embodiments, Ring B¹ is a 7-12 membered bridged or spirocyclic heterocyclyl having 1-2 heteroatoms independently selected from nitrogen, oxygen, and sulfur, substituted with j instances of R³.

In certain embodiments, Ring B¹ is a partially aromatic 9-10 membered bicyclic ring containing 2, 3, or 4 heteroatoms independently selected from nitrogen, oxygen, and sulfur, substituted with j instances of R³.

In certain embodiments, Ring B¹ is phenyl substituted with k instances of R³. In certain embodiments, Ring B¹ is phenylene.

In certain embodiments, Ring B¹ is pyridinyl, pyrazolyl, imidazolyl, piperazinyl, 2,5,-diazabicyclo[2.2.1]heptanyl, 3,6-diazabicyclo[3.1.1]heptanyl, 5,6-dihydro-8H-7-[1,2,4]triazolo[3,4-c]pyrazinyl, 2,6-diazaspiro[3.4]octanyl, 2,6-diazaspiro[3.3]heptanyl, or azetidinyl, wherein each ring is substituted with j instances of R³. In certain embodiments, Ring B¹ is pyridinyl, substituted with j instances of R³. In certain embodiments, Ring B¹ is pyrazolyl, substituted with j instances of R³. In certain embodiments, Ring B¹ is imidazolyl, substituted with j instances of R³. In certain embodiments, Ring B1 is piperazinyl, substituted with j instances of R³. In certain embodiments, Ring B¹ is 2,5,-diazabicyclo[2.2.1]heptanyl, substituted with j instances of R³. In certain embodiments, Ring B¹ is 3,6-diazabicyclo[3.1.1]heptanyl, substituted with j instances of R³. In certain embodiments, Ring B¹ is 5,6-dihydro-8H-7-[1,2,4]triazolo[3,4-c]pyrazinyl, substituted with j instances of R³. In certain embodiments, Ring B¹ is 2,6-diazaspiro[3.4]octanyl, substituted with j instances of R³. In certain embodiments, Ring B¹ is 2,6-diazaspiro[3.3]heptanyl, substituted with j instances of R³. In certain embodiments, Ring B¹ is azetidinyl, substituted with j instances of R³.

In certain embodiments, Ring B¹ is selected from the groups depicted in the compounds in Table 1, below.

As defined generally above, Ring C¹ is phenylene, a 5 or 6-membered heteroarylene containing 1 or 2 heteroatoms selected from nitrogen, oxygen, and sulfur, or pyridinylene-N-oxide. In certain embodiments, Ring C¹ is phenylene. In certain embodiments, Ring C¹ is a 5 or 6-membered heteroarylene containing 1 or 2 heteroatoms selected from nitrogen, oxygen, and sulfur. In certain embodiments, Ring C¹ is pyridinylene-N-oxide. In certain embodiments, Ring C¹ is a 5-membered heteroarylene containing 1 or 2 heteroatoms selected from nitrogen, oxygen, and sulfur. In certain embodiments, Ring C¹ is a 6-membered heteroarylene containing 1 or 2 heteroatoms selected from nitrogen, oxygen and sulfur. In certain embodiments, Ring C¹ is a 6-membered heteroarylene containing 1 or 2 heteroatoms selected from nitrogen. In certain embodiments, Ring C¹ is phenylene or pyridinylene. In certain embodiments, Ring C¹ is pyridinylene. In certain embodiments, Ring C¹ is pyridinylene. In certain embodiments, Ring C¹ is pyrimidinylene. In certain embodiments, Ring C¹ is pyridinylene or pyrimidinylene. In certain embodiments, Ring C¹ is selected from the groups depicted in the compounds in Table 1, below.

As defined generally above, R¹ is —N(R⁹)—SO₂—R⁶, —SO₂—N(R⁹)—R⁶, —SO₂—N(R⁷)₂, —S(NR⁹)(O)—R⁶, —N═S(O)(R⁹)₂, —S(O₂)—R⁶, or

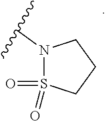 [SMILES: *N1CCCS1(=O)=O]

In certain embodiments, R¹ is —N(R⁹)—SO₂—R⁶, —SO₂—N(R⁹)—R⁶, —S(NR⁹)(O)—R⁶, or —S(O₂)—R⁶. In certain embodiments, R¹ is —N(R⁹)—SO₂—R⁶. In certain embodiments, R¹ is —SO₂—N(R⁹)—R⁶. In certain embodiments, R¹ is —SO₂—N(R⁷)₂. In certain embodiments, R¹ is —S(NR⁹)(O)—R⁶. In certain embodiments, R¹ is, —N═S(O)(R⁹)₂. In certain embodiments, R¹ is —S(O₂)—R⁶. In certain embodiments, R¹ is

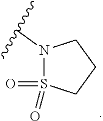 [SMILES: *N1CCCS1(=O)=O]

In certain embodiments, R¹ is —NH—SO₂—CH₃. In certain embodiments, R¹ is selected from the groups depicted in the compounds in Table 1, below.

As defined generally above, R² represents independently for each occurrence hydroxyl, C₁₋₃ alkyl, halo, C₁₋₃ haloalkyl, C₁₋₃ hydroxyalkyl, cyano, C₃₋₄ cycloalkyl-O—R¹¹, a 3-5 membered heterocyclyl having 1-2 heteroatoms independently selected from nitrogen, oxygen, and sulfur, or a 7-12 membered bridged or spirocyclic heterocyclyl having 1-2 heteroatoms independently selected from nitrogen, oxygen, and sulfur, wherein each cyclokalkyl and heterocyclyl is substituted with q instances of R¹²; or two vicinal occurrences of R² are taken together with the atoms to which they are attached to form a 3-6 membered ring having 0, 1, or 2 heteroatoms independently selected from nitrogen, oxygen, and sulfur. In certain embodiments, R² represents independently for each occurrence C₁₋₃ alkyl, halo, C₁₋₄ alkoxyl, C₁₋₃ haloalkyl, C₁₋₃ hydroxyalkyl, or cyano.

In certain embodiments, R² represents independently for each occurrence C₁₋₃ alkyl. In certain embodiments, R² represents independently for each occurrence halo. In certain embodiments, R² represents independently for each occurrence C₁₋₃ alkoxyl. In certain embodiments, R² represents independently for each occurrence C₁₋₃ haloalkyl. In certain embodiments, R² represents independently for each occurrence C₁₋₃ hydroxyalkyl. In certain embodiments, R² represents independently for each occurrence a 3-5 membered heterocyclyl having 1-2 heteroatoms independently selected from nitrogen, oxygen, and sulfur substituted with q instances of R¹². In certain embodiments, R² is C₃₋₄ cycloalkyl substituted with q instances of R¹². In certain embodiments, R² represents independently for each occurrence —O—R¹¹. In certain embodiments, R² represents independently for each occurrence a 7-12 membered bridged or spirocyclic heterocyclyl having 1-2 heteroatoms independently selected from nitrogen, oxygen, and sulfur substituted with q instances of R¹². In certain embodiments, R² is cyano. In certain embodiments, R² is methyl. In certain embodiments, R² is hydroxyl. In certain embodiments, R² represents independently for each occurrence methyl or cyclopropyl. In certain embodiments, or two vicinal occurrences of R² are taken together with the atoms to which they are attached to form a 3-6 membered ring having 0, 1, or 2 heteroatoms independently selected from nitrogen, oxygen, and sulfur.

In certain embodiments, R² represents independently for each occurrence C₁₋₃ alkyl, C₁₋₃ haloalkyl, —O—(C₁₋₄ haloalkyl), or C₃₋₄ cycloalkyl. In certain embodiments, R² represents independently for each occurrence methyl, cyclopropyl, O-cyclopropyl, —CHF₂, —CF₃, or —O—CF₃.

In certain embodiments, p is 3 and at least one R² is C₁₋₃ haloalkyl. In certain embodiments, p is 3 and at least two R² are C₁₋₃ haloalkyl. In certain embodiments, p is 3 and at least two R² are methyl. In certain embodiments, p is 3 and at least one R² is cyclopropyl. In certain embodiments, p is 3 and at least two R² are cyclopropyl.

In certain embodiments, R² is selected from the groups depicted in the compounds in Table 1, below.

As defined generally above, R³ represents independently for each occurrence halo, cyano, C₁₋₆ alkyl, C₁₋₆ haloalkyl, hydroxyl, (C₀₋₂ alkylene)-C₁₋₆ alkoxyl, C₁₋₃ hydroxyalkyl, —CON(R⁴)₂, —C(O)—R⁵, —C(O)—OR⁵, —S(O)₂—R⁵, —N(R⁴)—C(O)—R⁵, —S(NR⁴)(O)—R⁵, or C₁₋₆ haloalkoxyl, C₃₋₄ cycloalkyl, or a 3-6 membered heterocyclyl having 1-2 heteroatoms independently selected from nitrogen, oxygen, and sulfur, wherein each cycloalkyl and heterocyclyl is substituted with r instances of R¹³. In certain embodiments, R³ represents independently for each occurrence halo. In certain embodiments, R³ represents independently for each occurrence C₁₋₆ alkyl. In certain embodiments, R³ represents independently for each occurrence C₁₋₆ haloalkyl. In certain embodiments, R³ represents independently for each occurrence (C₀₋₂ alkylene)-C₁₋₆ alkoxyl. In certain embodiments, R³ represents independently for each occurrence C₁₋₃ hydroxyalkyl. In certain embodiments, R³ represents independently for each occurrence —CON(R⁴)₂. In certain embodiments, R³ represents independently for each occurrence —C(O)—R⁵. In certain embodiments, R³ represents independently for each occurrence —C(O)—OR⁵. In certain embodiments, R³ represents independently for each occurrence —S(O)₂—R⁵. In certain embodiments, R³ represents independently for each occurrence —N(R⁴)—C(O)—R⁵. In certain embodiments, R³ represents independently for each occurrence —S(NR⁴)(O)—R⁵. In certain embodiments, R³ represents independently for each occurrence C₁₋₆ haloalkoxyl. In certain embodiments, R³ represents independently for each occurrence C₃₋₄ cycloalkyl substituted with r instances of R¹³. In certain embodiments, R³ represents independently for each occurrence a 3-6 membered heterocyclyl having 1-2 heteroatoms independently selected from nitrogen, oxygen, and sulfur substituted with r instances of R¹³. In certain embodiments, R³ is cyano. In certain embodiments, R³ is hydroxyl. In certain embodiments, R³ represents independently for each occurrence halo, cyano, C₁₋₆ alkyl, C₁₋₆ haloalkyl, hydroxyl, or C₁₋₆ alkoxyl. In certain embodiments, R³ represents independently for each occurrence halo. In certain embodiments, R³ represents independently for each occurrence C₁₋₆ alkyl, —CON(R⁴)₂, —C(O)—R⁵, or —C(O)—OR⁵. In certain embodiments, R³ represents independently for each occurrence C₁₋₆ alkyl, —S(O)₂—R⁵, —N(R⁴)—C(O)—R⁵, or C₁₋₆ haloalkoxyl. In certain embodiments, R³ represents independently for each occurrence halo or cyano.

In certain embodiments, R³ is selected from the groups depicted in the compounds in Table 1, below.

As defined generally above, R⁴ represents independently for each occurrence hydrogen, C₁₋₄ alkyl, or cyclopropyl. In certain embodiments, R⁴ is hydrogen. In certain embodiments, R⁴ represents independently for each occurrence C₁₋₄ alkyl. In certain embodiments, R⁴ is cyclopropyl. In certain embodiments, R⁴ is selected from the groups depicted in the compounds in Table 1, below.

As defined generally above, R⁵ and R⁶ each represent independently for each occurrence C₁₋₄ alkyl, C₃₋₅ cycloalkyl, —(C₀₋₂ alkylene)-phenyl, —(C₀₋₂ alkylene)-(3-6 membered monocyclic heterocyclyl having 1-2 heteroatoms independently selected from nitrogen, oxygen, and sulfur), or —(C₀₋₂ alkylene)-(5-10 membered heteroaryl having 1-3 heteroatoms independently selected from nitrogen, oxygen, and sulfur), wherein the cycloalkyl, phenyl, heterocyclyl, and heteroaryl are substituted with m instances of R⁸, and wherein each alkylene is substituted with n instances of R⁸. In certain embodiments, R⁵ and R⁶ each represent independently for each occurrence C₁₋₄ alkyl. In certain embodiments, R⁵ and R⁶ each represent independently for each occurrence C₃₋₅ cycloalkyl substituted with m instances of R⁸. In certain embodiments, R⁵ and R⁶ each represent independently for each occurrence C₃₋₅ cycloalkyl. In certain embodiments, R⁵ and R⁶ each represent independently for each occurrence —(C₀₋₂ alkylene)-phenyl, wherein the phenyl is substituted with m instances of R⁸, and the alkylene is substituted with n instances of R⁸. In certain embodiments, R⁵ and R⁶ each represent independently for each occurrence —(C₀₋₂ alkylene)-phenyl.

In certain embodiments, R⁵ and R⁶ each represent independently for each occurrence —(C₀₋₂ alkylene)-(5-10 membered heteroaryl having 1-3 heteroatoms independently selected from nitrogen, oxygen, and sulfur), wherein the heteroaryl is substituted with m instances of R⁸, and wherein the alkylene is substituted with n instances of R⁸. In certain embodiments, R⁵ and R⁶ each represent independently for each occurrence —(C₀₋₂ alkylene)-(5-10 membered heteroaryl having 1-3 heteroatoms independently selected from nitrogen, oxygen, and sulfur). In certain embodiments, R⁵ and R⁶ each represent independently for each occurrence —(C₀₋₂ alkylene)-(3-6 membered monocyclic heterocyclyl having 1-2 heteroatoms independently selected from nitrogen, oxygen, and sulfur), wherein the heterocyclyl is substituted with m instances of R⁸, and wherein the alkylene is substituted with n instances of R⁸. In certain embodiments, R⁵ and R⁶ each represent independently for each occurrence —(C₀₋₂ alkylene)-(3-6 membered monocyclic heterocyclyl having 1-2 heteroatoms independently selected from nitrogen, oxygen, and sulfur).

In certain embodiments, R⁵ and R⁶ each represent independently for each occurrence C₁₋₄ alkyl, C₃₋₅ cycloalkyl, phenyl, a 3-6 membered monocyclic heterocyclyl having 1-2 heteroatoms independently selected from nitrogen, oxygen, and sulfur, or a 5-10 membered heteroaryl having 1-3 heteroatoms independently selected from nitrogen, oxygen, and sulfur, wherein the cycloalkyl, phenyl, heterocyclyl, and heteroaryl are substituted with m instances of R⁸. In certain embodiments, R⁵ and R⁶ each represent independently for each occurrence C₁₋₄ alkyl or C₃₋₅ cycloalkyl. In certain embodiments, R⁵ and R⁶ each represent independently methyl. In certain embodiments, R⁵ and R⁶ are selected from the groups depicted in the compounds in Table 1, below.

As defined generally above, R⁷ is hydrogen, or two R⁷ groups attached to the same nitrogen atom are taken together with the nitrogen to which they are attached to form a 3-6 membered saturated monocyclic ring having in addition to said nitrogen atom 0, 1, or 2 heteroatoms independently selected from nitrogen, oxygen, and sulfur. In certain embodiments, R⁷ is hydrogen. In certain embodiments, two R⁷ groups attached to the same nitrogen atom are taken together with the nitrogen to which they are attached to form a 3-6 membered saturated monocyclic ring having in addition to said nitrogen atom 0, 1, or 2 heteroatoms independently selected from nitrogen, oxygen, and sulfur. In certain embodiments, R⁷ is selected from the groups depicted in the compounds in Table 1, below.

As defined generally above, R⁸ represents independently for each occurrence C₁₋₄ alkyl, halo, C₁₋₃ haloalkyl, hydroxyl, C₁₋₄ alkoxy, cyano, or cyclopropyl. In certain embodiments, R⁸ represents independently for each occurrence C₁₋₄ alkyl. In certain embodiments, R⁸ represents independently for each occurrence halo. In certain embodiments, R⁸ represents independently for each occurrence C₁₋₃ haloalkyl. In certain embodiments, R⁸ represents independently for each occurrence C₁₋₄ alkoxy. In certain embodiments, R⁸ is cyano. In certain embodiments, R⁸ is hydroxyl. In certain embodiments, R⁸ is cyclopropyl. In certain embodiments, R⁸ is selected from the groups depicted in the compounds in Table 1, below.

As defined generally above, R⁹ represents independently for each occurrence hydrogen, C₁₋₃ alkyl, or cyclopropyl. In certain embodiments, R⁹ is hydrogen. In certain embodiments, R⁹ represents independently for each occurrence C₁₋₃ alkyl. In certain embodiments, R⁹ is cyclopropyl. In certain embodiments, R⁹ is selected from the groups depicted in the compounds in Table 1, below.

As defined generally above, R¹⁰ is C₁₋₃ alkyl, C₁₋₃ haloalkyl, halo, hydroxyl, or cyclopropyl. In certain embodiments, R¹⁰ is C₁₋₃ alkyl. In certain embodiments, R¹⁰ is C₁₋₃ haloalkyl. In certain embodiments, R¹⁰ is halo. In certain embodiments, R¹⁰ hydroxyl. In certain embodiments, R¹⁰ is cyclopropyl. In certain embodiments, R¹⁰ is methyl. In certain embodiments, R¹⁰ is trifluoromethyl. In certain embodiments, R¹⁰ is fluoro. In certain embodiments, R¹⁰ is selected from the groups depicted in the compounds in Table 1, below.

As defined generally above, R¹¹ is C₁₋₄ alkyl, C₁₋₄ haloalkyl, cyclopropyl, or a 3-5 membered saturated heterocyclyl having 1-2 heteroatoms independently selected from nitrogen, oxygen, and sulfur. In certain embodiments, R¹¹ is C₁₋₄ alkyl. In certain embodiments, R¹¹ is C₁₋₄ haloalkyl. In certain embodiments, R¹¹ is cyclopropyl. In certain embodiments, R¹¹ is a 3-5 membered saturated heterocyclyl having 1-2 heteroatoms independently selected from nitrogen, oxygen, and sulfur. In certain embodiments, R¹¹ is methyl. In certain embodiments, R¹¹ is trifluoromethyl. In certain embodiments, R¹¹ is oxetanyl. In certain embodiments, R¹¹ is selected from the groups depicted in the compounds in Table 1, below.

As defined generally above, R¹² is C₁₋₃ alkyl, halo, cyano, or —SO₂—(C₁₋₃ alkyl). In certain embodiments, R¹² is halo. In certain embodiments, R¹² is C₁₋₃ alkyl. In certain embodiments, R¹² is cyano. In certain embodiments, R¹² is —SO₂—(C₁₋₃ alkyl). In certain embodiments, R¹² is selected from the groups depicted in the compounds in Table 1, below.

As defined generally above, R¹³ is halo, C₁₋₃ alkyl, or C₁₋₃ alkoxyl. In certain embodiments, R¹³ is halo. In certain embodiments, R¹³ is C₁₋₃ alkyl. In certain embodiments, R¹³ is C₁₋₃ alkoxyl. In certain embodiments, R¹³ is selected from the groups depicted in the compounds in Table 1, below.

As defined generally above, h is 0, 1, or 2. In certain embodiments, h is 0. In certain embodiments, h is 1. In certain embodiments, h is 2. In certain embodiments, h is selected from the corresponding value represented in the compounds in Table 1, below.

As defined generally above, j is 0, 1, or 2. In certain embodiments, j is 0. In certain embodiments, j is 1. In certain embodiments, j is 2. In certain embodiments, j is selected from the corresponding value represented in the compounds in Table 1, below.

As defined generally above, k is 1 or 2. In certain embodiments, k is 1. In certain embodiments, k is 2. In certain embodiments, k is selected from the corresponding value represented in the compounds in Table 1, below.

As defined generally above, m is 0, 1, or 2. In certain embodiments, m is 0. In certain embodiments, m is 1. In certain embodiments, m is 2. In certain embodiments, m is selected from the corresponding value represented in the compounds in Table 1 below.

As defined generally above, n is 0, 1, 2, or 3. In certain embodiments, n is 0. In certain embodiments, n is 1. In certain embodiments, n is 2. In certain embodiments, n is 3. In certain embodiments, n is selected from the corresponding value represented in the compounds in Table 1 below.

As defined generally above, p is 1, 2, 3, or 4. In certain embodiments, p is 1. In certain embodiments, p is 2. In certain embodiments, p is 3. In certain embodiments, p is 4. In certain embodiments, p is selected from the corresponding value represented in the compounds in Table 1 below.

As defined generally above, q is 0, 1, or 2. In certain embodiments, q is 0. In certain embodiments, q is 1. In certain embodiments, q is 2. In certain embodiments, q is selected from the corresponding value represented in the compounds in Table 1, below.

As defined generally above, r is 0, 1, or 2. In certain embodiments, r is 0. In certain embodiments, r is 1. In certain embodiments, r is 2. In certain embodiments, r is selected from the corresponding value represented in the compounds in Table 1, below.

In certain embodiments, the compound of Formula I is further defined by Formula I-A, or a pharmaceutically acceptable salt thereof:

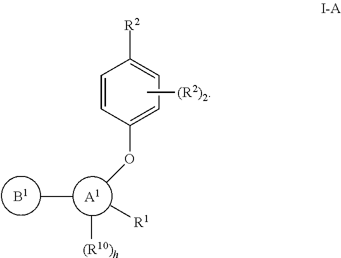

In certain embodiments, the compound of Formula I is further defined by Formula I-B, I-C, or I-D, or a pharmaceutically acceptable salt thereof:

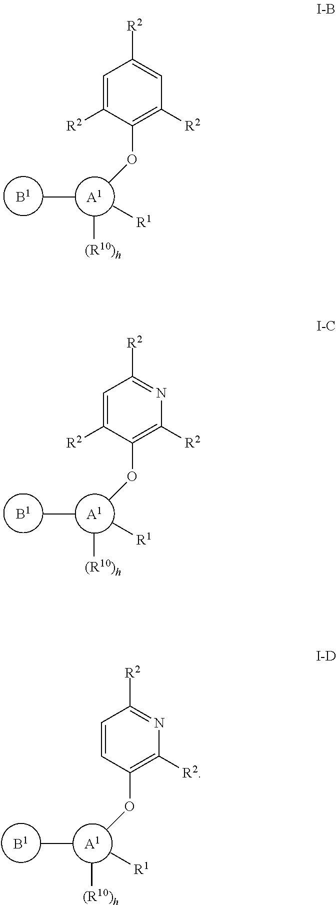

In certain embodiments, the compound of Formula I is further defined by Formula I-E, or a pharmaceutically acceptable salt thereof:

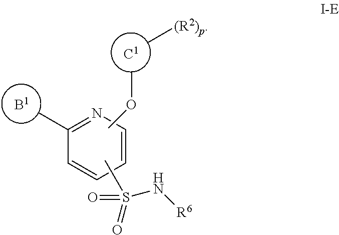

In certain embodiments, the compound of Formula I is further defined by Formula I-F, or a pharmaceutically acceptable salt thereof:

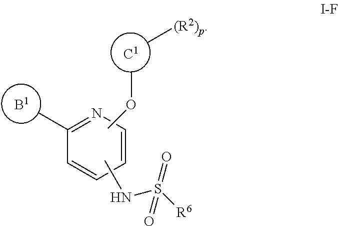

In certain embodiments, the compound of Formula I is further defined by Formula I-G, I-H, I-J, I-K, or I-L, or a pharmaceutically acceptable salt thereof:

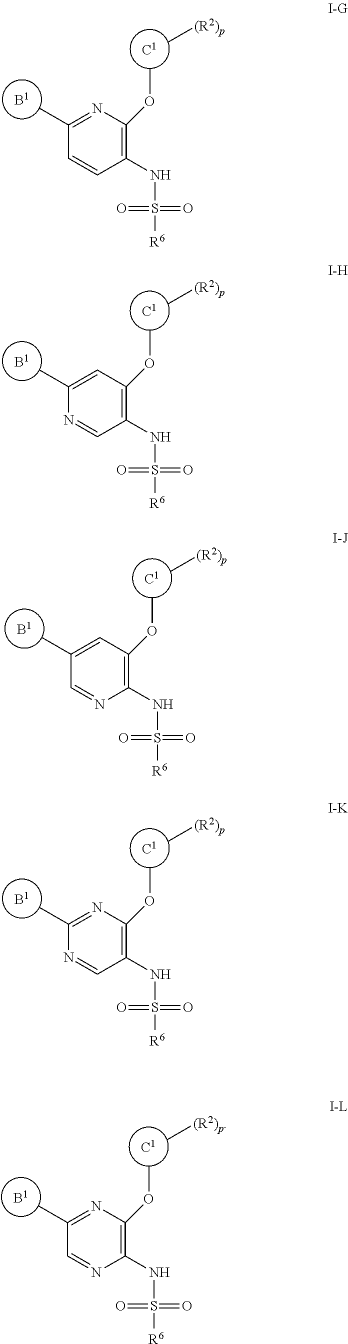

In certain embodiments, the compound of Formula I is further defined by Formula I-M, I-N, I-O, I-P, or I-Q, or a pharmaceutically acceptable salt thereof:

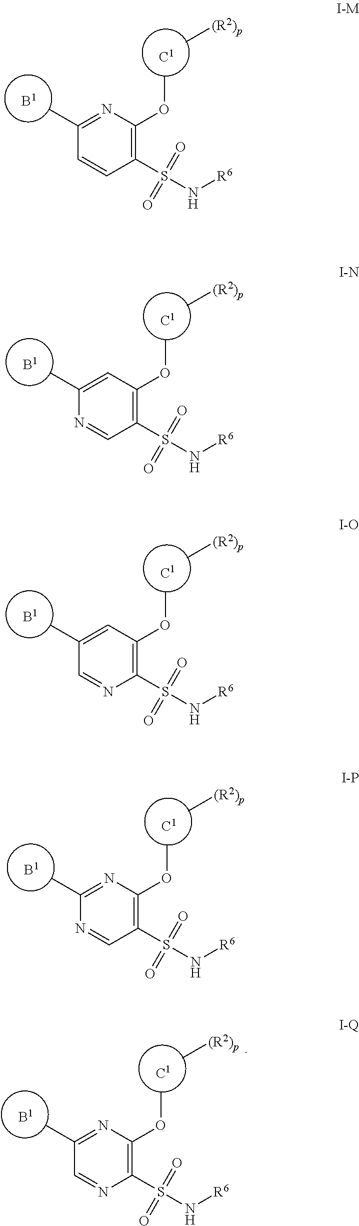

In certain embodiments, the compound of Formula I is further defined by Formula I-R, I-S, I-T, or I-U, or a pharmaceutically acceptable salt thereof:

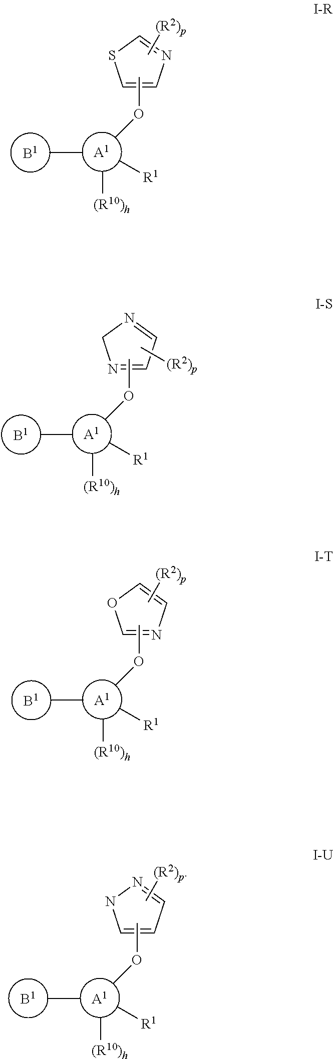

In certain embodiments, the compound of Formula I is further defined by Formula I-V, or a pharmaceutically acceptable salt thereof:

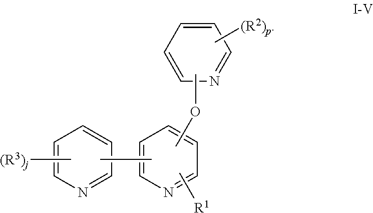

In certain embodiments, the compound of Formula I is further defined by Formula I-W, or a pharmaceutically acceptable salt thereof:

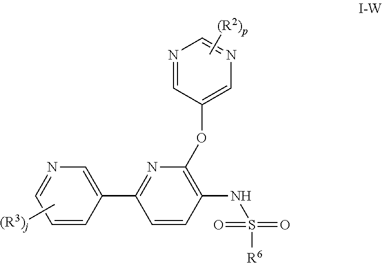

In certain embodiments, the compound of Formula I is further defined by Formula I-X, or a pharmaceutically acceptable salt thereof:

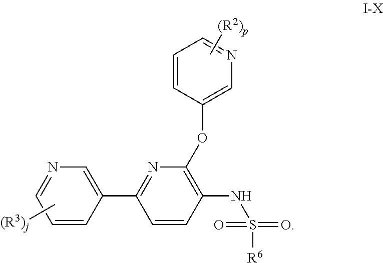

In certain embodiments, the compound of Formula I is further defined by Formula I-Y, or a pharmaceutically acceptable salt thereof:

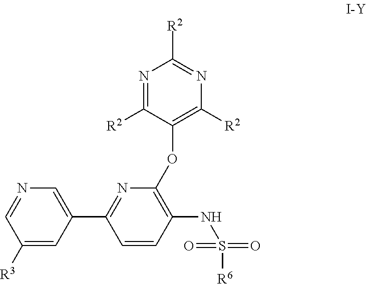

In certain embodiments, the compound of Formula I is further defined by Formula I-Z, or a pharmaceutically acceptable salt thereof:

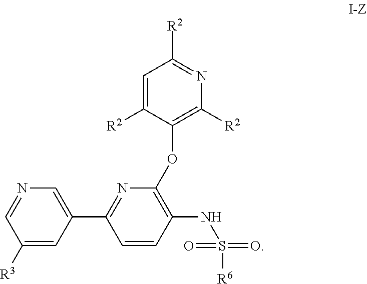

### I.2 Pyridinvloxypyridine Compounds of Formula (II)

One aspect of the invention provides pyridinyloxypyridine compounds. The compounds may be used in the pharmaceutical compositions and therapeutic methods described herein. Exemplary compounds are described in the following sections, along with exemplary procedures for making the compounds. One aspect of the invention provides a compound represented by Formula II:

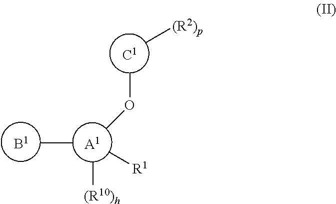

or a pharmaceutically acceptable salt thereof, wherein:
Ring A¹ is (i) a 6-membered heteroarylene having 1 or 2 heteroatoms selected from nitrogen or (ii) phenylene;
Ring B¹ is a 5-6 membered monocyclic heteroaryl having 1-3 heteroatoms independently selected from nitrogen, oxygen, and sulfur, a 3-7 membered monocyclic heterocyclyl having 1-2 heteroatoms independently selected from nitrogen, oxygen, and sulfur, a 3-7 membered monocyclic oxo-heterocyclyl having 1-2 heteroatoms independently selected from nitrogen, oxygen, and sulfur, a 7-12 membered bridged or spirocyclic heterocyclyl having 1-2 heteroatoms independently selected from nitrogen, oxygen, and sulfur, or a partially aromatic 9-10 membered bicyclic ring containing 2, 3, or 4 heteroatoms independently selected from nitrogen, oxygen, and sulfur, wherein each ring is substituted with j instances of R³; or Ring B¹ is phenyl substituted with k instances of R³;
Ring C¹ is phenylene or a 5 or 6-membered heteroarylene containing 1 or 2 heteroatoms selected from nitrogen, oxygen and sulfur;
R¹ is —N(R⁹)—SO₂—R⁶, —SO₂—N(R⁹)—R⁶, —SO₂—N(R⁷)₂, —S(NR⁹)(O)—R⁶, or —S(O₂)—R⁶.
R² represents independently for each occurrence hydroxyl, C₁₋₃ alkyl, halo, C₁₋₃ haloalkyl, C₁₋₃ hydroxyalkyl, cyano, cyclopropyl, —O—R¹¹, or a 3-5 membered heterocyclyl having 1-2 heteroatoms independently selected from nitrogen, oxygen, and sulfur; or two vicinal occurrences of R² are taken together with the atoms to which they are attached to form a 3-6 membered ring having 0, 1, or 2 heteroatoms independently selected from nitrogen, oxygen, and sulfur;
R³ represents independently for each occurrence halo, cyano, C₁₋₆ alkyl, C₁₋₆ haloalkyl, hydroxyl, C₁₋₆ alkoxyl, —CON(R⁴)₂, —C(O)—R⁵, —C(O)—OR⁵, —S(O)₂—R⁵, —N(R⁴)—C(O)—R⁵, —S(NR⁴)(O)—R⁵, or C₁₋₆ haloalkoxyl;
R⁴ represents independently for each occurrence hydrogen, C₁₋₄ alkyl, or cyclopropyl;
R⁵ and R⁶ each represent independently for each occurrence C₁₋₄ alkyl, C₃₋₅ cycloalkyl, —(C₀₋₂ alkylene)-phenyl, —(C₀₋₂ alkylene)-(3-5 membered monocyclic heterocyclyl having 1-2 heteroatoms independently selected from nitrogen, oxygen, and sulfur), or —(C₀₋₂ alkylene)-(5-10 membered heteroaryl having 1-3 heteroatoms independently selected from nitrogen, oxygen, and sulfur), wherein the cycloalkyl, phenyl, heterocyclyl, and heteroaryl are substituted with m instances of R⁸, and wherein each alkylene is substituted with n instances of R⁸;
R⁷ is hydrogen, or two R⁷ groups attached to the same nitrogen atom are taken together with the nitrogen to which they are attached to form a 3-6 membered saturated monocyclic ring having 0, 1, or 2 heteroatoms independently selected from nitrogen, oxygen, and sulfur;
R⁸ represents independently for each occurrence C₁₋₄ alkyl, halo, C₁₋₃ haloalkyl, hydroxyl, C₁₋₄ alkoxy, cyano, or cyclopropyl;
R⁹ is hydrogen, C₁₋₃ alkyl, or cyclopropyl;
R¹⁰ is C₁₋₃ alkyl, C₁₋₃ haloalkyl, halo, or cyclopropyl;
R¹¹ is C₁₋₄ alkyl, C₁₋₄ haloalkyl, cyclopropyl, or a 3-5 membered saturated heterocyclyl having 1-2 heteroatoms independently selected from nitrogen, oxygen, and sulfur;
h is 0, 1, or 2;
j is 0, 1, or 2;
k is 1 or 2;
m is 0, 1, or 2;
n is 0, 1, 2, or 3; and
p is 1, 2, 3, or 4.

The definitions of variables in Formula II above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula II.

As defined generally above, Ring A¹ is (i) a 6-membered heteroarylene having 1 or 2 heteroatoms selected from nitrogen or (ii) phenylene. In certain embodiments, Ring A¹ is phenylene. In certain embodiments, Ring A¹ is a 6-membered monocyclic heteroaromatic ring containing 1 or 2 heteroatoms selected from nitrogen. In certain embodiments, Ring A¹ is pyridinylene or pyrimidinylene. In certain embodiments, Ring A¹ is pyridinylene. In certain embodiments, Ring A¹ is pyrimidinylene. In certain embodiments, Ring A¹ is selected from the groups depicted in the compounds in Table 1, below.

As defined generally above, Ring B¹ is a 5-6 membered monocyclic heteroaryl having 1-3 heteroatoms independently selected from nitrogen, oxygen, and sulfur, a 3-7 membered monocyclic heterocyclyl having 1-2 heteroatoms independently selected from nitrogen, oxygen, and sulfur, a 3-7 membered monocyclic oxo-heterocyclyl having 1-2 heteroatoms independently selected from nitrogen, oxygen, and sulfur, a 7-12 membered bridged or spirocyclic heterocyclyl having 1-2 heteroatoms independently selected from nitrogen, oxygen, and sulfur, or a partially aromatic 9-10 membered bicyclic ring containing 2, 3, or 4 heteroatoms independently selected from nitrogen, oxygen, and sulfur, wherein each ring is substituted with j instances of R³; or Ring B¹ is phenyl substituted with k instances of R.

In certain embodiments, Ring B¹ is a 3-7 membered monocyclic heterocyclyl having 1-2 heteroatoms independently selected from nitrogen, oxygen, and sulfur, a 5-6 membered monocyclic heteroaryl having 1-3 heteroatoms independently selected from nitrogen, oxygen, and sulfur, or a 7-12 membered bridged heterocyclyl having 1-2 heteroatoms independently selected from nitrogen, oxygen, and sulfur, wherein each ring is substituted with j instances of R³; or Ring B¹ is phenylene substituted with k instances of R³.

In certain embodiments, Ring B¹ is a 3-7 membered monocyclic heterocyclyl having 1-2 heteroatoms independently selected from nitrogen, oxygen, and sulfur, a 5-6 membered monocyclic heteroaryl having 1-3 heteroatoms independently selected from nitrogen, oxygen, and sulfur, or a 7-12 membered bridged heterocyclyl having 1-2 heteroatoms independently selected from nitrogen, oxygen, and sulfur, wherein each ring is substituted with j instances of R³. In certain embodiments, Ring B¹ is a 3-7 membered monocyclic heterocyclyl having 1-2 heteroatoms independently selected from nitrogen, oxygen, and sulfur, a 5-6 membered monocyclic heteroaryl having 1-3 heteroatoms independently selected from nitrogen, oxygen, and sulfur, or a 7-12 membered bridged heterocyclyl having 1-2 heteroatoms independently selected from nitrogen, oxygen, and sulfur.

In certain embodiments, Ring B¹ is a 3-7 membered monocyclic heterocyclyl having 1-2 heteroatoms independently selected from nitrogen, oxygen, and sulfur, substituted with j instances of R³. In certain embodiments, Ring B¹ is a 5-6 membered monocyclic heteroaryl having 1-3 heteroatoms independently selected from nitrogen, oxygen, and sulfur, substituted with j instances of R³. In certain embodiments, Ring B¹ is a 7-12 membered bridged heterocyclyl having 1-2 heteroatoms independently selected from nitrogen, oxygen, and sulfur, substituted with j instances of R³.

In certain embodiments, Ring B¹ is a 3-7 membered monocyclic oxo-heterocyclyl having 1-2 heteroatoms independently selected from nitrogen, oxygen, and sulfur, substituted with j instances of R³.

In certain embodiments, Ring B¹ is a 3-7 membered monocyclic heterocyclyl having 1-2 heteroatoms independently selected from nitrogen, oxygen, and sulfur. In certain embodiments, Ring B¹ is a 5-6 membered monocyclic heteroaryl having 1-3 heteroatoms independently selected from nitrogen, oxygen, and sulfur. In certain embodiments, Ring B¹ is a 7-12 membered bridged heterocyclyl having 1-2 heteroatoms independently selected from nitrogen, oxygen, and sulfur.

In certain embodiments, Ring B¹ is a 7-12 membered bridged or spirocyclic heterocyclyl having 1-2 heteroatoms independently selected from nitrogen, oxygen, and sulfur, substituted with j instances of R³.

In certain embodiments, Ring B¹ is a partially aromatic 9-10 membered bicyclic ring containing 2, 3, or 4 heteroatoms independently selected from nitrogen, oxygen, and sulfur, substituted with j instances of R³.

In certain embodiments, Ring B¹ is phenyl substituted with k instances of R³. In certain embodiments, Ring B¹ is phenylene.

In certain embodiments, Ring B¹ is pyridinyl, pyrazolyl, imidazolyl, piperazinyl, 2,5,-diazabicyclo[2.2.1]heptanyl, 3,6-diazabicyclo[3.1.1]heptanyl, 5,6-dihydro-8H-7-[1,2,4]triazolo[3,4-c]pyrazinyl, 2,6-diazaspiro[3.4]octanyl, 2,6-diazaspiro[3.3]heptanyl, or azetidinyl, wherein each ring is substituted with j instances of R³. In certain embodiments, Ring B¹ is pyridinyl, substituted with j instances of R³. In certain embodiments, Ring B¹ is pyrazolyl, substituted with j instances of R³. In certain embodiments, Ring B¹ is imidazolyl, substituted with j instances of R³. In certain embodiments, Ring B¹ is piperazinyl, substituted with j instances of R³. In certain embodiments, Ring B¹ is 2,5,-diazabicyclo[2.2.1]heptanyl, substituted with j instances of R³. In certain embodiments, Ring B¹ is 3,6-diazabicyclo[3.1.1]heptanyl, substituted with j instances of R³. In certain embodiments, Ring B¹ is 5,6-dihydro-8H-7-[1,2,4]triazolo[3,4-c]pyrazinyl, substituted with j instances of R³. In certain embodiments, Ring B¹ is 2,6-diazaspiro[3.4]octanyl, substituted with j instances of R³. In certain embodiments, Ring B¹ is 2,6-diazaspiro[3.3]heptanyl, substituted with j instances of R³. In certain embodiments, Ring B¹ is azetidinyl, substituted with j instances of R³.

In certain embodiments, Ring B¹ is selected from the groups depicted in the compounds in Table 1, below.

As defined generally above, Ring C¹ is phenylene or a 5 or 6-membered heteroarylene containing 1 or 2 heteroatoms selected from nitrogen, oxygen, and sulfur. In certain embodiments, Ring C¹ is phenylene. In certain embodiments, Ring C¹ is a 5 or 6-membered heteroarylene containing 1 or 2 heteroatoms selected from nitrogen, oxygen, and sulfur. In certain embodiments, Ring C¹ is a 5-membered heteroarylene containing 1 or 2 heteroatoms selected from nitrogen, oxygen, and sulfur. In certain embodiments, Ring C¹ is a 6-membered heteroarylene containing 1 or 2 heteroatoms selected from nitrogen, oxygen and sulfur. In certain embodiments, Ring C¹ is a 6-membered heteroarylene containing 1 or 2 heteroatoms selected from nitrogen. In certain embodiments, Ring C¹ is phenylene or pyridinylene. In certain embodiments, Ring C¹ is pyridinylene. In certain embodiments, Ring C¹ is pyridinylene. In certain embodiments, Ring C¹ is selected from the groups depicted in the compounds in Table 1, below.

As defined generally above, R¹ is —N(R⁹)—SO₂—R⁶, —SO₂—N(R⁹)—R⁶, —SO₂—N(R⁷)₂, —S(NR⁹)(O)—R⁶, or —S(O₂)—R⁶. In certain embodiments, R¹ is —N(R⁹)—SO₂—R⁶, —SO₂—N(R⁹)—R⁶, —S(NR⁹)(O)—R⁶, or —S(O₂)—R⁶. In certain embodiments, R¹ is —N(R⁹)—SO₂—R⁶. In certain embodiments, R¹ is —SO₂—N(R⁹)—R⁶. In certain embodiments, R¹ is —SO₂—N(R⁷)₂. In certain embodiments, R¹ is —S(NR⁹)(O)—R⁶. In certain embodiments, R¹ is —S(O₂)—R⁶. In certain embodiments, R¹ is —NH—SO₂—CH₃. In certain embodiments, R¹ is selected from the groups depicted in the compounds in Table 1, below.

As defined generally above, R² represents independently for each occurrence hydroxyl, C₁₋₃ alkyl, halo, C₁₋₃ haloalkyl, C₁₋₃ hydroxyalkyl, cyano, cyclopropyl, —O—R¹¹, or a 3-5 membered heterocyclyl having 1-2 heteroatoms independently selected from nitrogen, oxygen, and sulfur; or two vicinal occurrences of R² are taken together with the atoms to which they are attached to form a 3-6 membered ring having 0, 1, or 2 heteroatoms independently selected from nitrogen, oxygen, and sulfur. In certain embodiments, R² represents independently for each occurrence C₁₋₃ alkyl, halo, C₁₋₄ alkoxyl, C₁₋₃ haloalkyl, C₁₋₃ hydroxyalkyl, or cyano.

In certain embodiments, R² represents independently for each occurrence C₁₋₃ alkyl. In certain embodiments, R² represents independently for each occurrence halo. In certain embodiments, R² represents independently for each occurrence C₁₋₃ alkoxyl. In certain embodiments, R² represents independently for each occurrence C₁₋₃ haloalkyl. In certain embodiments, R² represents independently for each occurrence C₁₋₃ hydroxyalkyl. In certain embodiments, R² represents independently for each occurrence a 3-5 membered heterocyclyl having 1-2 heteroatoms independently selected from nitrogen, oxygen, and sulfur. In certain embodiments, R² is cyclopropyl. In certain embodiments, R² represents independently for each occurrence —O—R¹¹. In certain embodiments, R² is cyano. In certain embodiments, R² is methyl. In certain embodiments, R² is hydroxyl. In certain embodiments, R² represents independently for each occurrence methyl or cyclopropyl. In certain embodiments, or two vicinal occurrences of R² are taken together with the atoms to which they are attached to form a 3-6 membered ring having 0, 1, or 2 heteroatoms independently selected from nitrogen, oxygen, and sulfur. In certain embodiments, R² is selected from the groups depicted in the compounds in Table 1, below.

As defined generally above, R³ represents independently for each occurrence halo, cyano, C₁₋₆ alkyl, C₁₋₆ haloalkyl, hydroxyl, C₁₋₆ alkoxyl, —CON(R⁴)₂, —C(O)—R⁵, —C(O)—OR⁵, —S(O)₂—R⁵, —N(R⁴)—C(O)—R⁵, —S(NR⁴)(O)—R⁵, or C₁₋₆ haloalkoxyl. In certain embodiments, R³ represents independently for each occurrence halo. In certain embodiments, R³ represents independently for each occurrence C₁₋₆ alkyl. In certain embodiments, R³ represents independently for each occurrence C₁₋₆ haloalkyl. In certain embodiments, R³ represents independently for each occurrence C₁₋₆ alkoxyl. In certain embodiments, R³ represents independently for each occurrence —CON(R⁴)₂. In certain embodiments, R³ represents independently for each occurrence —C(O)—R⁵. In certain embodiments, R³ represents independently for each occurrence —C(O)—OR⁵. In certain embodiments, R³ represents independently for each occurrence —S(O)₂—R⁵. In certain embodiments, R³ represents independently for each occurrence —N(R⁴)—C(O)—R⁵. In certain embodiments, R³ represents independently for each occurrence —S(NR⁴)(O)—R⁵. In certain embodiments, R³ represents independently for each occurrence C₁₋₆ haloalkoxyl. In certain embodiments, R³ is cyano. In certain embodiments, R³ is hydroxyl. In certain embodiments, R³ represents independently for each occurrence halo, cyano, C₁₋₆ alkyl, C₁₋₆ haloalkyl, hydroxyl, or C₁₋₆ alkoxyl. In certain embodiments, R³ represents independently for each occurrence halo. In certain embodiments, R³ represents independently for each occurrence C₁₋₆ alkyl, —CON(R⁴)₂, —C(O)—R⁵, or —C(O)—OR⁵. In certain embodiments, R³ represents independently for each occurrence C₁₋₆ alkyl, —S(O)₂—R⁵, —N(R⁴)—C(O)—R⁵, or C₁₋₆ haloalkoxyl.

In certain embodiments, R³ is selected from the groups depicted in the compounds in Table 1, below.

As defined generally above, R⁴ represents independently for each occurrence hydrogen, C₁₋₄ alkyl, or cyclopropyl. In certain embodiments, R⁴ is hydrogen. In certain embodiments, R⁴ represents independently for each occurrence C₁₋₄ alkyl. In certain embodiments, R⁴ represents independently for each occurrence cyclopropyl. In certain embodiments, R⁴ is selected from the groups depicted in the compounds in Table 1, below.

As defined generally above, R⁵ and R⁶ each represent independently for each occurrence C₁₋₄ alkyl, C₃₋₅ cycloalkyl, —(C₀₋₂ alkylene)-phenyl, —(C₀₋₂ alkylene)-(3-5 membered monocyclic heterocyclyl having 1-2 heteroatoms independently selected from nitrogen, oxygen, and sulfur), or —(C₀₋₂ alkylene)-(5-10 membered heteroaryl having 1-3 heteroatoms independently selected from nitrogen, oxygen, and sulfur), wherein the cycloalkyl, phenyl, heterocyclyl, and heteroaryl are substituted with m instances of R⁸, and wherein each alkylene is substituted with n instances of R⁸. In certain embodiments, R⁵ and R⁶ each represent independently for each occurrence C₁₋₄ alkyl. In certain embodiments, R⁵ and R⁶ each represent independently for each occurrence C₃₋₅ cycloalkyl substituted with m instances of R⁸. In certain embodiments, R⁵ and R⁶ each represent independently for each occurrence C₃₋₅ cycloalkyl. In certain embodiments, R⁵ and R⁶ each represent independently for each occurrence —(C₀₋₂ alkylene)-phenyl, wherein the phenyl is substituted with m instances of R⁸, and the alkylene is substituted with n instances of R⁸. In certain embodiments, R⁵ and R⁶ each represent independently for each occurrence —(C₀₋₂ alkylene)-phenyl.

In certain embodiments, R⁵ and R⁶ each represent independently for each occurrence —(C₀₋₂ alkylene)-(5-10 membered heteroaryl having 1-3 heteroatoms independently selected from nitrogen, oxygen, and sulfur), wherein the heteroaryl is substituted with m instances of R⁸, and wherein the alkylene is substituted with n instances of R⁸. In certain embodiments, R⁵ and R⁶ each represent independently for each occurrence —(C₀₋₂ alkylene)-(5-10 membered heteroaryl having 1-3 heteroatoms independently selected from nitrogen, oxygen, and sulfur). In certain embodiments, R⁵ and R⁶ each represent independently for each occurrence —(C₀₋₂ alkylene)-(3-5 membered monocyclic heterocyclyl having 1-2 heteroatoms independently selected from nitrogen, oxygen, and sulfur), wherein the heterocyclyl is substituted with m instances of R⁸, and wherein the alkylene is substituted with n instances of R⁸. In certain embodiments, R⁵ and R⁶ each represent independently for each occurrence —(C₀₋₂ alkylene)-(3-5 membered monocyclic heterocyclyl having 1-2 heteroatoms independently selected from nitrogen, oxygen, and sulfur).

In certain embodiments, R⁵ and R⁶ each represent independently for each occurrence C₁₋₄ alkyl, C₃₋₅ cycloalkyl, phenyl, a 3-5 membered monocyclic heterocyclyl having 1-2 heteroatoms independently selected from nitrogen, oxygen, and sulfur, or a 5-10 membered heteroaryl having 1-3 heteroatoms independently selected from nitrogen, oxygen, and sulfur, wherein the cycloalkyl, phenyl, heterocyclyl, and heteroaryl are substituted with m instances of R⁸. In certain embodiments, R⁵ and R⁶ each represent independently for each occurrence C₁₋₄ alkyl or C₃₋₅ cycloalkyl. In certain embodiments, R⁵ and R⁶ each represent independently methyl. In certain embodiments, R⁵ and R⁶ are selected from the groups depicted in the compounds in Table 1, below.

As defined generally above, R⁷ is hydrogen, or two R⁷ groups attached to the same nitrogen atom are taken together with the nitrogen to which they are attached to form a 3-6 membered saturated monocyclic ring having 0, 1, or 2 heteroatoms independently selected from nitrogen, oxygen, and sulfur. In certain embodiments, R⁷ is hydrogen. In certain embodiments, two R⁷ groups attached to the same nitrogen atom are taken together with the nitrogen to which they are attached to form a 3-6 membered saturated monocyclic ring having 0, 1, or 2 heteroatoms independently selected from nitrogen, oxygen, and sulfur. In certain embodiments, R⁷ is selected from the groups depicted in the compounds in Table 1, below.

As defined generally above, R⁸ represents independently for each occurrence C₁₋₄ alkyl, halo, C₁₋₃ haloalkyl, hydroxyl, C₁₋₄ alkoxy, cyano, or cyclopropyl. In certain embodiments, R⁸ represents independently for each occurrence C₁₋₄ alkyl. In certain embodiments, R⁸ represents independently for each occurrence halo. In certain embodiments, R⁸ represents independently for each occurrence C₁₋₃ haloalkyl. In certain embodiments, R⁸ represents independently for each occurrence C₁₋₄ alkoxy. In certain embodiments, R⁸ is cyano. In certain embodiments, R⁸ is hydroxyl. In certain embodiments, R⁸ is cyclopropyl. In certain embodiments, R⁸ is selected from the groups depicted in the compounds in Table 1, below.

As defined generally above, R⁹ is hydrogen, C₁₋₃ alkyl, or cyclopropyl. In certain embodiments, R⁹ is hydrogen. In certain embodiments, R⁹ is C₁₋₃ alkyl. In certain embodiments, R⁹ is cyclopropyl. In certain embodiments, R⁹ is selected from the groups depicted in the compounds in Table 1, below.

As defined generally above, R¹⁰ is C₁₋₃ alkyl, C₁₋₃ haloalkyl, halo, or cyclopropyl. In certain embodiments, R¹⁰ is C₁₋₃ alkyl. In certain embodiments, R¹⁰ is C₁₋₃ haloalkyl. In certain embodiments, R¹⁰ is halo. In certain embodiments, R¹⁰ is cyclopropyl. In certain embodiments, R¹⁰ is methyl. In certain embodiments, R¹⁰ is trifluoromethyl. In certain embodiments, R¹⁰ is fluoro. In certain embodiments, R¹⁰ is selected from the groups depicted in the compounds in Table 1, below.

As defined generally above, R¹¹ is C₁₋₄ alkyl, C₁₋₄ haloalkyl, cyclopropyl, or a 3-5 membered saturated heterocyclyl having 1-2 heteroatoms independently selected from nitrogen, oxygen, and sulfur. In certain embodiments, R¹¹ is C₁₋₄ alkyl. In certain embodiments, R¹¹ is C₁₋₄ haloalkyl. In certain embodiments, R¹¹ is cyclopropyl. In certain embodiments, R¹¹ is a 3-5 membered saturated heterocyclyl having 1-2 heteroatoms independently selected from nitrogen, oxygen, and sulfur. In certain embodiments, R¹¹ is methyl. In certain embodiments, R¹¹ is trifluoromethyl. In certain embodiments, R¹¹ is oxetanyl. In certain embodiments, R¹¹ is selected from the groups depicted in the compounds in Table 1, below.

As defined generally above, h is 0, 1, or 2. In certain embodiments, h is 0. In certain embodiments, h is 1. In certain embodiments, h is 2. In certain embodiments, h is selected from the corresponding value represented in the compounds in Table 1, below.

As defined generally above, j is 0, 1, or 2. In certain embodiments, j is 0. In certain embodiments, j is 1. In certain embodiments, j is 2. In certain embodiments, j is selected from the corresponding value represented in the compounds in Table 1, below.

As defined generally above, k is 1 or 2. In certain embodiments, k is 1. In certain embodiments, k is 2. In certain embodiments, k is selected from the corresponding value represented in the compounds in Table 1, below.

As defined generally above, m is 0, 1, or 2. In certain embodiments, m is 0. In certain embodiments, m is 1. In certain embodiments, m is 2. In certain embodiments, m is selected from the corresponding value represented in the compounds in Table 1 below.

As defined generally above, n is 0, 1, 2, or 3. In certain embodiments, n is 0. In certain embodiments, n is 1. In certain embodiments, n is 2. In certain embodiments, n is 3. In certain embodiments, n is selected from the corresponding value represented in the compounds in Table 1 below.

As defined generally above, p is 1, 2, 3, or 4. In certain embodiments, p is 1. In certain embodiments, p is 2. In certain embodiments, p is 3. In certain embodiments, p is 4. In certain embodiments, p is selected from the corresponding value represented in the compounds in Table 1 below.

In certain embodiments, the compound of Formula II is further defined by Formula II-A, or a pharmaceutically acceptable salt thereof:

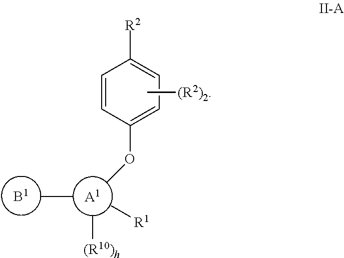

In certain embodiments, the compound of Formula II is further defined by Formula II-B, II-C, or II-D, or a pharmaceutically acceptable salt thereof:

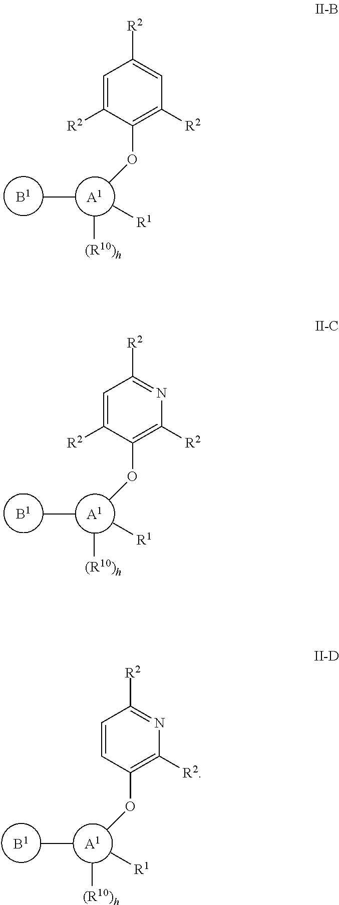

In certain embodiments, the compound of Formula II is further defined by Formula II-E, or a pharmaceutically acceptable salt thereof:

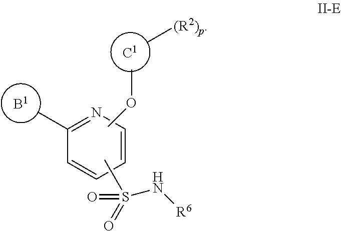

In certain embodiments, the compound of Formula II is further defined by Formula II-F, or a pharmaceutically acceptable salt thereof:

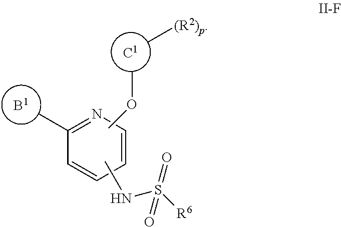

In certain embodiments, the compound of Formula II is further defined by Formula II-G, II-H, II-J, II-K, or II-L, or a pharmaceutically acceptable salt thereof:

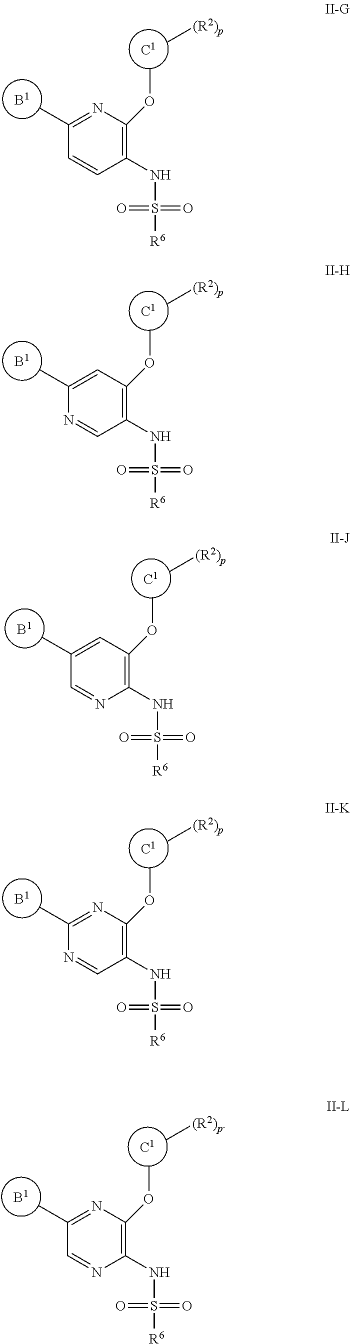

In certain embodiments, the compound of Formula II is further defined by Formula II-M, II-N, II-O, II-P, or II-Q, or a pharmaceutically acceptable salt thereof:

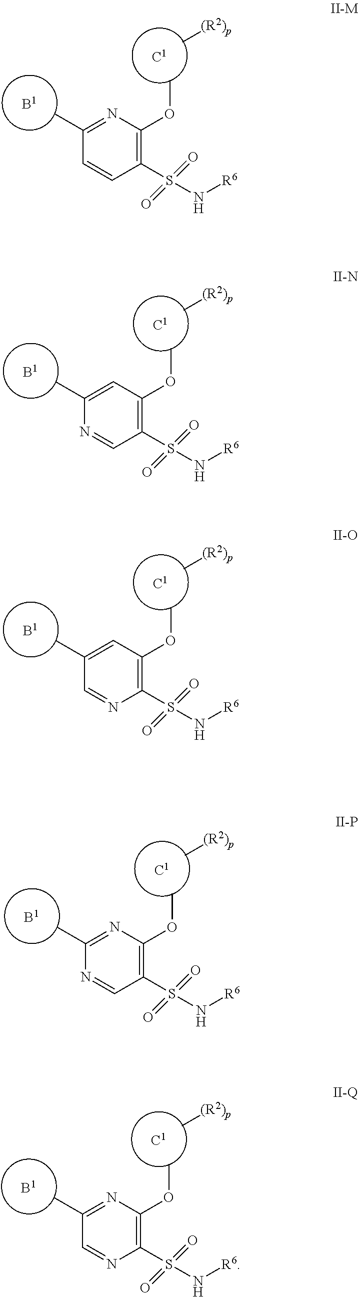

In certain embodiments, the compound of Formula II is further defined by Formula II-R, II-S, II-T, or II-U, or a pharmaceutically acceptable salt thereof:

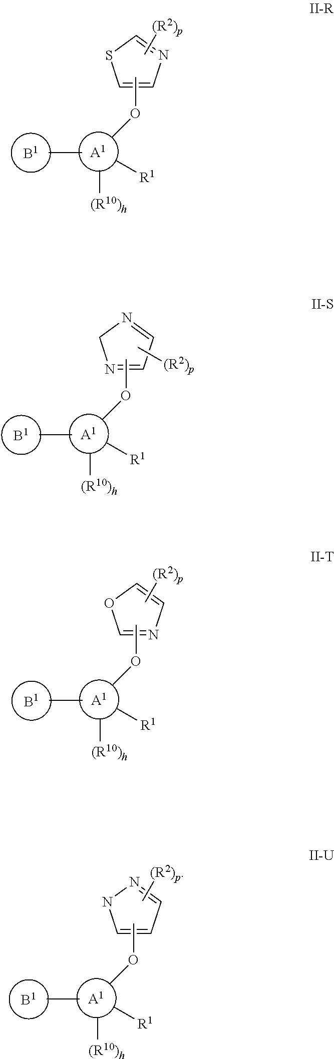

In certain embodiments, the compound of Formula II is further defined by Formula II-V, or a pharmaceutically acceptable salt thereof:

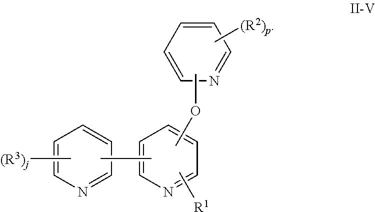

### Exemplary Specific Compounds

In certain embodiments, the compound is a compound in Table 1, or a pharmaceutically acceptable salt thereof. In certain embodiments, the compound is a compound in Table 1.

In certain embodiments, the compound is selected from Compounds I-1 to I-132 in Table 1, or a pharmaceutically acceptable salt thereof. In certain embodiments, the compound is selected from Compounds I-1 to I-132 in Table 1.

TABLE 1
Exemplary Compounds

| Com- | |
|---|---|
| pound | |
| No. | Structure |
| I-1 | 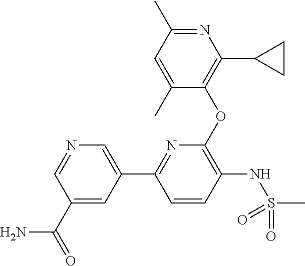 [SMILES: Cc1cc(C)c(Oc2nc(-c3cncc(C(N)=O)c3)ccc2NS(C)(=O)=O)c(C2CC2)n1] |
| I-2 | 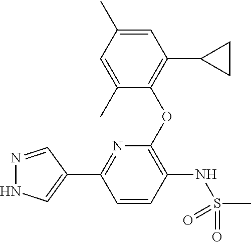 [SMILES: Cc1cc(C)c(Oc2nc(-c3cn[nH]c3)ccc2NS(C)(=O)=O)c(C2CC2)c1] |
| I-3 | 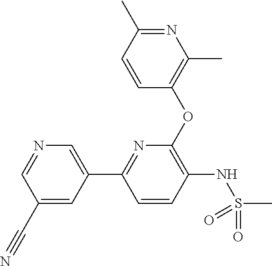 [SMILES: Cc1ccc(Oc2nc(-c3cncc(C#N)c3)ccc2NS(C)(=O)=O)c(C)n1] |
| I-4 | 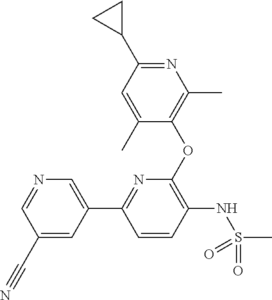 [SMILES: Cc1cc(C2CC2)nc(C)c1Oc1nc(-c2cncc(C#N)c2)ccc1NS(C)(=O)=O] |
| I-5 | 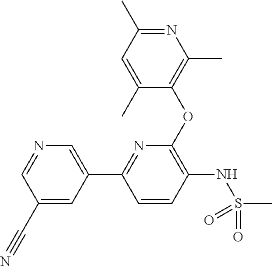 [SMILES: Cc1cc(C)c(Oc2nc(-c3cncc(C#N)c3)ccc2NS(C)(=O)=O)c(C)n1] |
| I-6 | 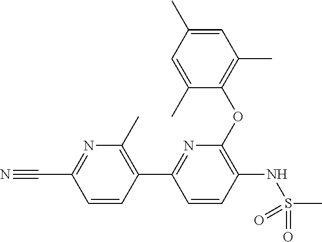 [SMILES: Cc1cc(C)c(Oc2nc(-c3ccc(C#N)nc3C)ccc2NS(C)(=O)=O)c(C)c1] |
| I-7 | 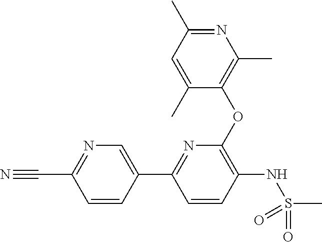 [SMILES: Cc1cc(C)c(Oc2nc(-c3ccc(C#N)nc3)ccc2NS(C)(=O)=O)c(C)n1] |
| I-8 | 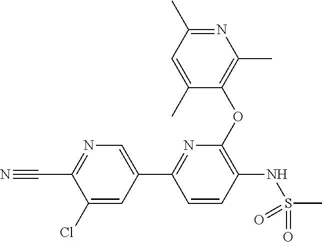 [SMILES: Cc1cc(C)c(Oc2nc(-c3cnc(C#N)c(Cl)c3)ccc2NS(C)(=O)=O)c(C)n1] |
| I-9 | 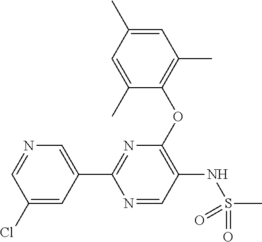 [SMILES: Cc1cc(C)c(Oc2nc(-c3cncc(Cl)c3)ncc2NS(C)(=O)=O)c(C)c1] |
| I-10 | 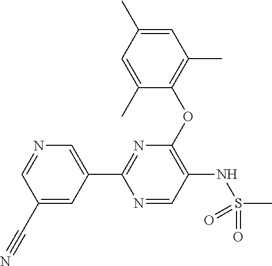 [SMILES: Cc1cc(C)c(Oc2nc(-c3cncc(C#N)c3)ncc2NS(C)(=O)=O)c(C)c1] |
| I-11 | 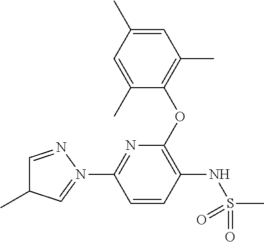 |
| I-12 | 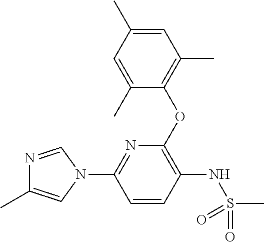 |
| I-13 | 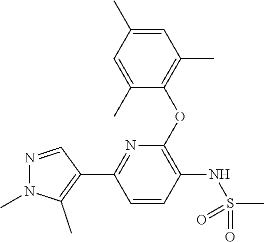 |
| I-14 | 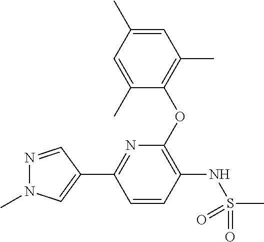 |
| I-15 | 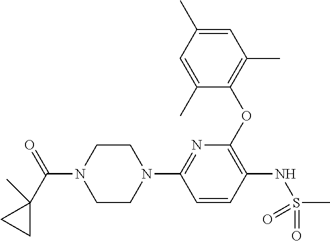 [SMILES: Cc1cc(C)c(Oc2nc(N3CCN(C(=O)C4(C)CC4)CC3)ccc2NS(C)(=O)=O)c(C)c1] |
| I-16 | 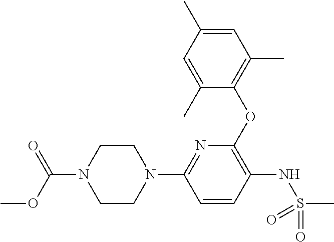 |
| I-17 | 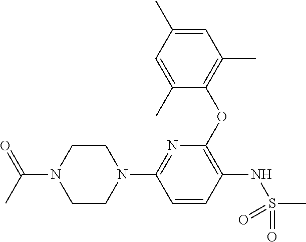 [SMILES: CC(=O)N1CCN(c2ccc(NS(C)(=O)=O)c(Oc3c(C)cc(C)cc3C)n2)CC1] |
| I-18 | 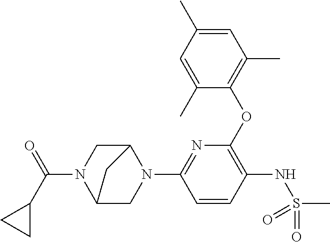 [SMILES: Cc1cc(C)c(Oc2nc(N3CC4CC3CN4C(=O)C3CC3)ccc2NS(C)(=O)=O)c(C)c1] |
| | Stereoisomer 1 |
| I-19 | 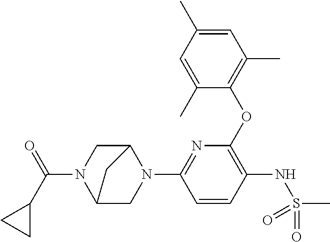 [SMILES: Cc1cc(C)c(Oc2nc(N3CC4CC3CN4C(=O)C3CC3)ccc2NS(C)(=O)=O)c(C)c1] |
| | Stereoisomer 2 |
| I-20 | 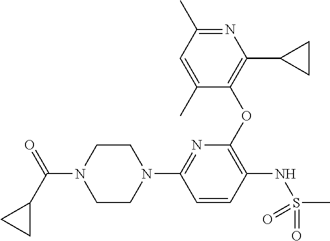 [SMILES: Cc1cc(C)c(Oc2nc(N3CCN(C(=O)C4CC4)CC3)ccc2NS(C)(=O)=O)c(C2CC2)n1] |
| I-21 | 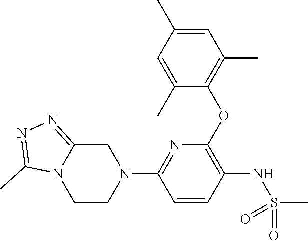 |
| I-22 | 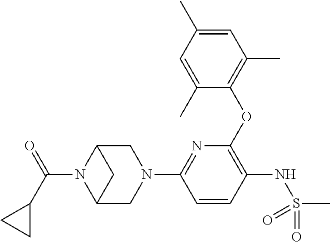 [SMILES: Cc1cc(C)c(Oc2nc(N3CC4CC(C3)N4C(=O)C3CC3)ccc2NS(C)(=O)=O)c(C)c1] |
| I-23 | 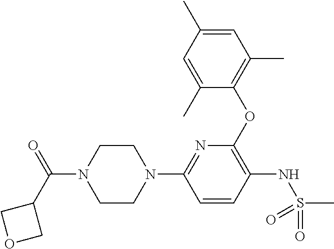 [SMILES: Cc1cc(C)c(Oc2nc(N3CCN(C(=O)C4COC4)CC3)ccc2NS(C)(=O)=O)c(C)c1] |
| I-24 | 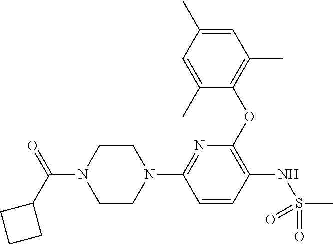 [SMILES: Cc1cc(C)c(Oc2nc(N3CCN(C(=O)C4CCC4)CC3)ccc2NS(C)(=O)=O)c(C)c1] |
| I-25 | 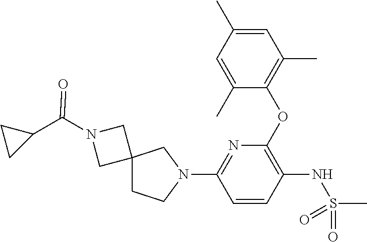 [SMILES: Cc1cc(C)c(Oc2nc(N3CCC4(CN(C(=O)C5CC5)C4)C3)ccc2NS(C)(=O)=O)c(C)c1] |
| I-26 | 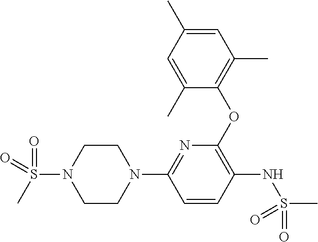 [SMILES: Cc1cc(C)c(Oc2nc(N3CCN(S(C)(=O)=O)CC3)ccc2NS(C)(=O)=O)c(C)c1] |
| I-27 | 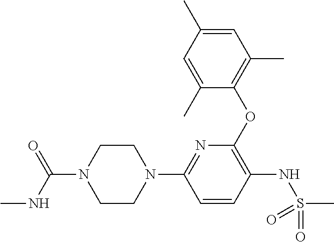 |
| I-28 | 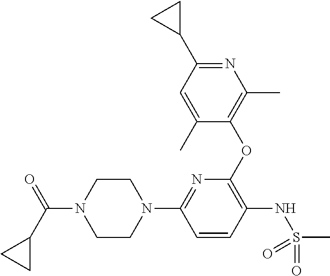 [SMILES: Cc1cc(C2CC2)nc(C)c1Oc1nc(N2CCN(C(=O)C3CC3)CC2)ccc1NS(C)(=O)=O] |
| I-29 | 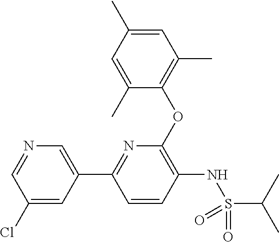 [SMILES: Cc1cc(C)c(Oc2nc(-c3cncc(Cl)c3)ccc2NS(=O)(=O)C(C)C)c(C)c1] |
| I-30 | 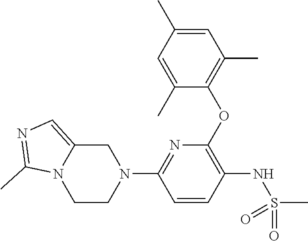 |
| I-31 | 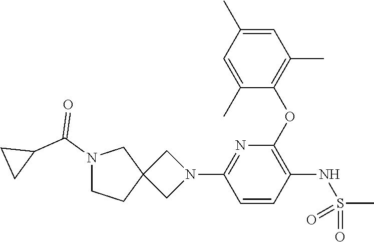 [SMILES: Cc1cc(C)c(Oc2nc(N3CC4(CCN(C(=O)C5CC5)C4)C3)ccc2NS(C)(=O)=O)c(C)c1] |
| I-32 | 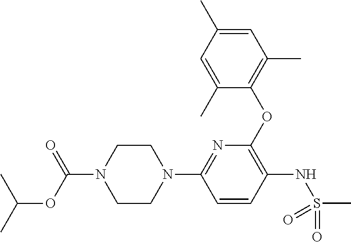 [SMILES: Cc1cc(C)c(Oc2nc(N3CCN(C(=O)OC(C)C)CC3)ccc2NS(C)(=O)=O)c(C)c1] |
| I-33 | 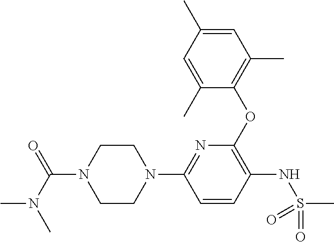 |
| I-34 | 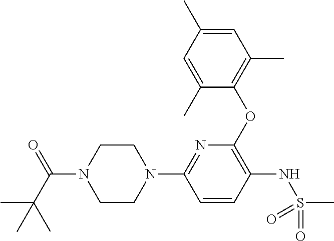 [SMILES: Cc1cc(C)c(Oc2nc(N3CCN(C(=O)C(C)(C)C)CC3)ccc2NS(C)(=O)=O)c(C)c1] |
| I-35 | 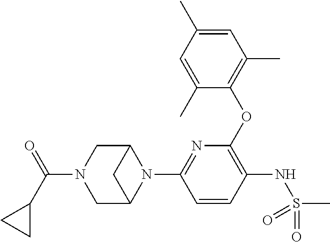 [SMILES: Cc1cc(C)c(Oc2nc(N3C4CC3CN(C(=O)C3CC3)C4)ccc2NS(C)(=O)=O)c(C)c1] |
| I-36 | 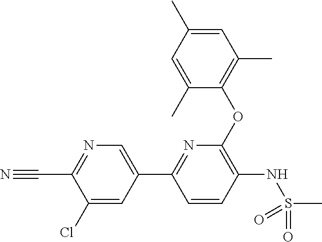 [SMILES: Cc1cc(C)c(Oc2nc(-c3cnc(C#N)c(Cl)c3)ccc2NS(C)(=O)=O)c(C)c1] |
| I-37 | 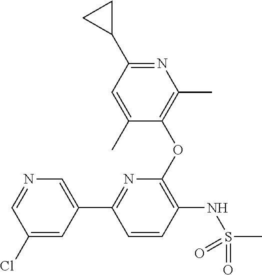 [SMILES: Cc1cc(C2CC2)nc(C)c1Oc1nc(-c2cncc(Cl)c2)ccc1NS(C)(=O)=O] |
| I-38 | 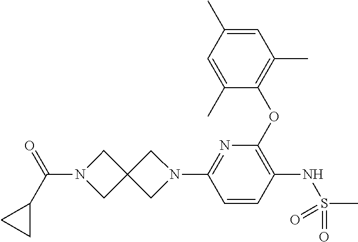 [SMILES: Cc1cc(C)c(Oc2nc(N3CC4(CN(C(=O)C5CC5)C4)C3)ccc2NS(C)(=O)=O)c(C)c1] |
| I-39 | 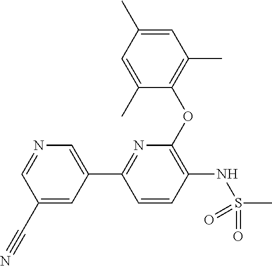 [SMILES: Cc1cc(C)c(Oc2nc(-c3cncc(C#N)c3)ccc2NS(C)(=O)=O)c(C)c1] |
| I-40 | 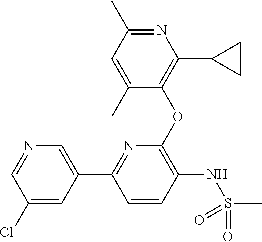 [SMILES: Cc1cc(C)c(Oc2nc(-c3cncc(Cl)c3)ccc2NS(C)(=O)=O)c(C2CC2)n1] |
| I-41 | 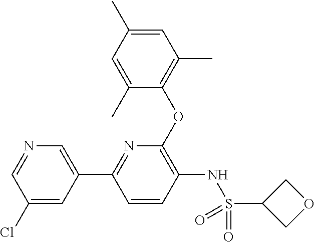 [SMILES: Cc1cc(C)c(Oc2nc(-c3cncc(Cl)c3)ccc2NS(=O)(=O)C2COC2)c(C)c1] |
| I-42 | 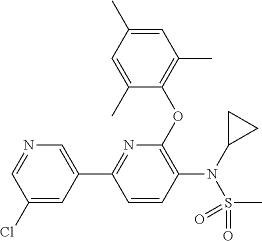 [SMILES: Cc1cc(C)c(Oc2nc(-c3cncc(Cl)c3)ccc2N(C2CC2)S(C)(=O)=O)c(C)c1] |
| I-43 | 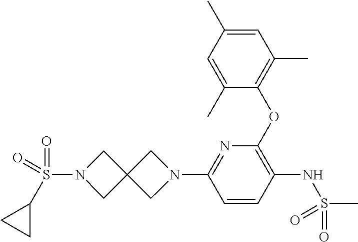 [SMILES: Cc1cc(C)c(Oc2nc(N3CC4(C3)CN(S(=O)(=O)C3CC3)C4)ccc2NS(C)(=O)=O)c(C)c1] |
| I-44 | 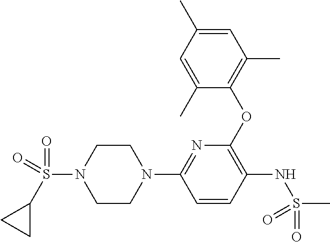 [SMILES: Cc1cc(C)c(Oc2nc(N3CCN(S(=O)(=O)C4CC4)CC3)ccc2NS(C)(=O)=O)c(C)c1] |
| I-45 | 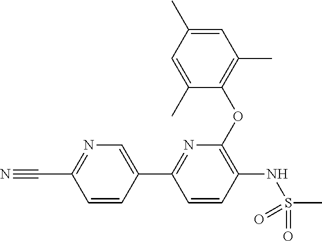 [SMILES: Cc1cc(C)c(Oc2nc(-c3ccc(C#N)nc3)ccc2NS(C)(=O)=O)c(C)c1] |
| I-46 | 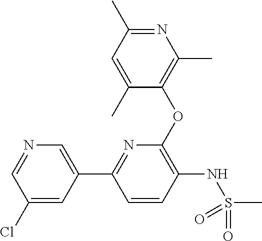 [SMILES: Cc1cc(C)c(Oc2nc(-c3cncc(Cl)c3)ccc2NS(C)(=O)=O)c(C)n1] |
| I-47 | 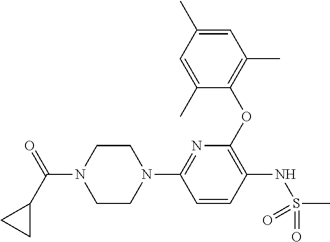 [SMILES: Cc1cc(C)c(Oc2nc(N3CCN(C(=O)C4CC4)CC3)ccc2NS(C)(=O)=O)c(C)c1] |
| I-48 | 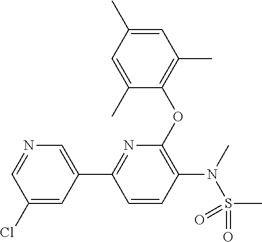 [SMILES: Cc1cc(C)c(Oc2nc(-c3cncc(Cl)c3)ccc2N(C)S(C)(=O)=O)c(C)c1] |
| I-49 | 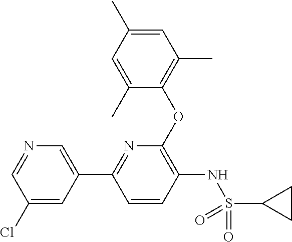 [SMILES: Cc1cc(C)c(Oc2nc(-c3cncc(Cl)c3)ccc2NS(=O)(=O)C2CC2)c(C)c1] |
| I-50 | 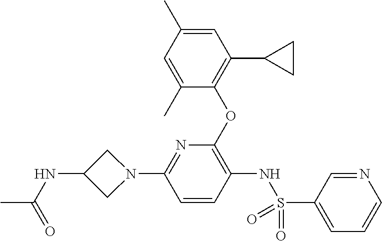 [SMILES: CC(=O)NC1CN(c2ccc(NS(=O)(=O)c3cccnc3)c(Oc3c(C)cc(C)cc3C3CC3)n2)C1] |
| I-51 | 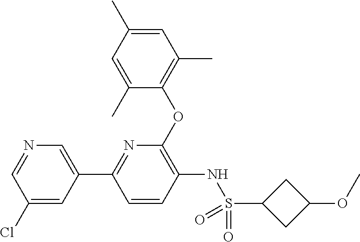 [SMILES: COC1CC(S(=O)(=O)Nc2ccc(-c3cncc(Cl)c3)nc2Oc2c(C)cc(C)cc2C)C1] |
| I-52 | 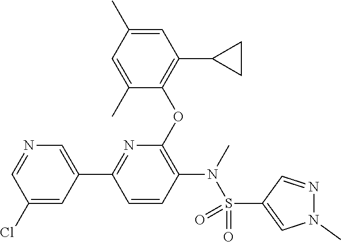 [SMILES: Cc1cc(C)c(Oc2nc(-c3cncc(Cl)c3)ccc2N(C)S(=O)(=O)c2cnn(C)c2)c(C2CC2)c1] |
| I-53 | 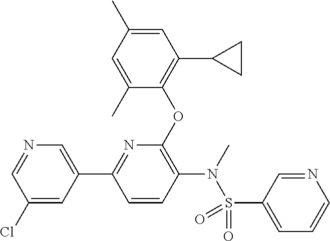 [SMILES: Cc1cc(C)c(Oc2nc(-c3cncc(Cl)c3)ccc2N(C)S(=O)(=O)c2cccnc2)c(C2CC2)c1] |
| I-54 | 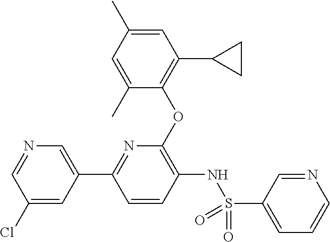 [SMILES: Cc1cc(C)c(Oc2nc(-c3cncc(Cl)c3)ccc2NS(=O)(=O)c2cccnc2)c(C2CC2)c1] |
| I-55 | 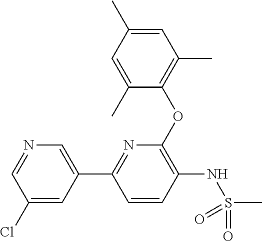 [SMILES: Cc1cc(C)c(Oc2nc(-c3cncc(Cl)c3)ccc2NS(C)(=O)=O)c(C)c1] |
| I-56 | 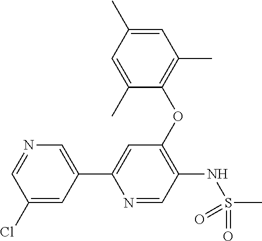 [SMILES: Cc1cc(C)c(Oc2cc(-c3cncc(Cl)c3)ncc2NS(C)(=O)=O)c(C)c1] |
| I-57 | 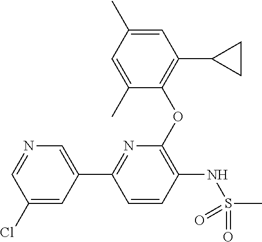 [SMILES: Cc1cc(C)c(Oc2nc(-c3cncc(Cl)c3)ccc2NS(C)(=O)=O)c(C2CC2)c1] |
| I-58 | 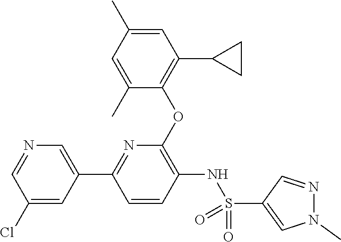 [SMILES: Cc1cc(C)c(Oc2nc(-c3cncc(Cl)c3)ccc2NS(=O)(=O)c2cnn(C)c2)c(C2CC2)c1] |
| I-59 | 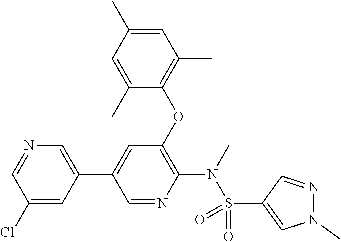 [SMILES: Cc1cc(C)c(Oc2cc(-c3cncc(Cl)c3)cnc2N(C)S(=O)(=O)c2cnn(C)c2)c(C)c1] |
| I-60 | 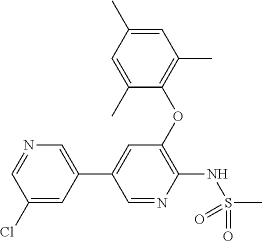 [SMILES: Cc1cc(C)c(Oc2cc(-c3cncc(Cl)c3)cnc2NS(C)(=O)=O)c(C)c1] |
| I-61 | 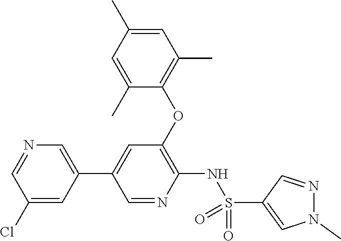 [SMILES: Cc1cc(C)c(Oc2cc(-c3cncc(Cl)c3)cnc2NS(=O)(=O)c2cnn(C)c2)c(C)c1] |
| I-62 | 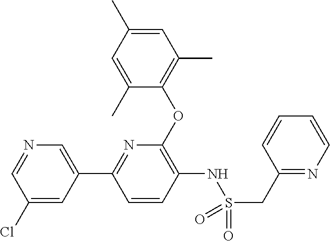 [SMILES: Cc1cc(C)c(Oc2nc(-c3cncc(Cl)c3)ccc2NS(=O)(=O)Cc2ccccn2)c(C)c1] |
| I-63 | 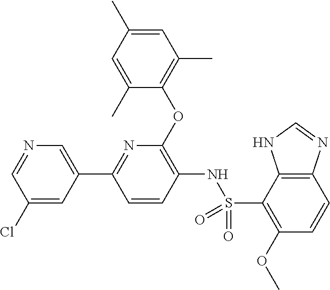 [SMILES: COc1ccc2nc[nH]c2c1S(=O)(=O)Nc1ccc(-c2cncc(Cl)c2)nc1Oc1c(C)cc(C)cc1C] |
| I-64 | 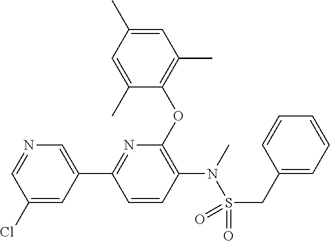 [SMILES: Cc1cc(C)c(Oc2nc(-c3cncc(Cl)c3)ccc2N(C)S(=O)(=O)Cc2ccccc2)c(C)c1] |
| I-65 | 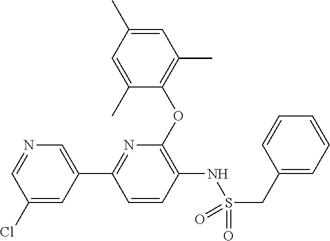 [SMILES: Cc1cc(C)c(Oc2nc(-c3cncc(Cl)c3)ccc2NS(=O)(=O)Cc2ccccc2)c(C)c1] |
| I-66 | 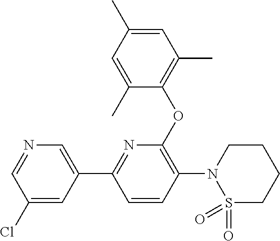 [SMILES: Cc1cc(C)c(Oc2nc(-c3cncc(Cl)c3)ccc2N2CCCCS2(=O)=O)c(C)c1] |
| I-67 | 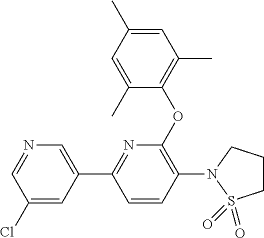 [SMILES: Cc1cc(C)c(Oc2nc(-c3cncc(Cl)c3)ccc2N2CCCS2(=O)=O)c(C)c1] |
| I-68 | 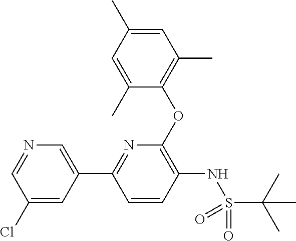 [SMILES: Cc1cc(C)c(Oc2nc(-c3cncc(Cl)c3)ccc2NS(=O)(=O)C(C)(C)C)c(C)c1] |
| I-69 | 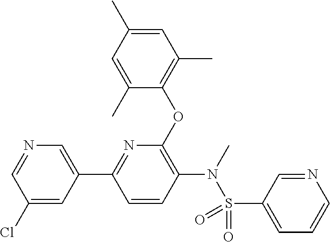 [SMILES: Cc1cc(C)c(Oc2nc(-c3cncc(Cl)c3)ccc2N(C)S(=O)(=O)c2cccnc2)c(C)c1] |
| I-70 | 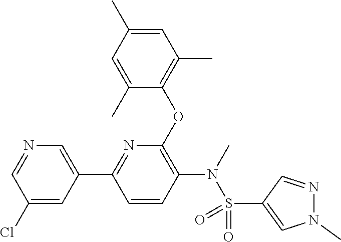 [SMILES: Cc1cc(C)c(Oc2nc(-c3cncc(Cl)c3)ccc2N(C)S(=O)(=O)c2cnn(C)c2)c(C)c1] |
| I-71 | 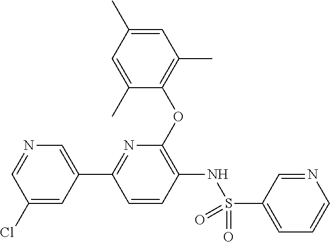 [SMILES: Cc1cc(C)c(Oc2nc(-c3cncc(Cl)c3)ccc2NS(=O)(=O)c2cccnc2)c(C)c1] |
| I-72 | 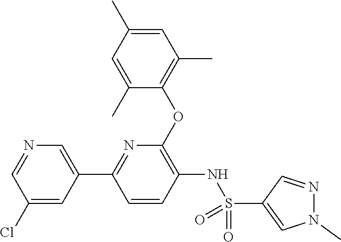 [SMILES: Cc1cc(C)c(Oc2nc(-c3cncc(Cl)c3)ccc2NS(=O)(=O)c2cnn(C)c2)c(C)c1] |
| I-73 | 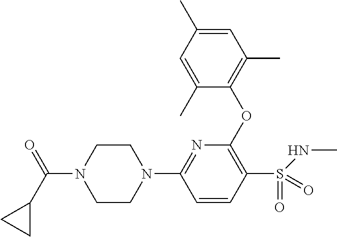 [SMILES: CNS(=O)(=O)c1ccc(N2CCN(C(=O)C3CC3)CC2)nc1Oc1c(C)cc(C)cc1C] |
| I-74 | 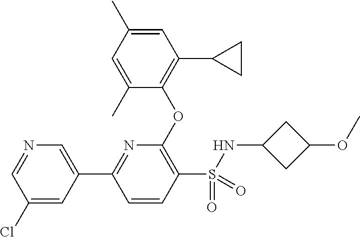 [SMILES: COC1CC(NS(=O)(=O)c2ccc(-c3cncc(Cl)c3)nc2Oc2c(C)cc(C)cc2C2CC2)C1] |
| I-75 | 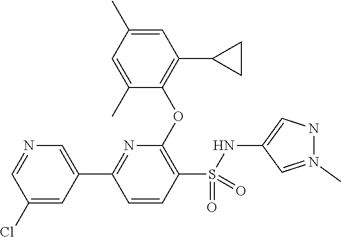 |
| I-76 | 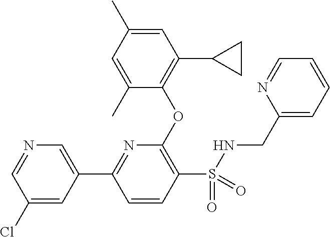 [SMILES: Cc1cc(C)c(Oc2nc(-c3cncc(Cl)c3)ccc2S(=O)(=O)NCc2ccccn2)c(C2CC2)c1] |
| I-77 | 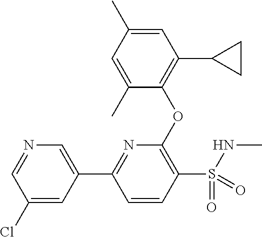 [SMILES: CNS(=O)(=O)c1ccc(-c2cncc(Cl)c2)nc1Oc1c(C)cc(C)cc1C1CC1] |
| I-78 | 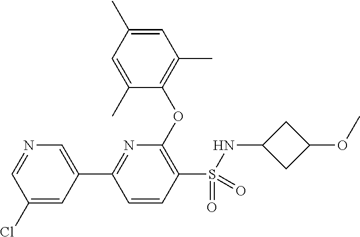 [SMILES: COC1CC(NS(=O)(=O)c2ccc(-c3cncc(Cl)c3)nc2Oc2c(C)cc(C)cc2C)C1] |
| I-79 | 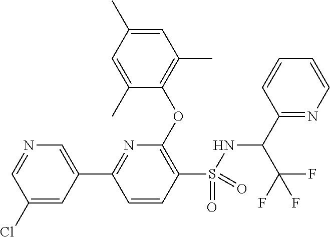 [SMILES: Cc1cc(C)c(Oc2nc(-c3cncc(Cl)c3)ccc2S(=O)(=O)NC(c2ccccn2)C(F)(F)F)c(C)c1] |
| | Stereoisomer 1 |
| I-80 | 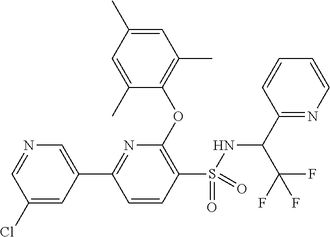 [SMILES: Cc1cc(C)c(Oc2nc(-c3cncc(Cl)c3)ccc2S(=O)(=O)NC(c2ccccn2)C(F)(F)F)c(C)c1] |
| | Stereoisomer 2 |
| I-81 | 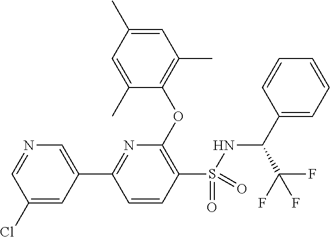 [SMILES: Cc1cc(C)c(Oc2nc(-c3cncc(Cl)c3)ccc2S(=O)(=O)N[C@H](c2ccccc2)C(F)(F)F)c(C)c1] |
| I-82 | 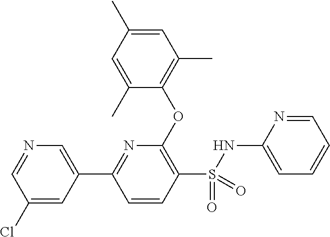 [SMILES: Cc1cc(C)c(Oc2nc(-c3cncc(Cl)c3)ccc2S(=O)(=O)Nc2ccccn2)c(C)c1] |
| I-83 | 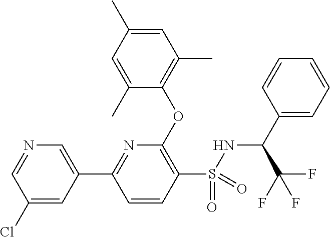 [SMILES: Cc1cc(C)c(Oc2nc(-c3cncc(Cl)c3)ccc2S(=O)(=O)N[C@@H](c2ccccc2)C(F)(F)F)c(C)c1] |
| I-84 | 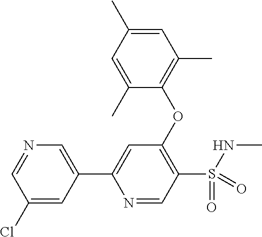 [SMILES: CNS(=O)(=O)c1cnc(-c2cncc(Cl)c2)cc1Oc1c(C)cc(C)cc1C] |
| I-85 | 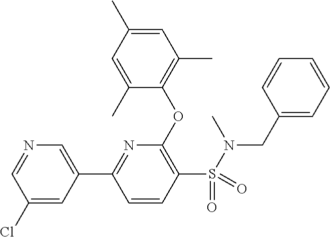 [SMILES: Cc1cc(C)c(Oc2nc(-c3cncc(Cl)c3)ccc2S(=O)(=O)N(C)Cc2ccccc2)c(C)c1] |
| I-86 | 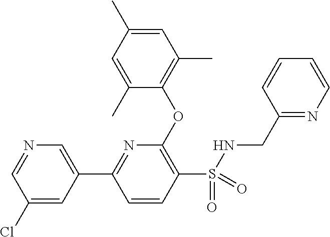 [SMILES: Cc1cc(C)c(Oc2nc(-c3cncc(Cl)c3)ccc2S(=O)(=O)NCc2ccccn2)c(C)c1] |
| I-87 | 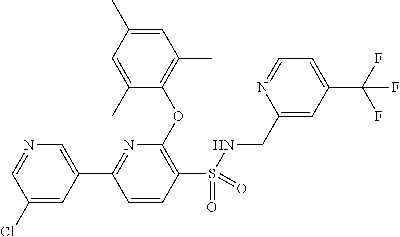 [SMILES: Cc1cc(C)c(Oc2nc(-c3cncc(Cl)c3)ccc2S(=O)(=O)NCc2cc(C(F)(F)F)ccn2)c(C)c1] |
| I-88 | 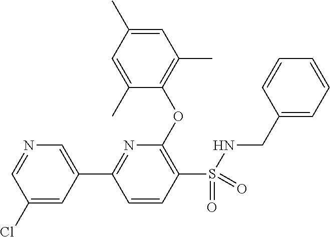 [SMILES: Cc1cc(C)c(Oc2nc(-c3cncc(Cl)c3)ccc2S(=O)(=O)NCc2ccccc2)c(C)c1] |
| I-89 | 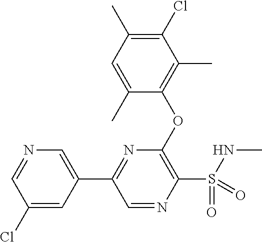 [SMILES: CNS(=O)(=O)c1ncc(-c2cncc(Cl)c2)nc1Oc1c(C)cc(C)c(Cl)c1C] |
| I-90 | 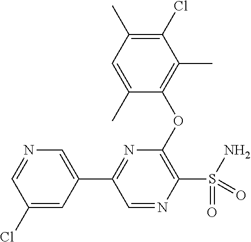 [SMILES: Cc1cc(C)c(Oc2nc(-c3cncc(Cl)c3)cnc2S(N)(=O)=O)c(C)c1Cl] |
| I-92 | 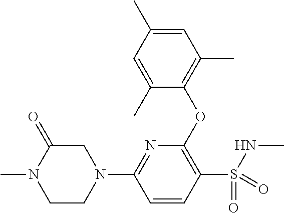 |
| I-93 | 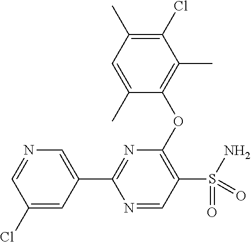 [SMILES: Cc1cc(C)c(Oc2nc(-c3cncc(Cl)c3)ncc2S(N)(=O)=O)c(C)c1Cl] |
| I-94 | 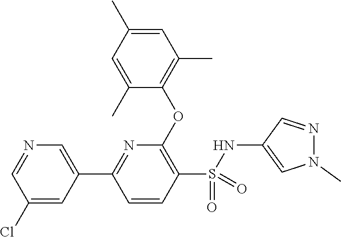 [SMILES: Cc1cc(C)c(Oc2nc(-c3cncc(Cl)c3)ccc2S(=O)(=O)Nc2cnn(C)c2)c(C)c1] |
| I-95 | 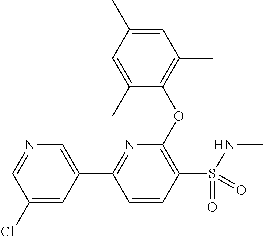 [SMILES: CNS(=O)(=O)c1ccc(-c2cncc(Cl)c2)nc1Oc1c(C)cc(C)cc1C] |
| I-96 | 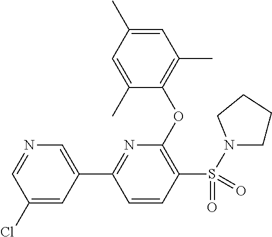 [SMILES: Cc1cc(C)c(Oc2nc(-c3cncc(Cl)c3)ccc2S(=O)(=O)N2CCCC2)c(C)c1] |
| I-97 | 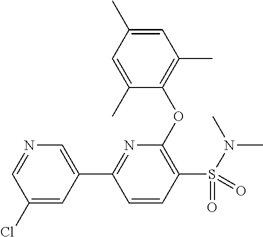 [SMILES: Cc1cc(C)c(Oc2nc(-c3cncc(Cl)c3)ccc2S(=O)(=O)N(C)C)c(C)c1] |
| I-98 | 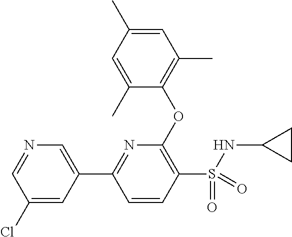 [SMILES: Cc1cc(C)c(Oc2nc(-c3cncc(Cl)c3)ccc2S(=O)(=O)NC2CC2)c(C)c1] |
| I-99 | 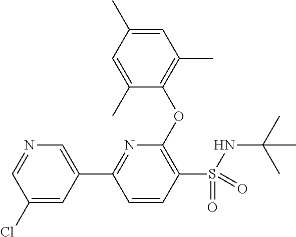 [SMILES: Cc1cc(C)c(Oc2nc(-c3cncc(Cl)c3)ccc2S(=O)(=O)NC(C)(C)C)c(C)c1] |
| I-100 | 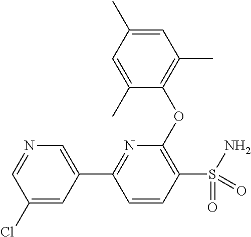 [SMILES: Cc1cc(C)c(Oc2nc(-c3cncc(Cl)c3)ccc2S(N)(=O)=O)c(C)c1] |
| I-101 | 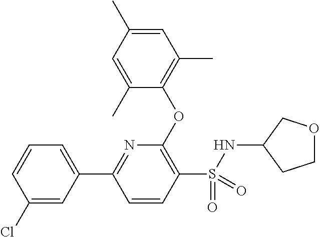 [SMILES: Cc1cc(C)c(Oc2nc(-c3cccc(Cl)c3)ccc2S(=O)(=O)NC2CCOC2)c(C)c1] |
| I-102 | 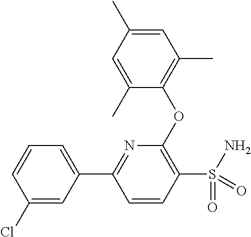 [SMILES: Cc1cc(C)c(Oc2nc(-c3cccc(Cl)c3)ccc2S(N)(=O)=O)c(C)c1] |
| I-103 | 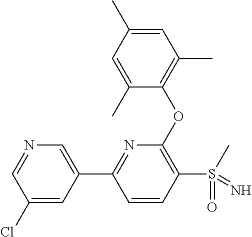 [SMILES: Cc1cc(C)c(Oc2nc(-c3cncc(Cl)c3)ccc2S(C)(=N)=O)c(C)c1] |
| I-104 | 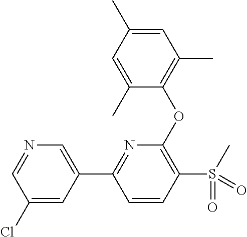 [SMILES: Cc1cc(C)c(Oc2nc(-c3cncc(Cl)c3)ccc2S(C)(=O)=O)c(C)c1] |
| I-105 | 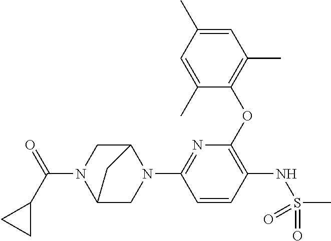 [SMILES: Cc1cc(C)c(Oc2nc(N3CC4CC3CN4C(=O)C3CC3)ccc2NS(C)(=O)=O)c(C)c1] |
| I-106 | 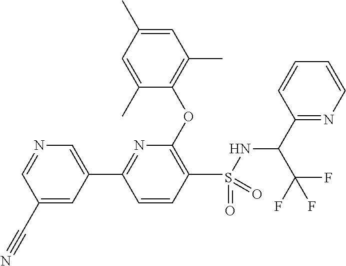 |
| I-107 | 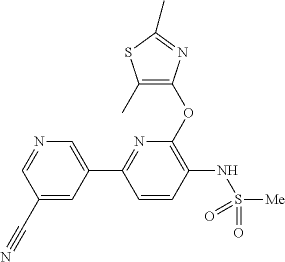 [SMILES: Cc1nc(Oc2nc(-c3cncc(C#N)c3)ccc2NS(C)(=O)=O)c(C)s1] |
| I-108 | 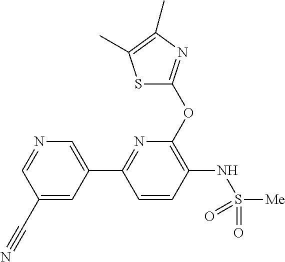 [SMILES: Cc1nc(Oc2nc(-c3cncc(C#N)c3)ccc2NS(C)(=O)=O)sc1C] |
| I-109 | 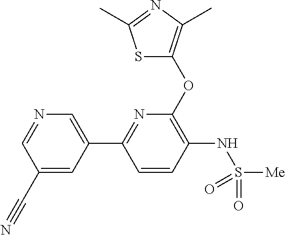 [SMILES: Cc1nc(C)c(Oc2nc(-c3cncc(C#N)c3)ccc2NS(C)(=O)=O)s1] |
| I-110 | 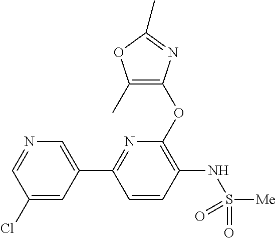 [SMILES: Cc1nc(Oc2nc(-c3cncc(Cl)c3)ccc2NS(C)(=O)=O)c(C)o1] |
| I-111 | 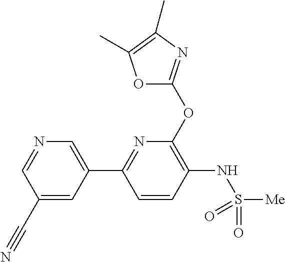 [SMILES: Cc1nc(Oc2nc(-c3cncc(C#N)c3)ccc2NS(C)(=O)=O)oc1C] |
| I-112 | 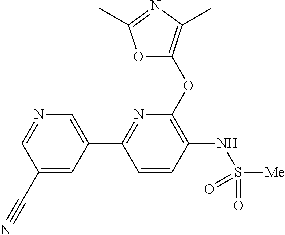 [SMILES: Cc1nc(C)c(Oc2nc(-c3cncc(C#N)c3)ccc2NS(C)(=O)=O)o1] |
| I-113 | 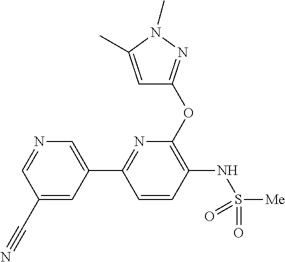 [SMILES: Cc1cc(Oc2nc(-c3cncc(C#N)c3)ccc2NS(C)(=O)=O)nn1C] |
| I-114 | 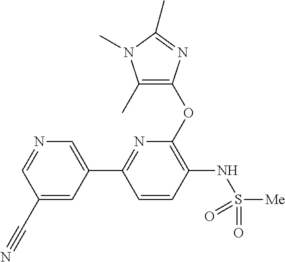 [SMILES: Cc1nc(Oc2nc(-c3cncc(C#N)c3)ccc2NS(C)(=O)=O)c(C)n1C] |
| I-115 | 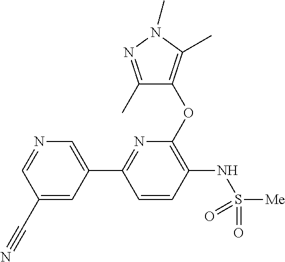 [SMILES: Cc1nn(C)c(C)c1Oc1nc(-c2cncc(C#N)c2)ccc1NS(C)(=O)=O] |
| I-116 | 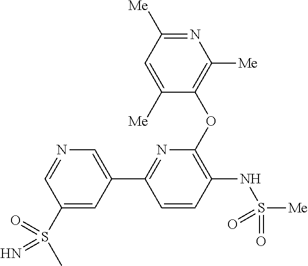 [SMILES: Cc1cc(C)c(Oc2nc(-c3cncc(S(C)(=N)=O)c3)ccc2NS(C)(=O)=O)c(C)n1] |
| I-117 | 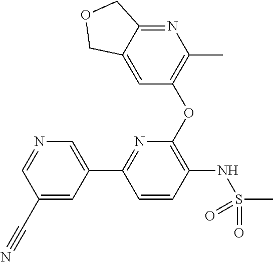 [SMILES: Cc1nc2c(cc1Oc1nc(-c3cncc(C#N)c3)ccc1NS(C)(=O)=O)COC2] |
| I-118 | 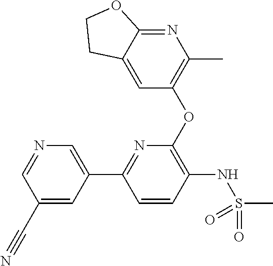 [SMILES: Cc1nc2c(cc1Oc1nc(-c3cncc(C#N)c3)ccc1NS(C)(=O)=O)CCO2] |
| I-119 | 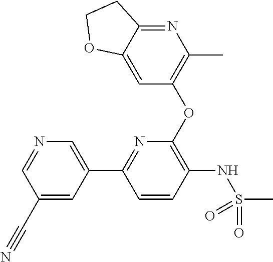 [SMILES: Cc1nc2c(cc1Oc1nc(-c3cncc(C#N)c3)ccc1NS(C)(=O)=O)OCC2] |
| I-120 | 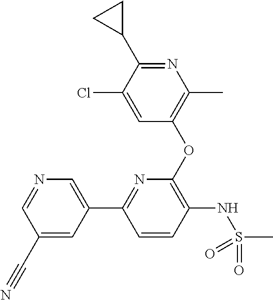 [SMILES: Cc1nc(C2CC2)c(Cl)cc1Oc1nc(-c2cncc(C#N)c2)ccc1NS(C)(=O)=O] |
| I-121 | 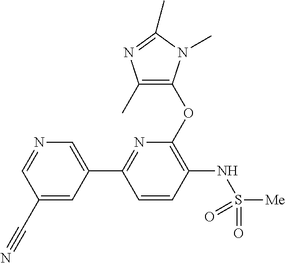 [SMILES: Cc1nc(C)n(C)c1Oc1nc(-c2cncc(C#N)c2)ccc1NS(C)(=O)=O] |
| I-122 | 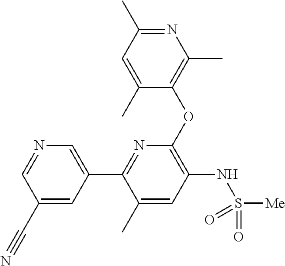 [SMILES: Cc1cc(C)c(Oc2nc(-c3cncc(C#N)c3)c(C)cc2NS(C)(=O)=O)c(C)n1] |
| I-123 | 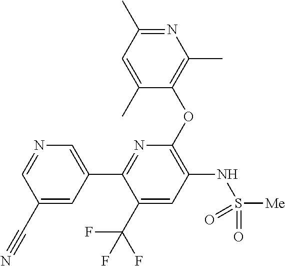 [SMILES: Cc1cc(C)c(Oc2nc(-c3cncc(C#N)c3)c(C(F)(F)F)cc2NS(C)(=O)=O)c(C)n1] |
| I-124 | 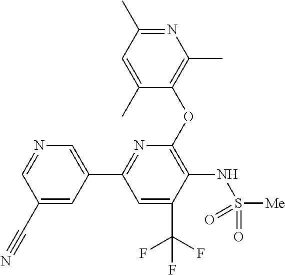 [SMILES: Cc1cc(C)c(Oc2nc(-c3cncc(C#N)c3)cc(C(F)(F)F)c2NS(C)(=O)=O)c(C)n1] |
| I-125 | 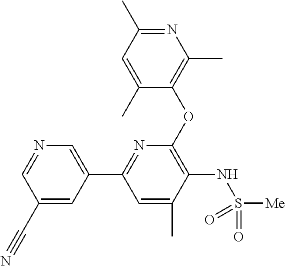 [SMILES: Cc1cc(C)c(Oc2nc(-c3cncc(C#N)c3)cc(C)c2NS(C)(=O)=O)c(C)n1] |
| I-126 | 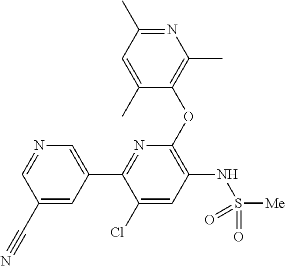 [SMILES: Cc1cc(C)c(Oc2nc(-c3cncc(C#N)c3)c(Cl)cc2NS(C)(=O)=O)c(C)n1] |
| I-127 | 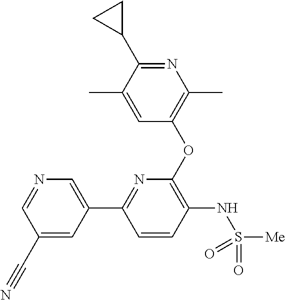 [SMILES: Cc1cc(Oc2nc(-c3cncc(C#N)c3)ccc2NS(C)(=O)=O)c(C)nc1C1CC1] |
| I-128 | 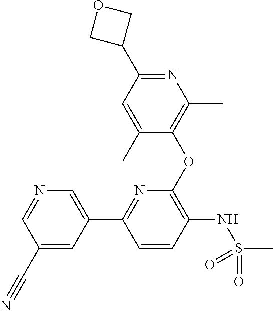 [SMILES: Cc1cc(C2COC2)nc(C)c1Oc1nc(-c2cncc(C#N)c2)ccc1NS(C)(=O)=O] |
| I-129 | 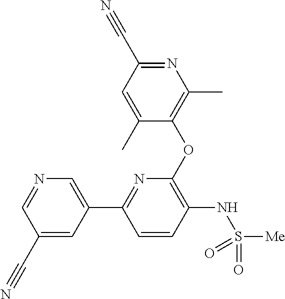 [SMILES: Cc1cc(C#N)nc(C)c1Oc1nc(-c2cncc(C#N)c2)ccc1NS(C)(=O)=O] |
| I-130 | 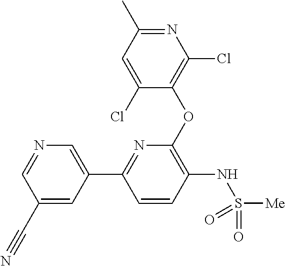 [SMILES: Cc1cc(Cl)c(Oc2nc(-c3cncc(C#N)c3)ccc2NS(C)(=O)=O)c(Cl)n1] |
| I-131 | 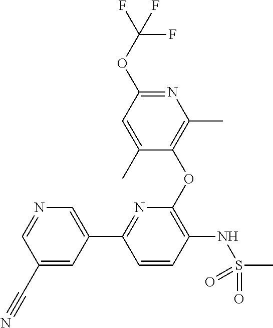 [SMILES: Cc1cc(OC(F)(F)F)nc(C)c1Oc1nc(-c2cncc(C#N)c2)ccc1NS(C)(=O)=O] |
| I-132 | 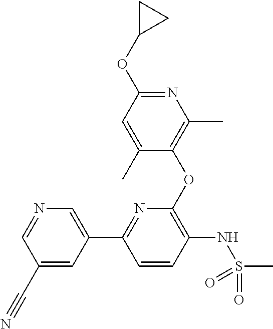 [SMILES: Cc1cc(OC2CC2)nc(C)c1Oc1nc(-c2cncc(C#N)c2)ccc1NS(C)(=O)=O] |
| I-133 | 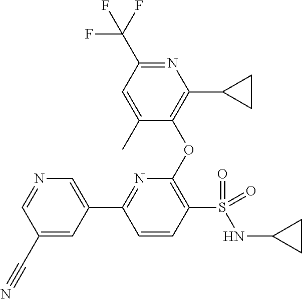 [SMILES: Cc1cc(C(F)(F)F)nc(C2CC2)c1Oc1nc(-c2cncc(C#N)c2)ccc1S(=O)(=O)NC1CC1] |
| I-134 | 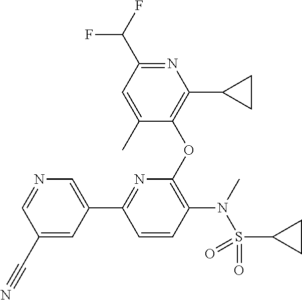 [SMILES: Cc1cc(C(F)F)nc(C2CC2)c1Oc1nc(-c2cncc(C#N)c2)ccc1N(C)S(=O)(=O)C1CC1] |
| I-135 | 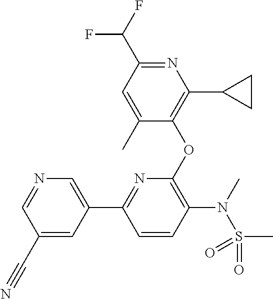 [SMILES: Cc1cc(C(F)F)nc(C2CC2)c1Oc1nc(-c2cncc(C#N)c2)ccc1N(C)S(C)(=O)=O] |
| I-136 | 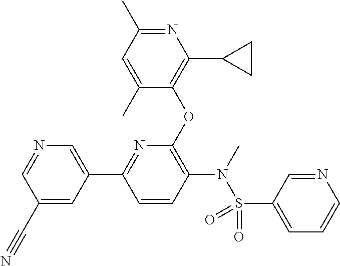 [SMILES: Cc1cc(C)c(Oc2nc(-c3cncc(C#N)c3)ccc2N(C)S(=O)(=O)c2cccnc2)c(C2CC2)n1] |
| I-137 | 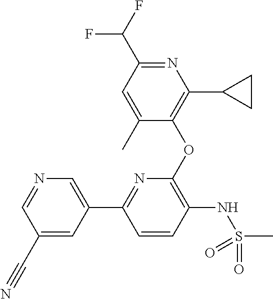 [SMILES: Cc1cc(C(F)F)nc(C2CC2)c1Oc1nc(-c2cncc(C#N)c2)ccc1NS(C)(=O)=O] |
| I-138 | 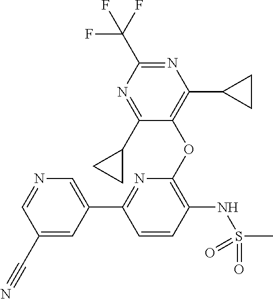 [SMILES: CS(=O)(=O)Nc1ccc(-c2cncc(C#N)c2)nc1Oc1c(C2CC2)nc(C(F)(F)F)nc1C1CC1] |
| I-139 | 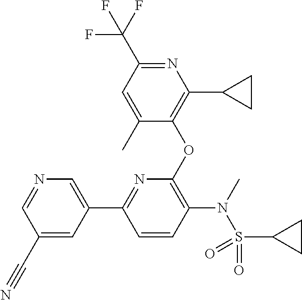 [SMILES: Cc1cc(C(F)(F)F)nc(C2CC2)c1Oc1nc(-c2cncc(C#N)c2)ccc1N(C)S(=O)(=O)C1CC1] |
| I-140 | 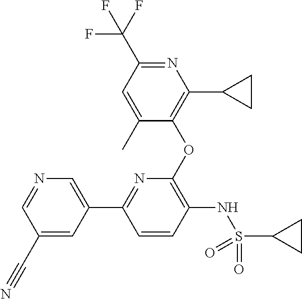 [SMILES: Cc1cc(C(F)(F)F)nc(C2CC2)c1Oc1nc(-c2cncc(C#N)c2)ccc1NS(=O)(=O)C1CC1] |
| I-141 | 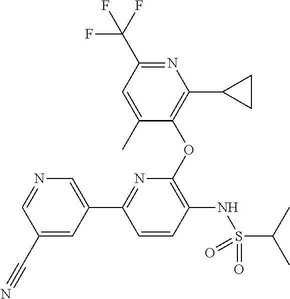 [SMILES: Cc1cc(C(F)(F)F)nc(C2CC2)c1Oc1nc(-c2cncc(C#N)c2)ccc1NS(=O)(=O)C(C)C] |
| I-142 | 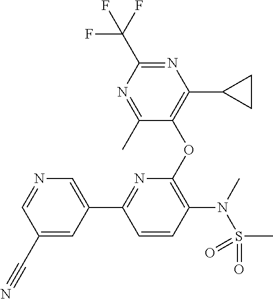 [SMILES: Cc1nc(C(F)(F)F)nc(C2CC2)c1Oc1nc(-c2cncc(C#N)c2)ccc1N(C)S(C)(=O)=O] |
| I-143 | 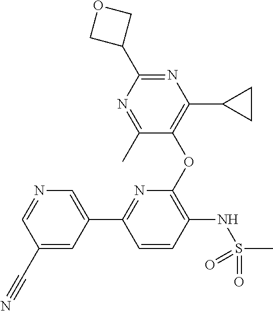 [SMILES: Cc1nc(C2COC2)nc(C2CC2)c1Oc1nc(-c2cncc(C#N)c2)ccc1NS(C)(=O)=O] |
| I-144 | 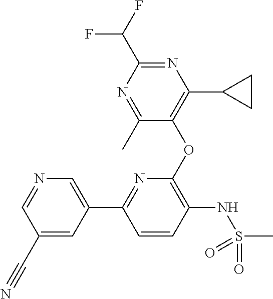 [SMILES: Cc1nc(C(F)F)nc(C2CC2)c1Oc1nc(-c2cncc(C#N)c2)ccc1NS(C)(=O)=O] |
| I-145 | 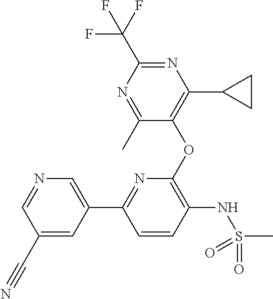 [SMILES: Cc1nc(C(F)(F)F)nc(C2CC2)c1Oc1nc(-c2cncc(C#N)c2)ccc1NS(C)(=O)=O] |
| I-146 | 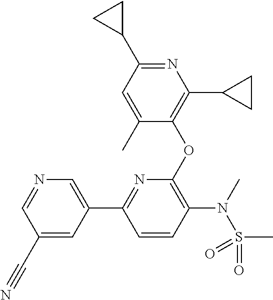 [SMILES: Cc1cc(C2CC2)nc(C2CC2)c1Oc1nc(-c2cncc(C#N)c2)ccc1N(C)S(C)(=O)=O] |
| I-147 | 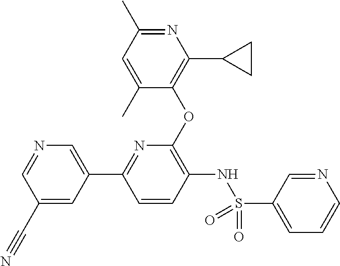 [SMILES: Cc1cc(C)c(Oc2nc(-c3cncc(C#N)c3)ccc2NS(=O)(=O)c2cccnc2)c(C2CC2)n1] |
| I-148 | 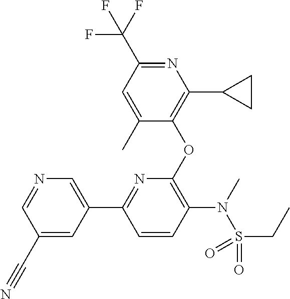 [SMILES: CCS(=O)(=O)N(C)c1ccc(-c2cncc(C#N)c2)nc1Oc1c(C)cc(C(F)(F)F)nc1C1CC1] |
| I-149 | 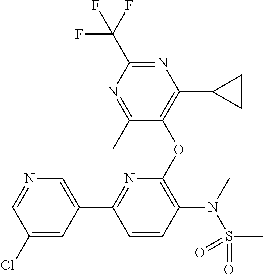 [SMILES: Cc1nc(C(F)(F)F)nc(C2CC2)c1Oc1nc(-c2cncc(Cl)c2)ccc1N(C)S(C)(=O)=O] |
| I-150 | 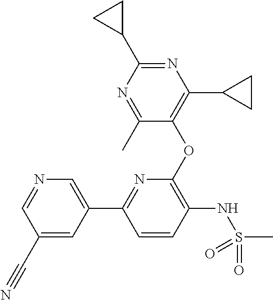 [SMILES: Cc1nc(C2CC2)nc(C2CC2)c1Oc1nc(-c2cncc(C#N)c2)ccc1NS(C)(=O)=O] |
| I-151 | 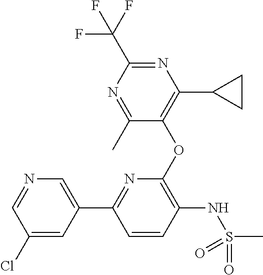 [SMILES: Cc1nc(C(F)(F)F)nc(C2CC2)c1Oc1nc(-c2cncc(Cl)c2)ccc1NS(C)(=O)=O] |
| I-152 | 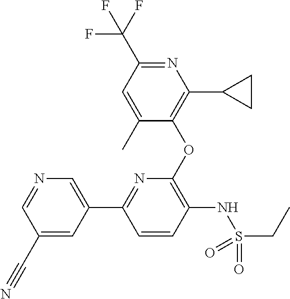 [SMILES: CCS(=O)(=O)Nc1ccc(-c2cncc(C#N)c2)nc1Oc1c(C)cc(C(F)(F)F)nc1C1CC1] |
| I-153 | 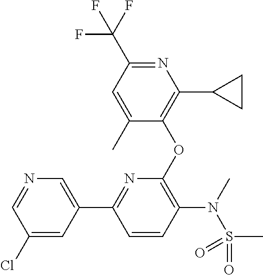 [SMILES: Cc1cc(C(F)(F)F)nc(C2CC2)c1Oc1nc(-c2cncc(Cl)c2)ccc1N(C)S(C)(=O)=O] |
| I-154 | 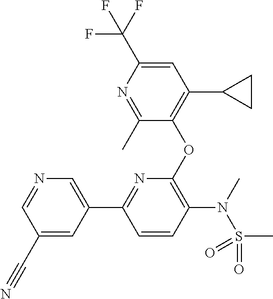 [SMILES: Cc1nc(C(F)(F)F)cc(C2CC2)c1Oc1nc(-c2cncc(C#N)c2)ccc1N(C)S(C)(=O)=O] |
| I-155 | 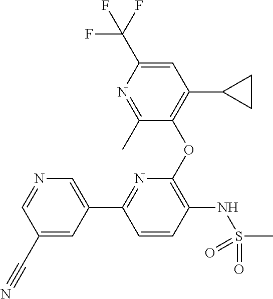 [SMILES: Cc1nc(C(F)(F)F)cc(C2CC2)c1Oc1nc(-c2cncc(C#N)c2)ccc1NS(C)(=O)=O] |
| I-156 | 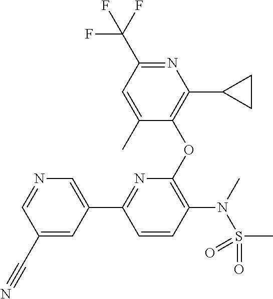 [SMILES: Cc1cc(C(F)(F)F)nc(C2CC2)c1Oc1nc(-c2cncc(C#N)c2)ccc1N(C)S(C)(=O)=O] |
| I-157 | 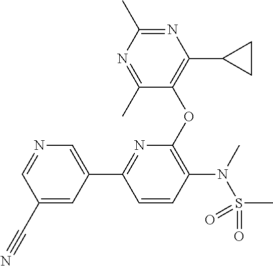 [SMILES: Cc1nc(C)c(Oc2nc(-c3cncc(C#N)c3)ccc2N(C)S(C)(=O)=O)c(C2CC2)n1] |
| I-158 | 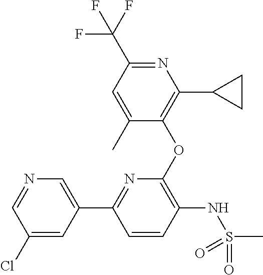 [SMILES: Cc1cc(C(F)(F)F)nc(C2CC2)c1Oc1nc(-c2cncc(Cl)c2)ccc1NS(C)(=O)=O] |
| I-159 | 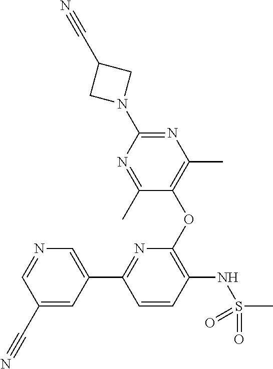 [SMILES: Cc1nc(N2CC(C#N)C2)nc(C)c1Oc1nc(-c2cncc(C#N)c2)ccc1NS(C)(=O)=O] |
| I-160 | 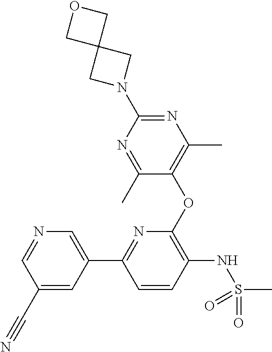 [SMILES: Cc1nc(N2CC3(COC3)C2)nc(C)c1Oc1nc(-c2cncc(C#N)c2)ccc1NS(C)(=O)=O] |
| I-161 | 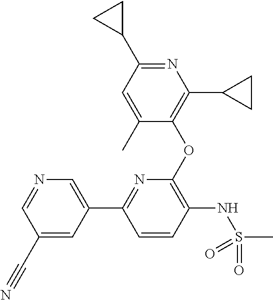 [SMILES: Cc1cc(C2CC2)nc(C2CC2)c1Oc1nc(-c2cncc(C#N)c2)ccc1NS(C)(=O)=O] |
| I-162 | 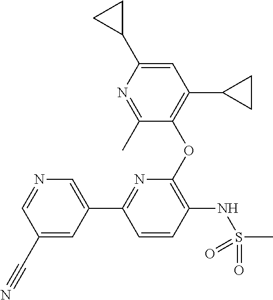 [SMILES: Cc1nc(C2CC2)cc(C2CC2)c1Oc1nc(-c2cncc(C#N)c2)ccc1NS(C)(=O)=O] |
| I-163 | 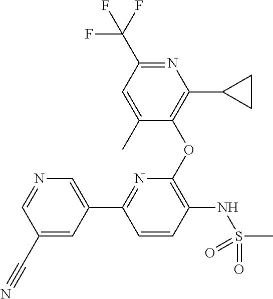 [SMILES: Cc1cc(C(F)(F)F)nc(C2CC2)c1Oc1nc(-c2cncc(C#N)c2)ccc1NS(C)(=O)=O] |
| I-164 | 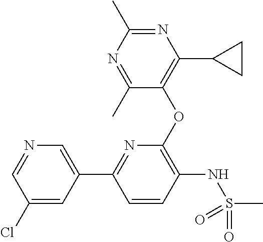 [SMILES: Cc1nc(C)c(Oc2nc(-c3cncc(Cl)c3)ccc2NS(C)(=O)=O)c(C2CC2)n1] |
| I-165 | 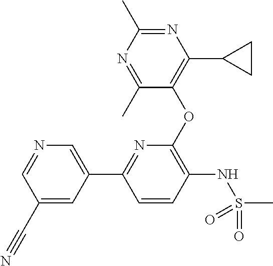 [SMILES: Cc1nc(C)c(Oc2nc(-c3cncc(C#N)c3)ccc2NS(C)(=O)=O)c(C2CC2)n1] |
| I-166 | 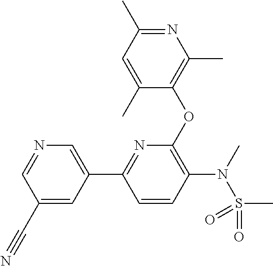 [SMILES: Cc1cc(C)c(Oc2nc(-c3cncc(C#N)c3)ccc2N(C)S(C)(=O)=O)c(C)n1] |
| I-167 | 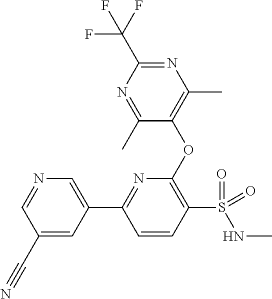 [SMILES: CNS(=O)(=O)c1ccc(-c2cncc(C#N)c2)nc1Oc1c(C)nc(C(F)(F)F)nc1C] |
| I-168 | 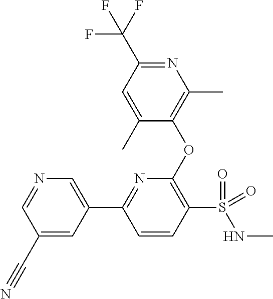 [SMILES: CNS(=O)(=O)c1ccc(-c2cncc(C#N)c2)nc1Oc1c(C)cc(C(F)(F)F)nc1C] |
| I-169 | 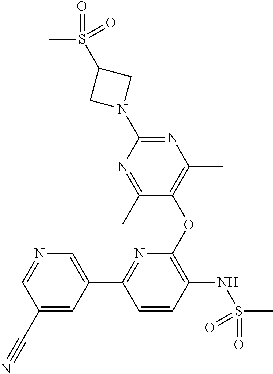 [SMILES: Cc1nc(N2CC(S(C)(=O)=O)C2)nc(C)c1Oc1nc(-c2cncc(C#N)c2)ccc1NS(C)(=O)=O] |
| I-170 | 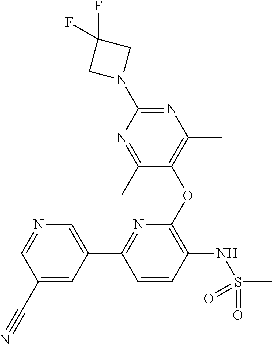 [SMILES: Cc1nc(N2CC(F)(F)C2)nc(C)c1Oc1nc(-c2cncc(C#N)c2)ccc1NS(C)(=O)=O] |
| I-171 | 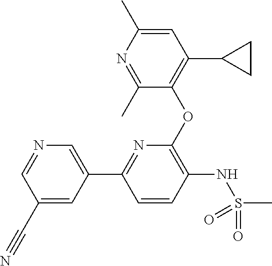 [SMILES: Cc1cc(C2CC2)c(Oc2nc(-c3cncc(C#N)c3)ccc2NS(C)(=O)=O)c(C)n1] |
| I-172 | 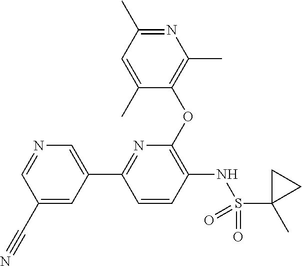 [SMILES: Cc1cc(C)c(Oc2nc(-c3cncc(C#N)c3)ccc2NS(=O)(=O)C2(C)CC2)c(C)n1] |
| I-173 | 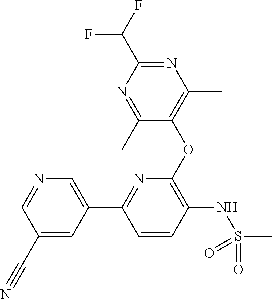 [SMILES: Cc1nc(C(F)F)nc(C)c1Oc1nc(-c2cncc(C#N)c2)ccc1NS(C)(=O)=O] |
| I-174 | 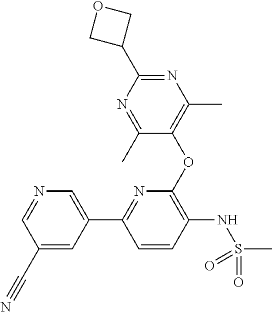 [SMILES: Cc1nc(C2COC2)nc(C)c1Oc1nc(-c2cncc(C#N)c2)ccc1NS(C)(=O)=O] |
| I-175 | 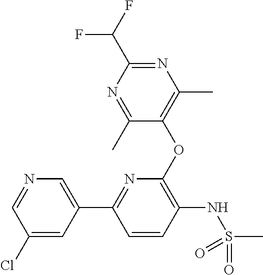 [SMILES: Cc1nc(C(F)F)nc(C)c1Oc1nc(-c2cncc(Cl)c2)ccc1NS(C)(=O)=O] |
| I-176 | 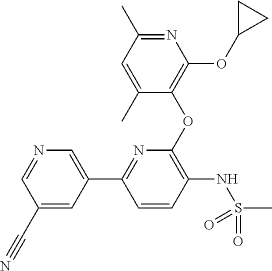 [SMILES: Cc1cc(C)c(Oc2nc(-c3cncc(C#N)c3)ccc2NS(C)(=O)=O)c(OC2CC2)n1] |
| I-177 | 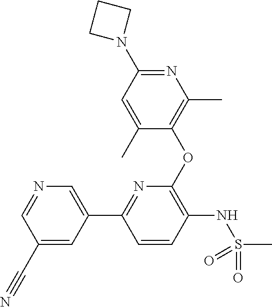 [SMILES: Cc1cc(N2CCC2)nc(C)c1Oc1nc(-c2cncc(C#N)c2)ccc1NS(C)(=O)=O] |
| I-178 | 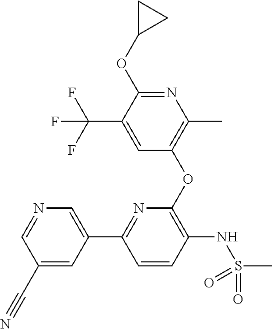 [SMILES: Cc1nc(OC2CC2)c(C(F)(F)F)cc1Oc1nc(-c2cncc(C#N)c2)ccc1NS(C)(=O)=O] |
| I-179 | 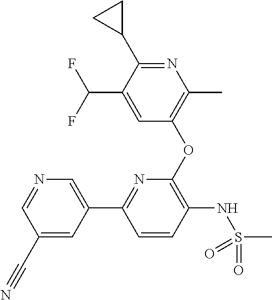 [SMILES: Cc1nc(C2CC2)c(C(F)F)cc1Oc1nc(-c2cncc(C#N)c2)ccc1NS(C)(=O)=O] |
| I-180 | 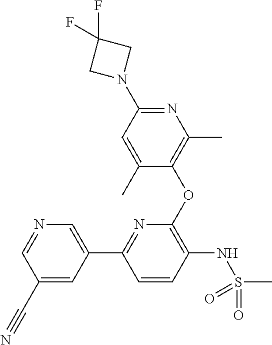 [SMILES: Cc1cc(N2CC(F)(F)C2)nc(C)c1Oc1nc(-c2cncc(C#N)c2)ccc1NS(C)(=O)=O] |
| I-181 | 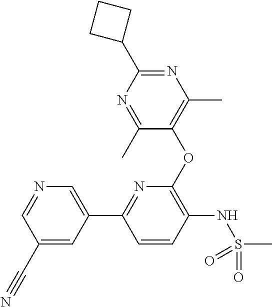 [SMILES: Cc1nc(C2CCC2)nc(C)c1Oc1nc(-c2cncc(C#N)c2)ccc1NS(C)(=O)=O] |
| I-182 | 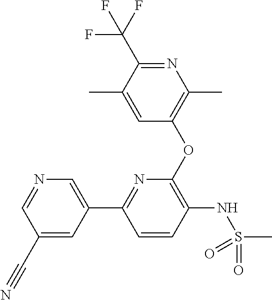 [SMILES: Cc1cc(Oc2nc(-c3cncc(C#N)c3)ccc2NS(C)(=O)=O)c(C)nc1C(F)(F)F] |
| I-183 | 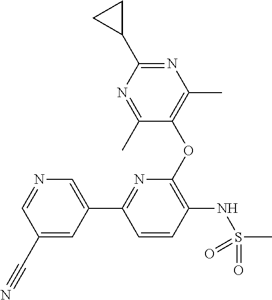 [SMILES: Cc1nc(C2CC2)nc(C)c1Oc1nc(-c2cncc(C#N)c2)ccc1NS(C)(=O)=O] |
| I-184 | 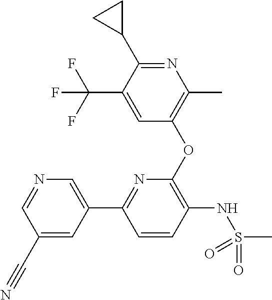 [SMILES: Cc1nc(C2CC2)c(C(F)(F)F)cc1Oc1nc(-c2cncc(C#N)c2)ccc1NS(C)(=O)=O] |
| I-185 | 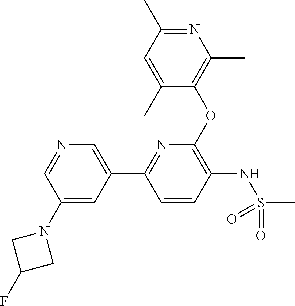 [SMILES: Cc1cc(C)c(Oc2nc(-c3cncc(N4CC(F)C4)c3)ccc2NS(C)(=O)=O)c(C)n1] |
| I-186 | 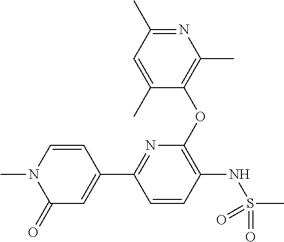 |
| I-187 | 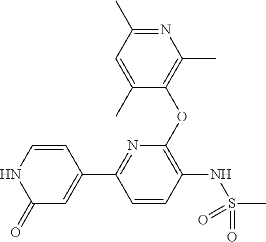 [SMILES: Cc1cc(C)c(Oc2nc(-c3cc[nH]c(=O)c3)ccc2NS(C)(=O)=O)c(C)n1] |
| I-188 | 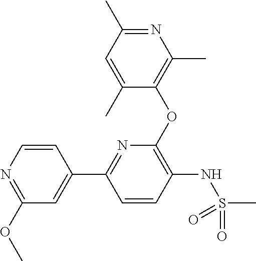 [SMILES: COc1cc(-c2ccc(NS(C)(=O)=O)c(Oc3c(C)cc(C)nc3C)n2)ccn1] |
| I-190 | 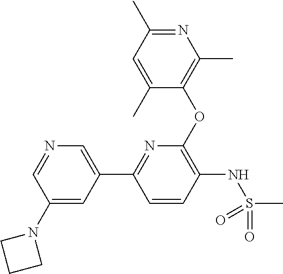 [SMILES: Cc1cc(C)c(Oc2nc(-c3cncc(N4CCC4)c3)ccc2NS(C)(=O)=O)c(C)n1] |
| I-191 | 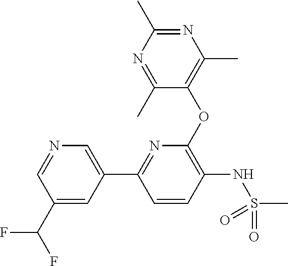 [SMILES: Cc1nc(C)c(Oc2nc(-c3cncc(C(F)F)c3)ccc2NS(C)(=O)=O)c(C)n1] |
| I-192 | 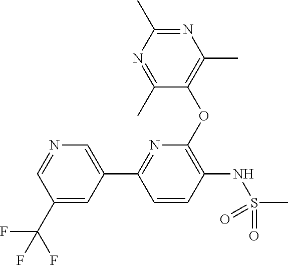 [SMILES: Cc1nc(C)c(Oc2nc(-c3cncc(C(F)(F)F)c3)ccc2NS(C)(=O)=O)c(C)n1] |
| I-193 | 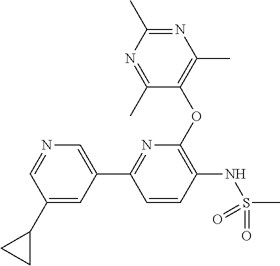 [SMILES: Cc1nc(C)c(Oc2nc(-c3cncc(C4CC4)c3)ccc2NS(C)(=O)=O)c(C)n1] |
| I-194 | 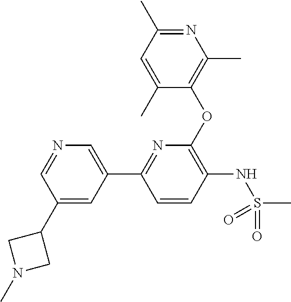 |
| I-195 | 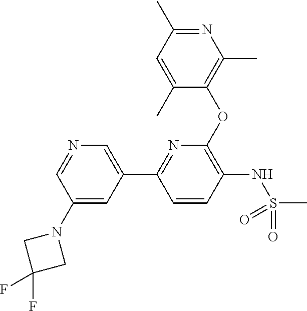 [SMILES: Cc1cc(C)c(Oc2nc(-c3cncc(N4CC(F)(F)C4)c3)ccc2NS(C)(=O)=O)c(C)n1] |
| I-196 | 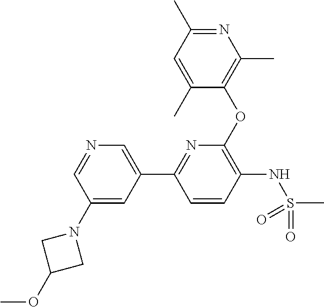 |
| I-197 | 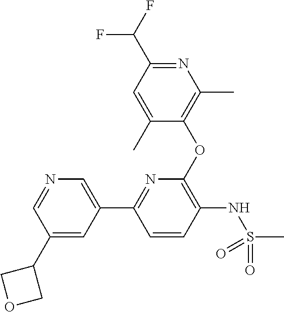 [SMILES: Cc1cc(C(F)F)nc(C)c1Oc1nc(-c2cncc(C3COC3)c2)ccc1NS(C)(=O)=O] |
| I-198 | 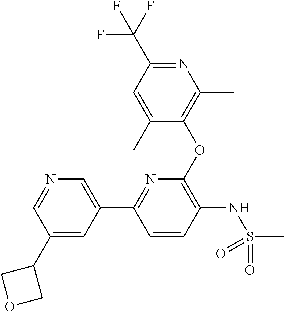 [SMILES: Cc1cc(C(F)(F)F)nc(C)c1Oc1nc(-c2cncc(C3COC3)c2)ccc1NS(C)(=O)=O] |
| I-199 | 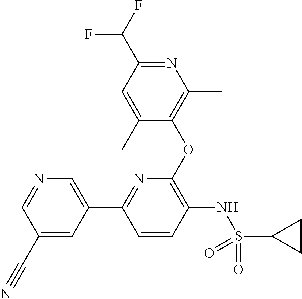 [SMILES: Cc1cc(C(F)F)nc(C)c1Oc1nc(-c2cncc(C#N)c2)ccc1NS(=O)(=O)C1CC1] |
| I-200 | 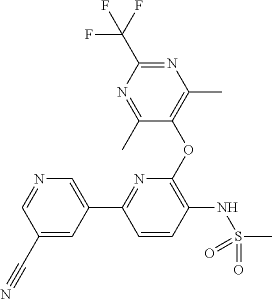 [SMILES: Cc1nc(C(F)(F)F)nc(C)c1Oc1nc(-c2cncc(C#N)c2)ccc1NS(C)(=O)=O] |
| I-201 | 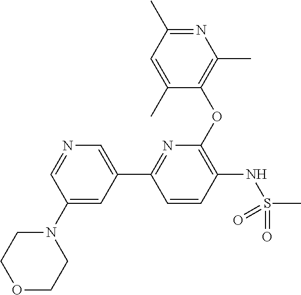 [SMILES: Cc1cc(C)c(Oc2nc(-c3cncc(N4CCOCC4)c3)ccc2NS(C)(=O)=O)c(C)n1] |
| I-202 | 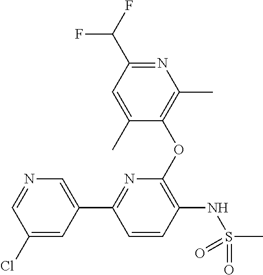 [SMILES: Cc1cc(C(F)F)nc(C)c1Oc1nc(-c2cncc(Cl)c2)ccc1NS(C)(=O)=O] |
| I-203 | 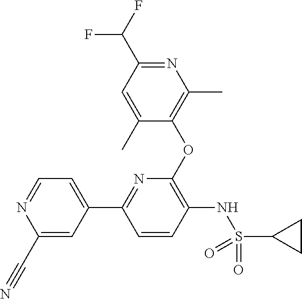 [SMILES: Cc1cc(C(F)F)nc(C)c1Oc1nc(-c2ccnc(C#N)c2)ccc1NS(=O)(=O)C1CC1] |
| I-204 | 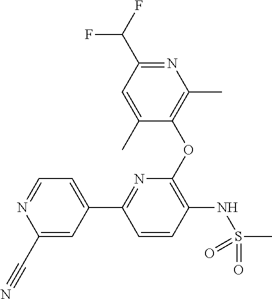 [SMILES: Cc1cc(C(F)F)nc(C)c1Oc1nc(-c2ccnc(C#N)c2)ccc1NS(C)(=O)=O] |
| I-205 | 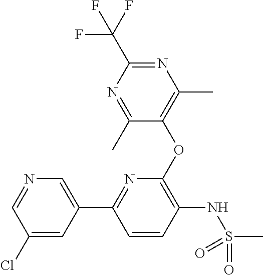 [SMILES: Cc1nc(C(F)(F)F)nc(C)c1Oc1nc(-c2cncc(Cl)c2)ccc1NS(C)(=O)=O] |
| I-206 | 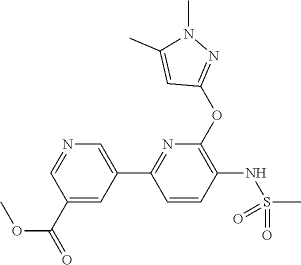 |
| I-208 | 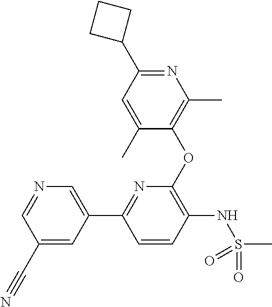 [SMILES: Cc1cc(C2CCC2)nc(C)c1Oc1nc(-c2cncc(C#N)c2)ccc1NS(C)(=O)=O] |
| I-209 | 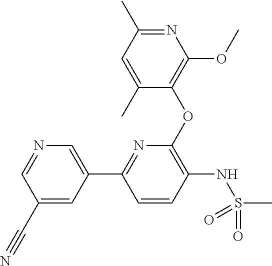 [SMILES: COc1nc(C)cc(C)c1Oc1nc(-c2cncc(C#N)c2)ccc1NS(C)(=O)=O] |
| I-211 | 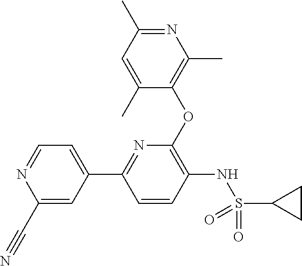 [SMILES: Cc1cc(C)c(Oc2nc(-c3ccnc(C#N)c3)ccc2NS(=O)(=O)C2CC2)c(C)n1] |
| I-212 | 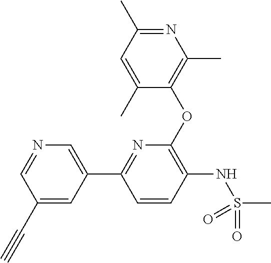 [SMILES: C#Cc1cncc(-c2ccc(NS(C)(=O)=O)c(Oc3c(C)cc(C)nc3C)n2)c1] |
| I-213 | 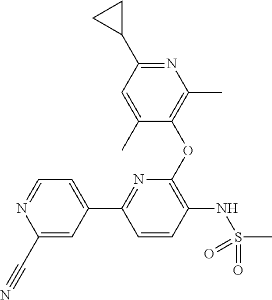 [SMILES: Cc1cc(C2CC2)nc(C)c1Oc1nc(-c2ccnc(C#N)c2)ccc1NS(C)(=O)=O] |
| I-216 | 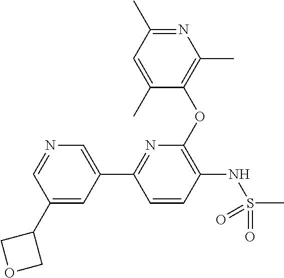 [SMILES: Cc1cc(C)c(Oc2nc(-c3cncc(C4COC4)c3)ccc2NS(C)(=O)=O)c(C)n1] |
| I-217 | 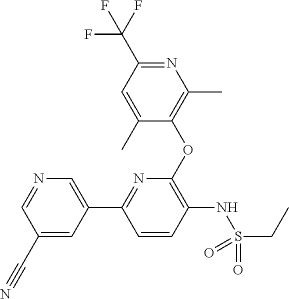 [SMILES: CCS(=O)(=O)Nc1ccc(-c2cncc(C#N)c2)nc1Oc1c(C)cc(C(F)(F)F)nc1C] |
| I-218 | 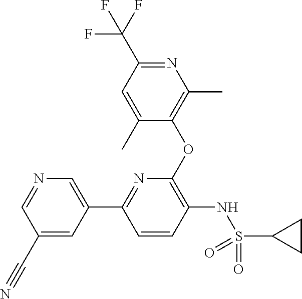 [SMILES: Cc1cc(C(F)(F)F)nc(C)c1Oc1nc(-c2cncc(C#N)c2)ccc1NS(=O)(=O)C1CC1] |
| I-219 | 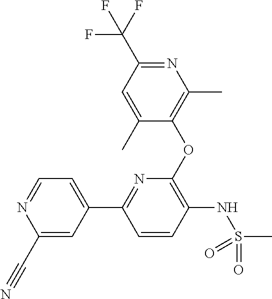 [SMILES: Cc1cc(C(F)(F)F)nc(C)c1Oc1nc(-c2ccnc(C#N)c2)ccc1NS(C)(=O)=O] |
| I-220 | 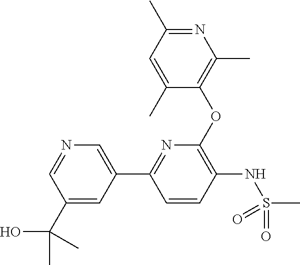 [SMILES: Cc1cc(C)c(Oc2nc(-c3cncc(C(C)(C)O)c3)ccc2NS(C)(=O)=O)c(C)n1] |
| I-221 | 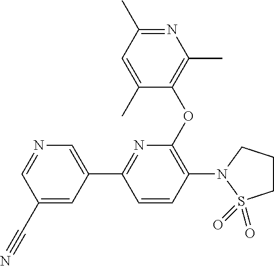 [SMILES: Cc1cc(C)c(Oc2nc(-c3cncc(C#N)c3)ccc2N2CCCS2(=O)=O)c(C)n1] |
| I-222 | 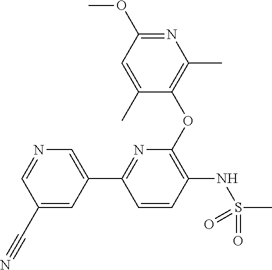 [SMILES: COc1cc(C)c(Oc2nc(-c3cncc(C#N)c3)ccc2NS(C)(=O)=O)c(C)n1] |
| I-225 | 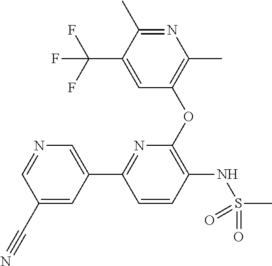 [SMILES: Cc1nc(C)c(C(F)(F)F)cc1Oc1nc(-c2cncc(C#N)c2)ccc1NS(C)(=O)=O] |
| I-226 | 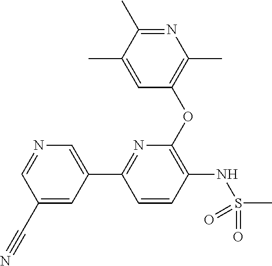 [SMILES: Cc1cc(Oc2nc(-c3cncc(C#N)c3)ccc2NS(C)(=O)=O)c(C)nc1C] |
| I-227 | 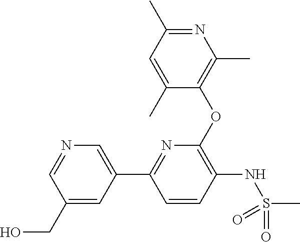 [SMILES: Cc1cc(C)c(Oc2nc(-c3cncc(CO)c3)ccc2NS(C)(=O)=O)c(C)n1] |
| I-228 | 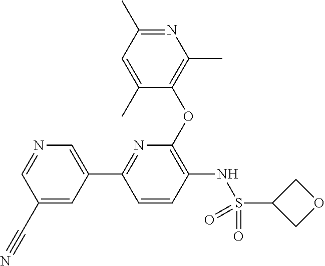 [SMILES: Cc1cc(C)c(Oc2nc(-c3cncc(C#N)c3)ccc2NS(=O)(=O)C2COC2)c(C)n1] |
| I-229 | 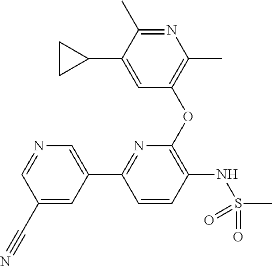 [SMILES: Cc1nc(C)c(C2CC2)cc1Oc1nc(-c2cncc(C#N)c2)ccc1NS(C)(=O)=O] |
| I-230 | 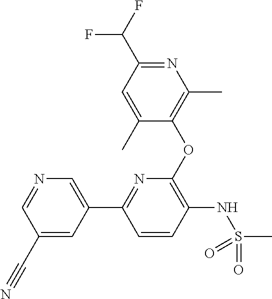 [SMILES: Cc1cc(C(F)F)nc(C)c1Oc1nc(-c2cncc(C#N)c2)ccc1NS(C)(=O)=O] |
| I-231 | 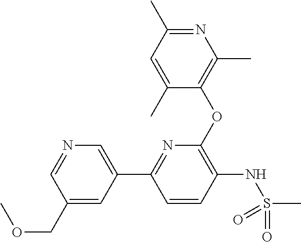 |
| I-232 | 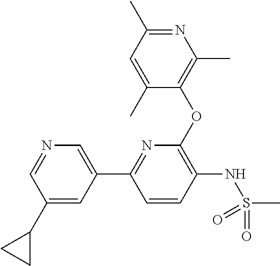 [SMILES: Cc1cc(C)c(Oc2nc(-c3cncc(C4CC4)c3)ccc2NS(C)(=O)=O)c(C)n1] |
| I-234 | 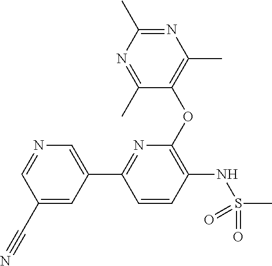 [SMILES: Cc1nc(C)c(Oc2nc(-c3cncc(C#N)c3)ccc2NS(C)(=O)=O)c(C)n1] |
| I-235 | 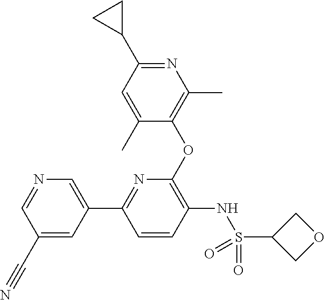 [SMILES: Cc1cc(C2CC2)nc(C)c1Oc1nc(-c2cncc(C#N)c2)ccc1NS(=O)(=O)C1COC1] |
| I-236 | 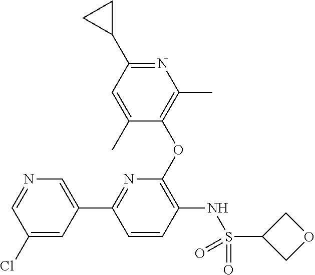 [SMILES: Cc1cc(C2CC2)nc(C)c1Oc1nc(-c2cncc(Cl)c2)ccc1NS(=O)(=O)C1COC1] |
| I-237 | 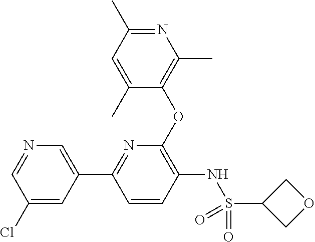 [SMILES: Cc1cc(C)c(Oc2nc(-c3cncc(Cl)c3)ccc2NS(=O)(=O)C2COC2)c(C)n1] |
| I-238 | 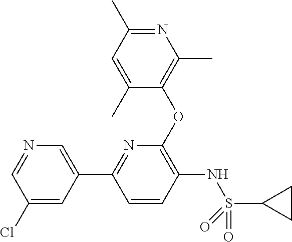 [SMILES: Cc1cc(C)c(Oc2nc(-c3cncc(Cl)c3)ccc2NS(=O)(=O)C2CC2)c(C)n1] |
| I-239 | 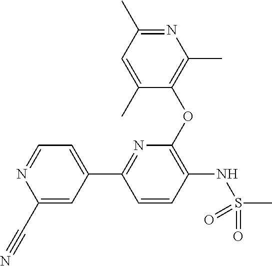 [SMILES: Cc1cc(C)c(Oc2nc(-c3ccnc(C#N)c3)ccc2NS(C)(=O)=O)c(C)n1] |
| I-240 | 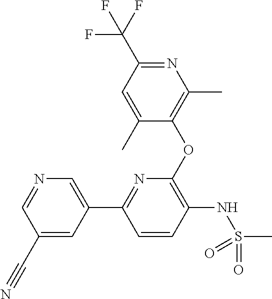 [SMILES: Cc1cc(C(F)(F)F)nc(C)c1Oc1nc(-c2cncc(C#N)c2)ccc1NS(C)(=O)=O] |
| I-241 | 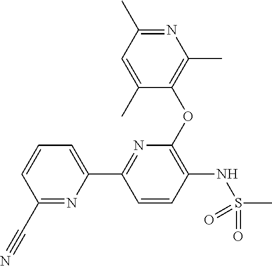 [SMILES: Cc1cc(C)c(Oc2nc(-c3cccc(C#N)n3)ccc2NS(C)(=O)=O)c(C)n1] |
| I-242 | 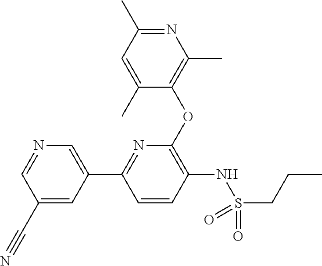 [SMILES: CCCS(=O)(=O)Nc1ccc(-c2cncc(C#N)c2)nc1Oc1c(C)cc(C)nc1C] |
| I-243 | 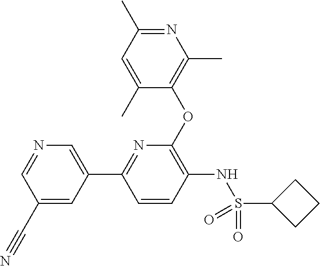 [SMILES: Cc1cc(C)c(Oc2nc(-c3cncc(C#N)c3)ccc2NS(=O)(=O)C2CCC2)c(C)n1] |
| I-244 | 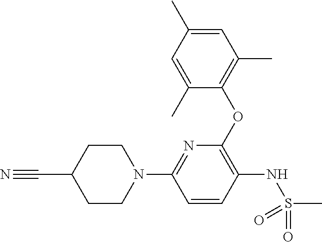 [SMILES: Cc1cc(C)c(Oc2nc(N3CCC(C#N)CC3)ccc2NS(C)(=O)=O)c(C)c1] |
| I-245 | 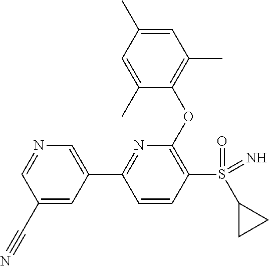 [SMILES: Cc1cc(C)c(Oc2nc(-c3cncc(C#N)c3)ccc2S(=N)(=O)C2CC2)c(C)c1] |
| I-246 | 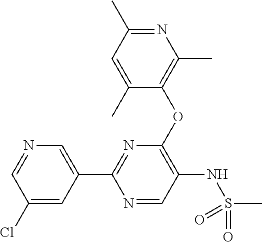 [SMILES: Cc1cc(C)c(Oc2nc(-c3cncc(Cl)c3)ncc2NS(C)(=O)=O)c(C)n1] |
| I-247 | 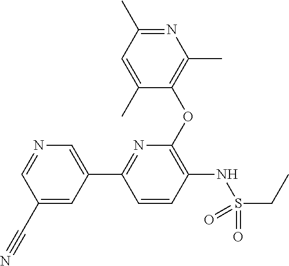 [SMILES: CCS(=O)(=O)Nc1ccc(-c2cncc(C#N)c2)nc1Oc1c(C)cc(C)nc1C] |
| I-248 | 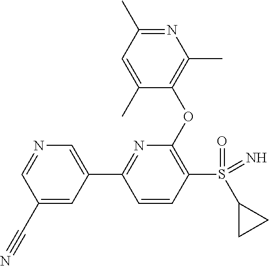 [SMILES: Cc1cc(C)c(Oc2nc(-c3cncc(C#N)c3)ccc2S(=N)(=O)C2CC2)c(C)n1] |
| I-249 | 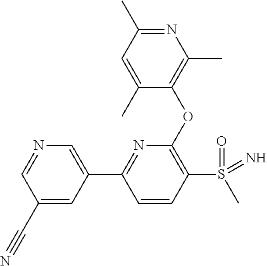 [SMILES: Cc1cc(C)c(Oc2nc(-c3cncc(C#N)c3)ccc2S(C)(=N)=O)c(C)n1] |
| I-250 | 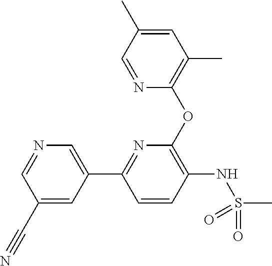 [SMILES: Cc1cnc(Oc2nc(-c3cncc(C#N)c3)ccc2NS(C)(=O)=O)c(C)c1] |
| I-251 | 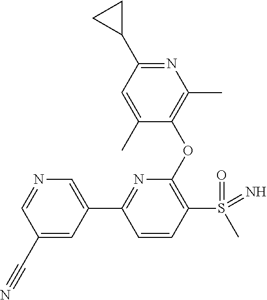 [SMILES: Cc1cc(C2CC2)nc(C)c1Oc1nc(-c2cncc(C#N)c2)ccc1S(C)(=N)=O] |
| I-252 | 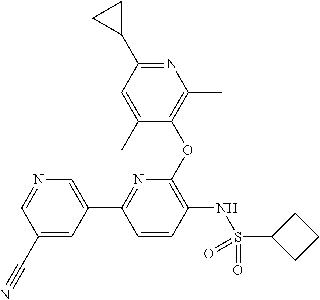 [SMILES: Cc1cc(C2CC2)nc(C)c1Oc1nc(-c2cncc(C#N)c2)ccc1NS(=O)(=O)C1CCC1] |
| I-253 | 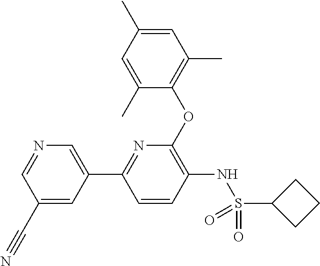 [SMILES: Cc1cc(C)c(Oc2nc(-c3cncc(C#N)c3)ccc2NS(=O)(=O)C2CCC2)c(C)c1] |
| I-254 | 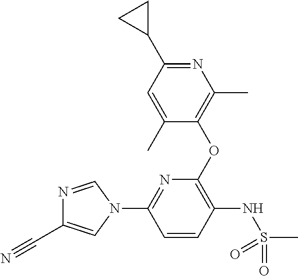 [SMILES: Cc1cc(C2CC2)nc(C)c1Oc1nc(-n2cnc(C#N)c2)ccc1NS(C)(=O)=O] |
| I-255 | 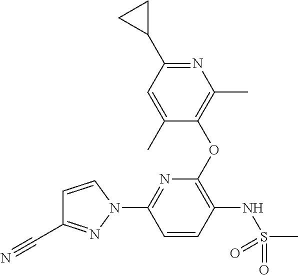 [SMILES: Cc1cc(C2CC2)nc(C)c1Oc1nc(-n2ccc(C#N)n2)ccc1NS(C)(=O)=O] |
| I-256 | 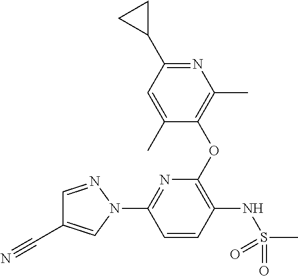 [SMILES: Cc1cc(C2CC2)nc(C)c1Oc1nc(-n2cc(C#N)cn2)ccc1NS(C)(=O)=O] |
| I-257 | 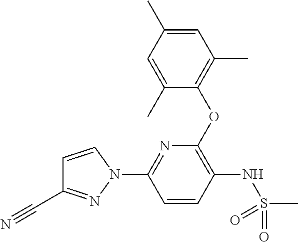 [SMILES: Cc1cc(C)c(Oc2nc(-n3ccc(C#N)n3)ccc2NS(C)(=O)=O)c(C)c1] |
| I-258 | 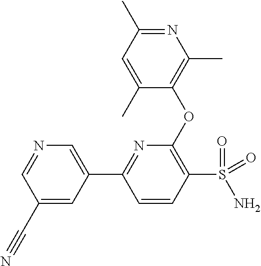 [SMILES: Cc1cc(C)c(Oc2nc(-c3cncc(C#N)c3)ccc2S(N)(=O)=O)c(C)n1] |
| I-259 | 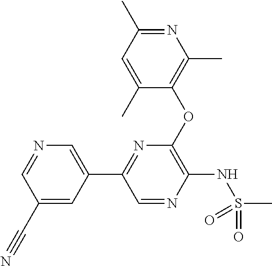 [SMILES: Cc1cc(C)c(Oc2nc(-c3cncc(C#N)c3)cnc2NS(C)(=O)=O)c(C)n1] |
| I-260 | 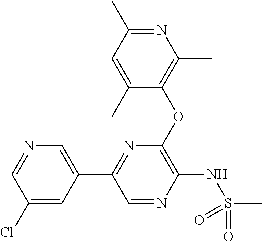 [SMILES: Cc1cc(C)c(Oc2nc(-c3cncc(Cl)c3)cnc2NS(C)(=O)=O)c(C)n1] |
| I-261 | 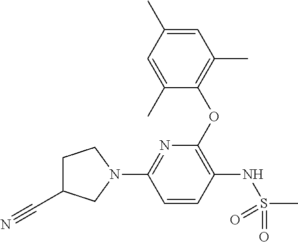 [SMILES: Cc1cc(C)c(Oc2nc(N3CCC(C#N)C3)ccc2NS(C)(=O)=O)c(C)c1] |
| | Stereoisomer I |
| I-262 | 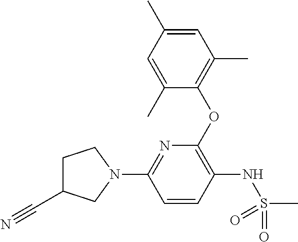 [SMILES: Cc1cc(C)c(Oc2nc(N3CCC(C#N)C3)ccc2NS(C)(=O)=O)c(C)c1] |
| | Stereoisomer II |
| I-263 | 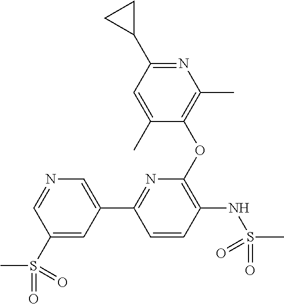 |
| I-264 | 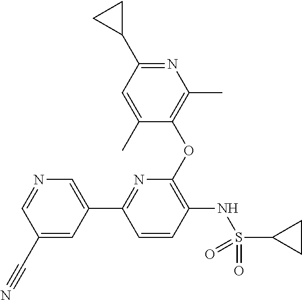 [SMILES: Cc1cc(C2CC2)nc(C)c1Oc1nc(-c2cncc(C#N)c2)ccc1NS(=O)(=O)C1CC1] |
| I-265 | 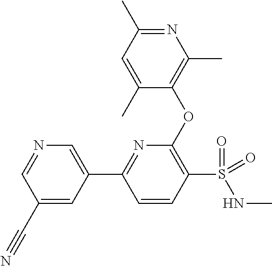 [SMILES: CNS(=O)(=O)c1ccc(-c2cncc(C#N)c2)nc1Oc1c(C)cc(C)nc1C] |
| I-266 | 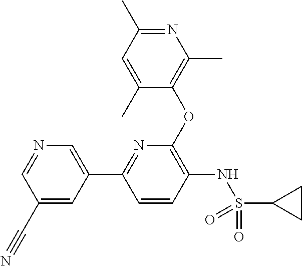 [SMILES: Cc1cc(C)c(Oc2nc(-c3cncc(C#N)c3)ccc2NS(=O)(=O)C2CC2)c(C)n1] |
| I-267 | 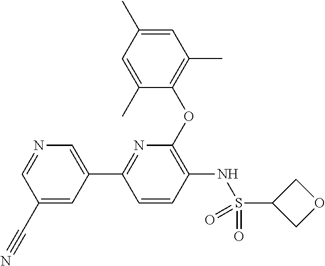 [SMILES: Cc1cc(C)c(Oc2nc(-c3cncc(C#N)c3)ccc2NS(=O)(=O)C2COC2)c(C)c1] |
| I-268 | 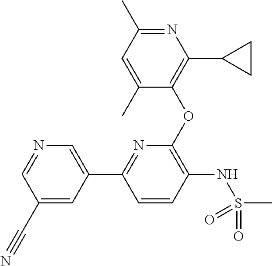 [SMILES: Cc1cc(C)c(Oc2nc(-c3cncc(C#N)c3)ccc2NS(C)(=O)=O)c(C2CC2)n1] |
| I-269 | 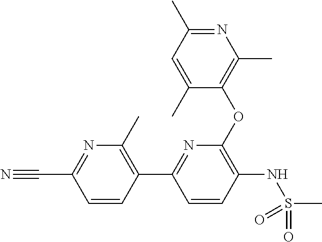 [SMILES: Cc1cc(C)c(Oc2nc(-c3ccc(C#N)nc3C)ccc2NS(C)(=O)=O)c(C)n1] |
| I-270 | 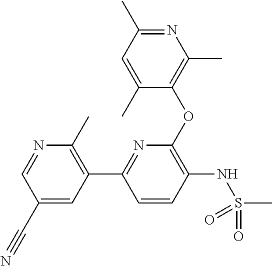 [SMILES: Cc1cc(C)c(Oc2nc(-c3cc(C#N)cnc3C)ccc2NS(C)(=O)=O)c(C)n1] |
| I-271 | 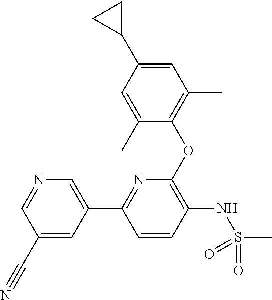 [SMILES: Cc1cc(C2CC2)cc(C)c1Oc1nc(-c2cncc(C#N)c2)ccc1NS(C)(=O)=O] |
| I-272 | 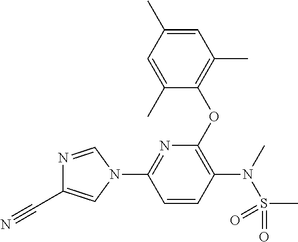 [SMILES: Cc1cc(C)c(Oc2nc(-n3cnc(C#N)c3)ccc2N(C)S(C)(=O)=O)c(C)c1] |
| I-273 | 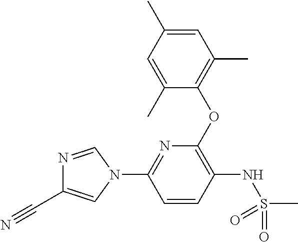 [SMILES: Cc1cc(C)c(Oc2nc(-n3cnc(C#N)c3)ccc2NS(C)(=O)=O)c(C)c1] |
| I-274 | 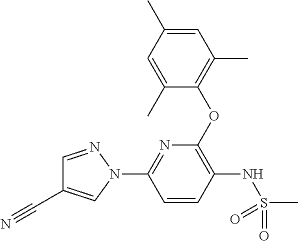 [SMILES: Cc1cc(C)c(Oc2nc(-n3cc(C#N)cn3)ccc2NS(C)(=O)=O)c(C)c1] |
| I-275 | 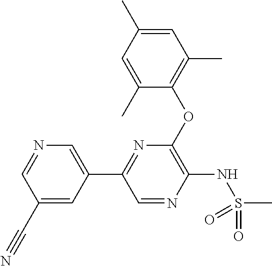 [SMILES: Cc1cc(C)c(Oc2nc(-c3cncc(C#N)c3)cnc2NS(C)(=O)=O)c(C)c1] |
| I-276 | 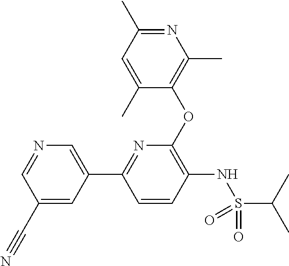 [SMILES: Cc1cc(C)c(Oc2nc(-c3cncc(C#N)c3)ccc2NS(=O)(=O)C(C)C)c(C)n1] |
| I-277 | 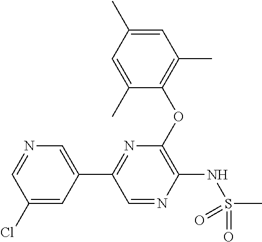 [SMILES: Cc1cc(C)c(Oc2nc(-c3cncc(Cl)c3)cnc2NS(C)(=O)=O)c(C)c1] |
| I-278 | 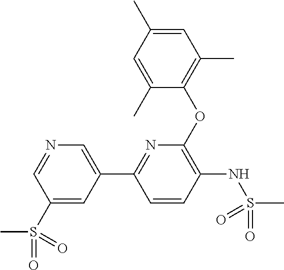 |
| I-279 | 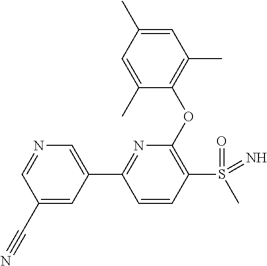 [SMILES: Cc1cc(C)c(Oc2nc(-c3cncc(C#N)c3)ccc2S(C)(=N)=O)c(C)c1] |
| I-280 | 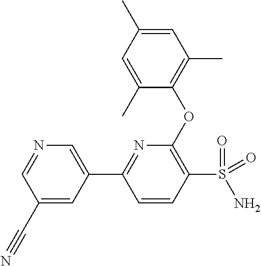 [SMILES: Cc1cc(C)c(Oc2nc(-c3cncc(C#N)c3)ccc2S(N)(=O)=O)c(C)c1] |
| I-281 | 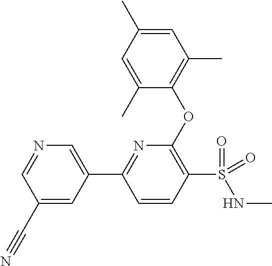 [SMILES: CNS(=O)(=O)c1ccc(-c2cncc(C#N)c2)nc1Oc1c(C)cc(C)cc1C] |
| I-282 | 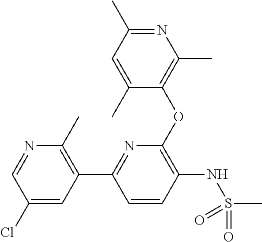 [SMILES: Cc1cc(C)c(Oc2nc(-c3cc(Cl)cnc3C)ccc2NS(C)(=O)=O)c(C)n1] |
| I-283 | 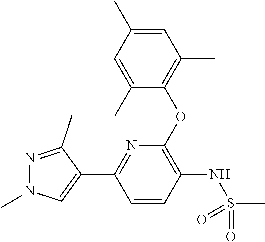 |
| I-284 | 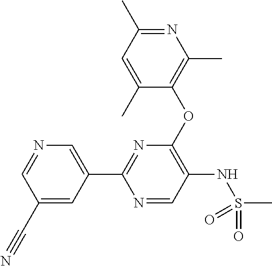 [SMILES: Cc1cc(C)c(Oc2nc(-c3cncc(C#N)c3)ncc2NS(C)(=O)=O)c(C)n1] |
| I-285 | 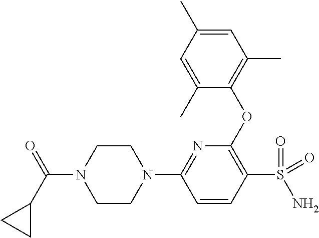 [SMILES: Cc1cc(C)c(Oc2nc(N3CCN(C(=O)C4CC4)CC3)ccc2S(N)(=O)=O)c(C)c1] |
| I-286 | 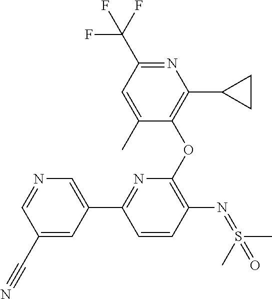 [SMILES: Cc1cc(C(F)(F)F)nc(C2CC2)c1Oc1nc(-c2cncc(C#N)c2)ccc1N=S(C)(C)=O] |
| I-287 | 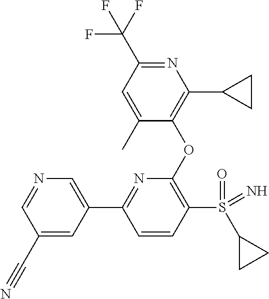 [SMILES: Cc1cc(C(F)(F)F)nc(C2CC2)c1Oc1nc(-c2cncc(C#N)c2)ccc1S(=N)(=O)C1CC1] |
| I-288 | 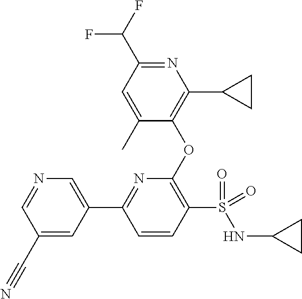 [SMILES: Cc1cc(C(F)F)nc(C2CC2)c1Oc1nc(-c2cncc(C#N)c2)ccc1S(=O)(=O)NC1CC1] |
| I-289 | 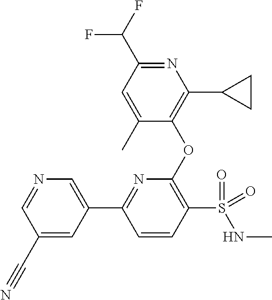 [SMILES: CNS(=O)(=O)c1ccc(-c2cncc(C#N)c2)nc1Oc1c(C)cc(C(F)F)nc1C1CC1] |
| I-290 | 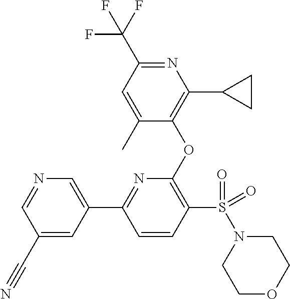 [SMILES: Cc1cc(C(F)(F)F)nc(C2CC2)c1Oc1nc(-c2cncc(C#N)c2)ccc1S(=O)(=O)N1CCOCC1] |
| I-291 | 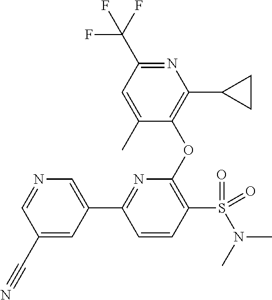 [SMILES: Cc1cc(C(F)(F)F)nc(C2CC2)c1Oc1nc(-c2cncc(C#N)c2)ccc1S(=O)(=O)N(C)C] |
| I-292 | 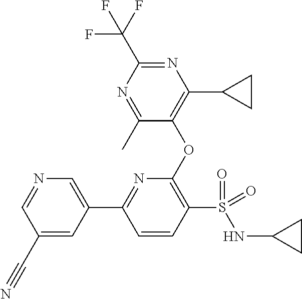 [SMILES: Cc1nc(C(F)(F)F)nc(C2CC2)c1Oc1nc(-c2cncc(C#N)c2)ccc1S(=O)(=O)NC1CC1] |
| I-293 | 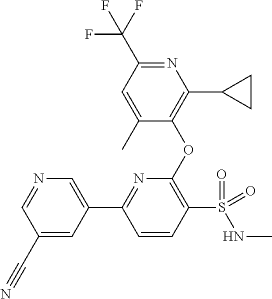 [SMILES: CNS(=O)(=O)c1ccc(-c2cncc(C#N)c2)nc1Oc1c(C)cc(C(F)(F)F)nc1C1CC1] |
| I-294 | 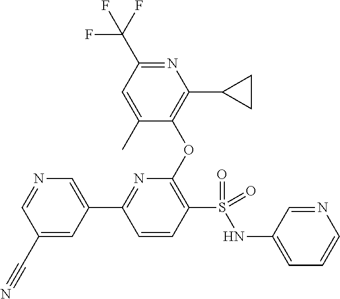 [SMILES: Cc1cc(C(F)(F)F)nc(C2CC2)c1Oc1nc(-c2cncc(C#N)c2)ccc1S(=O)(=O)Nc1cccnc1] |
| I-295 | 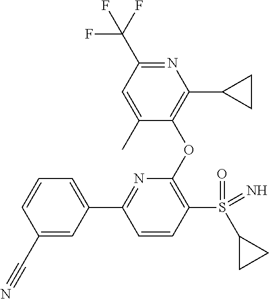 [SMILES: Cc1cc(C(F)(F)F)nc(C2CC2)c1Oc1nc(-c2cccc(C#N)c2)ccc1S(=N)(=O)C1CC1] |
| I-296 | 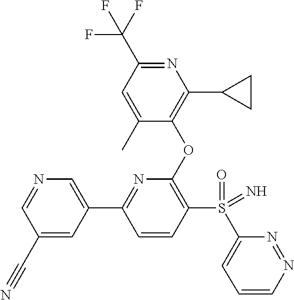 [SMILES: Cc1cc(C(F)(F)F)nc(C2CC2)c1Oc1nc(-c2cncc(C#N)c2)ccc1S(=N)(=O)c1cccnn1] |
| I-297 | 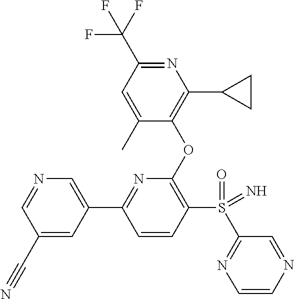 [SMILES: Cc1cc(C(F)(F)F)nc(C2CC2)c1Oc1nc(-c2cncc(C#N)c2)ccc1S(=N)(=O)c1cnccn1] |
| I-298 | 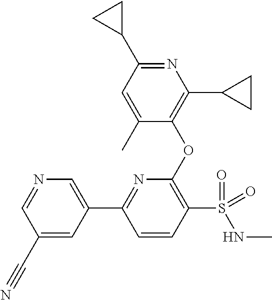 [SMILES: CNS(=O)(=O)c1ccc(-c2cncc(C#N)c2)nc1Oc1c(C)cc(C2CC2)nc1C1CC1] |
| I-299 | 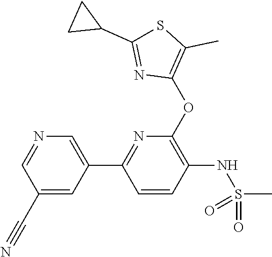 [SMILES: Cc1sc(C2CC2)nc1Oc1nc(-c2cncc(C#N)c2)ccc1NS(C)(=O)=O] |
| I-300 | 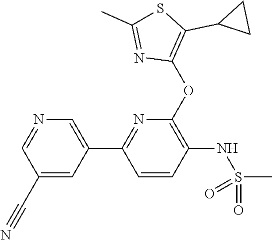 [SMILES: Cc1nc(Oc2nc(-c3cncc(C#N)c3)ccc2NS(C)(=O)=O)c(C2CC2)s1] |
| I-301 | 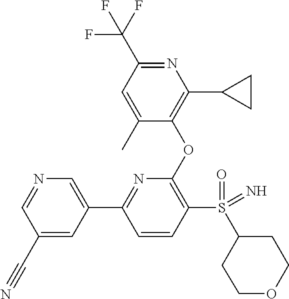 [SMILES: Cc1cc(C(F)(F)F)nc(C2CC2)c1Oc1nc(-c2cncc(C#N)c2)ccc1S(=N)(=O)C1CCOCC1] |
| I-302 | 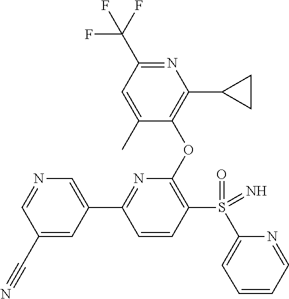 [SMILES: Cc1cc(C(F)(F)F)nc(C2CC2)c1Oc1nc(-c2cncc(C#N)c2)ccc1S(=N)(=O)c1ccccn1] |
| I-303 | 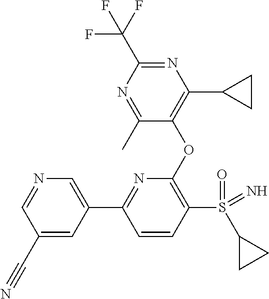 [SMILES: Cc1nc(C(F)(F)F)nc(C2CC2)c1Oc1nc(-c2cncc(C#N)c2)ccc1S(=N)(=O)C1CC1] |
| I-304 | 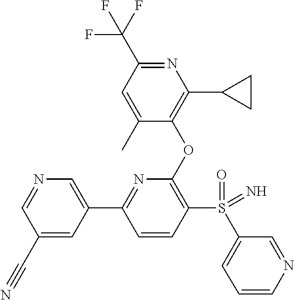 [SMILES: Cc1cc(C(F)(F)F)nc(C2CC2)c1Oc1nc(-c2cncc(C#N)c2)ccc1S(=N)(=O)c1cccnc1] |
| I-305 | 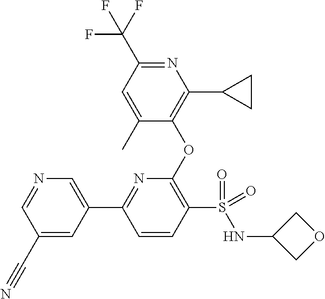 [SMILES: Cc1cc(C(F)(F)F)nc(C2CC2)c1Oc1nc(-c2cncc(C#N)c2)ccc1S(=O)(=O)NC1COC1] |
| I-306 | 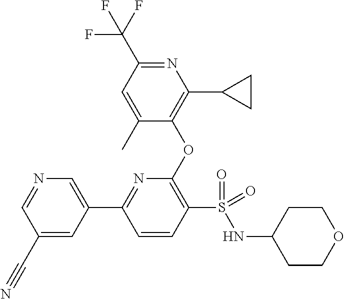 [SMILES: Cc1cc(C(F)(F)F)nc(C2CC2)c1Oc1nc(-c2cncc(C#N)c2)ccc1S(=O)(=O)NC1CCOCC1] |
| I-307 | 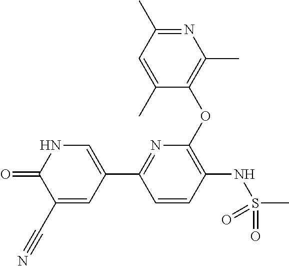 [SMILES: Cc1cc(C)c(Oc2nc(-c3c[nH]c(=O)c(C#N)c3)ccc2NS(C)(=O)=O)c(C)n1] |
| I-308 | 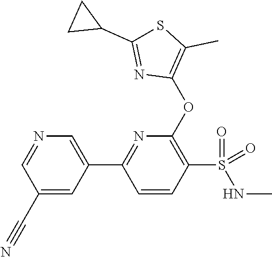 [SMILES: CNS(=O)(=O)c1ccc(-c2cncc(C#N)c2)nc1Oc1nc(C2CC2)sc1C] |
| I-309 | 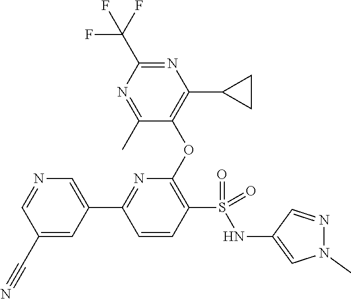 [SMILES: Cc1nc(C(F)(F)F)nc(C2CC2)c1Oc1nc(-c2cncc(C#N)c2)ccc1S(=O)(=O)Nc1cnn(C)c1] |
| I-310 | 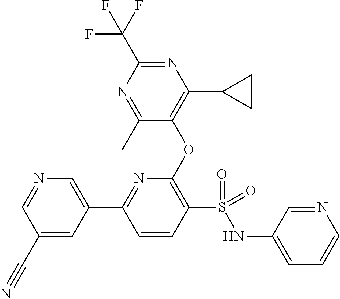 [SMILES: Cc1nc(C(F)(F)F)nc(C2CC2)c1Oc1nc(-c2cncc(C#N)c2)ccc1S(=O)(=O)Nc1cccnc1] |
| I-311 | 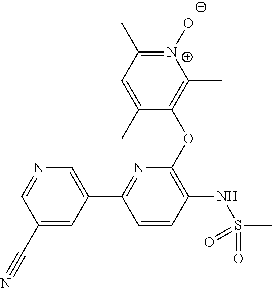 [SMILES: Cc1cc(C)[n+]([O-])c(C)c1Oc1nc(-c2cncc(C#N)c2)ccc1NS(C)(=O)=O] |
| I-312 | 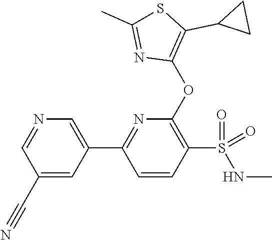 [SMILES: CNS(=O)(=O)c1ccc(-c2cncc(C#N)c2)nc1Oc1nc(C)sc1C1CC1] |
| I-313 | 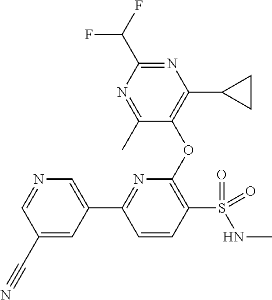 [SMILES: CNS(=O)(=O)c1ccc(-c2cncc(C#N)c2)nc1Oc1c(C)nc(C(F)F)nc1C1CC1] |
| I-314 | 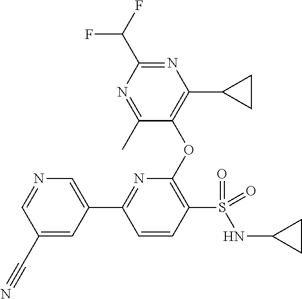 [SMILES: Cc1nc(C(F)F)nc(C2CC2)c1Oc1nc(-c2cncc(C#N)c2)ccc1S(=O)(=O)NC1CC1] |
| I-315 | 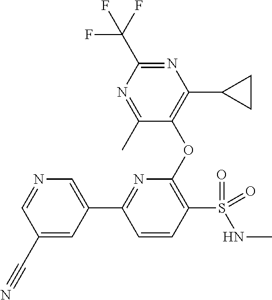 [SMILES: CNS(=O)(=O)c1ccc(-c2cncc(C#N)c2)nc1Oc1c(C)nc(C(F)(F)F)nc1C1CC1] |
| I-316 | 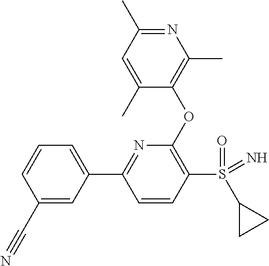 [SMILES: Cc1cc(C)c(Oc2nc(-c3cccc(C#N)c3)ccc2S(=N)(=O)C2CC2)c(C)n1] |
| I-317 | 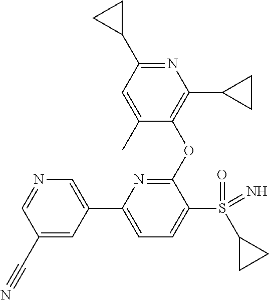 [SMILES: Cc1cc(C2CC2)nc(C2CC2)c1Oc1nc(-c2cncc(C#N)c2)ccc1S(=N)(=O)C1CC1] |
| I-318 | 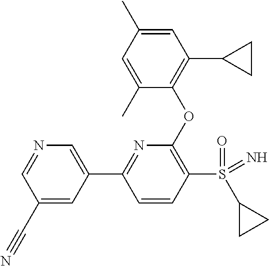 [SMILES: Cc1cc(C)c(Oc2nc(-c3cncc(C#N)c3)ccc2S(=N)(=O)C2CC2)c(C2CC2)c1] |
| I-319 | 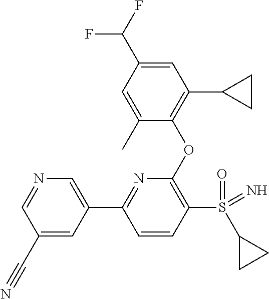 [SMILES: Cc1cc(C(F)F)cc(C2CC2)c1Oc1nc(-c2cncc(C#N)c2)ccc1S(=N)(=O)C1CC1] |
| I-320 | 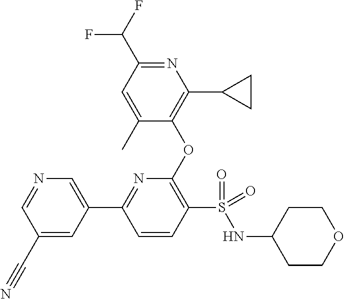 [SMILES: Cc1cc(C(F)F)nc(C2CC2)c1Oc1nc(-c2cncc(C#N)c2)ccc1S(=O)(=O)NC1CCOCC1] |
| I-321 | 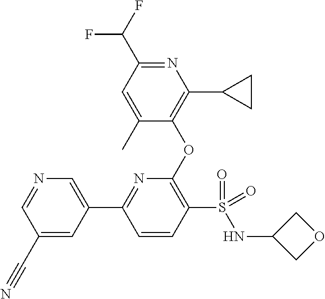 [SMILES: Cc1cc(C(F)F)nc(C2CC2)c1Oc1nc(-c2cncc(C#N)c2)ccc1S(=O)(=O)NC1COC1] |
| I-322 | 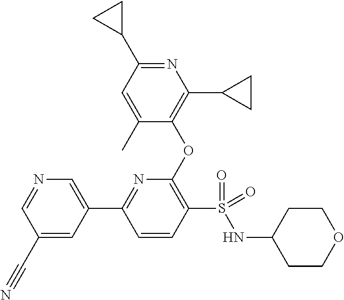 [SMILES: Cc1cc(C2CC2)nc(C2CC2)c1Oc1nc(-c2cncc(C#N)c2)ccc1S(=O)(=O)NC1CCOCC1] |
| I-323 | 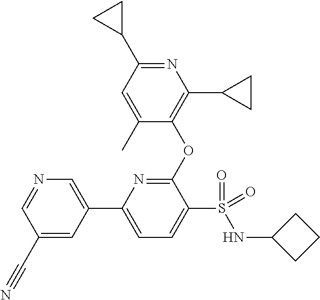 [SMILES: Cc1cc(C2CC2)nc(C2CC2)c1Oc1nc(-c2cncc(C#N)c2)ccc1S(=O)(=O)NC1CCC1] |
| I-324 | 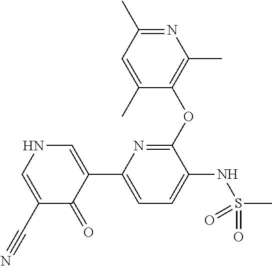 [SMILES: Cc1cc(C)c(Oc2nc(-c3c[nH]cc(C#N)c3=O)ccc2NS(C)(=O)=O)c(C)n1] |
| I-325 | 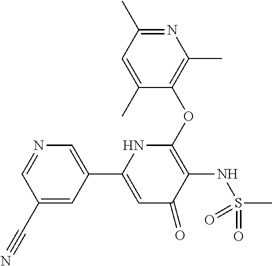 [SMILES: Cc1cc(C)c(Oc2[nH]c(-c3cncc(C#N)c3)cc(=O)c2NS(C)(=O)=O)c(C)n1] |
| I-326 | 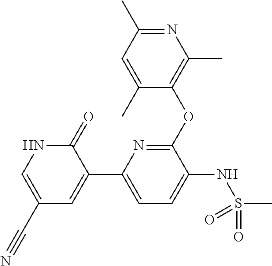 [SMILES: Cc1cc(C)c(Oc2nc(-c3cc(C#N)c[nH]c3=O)ccc2NS(C)(=O)=O)c(C)n1] |
| I-327 | 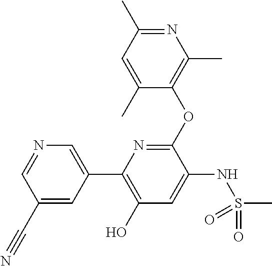 [SMILES: Cc1cc(C)c(Oc2nc(-c3cncc(C#N)c3)c(O)cc2NS(C)(=O)=O)c(C)n1] |

In certain embodiments, the present invention provides a compound of formula I as defined above, or a pharmaceutically acceptable salt thereof, or a pharmaceutical composition comprising a compound of formula I as defined above, or a pharmaceutically acceptable salt thereof, and a pharmaceutically acceptable carrier, adjuvant, or vehicle for use as a medicament.

In certain embodiments, the present invention provides a pharmaceutical composition comprising a compound set forth in Table 1 above, or a pharmaceutically acceptable salt thereof, together with a pharmaceutically acceptable carrier, excipient, or diluent.

### II. Therapeutic Applications

In certain embodiments, the present invention provides a method of treating a disorder in which enhanced ABC transporter function is of clinical benefit. For instance, in certain embodiments, a disorder is one in which ABC transporter dysfunction is etiological for disease. In certain embodiments, correction of one or more underlying mutations associated with ABC transporter dysfunction is rationalized. In certain embodiments, methods of the present invention provide enhancement of one or more non-mutated forms of an ABC transporter.

In certain embodiments, the invention provides a method of treating a disorder in which enhanced ABC transporter function is of clinical benefit, wherein the method comprises administering to a subject in need thereof a therapeutically effective amount of a compound described herein, such as a compound of Formula I or Formula II, to treat the disorder in which enhanced ABC transporter function is of clinical benefit. In certain embodiments, the particular compound of Formula I or Formula II is a compound defined by one of the embodiments described in Section I, above.

In certain embodiments, the invention provides a method of treating a disorder associated with ABC transporter dysfunction, wherein the method comprises administering to a subject in need thereof a therapeutically effective amount of a compound described herein, such as a compound of Formula I or Formula II, to treat the disorder associated with ABC transporter dysfunction. In certain embodiments, the particular compound of Formula I or Formula II is a compound defined by one of the embodiments described in Section I, above.

In certain embodiments, the present invention provides a method of modulating function of an ABC transporter in a subject, comprising administering to the subject an effective amount of a compound of Formula I or Formula II to thereby modulate function of the ABC transporter in the subject. In certain embodiments, the ABC transporter is selected from one or more of ABCA1, ABCA2, ABCA3, ABCA4, ABCA5, ABCA7, ABCA12, ABCB2, ABCB3, ABCB4, ABCB6, ABCB7, ABCB10, ABCB11, ABCC1, ABCC2, ABCC4, ABCC5, ABCC6 ABCC7, ABCC8, ABCC9, ABCC12, ABCD1, ABCD2, ABCD3, ABCD4, ABCG5, ABCG8, ABCG1, and ABCG4.

Another aspect of the invention provides a method of increasing the expression of a protein selected from ABCB11, ABCB11 E297G, ABCC6, ABCB4, ABCA4 P1380L, and ABCD2 in a subject. Another aspect of the invention provides a method of increasing the expression of a protein selected from ABCB11, ABCB11 E297G, ABCC6, ABCB4, ABCA4 P1380L, ABCD1, and ABCD2 in a subject. The method comprises administering to a subject in need thereof an effective amount of a compound described herein, such as a compound of Formula I or Formula II, to thereby increase the expression of said protein, as further described in the detailed description.

In certain embodiments, the present invention provides a method of alleviating one or more symptoms of a disorder associated with ABC transporter dysfunction, wherein the method comprises administering to a subject in need thereof a therapeutically effective amount of a compound described herein, such as a compound of Formula I or Formula II, to treat the disorder associated with ABC transporter dysfunction. In certain embodiments, the particular compound of Formula I or Formula II is a compound defined by one of the embodiments described in Section I, above.

In certain embodiments, the disorder associated with ABC transporter dysfunction is characterized by dysfunction in a transporter selected from one or more of ABCA1, ABCA2, ABCA3, ABCA4, ABCA5, ABCA7, ABCA12, ABCB2, ABCB3, ABCB4, ABCB6, ABCB7, ABCB10, ABCB11, ABCC1, ABCC2, ABCC4, ABCC5, ABCC6, ABCC7, ABCC8, ABCC9, ABCC12, ABCD1, ABCD2, ABCD3, ABCD4, ABCG5, ABCG8, ABCG1, and ABCG4.

In certain embodiments, the present invention provides methods of treating a disorder selected from Tangier disease, Surfactant metabolism dysfunction pulmonary 3, autosomal recessive Ichthyosis congenital 4A (ARCI), Bare lymphocyte syndrome type I, Bare lymphocyte syndrome type I due to TAP2 deficiency, Dyschromatosis universalis hereditaria 3, X-linked sideroblastic anemia with ataxia, Dubin-Johnson Syndrome, Cystic fibrosis (CF), Familial Hyperinsulinemic Hypoglycemia 1, Intellectual disability Myopathy Syndrome, Congenital bile acid synthesis defect 5, Methylmalonic aciduria and homocystinuria cblJ type, Sitostrolemia, Stargardt disease, PFIC3, PFIC2, Pseudoxanthoma Elasticum, X-linked adrenoleukodystrophy (ALD), Cholestasis, Hyperbilirubinemia, Intrahepatic cholestasis of pregnancy, Biliary atresia, Alagille syndrome, primary biliary cholangitis, primary sclerosing cholangitis, NAFLD/NASH, Alzheimer's disease, Huntington's disease, Multiple sclerosis, Parkinson's disease, Hirschsprung disease, Zellweger syndrome, Type 2 diabetes, Obesity, Type 1 diabetes, Atherosclerosis, Dyslipidemia, Generalized arterial calcification of infancy, Calciphylaxis, Autosomal recessive cone-rod dystrophy, Gout, PFIC1, Myo5B deficiency cholestasis, PFIC4, Low phospholipid associated cholelithiasis, intrahepatic microlithiasis, hepatolithiasis, Non-anastomotic biliary strictures, Benign recurrent intrahepatic cholestasis, vascular calcification, vascular calcification associated with CKD, vascular calcification associated with T2D, and Calcific uremic atreriolopathy.

In certain embodiments, the present invention provides methods of treating or preventing a disorder selected from Tangier disease, Surfactant metabolism dysfunction pulmonary 3, autosomal recessive Ichthyosis congenital 4A (ARCI), Bare lymphocyte syndrome type I, Bare lymphocyte syndrome type I due to TAP2 deficiency, Dyschromatosis universalis hereditaria 3, X-linked sideroblastic anemia with ataxia, Dubin-Johnson Syndrome, Cystic fibrosis (CF), Familial Hyperinsulinemic Hypoglycemia 1, Intellectual disability Myopathy Syndrome, Congenital bile acid synthesis defect 5, Methylmalonic aciduria and homocystinuria cblJ type, Sitostrolemia, Stargardt disease, PFIC3, PFIC2, Pseudoxanthoma Elasticum, X-linked adrenoleukodystrophy (ALD), Cholestasis, Hyperbilirubinemia, Intrahepatic cholestasis of pregnancy, Biliary atresia, Alagille syndrome, primary biliary cholangitis, primary sclerosing cholangitis, NAFLD/NASH, Alzheimer's disease, Huntington's disease, Multiple sclerosis, Parkinson's disease, Hirschsprung disease, Zellweger syndrome, Type 2 diabetes, Obesity, Type 1 diabetes, Atherosclerosis, Dyslipidemia, Generalized arterial calcification of infancy, Calciphylaxis, Autosomal recessive cone-rod dystrophy, Gout, PFIC1, Myo5B deficiency cholestasis, PFIC4, and Low phospholipid associated cholelithiasis.

In certain embodiments, the present invention provides methods of treating or preventing a disorder selected from Tangier disease, Surfactant metabolism dysfunction pulmonary 3, autosomal recessive Ichthyosis congenital 4A (ARCI), Bare lymphocyte syndrome type I, Bare lymphocyte syndrome type I due to TAP2 deficiency, Dyschromatosis universalis hereditaria 3, X-linked sideroblastic anemia with ataxia, Dubin-Johnson Syndrome, Cystic fibrosis (CF), Familial Hyperinsulinemic Hypoglycemia 1, Intellectual disability Myopathy Syndrome, congenital bile acid synthesis defect 5, Methylmalonic aciduria and homocystinuria cblJ type, Sitostrolemia, Stargardt disease, PFIC3, PFIC2, Pseudoxanthoma Elasticum, X-linked adrenoleukodystrophy (ALD), Cholestasis, Hyperbilirubinemia, Intrahepatic cholestasis of pregnancy, Biliary atresia, Alagille syndrome, primary biliary cholangitis, primary sclerosing cholangitis, NAFLD/NASH, Alzheimer's disease, Huntington's disease, Multiple sclerosis, Parkinson's disease, Hirschsprung disease, Zellweger syndrome, Type 2 diabetes, Obesity, Type 1 diabetes, Atherosclerosis, Dyslipidemia, generalized arterial calcification of infancy, calciphylaxis, Autosomal recessive cone-rod dystrophy, Gout, PFIC1, Myo5B deficiency cholestasis, PFIC4, low phospholipid associated cholelithiasis, chronic kidney disease, Progeria (Hutchinson-Gilford progeria syndrome), hemodialysis, end stage renal disease, aortic stenosis, peripheral arterial disease, or ischemic stroke.

In certain embodiments, the present invention provides methods of treating a disorder selected from calciphylaxis, pseudoxanthoma elasticum, generalized arterial calcification of infancy, chronic kidney disease, Progeria (Hutchinson-Gilford progeria syndrome), hemodialysis, end stage renal disease, aortic stenosis, peripheral arterial disease, or ischemic stroke.

In certain embodiments, the disorder associated with ABC transporter dysfunction is cystic fibrosis (CF). Accordingly, in certain embodiments, the present invention provides a method of treating cystic fibrosis, wherein the method comprises administering to a subject in need thereof a therapeutically effective amount of a compound described herein, such as a compound of Formula I or Formula II. In some such embodiments, the method further comprises administering one or more additional therapeutic agents, described further below and herein.

In certain embodiments, the disorder associated with ABC transporter dysfunction is cholestasis. Accordingly, in certain embodiments, the present invention provides a method of treating cholestasis, wherein the method comprises administering to a subject in need thereof a therapeutically effective amount of a compound described herein, such as a compound of Formula I or Formula II. One of skill in the medical arts will recognize that there are various forms of cholestasis, all of which are contemplated herein for treatment with methods and compounds of the present invention. In certain embodiments, the cholestasis is intrahepatic. In certain embodiments, the cholestasis is extrahepatic. In certain embodiments, the cholestasis is any of those described above and herein.

### Subjects

In certain embodiments, the subject is a human. In certain embodiments, the subject is an adult human. In certain embodiments, the subject is a pediatric human.

### Medical Uses

Another aspect of the invention provides for the use of a compound described herein (such as a compound of Formula I or Formula II, or other compounds in Section I) in the manufacture of a medicament. In certain embodiments, the medicament is for treating a disorder described herein, such as a disorder associated with ABC transporter dysfunction. Exemplary such disorders are described above and herein.

Another aspect of the invention provides for the use of a compound described herein (such as a compound of Formula I or Formula II, or other compounds in Section I) for treating a medical disorder, such as disorder associated with ABC transporter dysfunction. Exemplary such disorders are described above and herein.

### III. Combination Therapy

Another aspect of the invention provides for combination therapy. Compounds described herein (such as a compound of Formula I or Formula II, or other compounds in Section I) or their pharmaceutically acceptable salts may be used in combination with additional therapeutic agents to treat medical disorders, such as an autoimmune disorder, cancer, etc.

In certain embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In certain embodiments, the method includes co-administering one additional therapeutic agent. In certain embodiments, the method includes co-administering two additional therapeutic agents. In certain embodiments, the combination of the disclosed compound and the additional therapeutic agent or agents acts synergistically.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 19, 20, 21, 22, 23, or 24 hours from one another. In certain embodiments, one or more other therapeutic agent and a compound or composition of the invention are administered as a multiple dosage regimen more than 24 hours apart.

In certain embodiments, the present invention provides a method of treating cystic fibrosis (CF) comprising administering a compound of the present invention with one or more additional therapeutic agents. In certain embodiments, the one or more additional therapeutic agents are selected from a mucolytic agent, a bronchodialator, an antibiotic, an anti-infective agent, an anti-inflammatory agent, a cystic fibrosis transmembrane conductance (CFTR) modulator, a nutritional agent, or any agent known to treat CF.

In some embodiments, the one or more additional therapeutic agents is an antibiotic. In some embodiments the antibiotic is selected from a penicillin, a cephalosporin, a tetracycline, a macrolide, a fluoroquinolone, a sulfonamide, a glycopeptide, or a rifamycin. In certain embodiments, the antibiotic is selected from phenoxymethyl penicillin, dicloxacillin, amoxicillin with clavulanic acid, ampicillin, nafcillin, oxacillin, penicillin V, penicillin G, cefaclor, cefazolin, cefadroxil, cephalexin, cefuroxime, cefixime, cefoxitin, ceftriaxone, doxycycline, minocycline, sarecycline, erythromycin, clarithromycin, azithromycin, fidaxomicin, roxithromycin, ciprofloxacin, ofloxacin, levofloxacin, moxifloxacin, sulfamethoxazole with trimethoprim, sulfasalazine, sulfacetamide, sulfadiazine silver, vancomycin, dalbavancin, oritavancin, telavancin, and rifaximin.

In certain embodiments, the one or more additional therapeutic agents is an S-nitrosoglutathione reductase (GSNOR) inhibitor. In certain embodiments, the GSNOR inhibitor is selected from a GSNOR inhibitor disclosed in WO2010/019903, U.S. Pat. Nos. 8,470,857, 8,642,628, WO2010/019910, U.S. Pat. No. 8,586,624, WO2011/100433, U.S. Pat. No. 8,481,590, WO2012/048181, WO2012/083165, WO2012/083171, or WO 2012/170371.

In certain embodiments, the one or more additional therapeutic agents is an ileal bile transport (IBAT) inhibitor. In certain embodiments, the IBAT inhibitor is selected from an IBAT inhibitor disclosed in AU2011326873, US2020/0330545, WO2012/064266, WO2020/167964, or Front. Pharmacol. 2018; 9: 931 (Al-Dury et al., published online Aug. 21, 2018). Exemplary IBAT inhibitors include, but are not limited to odevixibat, elobixivat, maralixibat, linerixibat, GSK2330672, SHP626 (volixibat), A4250, etc.

In some embodiments, the one or more additional therapeutic agents is an ileal bile transport (IBAT) inhibitor. In some embodiments, the IBAT inhibitor is selected from an IBAT inhibitor disclosed in AU2011326873, US2020/0330545, WO2012/064266, WO2020/167964, or Front. Pharmacol. 2018; 9: 931 (Al-Dury et al., published online Aug. 21, 2018). Exemplary IBAT inhibitors include, but are not limited to odevixibat, elobixivat, maralixibat, linerixibat, GSK2330672, SHP626 (volixibat), A4250, etc. In some embodiments, the one or more additional therapeutic agents is selected from odevixibat, elobixivat, maralixibat, linerixibat, GSK2330672, SHP626 (volixibat), and A4250.

In some embodiments, the one or more additional therapeutic agents is a peroxisome proliferator-activated receptors (PPAR) agonist. In some embodiments, the one or more additional therapeutic agents is a dual PPAR agonist, a PPAR-α agonist, a PPAR-γ agonist, or a PPAR-δ agonist. In some embodiments, the one or more additional therapeutic agents is a PPAR-α agonist. In some embodiments, the one or more additional therapeutic agents is a PPAR-γ agonist. In some embodiments, the one or more additional therapeutic agents is a PPAR-δ agonist. In some embodiments, the PPAR agonist is selected from elafibrinor, clofibrate, gemfibrozil, ciprofibrate, bezafibrate, fenofibrate, GW-9662, GW501516, GFT1007, aleglitazar, muraglitazar, tesaglitazar, saroglitazar, and seladelpar.

In some embodiments, the one or more additional therapeutic agents is an HMG-CoA reductase inhibitor. In some embodiments, the HMG-CoA reductase inhibitor is a statin. In certain embodiments, the statin is selected from atorvastatin, fluvastatin, lovastatin, pitavastatin, pravastatin, rosuvastatin, and simvastatin.

In some embodiments, the one or more additional therapeutic agents is a disease-modifying antirheumatic drug (DMARD). In certain embodiments the DMARD is selected from azathioprine, hydroxychloroquine, leflunomide, methotrexate, and sulfasalazine

In some embodiments, the one or more additional therapeutic agents is DNA methyltransferase inhibitor. In some embodiments, the DNA methyltransferase inhibitor is selected from azacitidine, decitabine, zebularine, hydralazine, procaine, MG98, genistein, bobcat339 hydrochloride, hinokitiol, CM-272, and larsucosterol.

In some embodiments, the one or more additional therapeutic agents is an anti-interleukin-17A biological agent. In some embodiments, the anti-interleukin-17A biological agent is selected from secukinumab, ixekizumab, bimekizumab, brodalumab, and nekalimumab.

In some embodiments, the one or more additional therapeutic agents is an anti-interleukin-23 inhibitor. In certain embodiments the anti-interleukin-23 inhibitor is selected from guselkumab, risankizumab, tildrakizumab, and ustekinumab.

In some embodiments, the one or more additional therapeutic agents is a neutrophil elastase inhibitor. In some embodiments, the neutrophil elastase inhibitor is selected from sivelestat sodium hydrate, AvKTI, and a flavonoid.

In some embodiments, the one or more additional therapeutic agents is a corticosteroid. In certain embodiments, the corticosteroid is selected from flugestone, fluorometholone, medrysone, prebediolone acetate, chloroprednisone, cloprednol, difluprednate, fludrocortisone, fluocinolone, fluperolone, fluprednisolone, loteprednol, methylprednisolone, prednicarbate, prednisolone, prednisone, tixocortol, triamcinolone, dexamethasone, alclometasone, beclometasone, betamethasone, clobetasol, clobetasone, clocortolone, desoximetasone, dexamethasone, diflorasone, difluocortolone, fluclorolone, flumetasone, fluocortin, fluocortolone, fluprednidene, fluticasone, fluticasone furoate, halometasone, meprednisone, mometasone, mometasone furoate, paramethasone, prednylidene, rimexolone, ulobetasol, amcinonide, budesonide, ciclesonide, deflazacort, desonide, formocortal, fluclorolone acetonide, fludroxycortide, flunisolide, fluocinolone acetonide, fluocinonide, halcinonide, triamcinolone acetonide, cortivazol, and RU-28362.

In some embodiments, the one or more additional therapeutic agents is a protein arginine deiminase 4 (PAD4) inhibitor. In certain embodiments, the PAD4 inhibitor is selected from JBI-589, GSK484, Cl-Amidine, Azithromycin, Clindamycin, Leflunomide, and Methotrexate.

In some embodiments, the one or more additional therapeutic agents is an apical sodium-dependent BA transporter (ASBT) inhibitor. In some embodiments, the ASBT inhibitor is A3907.

In some embodiments, the one or more additional therapeutic agents is a thyroid hormone receptor beta (THR-β) agonist. In some embodiments, the THR-β) agonist is selected from resmetirom, VK2809, and cs27109.

In some embodiments, the one or more additional therapeutic agents is an immune checkpoint inhibitor. In certain embodiments, the immune checkpoint inhibitor is selected from pembrolizumab, nivolumab, cemiplimab, ipilimumab, atezolizumab, avelumab, and durvalumab.

In some embodiments, the one or more additional therapeutic agents is a bile acid conjugate. In certain embodiments, the bile acid conjugate is berberine ursodeoxycholate, ursodeoxycholate, and nor-ursodeoxycholate.

In some embodiments, the one or more additional therapeutic agents is an integrin inhibitor. In some embodiments, the integrin is a subtype selected from α5β1, α8β1, αvβ1, αvβ3, αvβ5, αvβ6, αvβ8, and αIIbβ3. In some embodiments, the integrin inhibitor is selected from bexotegrast, natalizumab, vedolizumab, PLN-1474, risuteganib, THR-687, OT-166, AXT107, tirofiban, eptifibatide, abciximab, MORF-057, 7HP349, efalizumab, and lifitegrast.

In some embodiments, the one or more additional therapeutic agents is an endocrine fibroblast growth factor (FGF) analog. In some embodiments, the endocrine FGF analog is selected from FGF19, FGF21 and FGF23. In some embodiments, the endocrine FGF analog is aldafermin.

In some embodiments, the one or more additional therapeutic agents is a monoclonal antibody. In some embodiments, the monoclonal antibody targets CCL24, lysyl oxidase-like 2 (LOXL2), or vascular adhesion protein (VAP). In some embodiments, the monoclonal antibody is CM-101, simtuzumab, or BTT1023.

In some embodiments, the one or more additional therapeutic agents is an antagonist of MAS Related GPR Family Member X4 (MRGPRX4). In some embodiments, the monoclonal antibody is EP547.

In some embodiments, the one or more additional therapeutic agents is an apical sodium-dependent bile acid transporter (ASBT) inhibitor. In some embodiments, the ASBT inhibitor is selected from resveratrol, elobixibat, A4250, 264W94, 216U90, GSK2330672, lopixibat, SC-435, S-1647, IMB17-15, baribixibat, S-8921, S-8921G, R-146224, BRL-39924A, S0960volixibat and ritivixibat.

In some embodiments, the one or more additional therapeutic agents is a farnesoid X receptor (FXR) agonist. In some embodiments, the FXR agonist is selected from OCA, CS0159, tropifexor, vonafexor, and cilofexor.

In some embodiments, the one or more additional therapeutic agents is a glucagon-like peptide-1 receptor agonists (GLP-1RA). In some embodiments, the GLP-1RA is selected from dulaglutide, exenatide, liraglutide, liraglutide/insulin degludec, lixisenatide/insulin glargine, semaglutide, and tirzepatide.

In some embodiments, the one or more additional therapeutic agents is a bile acid or analog thereof. In some embodiments, the bile acid or analog thereof is selected from ursodeoxycholic acid and 24-norursodeoxycholic acid.

In some embodiments, the one or more additional therapeutic agents is a dihydroorotate dehydrogenase inhibitor. In some embodiments, the dihydroorotate dehydrogenase inhibitor is selected from brequinar sodium, ASLAN003, ML390, BAY2402234, PTC299, letlunomuide, vidofludimus calcium, teriflunomide, and IMU-838.

In some embodiments, the one or more additional therapeutic agents is sodium thiosulfate.

In some embodiments, the one or more additional therapeutic agents is a calcium reducer. In some embodiments, the one or more additional therapeutic agents is cinacalcet.

In some embodiments, the one or more additional therapeutic agents is a phosphorous reducer. In some embodiments, the one or more additional therapeutic agents is sevelamer.

In some embodiments, the one or more additional therapeutic agents is an enzyme. In some embodiments, the one or more additional therapeutic agents is a recombinant human ENPP1 protein. In some embodiments, the one or more additional therapeutic agents is INZ-701.

In some embodiments, the one or more additional therapeutic agents is a TNAP inhibitor. In some embodiments, the TNAP inhibitor is selected from MLS-0038949, Levamisole, and DS-1211.

In some embodiments, the one or more additional therapeutic agents is a GLP1 agonist or a GLP1 dual agonist. In some embodiments, the GLP1 agonist or GLP1 dual agonist is selected from Exenatide (Byetta, Bydureon), Liraglutide (Victoza, Saxenda), Albiglutide (Tanzeum), Dulaglutide (Trulicity), Semaglutide (Ozempic, Rybelsus), and Tirzepatide (Mounjaro).

In some embodiments, the one or more additional therapeutic agents is a SGLT2 inhibitor. In some embodiments, the SGLT2 inhibitor is selected from Canagliflozin, Dapagliflozin, Empagliflozin, Ertugliflozin, Bexagliflozin, and Sotagliflozin.

In some embodiments, the one or more additional therapeutic agents is an anti-coagulant. In some embodiments, the anti-coagulant is selected from apixaban (Eliquis), dabigatran (Pradaxa), edoxaban (Lixiana), rivaroxaban (Xarelto), and warfarin (Coumadin).

In some embodiments, the one or more additional therapeutic agents is a statin. In some embodiments, the statin is selected from Atorvastatin (Atorvaliq, Lipitor), Fluvastatin (Lescol), Lovastatin, Pitavastatin (Livalo), Pravastatin (Pravachol), Rosuvastatin calcium (Crestor), Simvastatin (Zocor).

In some embodiments, the one or more additional therapeutic agents is an angiotensin-converting enzyme inhibitors. In some embodiments, the angiotensin-converting enzyme inhibitor is selected from Benazepril (Lotensin), Captopril (Capoten), Enalapril (Vasotec), Enalaprilat (Vasotec), Fosinopril (Monopril), Lisinopril (Zestril, Prinivil), Moexipril (Univasc), Perindopril (Coversyl), Quinapril (Accupril), Ramipril (Altace), and Trandolapril (Mavik).

In some embodiments, the one or more additional therapeutic agents is a renin-angiotensin inhibitor. In some embodiments, the renin-angiotensin inhibitor is selected from aliskiren, ciprokiren, ditekiren, enalkiren, remikiren, rasilez, terlakiren, and zankiren.

In some embodiments, the one or more additional therapeutic agents is an aldosterone inhibitor. In some embodiments, the aldosterone inhibitor is selected from spironolactone systemic, eplerenone systemic, and finerenone systemic.

### Additional Considerations

The doses and dosage regimen of the active ingredients used in the combination therapy may be determined by an attending clinician. In certain embodiments, the compound described herein (such as a compound of Formula I or Formula II, or other compounds in Section I) and the additional therapeutic agent(s) are administered in doses commonly employed when such agents are used as monotherapy for treating the disorder. In other embodiments, the compound described herein (such as a compound of Formula I or Formula II, or other compounds in Section I) and the additional therapeutic agent(s) are administered in doses lower than the doses commonly employed when such agents are used as monotherapy for treating the disorder. In certain embodiments, the compound described herein (such as a compound of Formula I or Formula II, or other compounds in Section I) and the additional therapeutic agent(s) are present in the same composition, which is suitable for oral administration.

In certain embodiments, the compound described herein (such as a compound of Formula I or Formula II, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of the compound described herein (such as a compound of Formula I or Formula II, or other compounds in Section I), a pharmaceutically acceptable carrier, vehicle or diluent, and optionally at least one additional therapeutic agent listed above.

### IV. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (such as a compound of Formula I or Formula II, or other compounds in Section I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a compound described herein in a therapeutically effective amount for the treatment of a medical disorder described herein.

### EXAMPLES

The invention now being generally described, will be more readily understood by reference to the following examples, which are included merely for purposes of illustration of certain aspects and embodiments of the present invention, and is not intended to limit the invention.

### Synthetic Methods

Methods for preparing compounds described herein are illustrated in the following synthetic Scheme. The Scheme is given for the purpose of illustrating the invention, and not intended to limit the scope or spirit of the invention. Starting materials shown in the Scheme can be obtained from commercial sources or can be prepared based on procedures described in the literature.

In the Schemes, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in “Protecting Groups in Organic Synthesis”, T. W. Greene and P. G. M. Wuts, 3^{rd} edition, John Wiley & Sons, 1999, the entire contents of which are hereby incorporated by reference. The modular synthetic route illustrated in Scheme 1 can also be readily modified by one of skill in the art to provide additional compounds by conducting functional group transformations on the intermediate and final compounds. Such functional group transformations are well known in the art, as described in, for example, “Comprehensive Organic Synthesis” (B. M. Trost & I. Fleming, eds., 1991-1992).

The abbreviation “TFA” refers to trifluoroacetic acid. The abbreviation “FA” refers to formic acid.

### Preparative HPLC Methods:

### HPLC Method 1:

Instrument: Shimadzu LCMS system equipped with UV and MS detector
Mobile Phase: 0.225% formic acid in water (solvent A) and CH₃CN (solvent B),
Gradient: using the elution gradient 30%-70% (solvent B) over 10 minutes.
Flow rate: 35 ml/min;
Column: Phenomenex Gemini-NX 150*30 mm*5 um
Wavelength: UV 220 nm and 254 nm)
Column temperature: 30° C.;
MS ionization: ESI

### HPLC Method 2:

Instrument: GILSON 215 and Shimadzu LCMS 2010A
Column: Agela Durashell C18 250*20 mm*5 um
Mobile phase A: 0.1% ammonium bicarbonate in water (m/m)
Mobile phase B: Acetonitrile
Gradient: 80% A/20% B linear to 60% A/40% B in 10.0 min, hold at 0% A/100% B to 12.0 min.
Flow rate: 35 mL/min or 30 mL/min

### HPLC Method 3:

Instrument: Shimadzu LCMS system equipped with DAD detector
Mobile Phase: 1.5 mL/4 L TFA in water (solvent A) and 0.75 mL/4 L TFA in CH₃CN (solvent B),
Gradient: using the elution gradient 5%-95% (solvent B) over 0.7 minutes and holding at 95%
for 0.4 minutes at a flow rate of 1.5 ml/min;
Column: Chromolish.Flash RP-18e 25-2 mm
Wavelength: UV 220 nm (or 215 nm, 254 nm)
Column temperature: 50° C.;
MS ionization: ESI

### HPLC Method 4A:

Instrument: Gilson-GX281
Mobile Phase: H₂O (0.1% TFA) (solvent A) and CH₃CN (solvent B),
Gradient: using the elution gradient 5%-95% (solvent B) over 25 minutes.
Flow rate: 25 ml/min;
Column: Nanomicro-UniSil 5-120 C18, 5 um, 30*150 mm
Wavelength: UV 220 nm and 254 nm
Column temperature: 25° C.;
MS ionization: ESI.

### HPLC Method 4B:

Instrument: Gilson-GX281
Mobile Phase: H₂O (0.1% FA) (solvent A) and CH₃CN (solvent B),
Gradient: using the elution gradient 5%-95% (solvent B) over 25 minutes.
Flow rate: 25 ml/min;
Column: YMC-Actus Triart C18 250*20 mm*5 um
Wavelength: UV 220 nm and 254 nm
Column temperature: 25° C.;
MS ionization: ESI.

### HPLC Method 4C:

Instrument: Gilson-GX281
Mobile Phase: H₂O (0.1% TFA) (solvent A) and CH₃CN (solvent B),
Gradient: using the elution gradient 5%-95% (solvent B) over 25 minutes.
Flow rate: 25 ml/min;
Column: YMC-Actus Triart C18 250*20 mm*5 um
Wavelength: UV 220 nm and 254 nm
Column temperature: 25° C.;
MS ionization: ESI.

### HPLC Method 4D:

Instrument: LH-40
Mobile Phase: H₂O (0.10% NH₄HCO₃) (solvent A) and CH₃CN (solvent B),
Gradient: using the elution gradient 20%-95% (solvent B) over 15 minutes.
Flow rate: 18 ml/min;
Column: YMC-Actus Triart C18 250*20 mm*5 um
Wavelength: UV 220 nm and 254 nm)
Column temperature: 25° C.;
MS ionization: ESI.

### HPLC Method 5A:

Instrument: LC-8A
Mobile Phase: H₂O (0.1% FA) (solvent A) and CH₃CN (solvent B),
Gradient: using the elution gradient 20%-95% (solvent B) over 18 minutes.
Flow rate: 18 ml/min;
Column: Metasil Cloak C18 21.2*250 mm, 5 um
Wavelength: UV 220 nm and 254 nm
Column temperature: 25° C.;
MS ionization: ESI.

### HPLC Method 5B:

Instrument: LC-8A
Mobile Phase: H₂O (0.1% TFA) (solvent A) and CH₃CN (solvent B),
Gradient: using the elution gradient 30%-95% (solvent B) over 18 minutes.
Flow rate: 15 ml/min;
Column: Metasil Cloak C18 21.2*250 mm, 5 um
Wavelength: UV 220 nm and 254 nm
Column temperature: 25° C.;
MS ionization: ESI.

### HPLC Method 6A:

Instrument: LH-40
Mobile Phase: H₂O (0.1% FA) (solvent A) and CH₃CN (solvent B),
Gradient: using the elution gradient 5%-95% (solvent B) over 30 minutes
Flow rate: 15 ml/min;
Column: YMC-Actus TriartCl8 250*20 mm*5 um
Wavelength: UV 220 nm and 254 nm)
Column temperature: 25° C.;
MS ionization: ESI.

### HPLC Method 6B:

Instrument: LH-40
Mobile Phase: H₂O (0.1% TFA) (solvent A) and CH₃CN (solvent B),
Gradient: using the elution gradient 40%-95% (solvent B) over 20 minutes
Flow rate: 20 ml/min;
Column: YMC-Actus TriartCl8 250*20 mm*5 um
Wavelength: UV 220 nm and 254 nm)
Column temperature: 25° C.;
MS ionization: ESI.

### HPLC Method 6C:

Instrument: LH-40
Mobile Phase: H₂O (0.1% NH₄OH) (solvent A) and CH₃CN (solvent B),
Gradient: using the elution gradient 30%-98% (solvent B) over 20 minutes
Flow rate: 18 ml/min;
Column: YMC-ActusTriart C18 250*20 mm S-5 um, 12 nm
Wavelength: UV 220 nm and 254 nm)
Column temperature: 25° C.;
MS ionization: ESI.

### HPLC Method 7A:

Instrument: LC-LHS
Mobile Phase: H₂O (0.1% FA) (solvent A) and CH₃CN (solvent B),
Gradient: using the elution gradient 40%-95% (solvent B) over 22 minutes
Flow rate: 15 ml/min;
Column: YMC-Actus Triart C18/S-5 um/12 nm Lot No. 18849
Wavelength: UV 220 nm and 254 nm)
Column temperature: rt;
MS ionization: ESI.

### HPLC Method 7B:

Instrument: LC-LHS
Mobile Phase: H₂O (0.1% NH4OH) (solvent A) and CH₃CN (solvent B),
Gradient: using the elution gradient 30%-95% (solvent B) over 22 minutes
Flow rate: 15 ml/min;
Column: YMC-Actus Triart C18/S-5 um/12 nm Lot No. 18849
Wavelength: UV 220 nm and 254 nm)
Column temperature: rt;
MS ionization: ESI.

### HPLC Method 8A:

Instrument: LC-LHS
Mobile Phase: H₂O (0.1% FA) (solvent A) and CH₃CN (solvent B),
Gradient: using the elution gradient 20%-95% (solvent B) over 18 minutes.
Flow rate: 18 ml/min;
Column: Shim-pack Scepter C18 50*4.6 mm, 5 um, 5 um
Wavelength: UV 220 nm and 254 nm
Column temperature: 25° C.;
MS ionization: ESI.

### HPLC Method 9:

Instrument: LC-8A
Mobile Phase: H₂O (0.1% TFA) (solvent A) and CH₃CN (solvent B),
Gradient: using the elution gradient 35%-95% (solvent B) over 21 minutes.
Flow rate: 15 ml/min;
Column: welch xbidge xb 5 um 21.2*250 mm
Wavelength: UV 220 nm and 254 nm
Column temperature: 25° C.;
MS ionization: ESI.

### HPLC Method 10A:

Instrument: LC-8A
Mobile Phase: H₂O (0.1% TFA) (solvent A) and CH₃CN (solvent B),
Gradient: using the elution gradient 30%-95% (solvent B) over 22 minutes
Flow rate: 15 ml/min;
Column: welch xbidge xb 5 μm 21.2*250 mm
Wavelength: UV 220 nm and 254 nm
Column temperature: rt;
MS ionization: ESI.

### Analytical LCMS Methods:

### Analytical LC/MS Analysis Method A:

ESI+/− ion mode 150-850 Da
Wavelength: UV 220 nm
Column: Waters, Xbridge C18 50*2.1 mm, 5 um
Temperature: 40° C.
Gradient:

| | | 0.019% TFA in | |
|---|---|---|---|
| Time (min) | 0.038% TFA in water | acetonitrile | Flow (mL/min) |
| 0 | 99% | 1% | 0.8 |
| 3.85 | 0% | 100% | 0.8 |

### Analytical LC/MS Analysis Method B:

ESI+/− ion mode 150-850 Da
Wavelength: UV 220 nm
Column: Waters, Xbridge C18 50*2.1 mm, Sum
Temperature: 40° C.
Gradient:

| | | 0.019% TFA in | |
|---|---|---|---|
| Time (min) | 0.038% TFA in water | acetonitrile | Flow (mL/min) |
| 0 | 90% | 10% | 0.8 |
| 3.85 | 0% | 100% | 0.8 |

### Analytical LC/MS Analysis Method C:

(0-70% positive-negative)
ESI+/− ion mode 100-1000 Da
Wavelength: UV 220 nm/254 nm
Column: Shim-pack C18, 50*4.6 mm, 5 μm or Welch Xtimate C18, 250*4.6 mm, 5 μm or YMC-Triart C18, 50*4.6 mm, Sum
Temperature: 40° C.
Gradient:

| | | 0.1% FA in | |
|---|---|---|---|
| Time (min) | 0.1% FA in water | acetonitrile | Flow (mL/min) |
| 0.01 | 80% | 20% | 2.5 |
| 2.5 | 5% | 95% | 2.5 |

### Analytical LC/MS Analysis Method D:

(positive-negative 3 min-1)
ESI+/− ion mode 100-1000 Da
Wavelength: UV 220 nm/254 nm
Column: YMC-Triart C18, 50*4.6 mm, Sum or Shim-pack C18, 50*4.6 mm, 5 um
Temperature: 40° C.
Gradient:

| | | 0.1% FA in | |
|---|---|---|---|
| Time (min) | 0.1% FA in water | acetonitrile | Flow (mL/min) |
| 0.01 | 80% | 20% | 2.5 or 1.2 |
| 2.5 | 5% | 95% | 2.5 or 1.2 |

### Analytical LC/MS Analysis Method E:

ESI+/− ion mode 100-1000 Da
Wavelength: UV 220 nm/254 nm
Column: YMC-Triart C18, 50*4.6 mm, Sum
Temperature: 40° C.
Gradient:

| | | 0.1% TFA in | |
|---|---|---|---|
| Time (min) | 0.1% TFA in water | acetonitrile | Flow (mL/min) |
| 0.4 | 100% | 0% | 2.5 |
| 4.6 | 70% | 30% | 2.5 |

### Analytical LC/MS Analysis Method F:

ESI+/− ion mode 100-1000 Da
Wavelength: UV 220 nm/254 nm
Column: YMC-Triart C18, 50*4.6 mm, 5 um
Temperature: 40° C.
Gradient:

| | | 0.1% TFA in | |
|---|---|---|---|
| Time (min) | 0.1% TFA in water | acetonitrile | Flow (mL/min) |
| 0.4 | 80% | 20% | 2.5 |
| 2.5 | 5% | 95% | 2.5 |

### Analytical LC/MS Analysis Method G:

(0-50% positive 3 min)
ESI+/− ion mode 100-1000 Da
Wavelength: UV 220 nm/254 nm
Column: YMC-Triart C18, 50*4.6 mm, Sum or Shim-Pack Scepter C18, 33*3.0 mm, 3 um
Temperature: 40° C.
Gradient:

| | | 0.1% FA in | |
|---|---|---|---|
| Time (min) | 0.1% FA in water | acetonitrile | Flow (mL/min) |
| 0.01 | 100% | 0% | 2.3 |
| 2.5 | 50% | 50% | 2.3 |

### Analytical LC/MS Analysis Method H:

ESI+/− ion mode 100-1000 Da
Wavelength: UV 220 nm/254 nm
Column: YMC-Triart C18, 50×4.6 mm, 5 um
Temperature: 40° C.
Gradient:

| | | 0.1% TFA in | |
|---|---|---|---|
| Time (min) | 0.1% TFA in water | acetonitrile | Flow (mL/min) |
| 0.01 | 80% | 20% | 2.5 |
| 2.5 | 5% | 95% | 2.5 |

### Analytical LC/MS Analysis Method J:

ESI+/− ion mode 100-1000 Da
Column: Shim-Pack Scepter C18, 33*3.0 mm, 3 um
Temperature: 40° C.
Gradient:

| | | 0.1% FA in | |
|---|---|---|---|
| Time (min) | 0.1% FA in water | acetonitrile | Flow (mL/min) |
| 0.01 | 95% | 5% | 1.2 |
| 2.5 | 70% | 30% | 1.2 |

### Analytical LC/MS Analysis Method K:

ESI+/− ion mode 100-1000 Da
Column: Kromasil-C18, 50*4.6 mm, Sum or YMC-Triart C18, 50*4.6 mm, Sum
Temperature: 40° C.
Gradient:

| | | 0.1% FA in | |
|---|---|---|---|
| Time (min) | 0.1% FA in water | acetonitrile | Flow (mL/min) |
| 0.01 | 100% | 0% | 2.5 |
| 5.0 | 50% | 50% | 2.5 |

### Analytical LC/MS Analysis Method M:

ESI+/− ion mode 100-1000 Da
Column: Shim-Pack Scepter C18, 33*3.0 mm, 3 um
Temperature: 40° C.
Gradient:

| | | 0.1% FA in | |
|---|---|---|---|
| Time (min) | 0.1% FA in water | acetonitrile | Flow (mL/min) |
| 0.01 | 20% | 80% | 1.2 |
| 3.0 | 5% | 95% | 1.2 |

### Analytical LC/MS Analysis Method N:

ESI+/− ion mode 100-1000 Da
Column: YMC-Triart C18, 50*4.6 mm, 5 um
Temperature: 40° C.
Gradient:

| | | 0.1% FA in | |
|---|---|---|---|
| Time (min) | 0.1% FA in water | acetonitrile | Flow (mL/min) |
| 0.01 | 40% | 60% | 2.5 |
| 5.0 | 5% | 95% | 2.5 |

### Analytical LC/MS Analysis Method 0:

ESI+/− ion mode 100-1000 Da
Column: YMC-Triart C18, 50*4.6 mm, Sum
Temperature: 40° C.
Gradient:

| | | 0.1% FA in | |
|---|---|---|---|
| Time (min) | 0.1% FA in water | acetonitrile | Flow (mL/min) |
| 0.01 | 40% | 60% | 1.2 |
| 3.0 | 5% | 95% | 1.2 |

### Example 1: Method A: Synthesis of Compound I-55

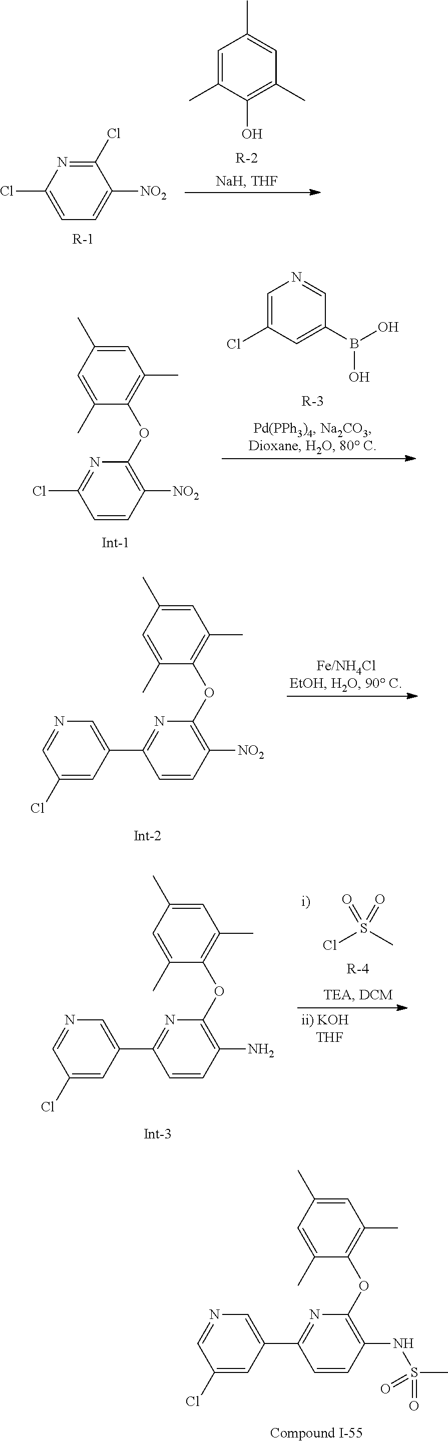

Step 1: To a stirred solution of 2,4,6-trimethylphenol (R-2, 1.36 g, 10.0 mmol, 1.0 eq.) in THE (20 mL) at 0° C. was slowly added NaH (480 mg, 12.0 mmol, 1.2 eq.). The resulting mixture was stirred at 0° C. under N₂ atmosphere for 30 min, whereupon 2, 6-dichloro-3-nitropyridine (R-1, 1.92 g, 10.0 mmol, 1.0 eq.) was added to the reaction mixture. The mixture was stirred at room temperature for 2 hrs, and then the reaction was quenched by saturated aqueous solution of ammonium chloride (20 mL). The layers were separated, and the aqueous layer was extracted by EtOAc (20 mL×3). The organic layers were combined, dried over anhydrous Na₂SO₄ and concentrated under reduced pressure. The residue was purified by flash column chromatography on silica gel (eluted with PE:EtOAc=100:1 to 10:1) to afford Int-1 (2.50 g, 8.56 mmol, 86%) as a yellow solid. LCMS (ESI): m/z=293 [M+H]+.

Step 2: To a mixture of Int-1 (2.50 g, 8.56 mmol, 1 eq.), (5-chloropyridin-3-yl)boronic acid (R-3, 2.1 g, 12.8 mmol, 1.5 eq.) and Na₂CO₃ (2.7 g, 25.7 mmol, 3.0 eq.) in dioxane (15 mL) and water (3 mL) was added Pd(PPh₃)₄ (989 mg, 0.856 mmol, 0.1 eq.). The resulting mixture was stirred at 80° C. under nitrogen atmosphere overnight. After completion, the reaction mixture was diluted with H₂O (30 mL) and extracted with EtOAc (30 mL×3). The combined organic layers were dried over anhydrous Na₂SO₄ and concentrated under reduced pressure. The residue was purified by flash column chromatography on silica gel (eluted with PE:EtOAc=100:1 to 10:1) to afford Int-2 (1.6 g, 4.34 mmol, 51%) as a yellow solid. LCMS (ESI): m/z=370 [M+H]+.

Step 3: To a stirred solution of Int-2 (1.6 g, 4.34 mmol, 1.0 eq.) in 20 mL of EtOH and 5 mL of H₂O were added Fe powder (1.2 g, 21.7 mmol, 5 eq.) and ammonium chloride (1.3 g, 21.7 mmol, 5 eq.) at room temperature. The resulting mixture was then warmed to 90° C. and stirred at that temperature for 2 hrs. After completion, the reaction mixture was filtered through a pad of Celite, and the filter cake was washed with MeOH (20 mL). The combined filtrates were concentrated to dryness, and the residue was diluted with water (30 mL), and then extracted with EtOAc (30 mL×3). The combined organic layers were dried over anhydrous Na₂SO₄ and concentrated under reduced pressure. The residue was purified by flash column chromatography on silica gel (eluted with PE:EtOAc=100:1 to 2:1) to afford Int-3 (1.2 g, 3.52 mmol, 82%) as a grey solid. LCMS (ESI): m/z=340 [M+H]+.

Step 4: A stirred solution of Int-3 (100 mg, 0.29 mmol, 1.0 eq.) and TEA (0.3 ml) in DCM (5 mL) was cooled to 0° C., and methane sulfonyl chloride (R-4, 84 mg, 0.73 mmol, 2.5 eq.) was added slowly. The resulting mixture was stirred at room temperature for 20 min. After completion, the reaction mixture was diluted with H₂O (10 mL) and extracted with EtOAc (10 mL×3). The organic layers were combined, dried over anhydrous Na₂SO₄, and concentrated under reduced pressure. The residue was dissolved with THF (10 mL), then to the solution was added aqueous solution KOH (1 M, 5 mL). The resulting mixture was stirred at room temperature for 30 min. After completion, the reaction mixture was diluted with H₂O (10 mL) and extracted with EtOAc (15 mL×3). The organic layers were combined, dried over anhydrous Na₂SO₄ and concentrated under reduced pressure. The residue was purified by prep-HPLC (Method 4B) to afford Compound I-55 (35 mg, 84 μmol, 28%) as a white solid. Compound I-55: Retention time: 1.613 min. LC-MS (ESI) m/z 418.0 [M+H]f; ¹H NMR (400 MHz, DMSO-d₆) δ 9.91 (s, 1H), 8.78 (d, J=1.6 Hz, 1H), 8.56 (d, J=1.9 Hz, 1H), 8.11 (s, 1H), 7.89 (d, J=8.1 Hz, 1H), 7.81 (d, J=8.0 Hz, 1H), 6.98 (s, 2H), 3.18 (s, 3H), 2.29 (s, 3H), 2.04 (s, 6H).

The following compounds were synthesized using Method A with the appropriate alcohols, boronic acids/boronates and sulfonyl chlorides: Compounds I-1, I-3, I-4, I-37, I-40, I-46, I-49, I-50, I-51, I-54, I-56, I-57, I-58, I-60, I-62, I-63, I-65, I-68, I-71, I-72, I-252, I-253, and I-283.

### Example 2: Method B: Synthesis of Compound I-35

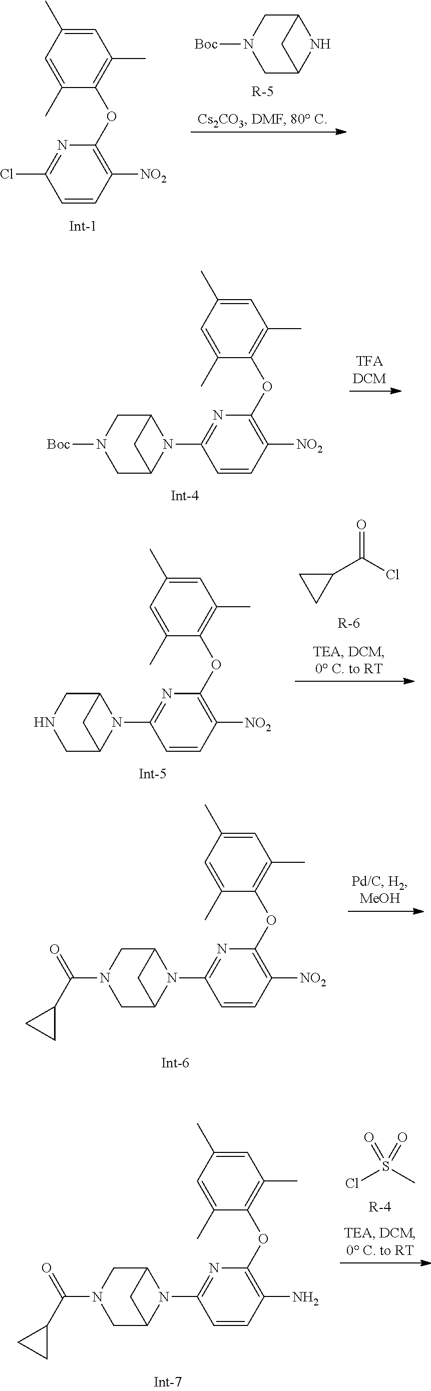

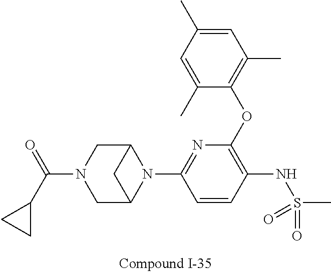 [SMILES: Cc1cc(C)c(Oc2nc(N3C4CC3CN(C(=O)C3CC3)C4)ccc2NS(C)(=O)=O)c(C)c1]

Step 1: To a stirred solution of 6-chloro-2-(mesityloxy)-3-nitropyridine (Int-1, 100 mg, 0.34 mmol, 1.0 eq.) and R-5 (81 mg, 0.41 mmol, 1.2 eq.) in DMF (5 mL) was added Cs₂CO₃ (168 mg, 0.51 mmol, 1.5 eq.) at room temperature. The resulting mixture was stirred at 80° C. for 2 hrs, then the reaction mixture was diluted with H₂O (30 mL) and extracted with EtOAc (30 mL×3). The organic layers were combined and washed with brine (20 mL×3), dried over anhydrous Na₂SO₄ and concentrated under reduced pressure to afford Int-4 (141 mg, quant.) as a yellow oil, which was used in the next step directly without further purification. LCMS (ESI): m/z=455 [M+H]+.

Step 2: To a stirred solution of Int-4 (141 mg, crude) in DCM (5 mL) was added TFA (2 mL) slowly at room temperature. The resulting mixture was stirred at room temperature for 20 min. After completion, the reaction mixture was concentrated under reduced pressure to afford Int-5 (110 mg, quant.) as a yellow oil, which was used in the next step directly without further purification. LCMS (ESI): m/z=355 [M+H]⁺.

Step 3: To a stirred solution of Int-5 (110 mg, crude) in DCM (5 mL) were added TEA (0.1 mL) and cyclopropanecarbonyl chloride (R-6, 0.5 mL) slowly at 0° C. The resulting mixture was stirred at room temperature for 20 min. After completion, the reaction mixture was diluted with H₂O (10 mL) and extracted with EtOAc (15 mL×3). The organic layers were combined, dried over anhydrous Na₂SO₄ and concentrated under reduced pressure. The residue was purified by flash column chromatography through silica gel (eluted with PE:EtOAc=100:1 to 3:1) to afford Int-6 (85 mg) as a yellow solid. LCMS (ESI): m/z=423 [M+H]⁺.

Step 4: To a stirred solution of Int-6 (85 mg, 0.20 mmol, 1.0 eq.) in MeOH (20 mL) was added Pd/C (10 mg, 10 wt %) at room temperature under nitrogen. The suspension was degassed under vacuum and purged with H₂ several times. The resulting mixture was stirred at room temperature for 0.5 hrs. After completion, the suspension was filtered through a pad of Celite, the filter cake was washed with MeOH (20 mL), and the combined filtrates were concentrated to dryness to afford Int-7 (62 mg, quant.) as a brown solid. LCMS (ESI): m/z=393 [M+H]⁺.

Step 5: A solution of Int-7 (62 mg, crude) and TEA (0.3 mL) in DCM (5 mL) was cooled to 0° C. and, then methanesulfonyl chloride (R-4, 0.5 mL) was added slowly. The resulting mixture was stirred at room temperature for 20 min, whereupon the reaction mixture was diluted with H₂O (10 mL) and extracted with EtOAc (15 mL×3). The organic layers were combined, dried over anhydrous Na₂SO₄ and concentrated under reduced pressure. The residue was purified by Prep-HPLC (Method 4B) to afford Compound I-35 (7.5 mg) as a white solid. Compound I-35: Retention time: 1.486 min. LCMS (ESI): m/z=471 [M+H]⁺; ¹H NMR (400 MHz, DMSO-d6) δ 9.14 (s, 1H), 7.48 (d, J=8.3 Hz, 1H), 6.88 (s, 2H), 6.21 (d, J=8.3 Hz, 1H), 4.14-4.03 (m, 1H), 3.98-3.93 (m, 1H), 3.92-3.84 (m, 1H), 3.69-3.61 (m, 1H), 3.55-3.46 (m, 1H), 3.21-3.12 (m, 1H), 3.01 (s, 3H), 2.55-2.51 (m, 1H), 2.23 (s, 3H), 1.95 (s, 6H), 1.69-1.57 (m, 1H), 1.40 (d, J=8.7 Hz, 1H), 0.76-0.62 (m, 3H), 0.62-0.52 (m, 1H).

The following compounds were synthesized using Method B with the appropriate amines, alcohols, boronic acids/boronates and sulfonyl chlorides: Compounds I-15, I-18, I-19, I-22, I-24, I-25, I-31, I-38, I-43 and I-44.

### Example 3: Method C: Synthesis of Compound I-77

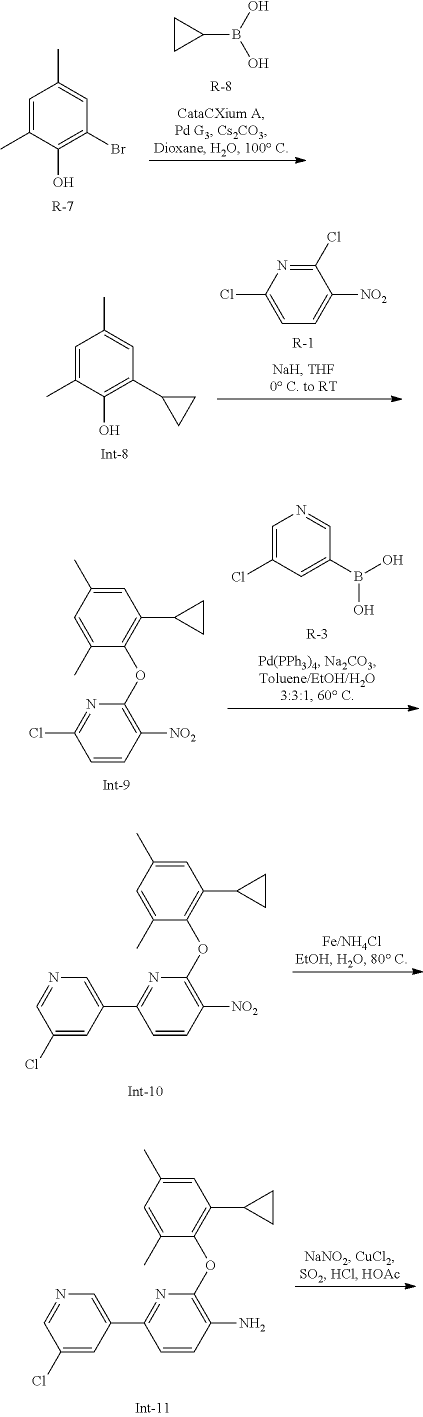

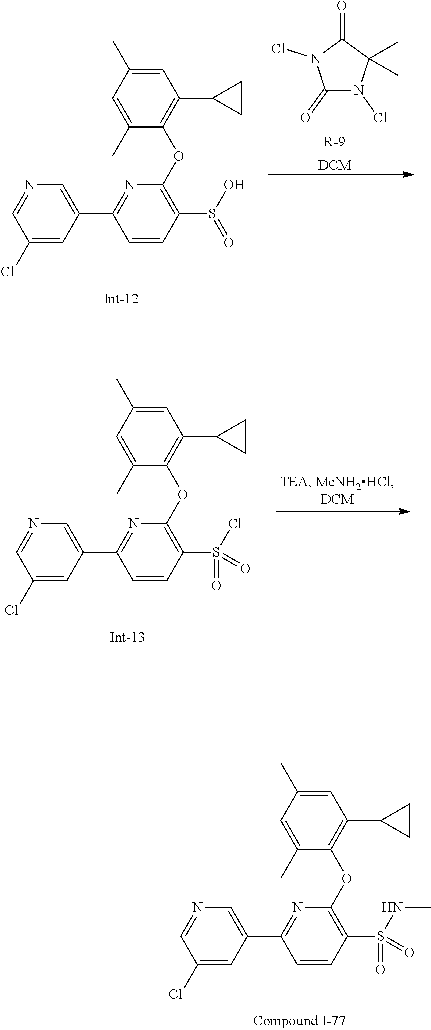

Step 1: To a mixture of 2-bromo-4,6-dimethylphenol (R-7, 10.0 g, 50.0 mmol, 1 eq.), cyclopropylboronic acid (R-8, 12.9 g, 150.0 mmol, 3 eq.) and Cs₂CO₃ (48.9 g, 150 mmol, 3 eq.) in dioxane (300 mL) and water (60 mL) was added CataCXium A Pd G3 (1.8 g, 2.5 mmol, 0.05 eq.). The resulting mixture was stirred at 100° C. overnight under nitrogen atmosphere. After completion, the reaction mixture was diluted with H₂O (500 mL) and extracted with EtOAc (500 mL×3). The combined organic layers were dried over anhydrous Na₂SO₄ and concentrated under reduced pressure. The residue was purified by flash column chromatography on silica gel (eluted with PE:EtOAc=4:1 to 3:1) to afford Int-8 (5.84 g, 36 mmol, 72%) as a white solid. LCMS (ESI): m/z=163 [M+H]⁺.

Step 2: A solution of Int-8 (500 mg, 3.09 mmol, 1.0 eq.) in THE (20 mL) was cooled to 0° C., and NaH (148 mg, 3.71 mmol, 1.2 eq.) was added slowly. The resulting mixture was stirred at room temperature under N₂ atmosphere for 30 min, and then 2,6-dichloro-3-nitropyridine (R-1, 592 mg, 3.09 mmol, 1.0 eq.) was added. The reaction mixture was stirred at room temperature for 2 hrs, whereupon the reaction was quenched by saturated aqueous solution of ammonium chloride (20 mL). The layers were separated, and the aqueous layer was extracted with EtOAc (20 mL×3). The organic layers were combined, dried over anhydrous Na₂SO₄ and concentrated under reduced pressure. The residue was purified by flash column chromatography on silica gel (eluted with PE:EtOAc=100:1 to 10:1) to afford Int-9 (687 mg, 2.16 mmol, 70%) as a yellow solid. LCMS (ESI): m/z=319 [M+H]⁺.

Step 3: To a mixture of Int-9 (687 mg, 2.16 mmol, 1 eq.), (5-chloropyridin-3-yl)boronic acid (R-3, 407 mg, 2.59 mmol, 1.2 eq.) and Na₂CO₃ (687 mg, 6.48 mmol, 3.0 eq.) in toluene (18 mL), EtOH (18 mL) and water (6 mL) was added Pd(PPh₃)₄ (249 mg, 0.216 mmol, 0.1 eq.). The resulting mixture was stirred at 60° C. under nitrogen atmosphere overnight. After completion, the reaction mixture was diluted with H₂O (20 mL) and extracted with EtOAc (20 mL×3). The combined organic layers were dried over anhydrous Na₂SO₄ and concentrated under reduced pressure. The residue was purified by flash column chromatography on silica gel (eluted with PE:EtOAc=100:1 to 10:1) to afford Int-10 (427 mg, 1.08 mmol, 50%) as a white solid. LCMS (ESI): m/z=396 [M+H]⁺.

Step 4: To a stirred solution of Int-10 (427 mg, 1.08 mmol, 1.0 eq.) in 10 mL of EtOH and 5 mL of H₂O were added Fe powder (302 mg, 5.4 mmol, 5 eq.) and ammonium chloride (292 mg, 5.4 mmol, 5 eq.) at room temperature. The resulting mixture was stirred at 80° C. for 5 hrs. After completion, the reaction mixture was filtered through a pad of Celite, the filter cake was washed with MeOH (20 mL), and the combined filtrates were concentrated to dryness. The residue was diluted with water (10 mL) and then extracted with EtOAc (20 mL×3). The combined organic layers were dried over anhydrous Na₂SO₄ and concentrated under reduced pressure. The residue was purified by flash column chromatography on silica gel (eluted with PE:EtOAc=100:1 to 2:1) to afford Int-11 (296 mg, 0.81 mmol, 75%) as a grey solid. LCMS (ESI): m/z=366 [M+H]⁺.

Step 5: A solution of Int-11 (300 mg, 0.82 mmol, 1.0 eq.) in HCl (10 mL) and HOAc (30 mL) was cooled to at −10° C., and then was treated with sodium nitrite (113 mg, 1.64 mmol, 2.0 eq.). The resulting mixture was stirred at −10° C. for 1 hr, then CuCl₂ (112 mg, 0.66 mmol, 0.80 eq.) was added and the resulting mixture was stirred at −10° C. for 0.5 hrs. Next, SO₂ gas was bubbled through the reaction mixture for 10 min. The resulting mixture was stirred at −10° C. for an additional 0.5 hrs. After completion, the reaction mixture was diluted with H₂O (20 mL) and extracted with EtOAc (20 mL×3). The combined organic layers were washed with brine (15 mL), dried over anhydrous Na₂SO₄ and concentrated under reduced pressure to afford Int-12 (300 mg, quant.) as a yellow solid, which was used in the next step directly without further purification. LCMS (ESI): m/z=415 [M+H]⁺.

Step 6: To a solution of Int-12 (300 mg, 0.72 mmol, 1.0 eq.) in DCM (3 mL) was added 1,3-dichloro-4,4-dimethyl-2-oxotetrahydro-1H-imidazol-5-one (R-9, 71 mg, 0.36 mmol, 0.5 eq.). The resulting mixture was stirred at 25° C. for 10 min, then the reaction mixture was diluted with H₂O (20 mL) and extracted with DCM (20 mL×3). The combined organic layers were washed with brine (15 mL), dried over anhydrous Na₂SO₄ and concentrated under reduced pressure. The residue was purified by flash column chromatography on silica gel (eluted with PE:EtOAc=5:1 to 4:1) to afford Int-13 (50 mg, 0.11 mmol, 15%) as a yellow oil.

Step 7: A solution of MeNH₂·HCl (4 mg, 47 μmol, 1.0 eq.) and TEA (19 mg, 188 μmol, 4.0 eq.) in DCM (5 mL) was cooled to 0° C. and then Int-13 (21 mg, 47 μmol, 1.0 eq.) was added. The reaction mixture was stirred for 30 min, then was diluted with H₂O (10 mL) and extracted with DCM (15 mL×3). The organic layers were combined, dried over anhydrous Na₂SO₄ and concentrated under reduced pressure. The residue was purified by reverse phase chromatography using a 40 g C18 cartridge eluting with a gradient of 35-95% MeCN in water (with 0.1% FA) to afford Compound I-77 (3.9 mg, 9 μmol, 19%) as a white solid. Compound I-77: Retention time: 1.913 min. LC-MS (ESI): m/z 444.1 [M+H]⁺; ¹H NMR (400 MHz, DMSO-d6) δ 8.84 (d, J=1.8 Hz, 1H), 8.66 (d, J=2.3 Hz, 1H), 8.34 (d, J=7.9 Hz, 1H), 8.24 (t, J=2.1 Hz, 1H), 7.99 (d, J=7.9 Hz, 1H), 7.84 (s, 1H), 6.99-6.94 (m, 1H), 6.72-6.67 (m, 1H), 2.59 (s, 3H), 2.29 (s, 3H), 2.05 (s, 3H), 1.91-1.81 (m, 1H), 0.79-0.70 (m, 2H), 0.60-0.51 (m, 1H), 0.36-0.24 (m, 1H).

The following compounds were synthesized using Method C with the appropriate reagents: Compounds I-74, I-75, I-76, I-78, I-79, I-80, I-81, I-82, I-83, I-84, I-85, I-86, I-87, and I-271.

### Example 4: Method D: Synthesis of Compound I-39

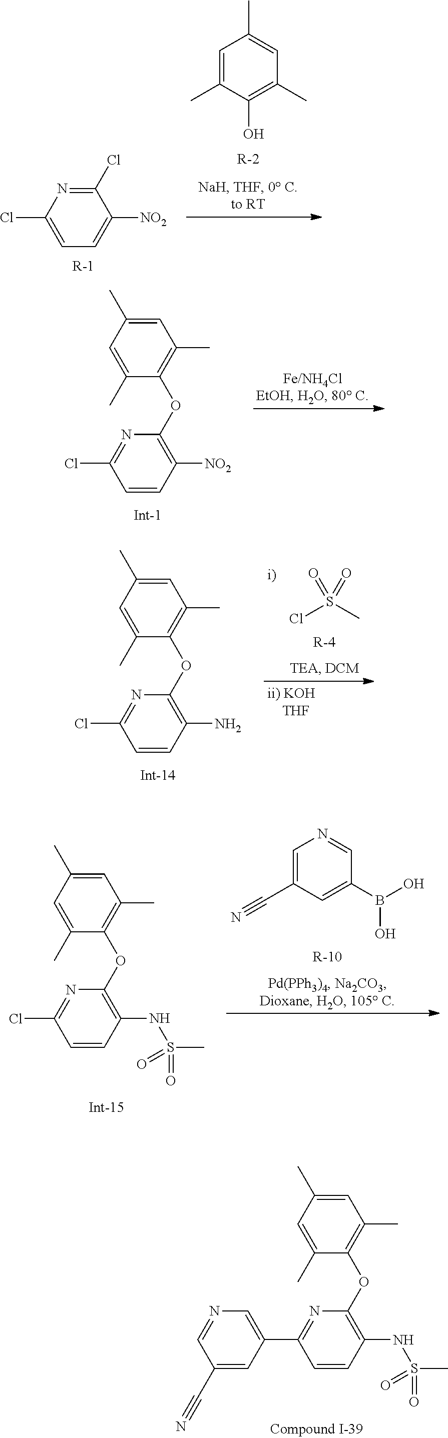

Step 1: A stirred solution of 2,4,6-trimethylphenol (R-2, 1.36 g, 10.0 mmol, 1.0 eq.) in THF (20 mL) was cooled to 0° C., and NaH (480 mg, 12.0 mmol, 1.2 eq.) was added slowly. The resulting mixture was stirred at room temperature under N₂ atmosphere for 30 min, then was treated with 2, 6-dichloro-3-nitropyridine (R-1, 1.92 g, 10.0 mmol, 1.0 eq.). The mixture was stirred at room temperature for 2 hrs, and then the reaction was quenched by the addition of saturated aqueous solution of ammonium chloride (20 mL). The layers were separated, and the aqueous layer was extracted with EtOAc (20 mL×3). The organic layers were combined, dried over anhydrous Na₂SO₄ and concentrated under reduced pressure. The residue was purified by flash column chromatography on silica gel (eluted with PE:EtOAc=100:1 to 10:1) to afford Int-1 (2.40 g, 8.22 mmol, 82%) as a yellow solid. LCMS (ESI): m/z=293 [M+H]⁺.

Step 2: To a stirred solution of Int-1 (292 mg, 1.0 mmol, 1.0 eq.) in 10 mL of EtOH and 5 mL of H₂O was added Fe powder (280 mg, 5.0 mmol, 5.0 eq.) and ammonium chloride (268 mg, 5.0 mmol, 5.0 eq.) at room temperature. The resulting mixture was stirred at 80° C. for 5 hrs. After completion, the reaction mixture was filtered through a pad of Celite, and the filter cake was washed with MeOH (20 mL). The combined filtrates were concentrated to dryness. The residue was diluted with water (10 mL) and then extracted with EtOAc (20 mL×3). The combined organic layers were dried over anhydrous Na₂SO₄ and concentrated under reduced pressure. The residue was purified by flash column chromatography on silica gel (eluted with PE:EtOAc=100:1 to 2:1) to afford Int-14 (197 mg, 0.75 mmol, 75%) as a grey solid. LCMS (ESI): m/z=263 [M+H]⁺.

Step 3: A stirred solution of Int-14 (197 mg, 0.75 mmol, 1.0 eq.) and TEA (0.3 mL) in DCM (5 mL) was cooled to 0° C., and methane sulfonyl chloride (R-4, 205 mg, 1.8 mmol, 2.5 eq.) was added slowly. The resulting mixture was stirred at room temperature for 20 min. After completion, the reaction mixture was diluted with H₂O (10 mL) and extracted with EtOAc (10 mL×3). The organic layers were combined and concentrated under reduced pressure. The resulting residue was dissolved with THE (10 mL), then to the solution was added an aqueous solution of KOH (1 M, 5 mL). The resulting mixture was stirred at room temperature for 30 min, and then the reaction mixture was diluted with H₂O (10 mL) and extracted with EtOAc (15 mL×3). The organic layers were combined, dried over anhydrous Na₂SO₄ and concentrated under reduced pressure. The residue was purified by flash column chromatography on silica gel (eluted with PE:EtOAc=100:1 to 4:1) to afford Int-15 (207 mg, 0.609 mmol, 81%) as a white solid. LCMS (ESI): m/z=341 [M+H]⁺.

Step 4: To a mixture of Int-15 (100 mg, 0.29 mmol, 1 eq.), (5-cyanopyridin-3-yl)boronic acid (R-10, 52 mg, 0.353 mmol, 1.2 eq.) and Na₂CO₃ (94 mg, 0.882 mmol, 3.0 eq.) in dioxane (5 mL) and water (1 mL) was added Pd(PPh₃)₄ (35 mg, 0.03 mmol, 0.1 eq.). The resulting mixture was stirred at 105° C. under nitrogen atmosphere overnight. After completion, the reaction mixture was diluted with H₂O (10 mL) and extracted with EtOAc (10 mL×3). The combined organic layers were dried over anhydrous Na₂SO₄ and concentrated under reduced pressure. The residue was purified by flash column chromatography on silica gel (eluted with PE:EtOAc=100:1 to 10:1) to afford Compound I-39 (35 mg, 86 μmol, 29%) as a white solid. Compound I-39: Retention time: 1.758 min. LCMS (ESI): m/z=409.3 [M+H]⁺; ¹H NMR (400 MHz, DMSO-d6) δ 9.92 (s, 1H), 9.00 (d, J=2.1 Hz, 1H), 8.95 (d, J=1.8 Hz, 1H), 8.56 (t, J=1.9 Hz, 1H), 7.94 (d, J=8.1 Hz, 1H), 7.85 (d, J=8.1 Hz, 1H), 6.98 (s, 2H), 3.21 (s, 3H), 2.29 (s, 3H), 2.04 (s, 6H).

The following compounds were synthesized using Method D, using the appropriate reagents: Compounds I-2, I-5, I-6, I-7, I-8, I-9, I-10, I-13, I-14, I-29, I-36, I-45, I-275, and I-277.

### Example 5: Method E: Synthesis of Compound I-47

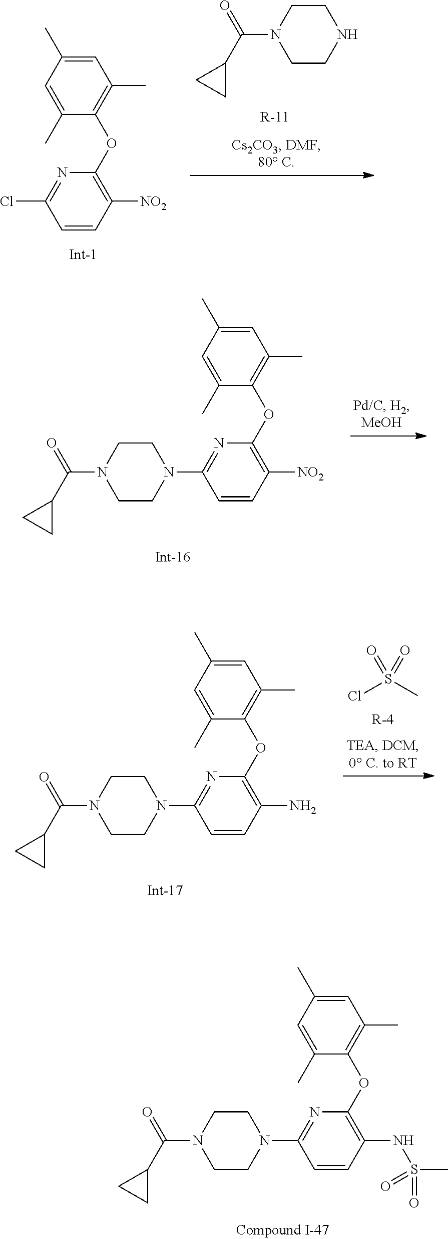

Step 1: To a stirred solution of 6-chloro-2-(mesityloxy)-3-nitropyridine (Int-1, 2.40 g, 8.22 mmol, 1.0 eq.) and cyclopropyl(piperazin-1-yl)methanone (R-11, 1.52 g, 9.86 mmol, 1.2 eq.) in DMF (20 mL) was added Cs₂CO₃ (4.0 g, 12.3 mmol, 1.5 eq.) at room temperature. The resulting mixture was stirred at 80° C. for 2 hrs. After completion, the reaction mixture was diluted with H₂O (30 mL) and extracted with EtOAc (30 mL×3). The organic layers were combined and washed with brine (20 mL×3), dried over anhydrous Na₂SO₄ and concentrated under reduced pressure. The residue was purified by flash column chromatography through silica gel (eluted with PE:EtOAc=4:1 to 3:1) to afford Int-16 (2.74 g, 6.68 mmol, 81%) as a yellow solid. LCMS (ESI): m/z=411 [M+H]⁺.

Step 2: To a stirred solution of Int-16 (2.74 g, 6.68 mmol, 1.0 eq.) in MeOH (20 mL) was added Pd/C (274 mg, 10 wt %) under nitrogen. The suspension was degassed under vacuum and purged with H₂ several times. The resulting mixture was stirred at room temperature for 2 hrs. After completion, the suspension was filtered through a pad of Celite, and the filter cake was washed with MeOH (20 mL). The combined filtrates were concentrated to dryness to give Int-17 (2.18 g, 5.74 mmol, 86%) as a brown solid. LCMS (ESI): m/z=381 [M+H]⁺.

Step 3: A stirred solution of Int-17 (100 mg, 0.263 mmol, 1.0 eq.) and TEA (79.8 mg, 0.789 mmol, 3.0 eq.) in DCM (5 mL) was cooled to 0° C., and then methanesulfonyl chloride (R-4, 36.0 mg, 0.316 mmol, 1.2 eq.) was added slowly. The resulting mixture was stirred at room temperature for 20 min. After completion, the reaction mixture was diluted with H₂O (10 mL) and extracted with EtOAc (15 mL×3). The organic layers were combined, dried over anhydrous Na₂SO₄ and concentrated under reduced pressure. The residue was purified by Prep-HPLC (Method 4B) to afford Compound I-47 (30.4 mg, 66 μmol, 25%) as a white solid. Compound I-47: Retention time: 1.381 min. LCMS (ESI): m/z=459.2 [M+H]⁺; ¹H NMR (400 MHz, DMSO-d₆) δ 9.13 (s, 1H), 7.51 (d, J=8.5 Hz, 1H), 6.90 (s, 2H), 6.42 (d, J=8.6 Hz, 1H), 3.67-3.55 (m, 2H), 3.47-3.39 (m, 2H), 3.28-3.11 (m, 4H), 3.02 (s, 3H), 2.25 (s, 3H), 2.01 (s, 6H), 1.98-1.92 (m, 1H), 0.77-0.63 (m, 4H).

The following compounds were synthesized using Method E using the appropriate amine reagents: Compounds I-11, I-12, I-16, I-17, I-20, I-21, I-26, I-27, I-28, I-30, I-32, I-33, I-34, I-244, I-254, I-255, I-256, I-257, I-261, I-262, I-273, and I-274.

### Example 6: Synthesis of Amine Reagent R-14 Required for Synthesis of Compound I-27 by Utilizing Method E

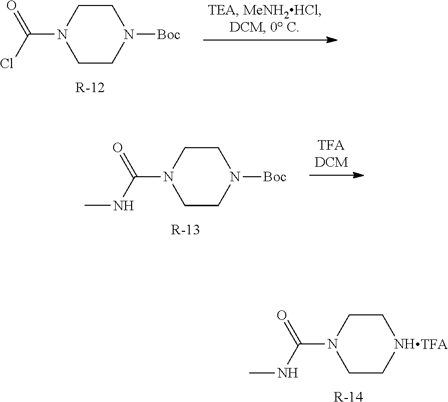

Step 1: A stirred solution of tert-butyl 4-(chlorocarbonyl)piperazine-1-carboxylate (R-12, 500 mg, 2.01 mmol, 1.0 eq.) in DCM (30 mL) was cooled to at 0° C., and then was treated successively with MeNH₂·HCl (269 mg, 4.02 mmol, 2.0 eq.) and TEA (610 mg, 6.03 mmol, 3.0 eq.). The reaction mixture was stirred for 30 min, then was diluted with H₂O (10 mL) and extracted with EtOAc (20 mL×3). The organic layers were combined, dried over anhydrous Na₂SO₄ and concentrated under reduced pressure to afford tert-butyl 4-(methylcarbamoyl)piperazine-1-carboxylate (R-13, 500 mg, quant.) as a yellow oil, which was used in the next step directly without further purification. LCMS (ESI): m/z=244 [M+H]⁺.

Step 2: To a stirred solution of tert-butyl 4-(methylcarbamoyl)piperazine-1-carboxylate (R-13, 500 mg, crude) in DCM (20 mL) was added TFA (10 mL). The resulting mixture was stirred at room temperature for 2 hrs. After completion, the reaction mixture was concentrated under reduced pressure to afford crude N-methylpiperazine-1-carboxamide (TFA salt) (R-14, 400 mg, quant.) as a yellow oil, which was used in the next step (Step 1 of Method E) directly without further purification. LCMS (ESI): m/z=144 [M+H]⁺.

### Example 7: Method F: Synthesis of Compound I-48

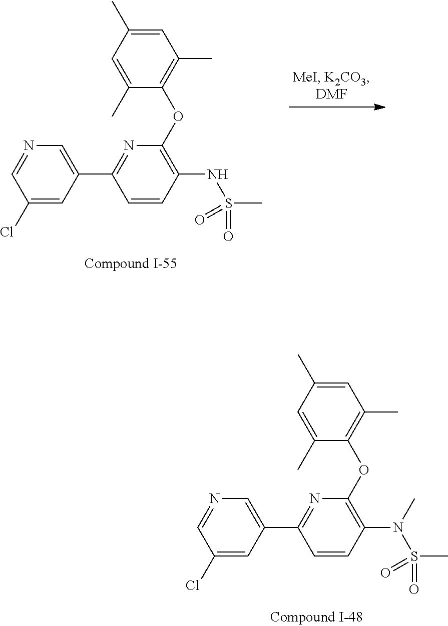 [SMILES: Cc1cc(C)c(Oc2nc(-c3cncc(Cl)c3)ccc2NS(C)(=O)=O)c(C)c1 ; Cc1cc(C)c(Oc2nc(-c3cncc(Cl)c3)ccc2N(C)S(C)(=O)=O)c(C)c1]

To a stirred solution of Compound I-55 (40 mg, 95 μmol, 1.0 eq, prepared by Method A) and K₂CO₃ (39 mg, 285 μmol, 3.0 eq.) in DMF (2 mL) was added Mel (0.1 mL). The resulting mixture was stirred at room temperature for 10 min. After completion, the reaction mixture was diluted with H₂O (10 mL) and extracted with EtOAc (15 mL×3). The organic layers were combined, dried over anhydrous Na₂SO₄ and concentrated under reduced pressure. The residue was purified by prep-HPLC (Method 4B) to afford Compound I-48 (7.5 mg, 17 μmol, 18%) as a white solid. Compound I-48: Retention time: 2.011 min. LC-MS (ESI) m/z 432.2 [M+H]⁺; ¹H NMR (400 MHz, DMSO-d₆) δ 8.80 (d, J=1.8 Hz, 1H), 8.61 (d, J=2.3 Hz, 1H), 8.16 (t, J=2.1 Hz, 1H), 8.05 (d, J=7.9 Hz, 1H), 7.87 (d, J=7.9 Hz, 1H), 7.00 (s, 2H), 3.35 (s, 3H), 3.22 (s, 3H), 2.29 (s, 3H), 2.03 (s, 6H).

The following compounds were synthesized from the appropriate starting materials using Method F: Compounds I-52, I-53, I-64, I-69, I-70, I-134, I-135, I-136, I-139, I-142, I-146, I-148, I-149, I-153, I-154, I-156, I-157, I-166, and I-272.

### Example 8: Method G: Synthesis of Compound I-41

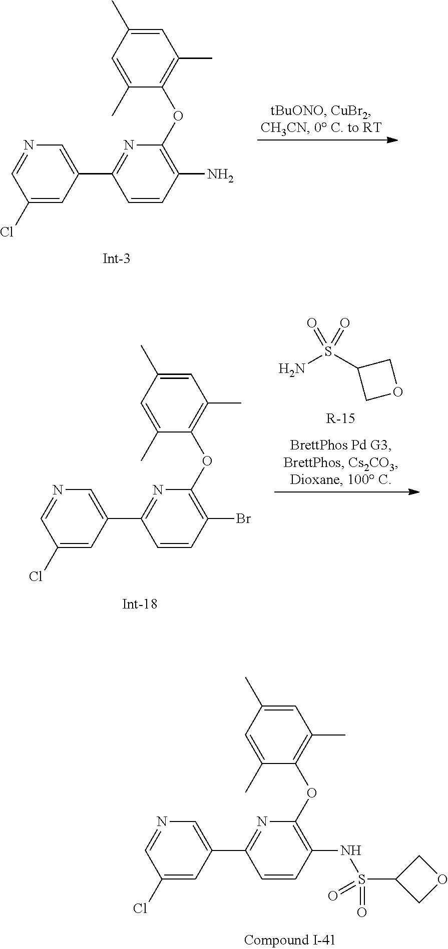

Step 1: A stirred solution of Int-3 (1.0 g, 2.95 mmol, 1.0 eq.) in ACN (30 mL) was cooled to 0° C., and then was treated with ^{t}BuONO (457 mg, 4.43 mmol, 1.5 eq.) and CuBr₂ (978 mg, 4.43 mmol, 1.5 eq.). The reaction mixture was stirred at room temperature for 3 hrs, then was diluted with water (50 mL) and extracted with EtOAc (50 mL×3). The organic layers were combined, dried over anhydrous Na₂SO₄ and concentrated under reduced pressure. The residue was purified by flash column chromatography through silica gel column (eluted with PE:EtOAc=3:1 to 2:1) to afford Int-18 (652 mg, 1.62 mmol, 55%) as a white solid. LC-MS (ESI) m/z 403 [M+H]⁺.

Step 2: To a mixture of Int-18 (100 mg, 0.222 mmol, 1 eq.), oxetane-3-sulfonamide (R-15, 31 mg, 0.222 mmol, 1.0 eq.), and Cs₂CO₃ (218.7 mg, 0.667 mmol, 3.0 eq.) in dioxane (10 mL) were added Brettphos Pd G3 (20 mg, 0.02 mmol, 0.1 eq.) and Brettphos (18 mg, 0.03 mmol, 0.15 eq.). The resulting mixture was stirred at 100° C. for 1 hr under nitrogen atmosphere, whereupon the reaction mixture was diluted with H₂O (10 mL) and extracted with EtOAc (10 mL×3). The combined organic layers were dried over anhydrous Na₂SO₄ and concentrated under reduced pressure. The residue was purified by prep-HPLC (Method 4C) to afford Compound I-41 (7.1 mg, 15 μmol, 7%) as a white solid. Compound I-41: Retention time: 1.795 min. LC-MS (ESI) m/z 460.3 [M+H]⁺; ¹H NMR (400 MHz, DMSO-d₆) δ 10.21 (s, 1H), 8.77 (d, J=1.7 Hz, 1H), 8.57 (d, J=2.2 Hz, 1H), 8.12 (s, 1H), 7.90 (d, J=8.1 Hz, 1H), 7.81 (d, J=8.1 Hz, 1H), 6.98 (s, 2H), 4.85-4.81 (m, 5H), 2.29 (s, 3H), 2.03 (s, 6H).

The following compounds were synthesized from the appropriate reagents using Method G: Compounds 1-228, 1-235, 1-236, 1-237, and I-267.

### Example 9: Method H: Synthesis of Compound I-67

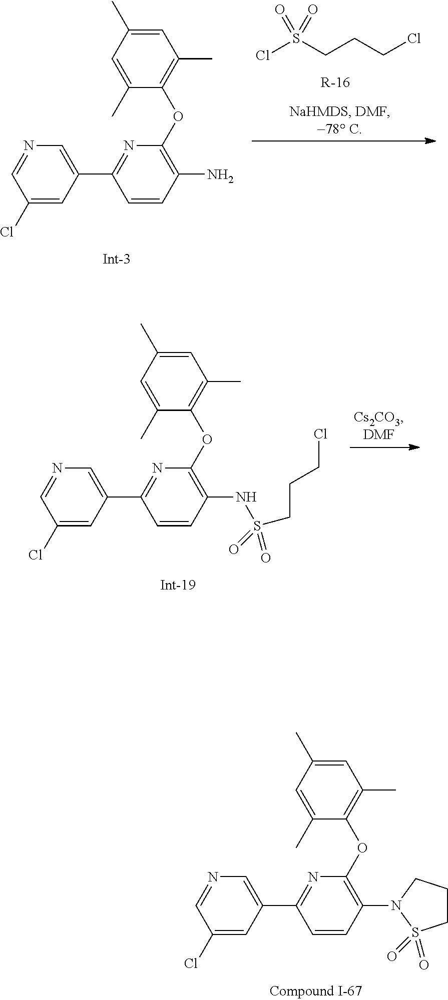

Step 1: To a stirred solution of Int-3 (100 mg, 0.30 mmol, 1 eq.) in DMF (10 mL) was added NaHMDS (0.6 mL, 1 M in THF, 2 eq.) at −78° C. The reaction mixture was stirred for 30 min, and then 3-chloropropane-1-sulfonyl chloride (R-16, 63 mg, 0.36 mmol, 1.2 eq.) was added, and the resulting mixture was stirred at room temperature for 5 hrs. After completion, the reaction mixture was quenched using a saturated aqueous solution of NH₄Cl (20 mL), and then was extracted with EtOAc (30 mL×3). The organic layers were combined, washed with brine (30 mL×3), dried over anhydrous Na₂SO₄ and concentrated under reduced pressure to afford Int-19 (150 mg, quant.) as a yellow oil, which was used in the next step directly without further purification. LCMS (ESI): m/z=480 [M+H]⁺.

Step 2: To a stirred solution of Int-19 (150 mg, crude) in DMF (10 mL) was added Cs₂CO₃ (200 mg, 0.62 mmol). The resulting mixture was stirred at room temperature overnight. After completion, the reaction mixture was diluted with H₂O (15 mL) and extracted with EtOAc (20 mL×3). The organic layers were combined, washed with brine (20 mL×3), dried over anhydrous Na₂SO₄ and concentrated under reduced pressure. The residue was purified by prep-HPLC (Method 4B) to afford Compound I-67 (15 mg, 0.034 mmol, 11% over two steps) as a white solid. Compound I-67: Retention time: 2.162 min. LC-MS (ESI) m/z 444.2 [M+H]⁺; ¹H NMR (400 MHz, DMSO-d₆) δ 8.79 (s, 1H), 8.60 (s, 1H), 8.15 (s, 1H), 7.96 (d, J=7.8 Hz, 1H), 7.87 (d, J=7.8 Hz, 1H), 6.98 (s, 2H), 3.95 (t, J=6.4 Hz, 2H), 3.46 (t, J=7.1 Hz, 2H), 2.29 (s, 3H), 2.03 (s, 6H), 1.32-1.20 (m, 2H).

The following compounds were synthesized from the appropriate reagents using Method H: Compound I-66 and I-221.

### Example 10: Method I: Synthesis of Compound I-42

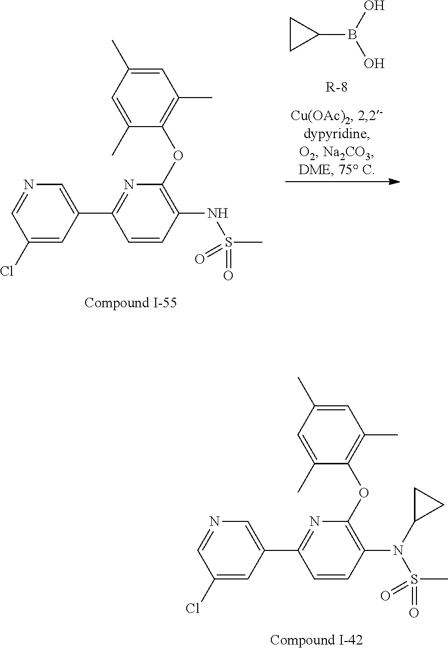

To a stirred solution of Compound I-55 (100 mg, 0.24 mmol, 1 eq.), cyclopropylboronic acid (R-8, 41 mg, 0.48 mmol, 2.0 eq.) and Na₂CO₃ (75 mg, 0.72 mmol, 3.0 eq.) in DME (10 mL) were added Cu(OAc)₂ (44 mg, 0.24 mmol, 1.0 eq.) and 2,2′-Dipyridine (37 mg, 0.24 mmol, 1.0 eq.). The resulting mixture was stirred at 75° C. for 16 hrs under O₂. After completion, the reaction mixture was diluted with H₂O (15 mL) and extracted with DCM (20 mL×3). The organic layers were combined and washed with brine (20 mL), dried over anhydrous Na₂SO₄ and concentrated under reduced pressure. The residue was purified by Prep-HPLC (Method 4B) to afford Compound I-42 (20 mg, 44 μmol, 18%) as a white solid. Compound I-42: Retention time: 2.002 min. LCMS (ESI): m/z=458.3 [M+H]⁺; ¹H NMR (400 MHz, DMSO-d₆) δ 8.80 (d, J=1.8 Hz, 1H), 8.60 (d, J=2.3 Hz, 1H), 8.16 (t, J=2.1 Hz, 1H), 7.96 (d, J=7.9 Hz, 1H), 7.86 (d, J=7.9 Hz, 1H), 6.99 (s, 2H), 3.38-3.34 (m, 1H), 3.28 (s, 3H), 2.29 (s, 3H), 2.02 (s, 6H), 0.87-0.83 (m, 4H).

### Example 11: Method J: Synthesis of Compound I-100

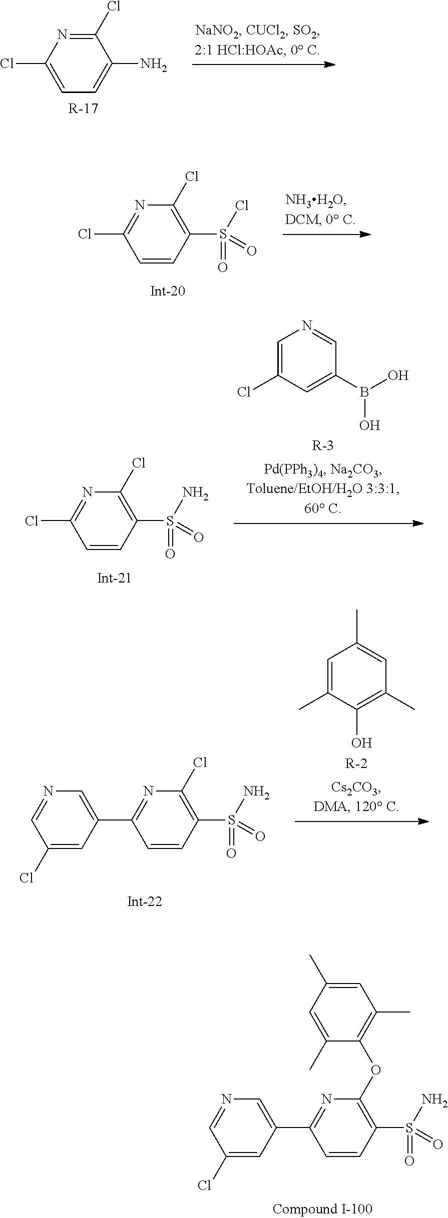

Step 1: 2, 6-dichloropyridin-3-amine (R-17, 16.2 g, 100 mmol, 1.0 eq.) was dissolved in a mixture of concentrated HCl (100 mL) and glacial acetic acid (50 mL). The resulting solution was cooled to 0° C., and an aqueous solution of NaNO₂ (50 mL, 0.28 g/mL, 2.0 eq.) was added slowly. The reaction mixture was stirred for 30 min, then CuCl₂ (13.3 g, 100 mmol, 1.0 eq.) was added, and SO₂ was bubbled through the reaction mixture at 0° C. for 15 min. After completion, the mixture was poured into ice water (150 mL) and extracted with EtOAc (300 mL×3). The combined organic layers were washed with saturated aqueous solution of NaHCO₃ (500 mL×3), dried over anhydrous Na₂SO₄ and concentrated under reduced pressure. The residue was purified by flash column chromatography through silica gel (eluted with PE:EtOAc=2:1 to 1:1) to afford Int-20 (10.0 g, 41.3 mmol, 41%) as a yellow solid.

Step 2: To a stirred solution of Int-20 (500 mg, 2.04 mmol, 1.0 eq.) in DCM (20 mL) was added ammonia solution (5 mL) at 0° C. The resulting mixture was stirred at room temperature for 30 min. After completion, the reaction mixture was diluted with H₂O (10 mL) and extracted with EtOAc (20 mL×3). The organic layers were combined, dried over anhydrous Na₂SO₄ and concentrated under reduced pressure. The residue was purified by flash column chromatography through silica gel (eluted with PE:EtOAc=1: 2 to 1:1) to afford Int-21 (289 mg, 1.28 mmol, 63%) as a white solid. LCMS (ESI): m/z=227 [M+H]⁺.

Step 3: To a mixture of Int-21 (452 mg, 2.0 mmol, 1.0 eq.), (5-chloropyridin-3-yl)boronic acid (R-3, 314 mg, 2.0 mmol, 1.0 eq.) and Na₂CO₃ (318 mg, 6.0 mmol, 3.0 eq.) in a mixed solvent of toluene., EtOH and water (20 mL, 3:3:1) was added Pd(PPh₃)₄ (231.1 mg, 0.2 mmol, 0.1 eq.). The resulting mixture was stirred at 60° C. for 6 hrs. After completion, the reaction mixture was diluted with H₂O (20 mL) and extracted with EtOAc (30 mL×3). The organic layers were combined, dried over anhydrous Na₂SO₄ and concentrated under reduced pressure. The residue was purified by flash column chromatography through silica gel (eluted with PE:EtOAc=100:1 to 3:1) to afford Int-22 (425 mg, 1.40 mmol, 70%) as a white solid. LCMS (ESI): m/z=304 [M+H]⁺.

Step 4: To a stirred solution of Int-22 (100 mg, 0.33 mmol, 1.0 eq.) and 2,4,6-trimethylphenol (R-2, 54 mg, 0.396 mmol, 1.2 eq.) in DMA (15 mL) was added Cs₂CO₃ (161 mg, 0.495 mmol, 1.5 eq.) at room temperature. The resulting mixture was stirred at 120° C. for 3 hrs. After completion, the reaction mixture was diluted with H₂O (30 mL) and extracted with EtOAc (30 mL×3). The organic layers were combined and washed with brine (20 mL×3), dried over anhydrous Na₂SO₄ and concentrated under reduced pressure. The residue was purified by flash column chromatography through silica gel (eluted with PE:EtOAc=5:1 to 3:1) to afford Compound I-100 (21 mg, 52 μmol, 16%) as a white solid. Compound I-100: Retention time: 1.594 min. LCMS (ESI): m/z=404.1 [M+H]f; ¹H NMR (400 MHz, CD₃OD-d₄) δ 8.85 (d, J=1.9 Hz, 1H), 8.54 (d, J=2.3 Hz, 1H), 8.41 (d, J=7.9 Hz, 1H), 8.19 (t, J=2.1 Hz, 1H), 7.81 (d, J=7.9 Hz, 1H), 6.98 (s, 2H), 2.33 (s, 3H), 2.11 (s, 6H).

The following compounds were synthesized from the appropriate reagents using Method J: Compound I-88, I-94, I-95, I-96, I-97, I-98, I-99, I-101 and I-102.

### Example 12: Synthesis of Dichloropyridine Reagent R-20 Required for Synthesis of Compound I-95 by Utilizing Method J

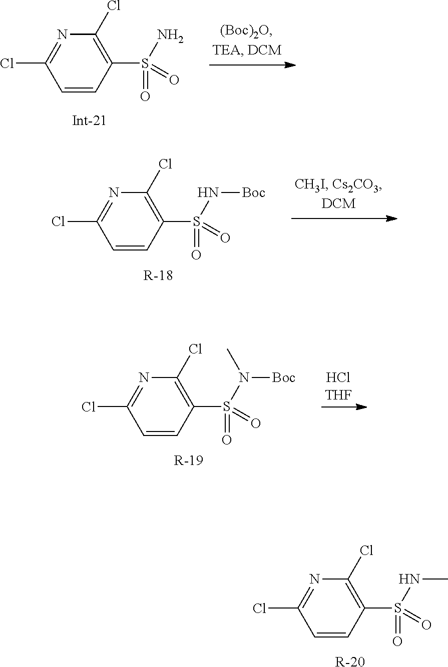

Step 1: To a stirred solution of Int-21 (452 mg, 2.0 mmol, 1.0 eq.) in DCM (20 mL) were added (Boc)₂O (654 mg, 3.0 mmol, 1.5 eq.) and TEA (405 mg, 4.0 mmol, 2.0 eq.). The resulting mixture was stirred at room temperature for 30 min. After completion, the reaction mixture was diluted with H₂O (10 mL) and extracted with EtOAc (20 mL×3). The organic layers were combined, dried over anhydrous Na₂SO₄ and concentrated under reduced pressure. The residue was purified by flash column chromatography through silica gel (eluted with PE:EtOAc=5:1 to 4:1) to afford R-18 (528 mg, 1.62 mmol, 81%) as a white solid. LCMS (ESI): m/z=327 [M+H]⁺.

Step 2: To a stirred solution of R-18 (528 mg, 1.62 mmol, 1.0 eq.) in DCM (20 mL) were added CH₃I (275 mg, 1.94 mmol, 1.2 eq.) and Cs₂CO₃ (792 mg, 2.43 mmol, 1.5 eq.). The resulting mixture was stirred at room temperature for 30 min. After completion, the mixture was diluted with H₂O (20 mL) and extracted with EtOAc (25 mL×3). The organic layers were combined and washed with brine (20 mL×3), dried over anhydrous Na₂SO₄ and concentrated under reduced pressure. The residue was purified by flash column chromatography on silica gel (eluted with PE:EtOAc=10:1 to 5:1) to afford R-19 (435 mg, 1.28 mmol, 79%) as a white solid. LCMS (ESI): m/z=341 [M+H]⁺.

Step 3: To a stirred solution of R-19 (435 mg, 1.28 mmol, 1 eq.) in THE (5 mL) was added concentrated HCl (5 mL). The resulting mixture was stirred at room temperature for 1 hr. After completion, the reaction mixture was diluted with H₂O (30 mL) and neutralized carefully with NaHCO₃ (aq.) until the pH was adjusted to pH=8-9. The resulting mixture was extracted with EtOAc (30 mL×3), the combined organic layers were dried over anhydrous Na₂SO₄ and concentrated under reduced pressure. The residue was purified by flash column chromatography through silica gel (eluted with PE:EtOAc=4:1 to 3:1) to afford R-20 (252 mg, 1.05 mmol, 82%) LCMS (ESI): m/z=241 [M+H]⁺.

### Example 13: Synthesis of Dichloropyridine Reagent R-21 Required for Synthesis of Compound I-97 by Utilizing Method J

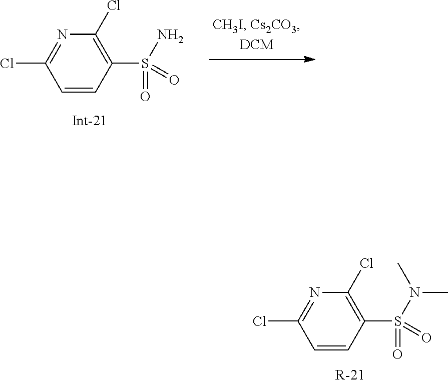

To a stirred solution of Int-21 (452 mg, 2.0 mmol, 1.0 eq.) in DCM (20 mL) were added CH₃I (625 mg, 4.4 mmol, 2.2 eq.) and Cs₂CO₃ (1.95 g, 6.0 mmol, 3.0 eq.). The resulting mixture was stirred at room temperature for 30 min. After completion, the mixture was diluted with H₂O (10 mL) and extracted with EtOAc (25 mL×3). The organic layers were combined, dried over anhydrous Na₂SO₄ and concentrated under reduced pressure. The residue was purified by flash column chromatography on silica gel (eluted with PE:EtOAc=4:1 to 3:1) to afford R-21 (411 mg, 1.62 mmol, 81%) as a white solid. LCMS (ESI): m/z=255 [M+H]⁺.

### Example 14: Method K: Synthesis of Compound I-61 and Compound I-59

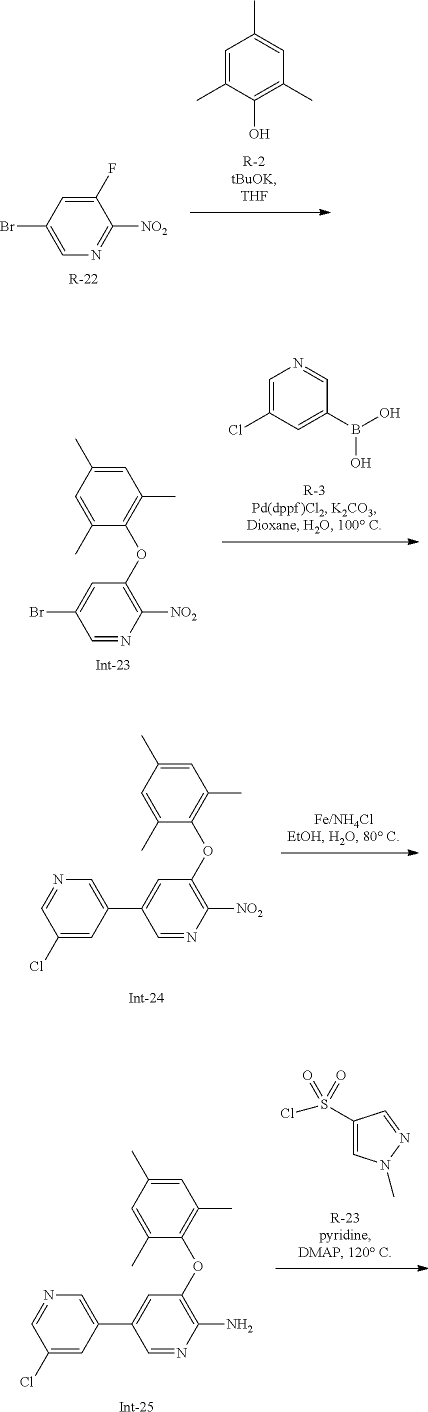

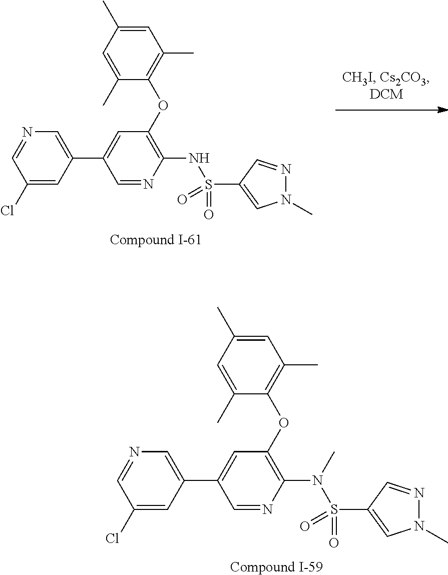 [SMILES: Cc1cc(C)c(Oc2cc(-c3cncc(Cl)c3)cnc2NS(=O)(=O)c2cnn(C)c2)c(C)c1 ; "Compound I-61" ; Cc1cc(C)c(Oc2cc(-c3cncc(Cl)c3)cnc2N(C)S(=O)(=O)c2cnn(C)c2)c(C)c1 ; "Compound I-59"]

Step 1: To a stirred solution of 5-bromo-3-fluoro-2-nitropyridine (R-22, 2.43 g, 11.0 mmol, 1.0 eq.) in THE (30 mL) were added 2,4,6-trimethylphenol (R-2, 1.5 g, 11.0 mmol, 1.0 eq.) and t-BuOK (4.93 g, 44.0 mmol, 4.0 eq.) at 25° C. slowly. The resulting mixture was stirred at 25° C. under N₂ atmosphere for 1 hr. After completion, the reaction was quenched with a saturated aqueous solution of ammonium chloride (20 mL). The layers were separated, and the aqueous layer was extracted with EtOAc (20 mL×3). The organic layers were combined, dried over anhydrous Na₂SO₄ and concentrated under reduced pressure. The residue was purified by flash column chromatography on silica gel (eluted with PE:EtOAc=4:1 to 3:1) to afford Int-23 (3.4 g, 10.1 mmol, 92%) as a yellow solid. LCMS (ESI): m/z=337 [M+H]⁺.

Step 2: To a mixture of Int-23 (3.3 g, 9.82 mmol, 1.0 eq.), (5-chloropyridin-3-yl)boronic acid (R-3, 2.31 g, 14.7 mmol, 1.5 eq.) and K₂CO₃ (5.41 g, 39.3 mmol, 4.0 eq.) in dioxane (50 mL) and water (10 mL) was added Pd(dppf)Cl₂ (717 mg, 0.98 mmol, 0.1 eq.). The resulting mixture was stirred at 100° C. for 1 hr under nitrogen atmosphere. After completion, the reaction mixture was diluted with H₂O (50 mL) and extracted with EtOAc (50 mL×3). The combined organic layers were washed with brine (50 mL×3), dried over anhydrous Na₂SO₄ and concentrated under reduced pressure. The residue was purified by flash column chromatography on silica gel (eluted with PE:EtOAc=4:1 to 3:1) to afford Int-24 (2.9 g, 7.84 mmol, 80%) as a white oil. LCMS (ESI): m/z=370 [M+H]⁺.

Step 3: To a stirred solution of Int-24 (2.8 g, 7.57 mmol, 1.0 eq.) in 30 mL of EtOH and 10 mL of H₂O was added Fe powder (4.23 g, 75.7 mmol, 10.0 eq.) and ammonium chloride (4.05 g, 75.7 mmol, 10.0 eq.) at room temperature. The resulting mixture was stirred at 80° C. for 2 hrs. After completion, the reaction mixture was filtered through a pad of Celite, and the filter cake was washed with MeOH (20 mL). The combined filtrates were concentrated to dryness. The residue was diluted with water (10 mL) and then extracted with EtOAc (30 mL×3). The combined organic layers were washed with brine (50 mL×3), dried over anhydrous Na₂SO₄ and concentrated under reduced pressure. The residue was purified by flash column chromatography on silica gel (eluted with PE:EtOAc=3:1 to 2:1) to afford Int-25 (2 g, 5.89 mmol, 78%) as a yellow solid. LCMS (ESI): m/z=340 [M+H]⁺.

Step 4: To a stirred solution of Int-25 (300 mg, 0.88 mmol, 1.0 eq.) in pyridine (3 mL) were added 1-methylpyrazole-4-sulfonyl chloride (R-23, 319 mg, 1.77 mmol, 2.0 eq.) and DMAP (5 mg, 0.044 mmol, 0.05 eq.). The resulting mixture was stirred at 120° C. for 15 hrs. After completion, the reaction mixture was diluted with H₂O (15 mL) and extracted with EtOAc (30 mL×3). The combined organic layers were washed with brine (20 mL×3), dried over anhydrous Na₂SO₄ and concentrated under reduced pressure. The residue was purified by flash column chromatography on silica gel (eluted with PE:EtOAc=3:1 to 2:1) to afford Compound I-61 (24 mg, 0.050 mmol, 5%) as a white solid. Compound I-61: Retention time: 1.686 min. LCMS (ESI): m/z=484 [M+H]⁺; ¹H NMR (400 MHz, DMSO-d₆) δ 10.87 (s, 1H), 8.60 (d, J=1.5 Hz, 1H), 8.56 (d, J=2.1 Hz, 1H), 8.40 (s, 1H), 8.27 (s, 1H), 8.05 (s, 1H), 7.91 (s, 1H), 6.98 (s, 2H), 6.73 (s, 1H), 3.88 (s, 3H), 2.26 (s, 3H), 2.03 (s, 6H).

Step 5: To a solution of Compound I-61 (40 mg, 0.083 mmol, 1.0 eq.) in DMF (1 mL) were added Cs₂CO₃ (81 mg, 0.25 mmol, 3.0 eq.) and iodomethane (17 mg, 0.12 mmol, 1.5 eq.). The resulting mixture was stirred at 25° C. for 1 hr. After completion, the reaction mixture was diluted with H₂O (15 mL) and extracted with EtOAc (15 mL×3). The combined organic layers were washed with brine (15 mL×3), dried over anhydrous Na₂SO₄ and concentrated under reduced pressure. The residue was purified by prep-HPLC (Method 4B) to afford Compound I-59 (7.3 mg, 0.015 mmol, 18%) as a white solid. Compound I-59: Retention time: 1.848 min. LCMS (ESI): m/z=498.2 [M+H]⁺; ¹H NMR (400 MHz, DMSO-d₆) δ 8.69 (d, J=1.9 Hz, 1H), 8.65 (d, J=2.3 Hz, 1H), 8.46 (d, J=2.1 Hz, 1H), 8.40 (s, 1H), 8.17 (t, J=2.1 Hz, 1H), 7.84 (s, 1H), 7.02 (s, 2H), 6.96 (d, J=2.1 Hz, 1H), 3.93 (s, 3H), 3.15 (s, 3H), 2.28 (s, 3H), 2.07 (s, 6H).

### Example 15: Method L: Synthesis of Compound I-93

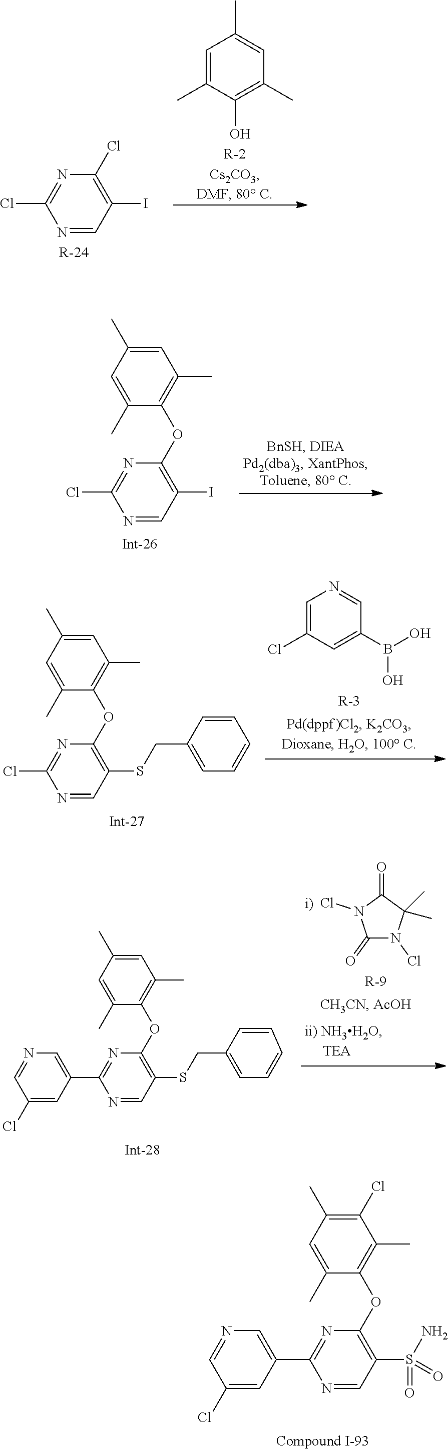

Step 1: To a stirred solution of 2, 4-dichloro-5-iodopyrimidine (R-24, 2.74 g, 10.0 mmol, 1.0 eq.) and 2,4,6-trimethylphenol (R-2, 1.36 g, 10.0 mmol, 1.0 eq.) in DMF (30 mL) was added Cs₂CO₃ (3.91 g, 12.0 mmol, 1.2 eq.) at room temperature. The resulting mixture was stirred at 80° C. for 5 hrs. After completion, the reaction mixture was diluted with H₂O (50 mL) and extracted with EtOAc (50 mL×3). The organic layers were combined and washed with brine (30 mL×3), dried over anhydrous Na₂SO₄ and concentrated under reduced pressure. The residue was purified by flash column chromatography on silica gel (eluted with PE:EtOAc=10:1 to 5:1) to afford Int-26 (2.92 g, 7.82 mmol, 78%) as a white solid. LCMS (ESI): m/z=375 [M+H]⁺.

Step 2: To a mixture of Int-26 (2.92 g, 7.82 mmol, 1 eq.) and phenylmethanethiol (970 mg, 7.82 mmol, 1.0 eq.) in toluene (20 mL) were added Pd₂(dba)₃ (714.3 mg, 0.78 mmol, 0.1 eq.), XantPhos (677.0 mg, 1.17 mmol, 0.15 eq.) and DIEA (302.4 mg, 2.34 mmol, 3.0 eq.). The resulting mixture was stirred at 80° C. under nitrogen atmosphere overnight. After completion, the reaction mixture was diluted with H₂O (30 mL) and extracted with EtOAc (30 mL×3). The combined organic layers were washed with brine (30 mL×3), dried over anhydrous Na₂SO₄ and concentrated under reduced pressure. The residue was purified by flash column chromatography through silica gel (eluted with PE:EtOAc=5:1 to 4:1) to afford Int-27 (2.09 g, 5.65 mmol, 72%) as a white solid. LCMS (ESI): m/z=371 [M+H]⁺.

Step 3: To a stirred solution of Int-27 (2.09 g, 5.65 mmol, 1.0 eq.), (5-chloropyridin-3-yl)boronic acid (R-3, 1.06 g, 6.78 mmol, 1.2 eq.) and K₂CO₃ (2.34 mg, 16.95 mmol, 3.0 eq.) in dioxane (20 mL) and water (4 mL) was added Pd(dppf)Cl₂ (417 mg, 0.57 mmol, 0.1 eq.). The resulting mixture was stirred at 80° C. under nitrogen atmosphere overnight. After completion, the reaction mixture was diluted with H₂O (30 mL) and extracted with EtOAc (30 mL×3). The combined organic layers were washed with brine (30 mL×3), dried over anhydrous Na₂SO₄ and concentrated under reduced pressure. The residue was purified by flash column chromatography through silica gel (eluted with PE:EtOAc=4:1 to 3:1) to afford Int-28 (1.73 g, 3.86 mmol, 68%) as a white solid. LCMS (ESI): m/z=448 [M+H]⁺.

Step 4: A solution of Int-28 (100 mg, 0.224 mmol, 1 eq.) in ACN (5 mL) was cooled to 0° C., and then was treated with an aqueous solution of acetic acid (0.13 mL, 50%, 5 eq.) and 1,3-dichloro-5,5-dimethylimidazolidine-2,4-dione (R-9, 87 mg, 0.447 mmol, 2 eq.). The reaction mixture was stirred for 30 min, whereupon NH₃·H₂O (2 mL) was added, followed by TEA (2 mL). The resulting mixture was stirred at 80° C. under nitrogen atmosphere overnight. After completion, the reaction mixture was diluted with H₂O (20 mL) and extracted with EtOAc (20 mL×3). The combined organic layers were washed with brine (20 mL), dried over anhydrous Na₂SO₄ and concentrated under reduced pressure. The residue was purified by prep-HPLC (Method 4B) to afford Compound I-93 (21.9 mg, 0.05 mmol, 23%) as a white solid. Compound I-93: Retention time: 1.662. LCMS (ESI): m/z=439.0 [M+H]f; ¹H NMR (400 MHz, DMSO-d₆) δ 9.14 (s, 1H), 8.95 (d, J=1.7 Hz, 1H), 8.79 (d, J=2.4 Hz, 1H), 8.30 (t, J=2.1 Hz, 1H), 8.05 (s, 2H), 7.28 (s, 1H), 2.39 (s, 3H), 2.16 (s, 3H), 2.07 (s, 3H).

The following compounds were synthesized from the appropriate starting materials using Method L: Compounds I-89 and I-90.

### Example 16: Method M: Synthesis of Compound I-104

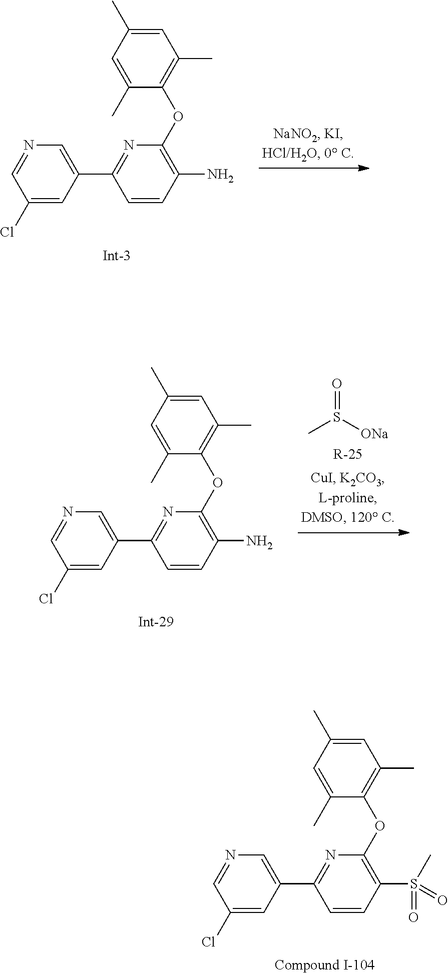

Step 1: A stirred solution of Int-3 (1.0 g, 2.95 mmol, 1.0 eq.) in HCl/H₂O (30 mL, 1 M) was cooled to 0° C. and then treated with aqueous NaNO₂ (306 mg, 4.43 mmol, 1.5 eq.) solution. The resulting mixture was stirred at 0° C. for 1 hr, and then KI (735 mg, 4.43 mmol, 1.5 eq.) was added at 0° C. The reaction mixture was stirred at 0° C. for an additional 1 hr, and then was was diluted with water (20 mL) and extracted with EtOAc (30 mL×3). The organic layers were combined and washed with brine (20 mL), dried over anhydrous Na₂SO₄ and concentrated under reduced pressure. The residue was purified by flash column chromatography through silica gel column (eluted with PE:EtOAc=3:1 to 2:1) to afford Int-29 (500 mg, 1.11 mmol, 38%) as a white solid. LC-MS (ESI) m/z 451 [M+H]⁺.

Step 2: To a stirred mixture of Int-29 (100 mg, 0.222 mmol, 1.0 eq.) in DMSO (10 mL) were added K₂CO₃ (61 mg, 0.44 mmol, 2.0 eq.), sodium methanesulfinate (R-25, 45 mg, 0.444 mmol, 2.0 eq.), CuI (4 mg, 0.022 mmol, 0.1 eq.) and L-proline (5 mg, 0.044 mmol, 0.2 eq.). The resulting mixture was stirred at 120° C. for 3 hrs. After completion, the reaction mixture was diluted with H₂O (20 mL) and extracted with EtOAc (20 mL×3). The organic layers were combined and washed with brine (20 mL×3), dried over anhydrous Na₂SO₄ and concentrated under reduced pressure. The residue was purified by flash column chromatography on silica gel (eluted with PE:EtOAc=5:1 to 4:1) to afford Compound I-104 (60 mg, 0.15 mmol, 67%) as a white solid. Compound I-104: Retention time: 1.897. LCMS (ESI): m/z=403.1 [M+H]⁺; ¹H NMR (400 MHz, DMSO-d₆) δ 8.84 (d, J=1.8 Hz, 1H), 8.69 (d, J=2.3 Hz, 1H), 8.42 (d, J=7.9 Hz, 1H), 8.25 (t, J=2.1 Hz, 1H), 8.07 (d, J=8.0 Hz, 1H), 7.02 (s, 2H), 3.51 (s, 3H), 2.30 (s, 3H), 2.05 (s, 6H).

### Example 17: Method N: Synthesis of Compound I-103

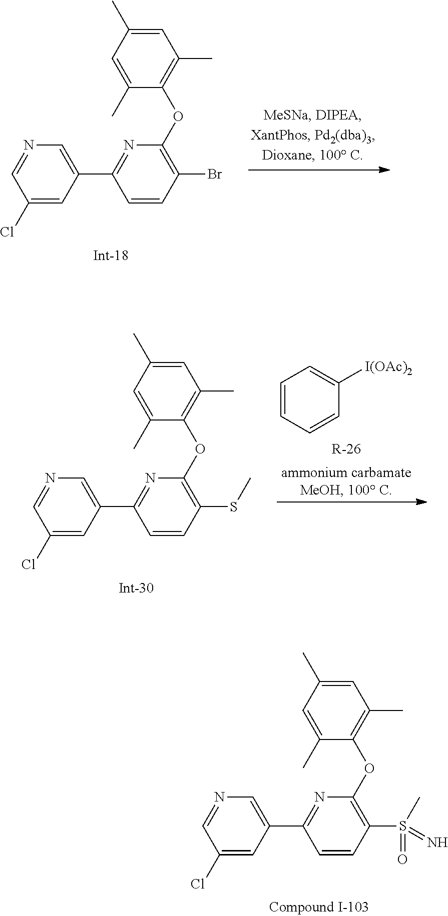

Step 1: To a stirred mixture of Int-18 (150 mg, 0.372 mmol, 1.0 eq.) in dioxane (20 mL) were added (methylsulfanyl)sodium (39 mg, 0.557 mmol, 1.5 eq.), Xantphos (64 mg, 0.111 mmol, 0.3 eq.), Pd₂(dba)₃ (68 mg, 0.074 mmol, 0.20 eq.) and DIEA (288 mg, 2.23 mmol, 6.0 eq.). The resulting mixture was stirred at 100° C. for 1.5 hrs. The reaction mixture was then diluted with H₂O (10 mL) and extracted with EtOAc (15 mL×3). The organic layers were combined, washed with brine (15 mL×3), dried over anhydrous Na₂SO₄ and concentrated under reduced pressure. The residue was purified by flash column chromatography on silica gel (eluted with PE:EtOAc=3:1 to 2:1) to afford Int-30 (80 mg, 0.216 mmol, 58%) as a white solid. LC-MS (ESI) m/z 371 [M+H]⁺.

Step 2: To a stirred mixture of Int-30 (90 mg, 0.243 mmol, 1.0 eq.) in MeOH (10 mL) were added (diacetoxyiodo)benzene (R-26, 236 mg, 0.728 mmol, 3.0 eq.) and ammonium carbamate (76 mg, 0.971 mmol, 4.0 eq.). The resulting mixture was stirred at 100° C. for 1 hr. After completion, the reaction mixture was diluted with H₂O (10 mL) and extracted with EtOAc (15 mL×3). The organic layers were combined, washed with brine (15 mL×3), dried over anhydrous Na₂SO₄ and concentrated under reduced pressure. The residue was purified by flash column chromatography on silica gel (eluted with PE:EtOAc=3:1 to 1:1) to afford Compound I-103 (30 mg, 0.075 mmol, 30%) as a white solid. Compound I-103: Retention time: 1.644 min. LC-MS (ESI) m/z 402.1 [M+H]^{●}; ¹H NMR (400 MHz, DMSO-d₆) δ 8.83 (d, J=1.8 Hz, 1H), 8.66 (d, J=2.3 Hz, 1H), 8.42 (d, J=7.9 Hz, 1H), 8.23 (t, J=2.1 Hz, 1H), 8.03 (d, J=7.9 Hz, 1H), 7.01 (s, 2H), 4.74 (s, 1H), 3.38 (s, 3H), 2.30 (s, 3H), 2.07 (s, 3H), 2.03 (s, 3H).

### Example 18: Method O: Synthesis of Compound I-23

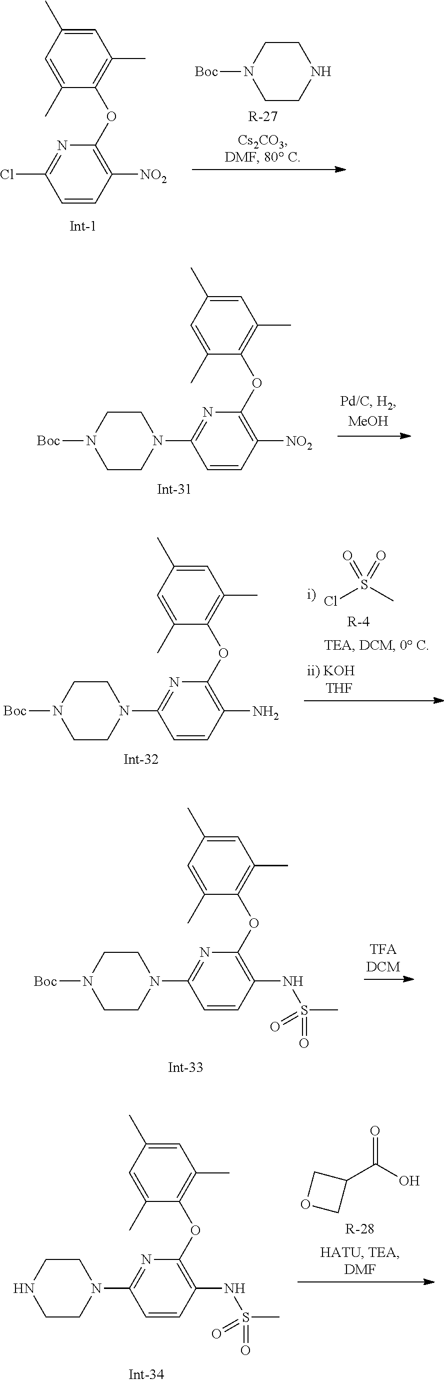

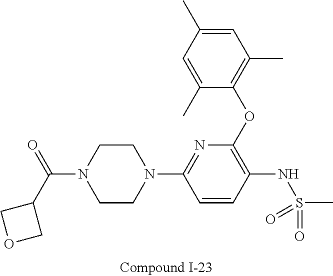 [SMILES: Cc1cc(C)c(Oc2nc(N3CCN(C(=O)C4COC4)CC3)ccc2NS(C)(=O)=O)c(C)c1]

Step 1: To a stirred solution of Int-1 (4.0 g, 13.7 mmol, 1.0 eq.) and tert-butyl piperazine-1-carboxylate (R-27, 3.05 g, 16.4 mmol, 1.2 eq.) in DMF (12 mL) was added Cs₂CO₃ (6.68 g, 20.5 mmol, 1.5 eq.) at room temperature. The resulting mixture was stirred at 80° C. for 2 hrs. After completion, the reaction mixture was diluted with H₂O (30 mL) and extracted with EtOAc (30 mL×3). The organic layers were combined and washed with brine (20 mL×3), dried over anhydrous Na₂SO₄ and concentrated under reduced pressure. The residue was purified by flash column chromatography through silica gel (eluted with PE:EtOAc=4:1 to 3:1) to afford Int-31 (5.8 g, 13.1 mmol, 96%) as a white solid. LCMS (ESI): m/z=443 [M+H]⁺.

Step 2: To a stirred solution of Int-31 (4.1 g, 9.27 mmol, 1.0 eq.) in MeOH (20 mL) was added Pd/C (274 mg, 10 wt %) at room temperature under nitrogen. The suspension was degassed under vacuum and purged with H₂ several times. The resulting mixture was stirred at room temperature for 2 hrs. After completion, the suspension was filtered through a pad of Celite, and the filter cake was washed with MeOH (20 mL). The combined filtrates were concentrated to dryness to give Int-32 (3.6 g, quant.) as a brown solid. LCMS (ESI): m/z=413 [M+H]⁺.

Step 3: A stirred solution of Int-32 (2.8 g, 6.79 mmol, 1.0 eq.) and TEA (0.3 mL) in DCM (30 mL) was cooled to 0° C. and methanesulfonyl chloride (R-4, 1.94 g, 17.0 mmol, 2.5 eq.) was added slowly. The resulting mixture was stirred at room temperature for 20 min. After completion, the reaction mixture was diluted with H₂O (20 mL) and extracted with EtOAc (30 mL×3). The organic layers were combined, dried over anhydrous Na₂SO₄ and concentrated under reduced pressure. The residue was dissolved with THE (10 mL), then to the solution was added an aqueous solution of KOH (1 M, 10 mL). The resulting mixture was stirred at room temperature for 30 min. After completion, the reaction mixture was diluted with H₂O (20 mL) and extracted with EtOAc (25 mL×3). The organic layers were combined, dried over anhydrous Na₂SO₄ and concentrated under reduced pressure. The residue was purified by flash column chromatography on silica gel (eluted with PE:EtOAc=100:1 to 4:1) to afford Int-33 (2.0 g, 4.076 mmol, 60%) as a white solid. LCMS (ESI): m/z=491 [M+H]⁺.

Step 4: To a stirred solution of Int-33 (2.0 g, 4.076 mmol, 1.0 eq.) in DCM (20 mL) was added TFA (10 mL). The resulting mixture was stirred at room temperature for 2 hrs. After completion, the reaction mixture was concentrated under reduced pressure to afford Int-34 as a TFA salt (1.2 g, quant.) as a yellow oil, which was used in the next step directly without further purification. LCMS (ESI): m/z=391 [M+H]⁺.

Step 5: To a stirred solution of oxetane-3-carboxylic acid (R-28, 16 mg, 0.15 mmol, 1.1 eq.) and HATU (49 mg, 0.13 mmol, 1.0 eq.) in DMF (3 mL) were added TEA (1 mL) and Int-34 TFA salt (50 mg, 0.13 mmol, 1.0 eq.). The mixture was stirred at room temperature for 30 min. After completion, the reaction mixture was purified by Prep-HPLC (Method 10A) to afford Compound I-23 (20 mg, 0.042 mmol, 33%). Compound I-23: Retention time: 1.418 min. LCMS (ESI): m/z=475.2 [M+H]⁺; ¹H NMR (400 MHz, DMSO-d₆) δ 9.12 (s, 1H), 7.50 (d, J=8.5 Hz, 1H), 6.90 (s, 2H), 6.41 (d, J=8.6 Hz, 1H), 4.69-4.57 (m, 4H), 4.15-4.08 (m, 1H), 3.45-3.38 (m, 2H), 3.24-3.06 (m, 6H), 3.01 (s, 3H), 2.25 (s, 3H), 1.99 (s, 6H).

### Example 19: Method P: Synthesis of Compound I-92

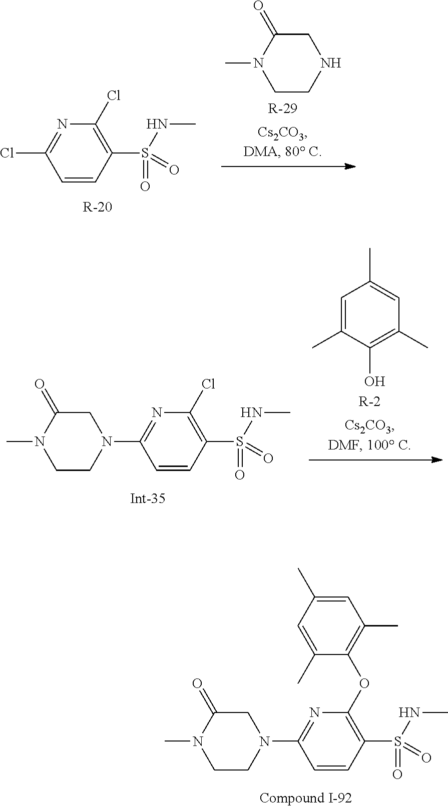

Step 1: To a stirred solution of R-20 (480 mg, 2.0 mmol, 1.0 eq.) in DMA (20 mL) were added 1-methylpiperazin-2-one (R-29, 228 mg, 2.0 mmol, 1.0 eq.) and Cs₂CO₃ (782 mg, 2.4 mmol, 1.2 eq.). The resulting mixture was stirred at 80° C. for 3 hrs. After completion, the reaction mixture was diluted with H₂O (30 mL) and extracted with EtOAc (30 mL×3). The organic layers were combined, dried over anhydrous Na₂SO₄ and concentrated under reduced pressure. The residue was purified by flash column chromatography through silica gel (eluted with PE:EtOAc=4:1 to 2:1) to afford Int-35 (363 mg, 1.14 mmol, 57%) as a white solid. LCMS (ESI): m/z=319 [M+H]⁺.

Step 2: To a solution of Int-35 (50 mg, 0.16 mmol, 1.0 eq.) in DMF (5 mL) were added Cs₂CO₃ (156 mg, 0.48 mmol, 3.0 eq.) and 2,4,6-trimethylphenol (R-2, 33 mg, 0.24 mmol, 1.5 eq.). The resulting mixture was stirred at 100° C. for 16 hrs. After completion, the reaction mixture was purified by prep-HPLC (Method 4B) to afford Compound I-92 (10 mg, 0.024 mmol, 15%) as a white solid. Compound I-92: Retention time: 1.186 min. LCMS (ESI): m/z=419.5 [M+H]⁺; ¹H NMR (400 MHz, CDCl3) δ 8.06 (d, J=8.6 Hz, 1H), 6.87 (s, 2H), 6.18 (d, J=8.6 Hz, 1H), 4.85 (d, J=5.8 Hz, 1H), 3.96 (s, 2H), 3.56 (t, J=5.4 Hz, 2H), 3.27 (t, J=5.4 Hz, 2H), 2.98 (s, 3H), 2.70 (d, J=4.4 Hz, 3H), 2.30 (s, 3H), 2.07 (s, 6H).

The following compounds were synthesized with the appropriate reagents using Method P: Compound I-73.

### Example 20: Method Q: Synthesis of Compound I-5

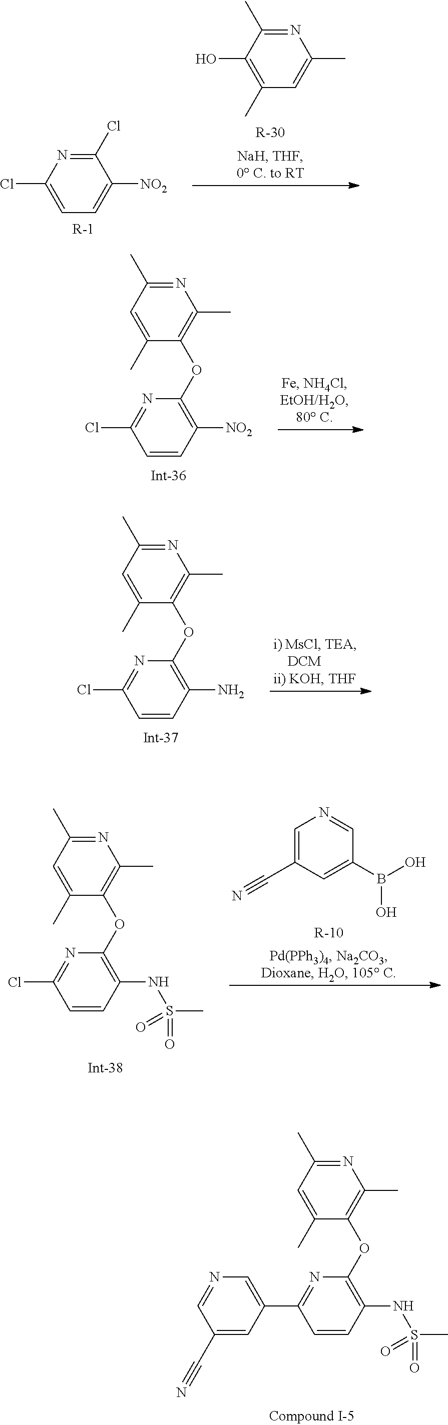

Step 1: To a stirred solution of 2,4,6-trimethylpyridin-3-ol (R-30, 1.38 g, 10.0 mmol, 1.0 eq.) in THE (50 mL) was added NaH (480 mg, 12.0 mmol, 1.2 eq.) at 0° C. slowly. The resulting mixture was stirred at room temperature under N₂ atmosphere for 30 min. 2,6-dichloro-3-nitropyridine (R-1, 1.92 g, 10.0 mmol, 1.0 eq.) was added. The mixture was stirred at room temperature for 2 hrs. After completion, the reaction was quenched by a saturated aqueous solution of ammonium chloride (20 mL). The mixture was extracted with EtOAc (20 mL×3). The organic layers were combined, washed with brine (20 mL×3), dried over anhydrous Na₂SO₄ and concentrated under reduced pressure. The residue was purified by flash column chromatography on silica gel (eluted with PE:EtOAc=100:1 to 10:1) to afford Int-36 (2.34 g, 8 mmol, 80%) as a yellow solid. LCMS (ESI): m/z=294 [M+H]⁺.

Step 2: To a stirred solution of Int-36 (294 mg, 1.0 mmol, 1.0 eq.) in 10 mL of EtOH and 5 mL of H₂O were added Fe powder (280 mg, 5.0 mmol, 5 eq.) and ammonium chloride (268 mg, 5.0 mmol, 5 eq.) at room temperature. The resulting mixture was stirred at 80° C. for 5 hrs. After completion, the reaction mixture was filtered through a pad of Celite and the filter cake was washed with MeOH (20 mL). The combined filtrates were concentrated to dryness. The residue was diluted with water (10 mL) and then extracted with EtOAc (20 mL×3). The combined organic layer was dried over anhydrous Na₂SO₄ and concentrated under reduced pressure. The residue was purified by flash column chromatography on silica gel (eluted with PE:EtOAc=100:1 to 2:1) to afford Int-37 (210.4 mg, 0.8 mmol, 80%) as a grey solid. LCMS (ESI): m/z=264 [M+H]⁺.

Step 3: To a stirred solution of Int-37 (210.4 mg, 0.8 mmol, 1.0 eq.) and TEA (0.3 mL) in DCM (5 mL) was added methane sulfonyl chloride (228 mg, 2.0 mmol, 2.5 eq.) at 0° C. slowly. The resulting mixture was stirred at room temperature for 20 min. After completion, the reaction mixture was diluted with H₂O (10 mL) and extracted with EtOAc (10 mL×3). The organic layers were combined and concentrated under reduced pressure.

The residue was dissolved with THE (10 mL) and aqueous solution KOH (1 M, 5 mL) was added. The resulting mixture was stirred at room temperature for 30 min. After completion, the reaction mixture was diluted with H₂O (10 mL) and extracted with EtOAc (15 mL×3). The organic layers were combined, dried over anhydrous Na₂SO₄ and concentrated under reduced pressure. The residue was purified by flash column chromatography on silica gel (eluted with PE:EtOAc=100:1 to 4:1) to afford Int-38 (218 mg, 0.636 mmol, 80%) as a white solid. LCMS (ESI): m/z=342 [M+H]⁺.

Step 4: To a mixture of Int-38 (100 mg, 0.293 mmol, 1 eq.), (5-cyanopyridin-3-yl)boronic acid (R-10, 52 mg, 0.351 mmol, 1.2 eq.) and Na₂CO₃ (93.2 mg, 0.879 mmol, 3.0 eq.) in dioxane (5 mL) and water (1 mL) was added Pd(PPh₃)₄ (35 mg, 0.03 mmol, 0.1 eq.). The resulting mixture was stirred at 105° C. under nitrogen atmosphere overnight. After completion, the reaction mixture was diluted with H₂O (10 mL) and extracted with EtOAc (10 mL×3). The combined organic phase was dried over anhydrous Na₂SO₄ and concentrated under reduced pressure. The residue was purified by prep-HPLC to afford Compound I-5 (8 mg, 0.019 mmol, 7%) as a white solid.

Compound I-5: Retention time: 1.829 min; LCMS (ESI): m/z=410.2 [M+H]⁺; ¹H NMR (400 MHz, DMSO-d₆) δ 8.98 (d, J=2.2 Hz, 1H), 8.93 (d, J=1.9 Hz, 1H), 8.52 (t, J=2.1 Hz, 1H), 7.91 (d, J=8.1 Hz, 1H), 7.84 (d, J=8.1 Hz, 1H), 7.10 (s, 1H), 3.14 (s, 3H), 2.43 (s, 3H), 2.22 (s, 3H), 2.07 (s, 3H).

The following compounds were synthesized from the appropriate alcohol, boronic acid/boronate and sulfonyl chloride reagents using Method Q: Compounds I-172, I-186, I-187, I-188, I-211, I-226, I-227, I-231, I-232, I-238, I-239, I-241, I-242, I-243, I-246, I-247, I-250, I-259, I-260, I-266, I-269, I-270, I-276, I-282, and I-284.

### Example 21: Method R: Synthesis of Compound 1-4

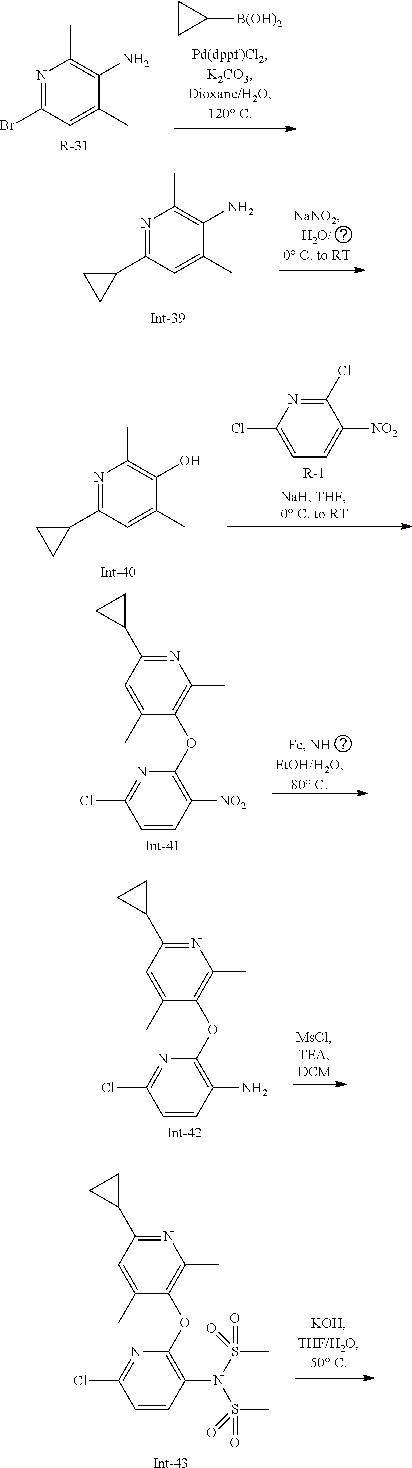

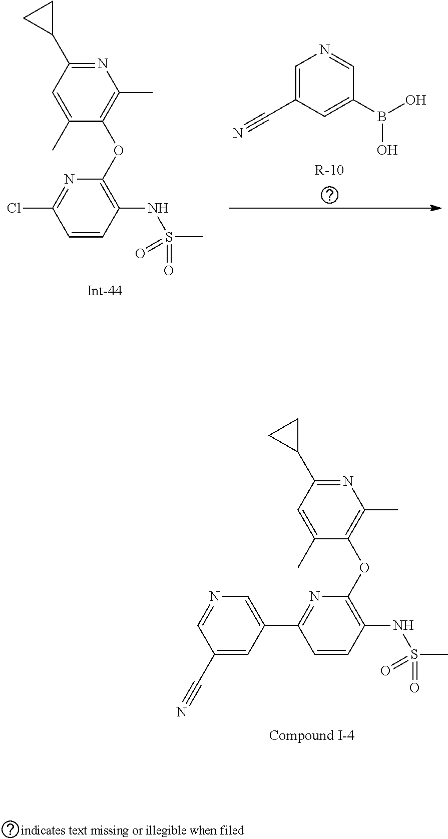 [SMILES: Cc1cc(C2CC2)nc(C)c1Oc1nc(Cl)ccc1NS(C)(=O)=O ; N#Cc1cncc(B(O)O)c1 ; "R-10" ; "Int-44" ; Cc1cc(C2CC2)nc(C)c1Oc1nc(-c2cncc(C#N)c2)ccc1NS(C)(=O)=O]

Step 1: To a stirred solution of R-31 (500 mg, 2.49 mmol, 1.0 eq.) in dioxane (30 mL) and H₂O (10 mL) were added K₂CO₃ (1.38 g, 9.96 mmol, 4.0 eq.), cyclopropylboranediol (320 mg, 3.73 mmol, 1.5 eq.) and Pd(dppf)Cl₂ (91 mg, 0.12 mmol, 0.05 eq.). The resulting mixture was stirred at 120° C. for 24 hrs under N₂. After completion, the reaction mixture was poured into water (10 mL) and then extracted with EtOAc (15 mL×3). The organic layers were combined and washed with brine (15 mL), dried over anhydrous Na₂SO₄, and concentrated under reduced pressure. The residue was purified by flash column chromatography on silica gel (eluted with PE:EtOAc=100:1 to 10:1) to afford Int-39 (200 mg, 1.23 mmol, 49%) as a white solid. LC-MS (ESI) m/z 163 [M+H]⁺.

Step 2: To a stirred solution of Int-39 (200 mg, 1.23 mmol, 1.0 eq.) in H₂SO₄/H₂O (2 mL/10 mL) was added sodium nitrite (170 mg, 2.47 mmol, 2.0 eq.) slowly at 0° C. After addition, the resulting mixture was stirred at 0° C. for 30 min. After completion, the reaction mixture was quenched with a sat. aq. NaHCO₃ solution (15 mL) and then extracted with EtOAc (20 mL×3). The organic layers were combined and washed with brine (15 mL), dried over anhydrous Na₂SO₄, and concentrated under reduced pressure to afford Int-40 (150 mg, 0.92 mmol, 75%) as a white solid. LC-MS (ESI) m/z 164 [M+H]⁺.

Step 3: To a stirred solution of Int-40 (100 mg, 0.61 mmol, 1.0 eq.) in THE (10 mL) was added NaH (61 mg×60 wt %, 1.53 mmol, 2.5 eq.) slowly at 0° C. The resulting mixture was stirred at 0° C. for 30 min. Then 2,6-dichloro-3-nitropyridine (R-1, 351 mg, 1.83 mmol, 3.0 eq.) was added and the resulting mixture was stirred at room temperature for 30 min. After completion, the reaction mixture was poured into water (10 mL) and then extracted with EtOAc (15 mL×3). The organic layers were combined and washed with brine (15 mL), dried over anhydrous Na₂SO₄, and concentrated under reduced pressure. The residue was purified by flash column chromatography on silica gel (eluted with PE:EtOAc=4:1 to 3:1) to afford Int-41 (50 mg, 0.16 mmol, 26%) as a white solid. LC-MS (ESI) m/z 320 [M+H]⁺.

Step 4: To a solution of Int-41 (120 mg, 0.38 mmol, 1.0 eq.) in EtOH (9 mL) and H₂O (3 mL) were added NH₄Cl (201 mg, 3.75 mmol, 10.0 eq.) and Fe (210 mg, 3.75 mmol, 10.0 eq.). The resulting mixture was stirred at 80° C. for 1 hr under N₂. After completion, the reaction mixture was filtered through a pad of Celite and the filter cake was washed with MeOH (20 mL). The combined filtrates were concentrated to dryness. The residue was diluted with water (10 mL) and then extracted with EtOAc (15 mL×3). The organic layers were combined and washed with brine (15 mL), dried over anhydrous Na₂SO₄, and concentrated under reduced pressure. The residue was purified by flash column chromatography on silica gel (eluted with PE:EtOAc=4:1 to 3:1) to afford Int-42 (70 mg, 0.24 mmol, 64%) as a white solid. LC-MS (ESI) m/z 290 [M+H]⁺.

Step 5: To a solution of Int-42 (45 mg, 0.16 mmol, 1.0 eq.) and TEA (47 mg, 0.47 mmol, 3.0 eq.) in DCM (4 mL) was added MsCl (17.75 mg, 0.16 mmol, 1.0 eq.) at 25° C. The resulting mixture was stirred at 25° C. for 1 hr. After completion, the reaction mixture was diluted with water (10 mL) and then extracted with EtOAc (15 mL×3). The organic layers were combined and washed with brine (15 mL), dried over anhydrous Na₂SO₄, and concentrated under reduced pressure to afford Int-43 (50 mg, 0.11 mmol, 70%) as a yellow oil. LC-MS (ESI) m/z 446 [M+H]⁺.

Step 6: To a stirred mixture of Int-43 (40 mg, 90 μmol, 1.0 eq.) in THE (3 mL) and H₂O (1 mL) was added KOH (30 mg, 0.54 mmol, 6.0 eq.) at room temperature. The resulting mixture was stirred at 50° C. for 2 hrs. After completion, the reaction mixture was diluted with water (10 mL) and then extracted with EtOAc (15 mL×3). The organic layers were combined and washed with brine (15 mL), dried over anhydrous Na₂SO₄, and concentrated under reduced pressure. The residue was purified by flash column chromatography on silica gel (eluted with PE:EtOAc=4:1 to 3:1) to afford Int-44 (30 mg, 82 μmol, 91%) as a white solid. LC-MS (ESI) m/z 368 [M+H]⁺.

Step 7: To a solution of Int-44 (30 mg, 82 μmol, 1.0 eq.) in dioxane (3 mL) and H₂O (1 mL) were added Na₂CO₃ (35 mg, 0.33 mmol, 4.0 eq.), (5-cyanopyridin-3-yl)boronic acid (18 mg, 123 μmol, 1.5 eq.) and Pd(PPh₃)₄ (9 mg, 8 μmol, 0.1 eq.). The reaction mixture was stirred at 60° C. under N₂ for 16 hrs. After completion, the reaction mixture was poured into water (10 mL) and then extracted with EtOAc (15 mL×3). The organic layers were combined and washed with brine (15 mL), dried over anhydrous Na₂SO₄ and concentrated under reduced pressure. The residue was purified by reverse phase chromatography using a 40 g C18 cartridge eluting with a gradient of 5-50% MeCN in water (with 0.1% FA) to afford Compound I-4 (4.6 mg, 10 μmol, 12%) as a yellow solid.

Compound I-4: Retention time: 0.55 min; LC-MS (ESI) m/z 436.3 [M+H]⁺; ¹H NMR (400 MHz, DMSO-d₆) δ 8.92 (s, 1H), 8.76 (s, 1H), 8.33 (s, 1H), 7.63 (s, 2H), 7.05 (s, 1H), 2.16 (s, 3H), 2.04 (s, 3H), 1.36-1.33 (m, 1H), 1.23 (s, 3H), 0.91-0.86 (m, 4H).

The following compounds were synthesized from the appropriate reagents using Method R: Compounds I-120, I-127, I-147, I-208, I-213, I-229, I-264, and I-268.

### Example 22: Method S: Synthesis of Compound I-167

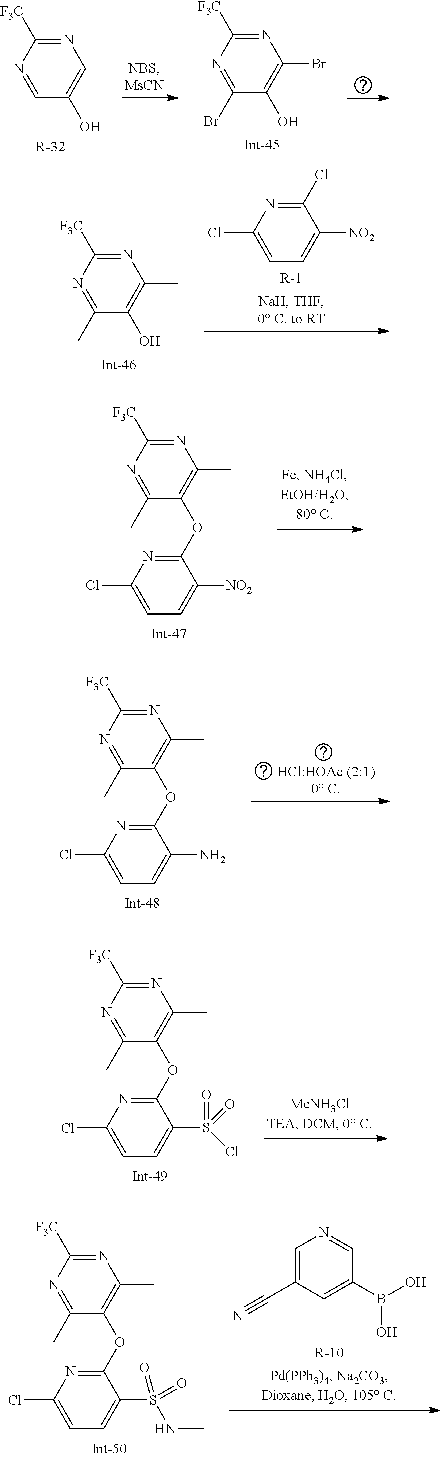

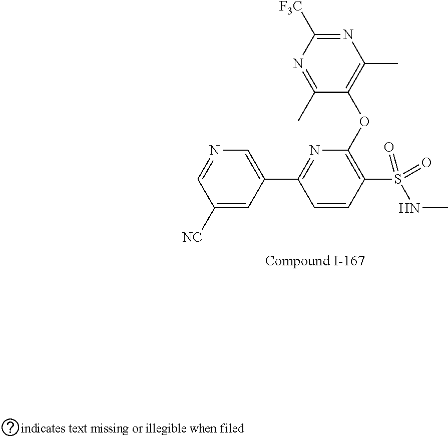 [SMILES: CNS(=O)(=O)c1ccc(-c2cncc(C#N)c2)nc1Oc1c(C)nc(C(F)(F)F)nc1C]

Step 1: To a stirred solution of 2-(trifluoromethyl)pyrimidin-5-ol (R-32, 1.0 g, 6.1 mmol, 1.0 eq.) in MeCN (30 mL) was added NBS (2.7 g, 15.3 mmol, 2.5 eq.), slowly. After stirring at room temperature for 2 hrs, the reaction was diluted with H₂O (30 mL) and extracted with EtOAc (30 mL×3). The combined organic phase was dried over anhydrous Na₂SO₄ and concentrated under reduced pressure. The residue was purified by flash column chromatography on silica gel (eluted with PE:EtOAc=0 to 5:1) to afford Int-45 (897.5 mg, 2.8 mmol, 46%) as a white solid. LCMS (ESI): m/z=321 [M+H]⁺.

Step 2: A mixture of Int-45 (897.5 mg, 2.8 mmol, 1 eq.), methylboronic acid (838.0 mg, 14.0 mmol, 5.0 eq.), cataCXium A Pd G3 (203.9 mg, 0.28 mmol, 0.1 eq.) and K₂CO₃ (1.55 g, 11.2 mmol, 4.0 eq.) in dioxane (20 mL) and water (4 mL) was stirred at 80° C. under N₂ atmosphere overnight. After completion, the mixture was diluted with H₂O (70 mL) and extracted with EtOAc (20 mL×3). The combined organic phase was dried over anhydrous Na₂SO₄ and concentrated under reduced pressure. The residue was purified by flash column chromatography on silica gel (eluted with PE:EtOAc=4:1) to afford Int-46 (371 mg, 1.9 mmol, 68%) as a yellow solid. LCMS (ESI): m/z=193 [M+H]⁺.

Step 3: To a stirred solution of Int-46 (371 mg, 1.9 mmol, 1.0 eq.) in THE (10 mL) was added NaH (91.2 mg, 2.3 mmol, 1.2 eq.) at 0° C. under N₂ atmosphere, slowly. After stirring for 30 min, to the mixture was added 2,6-dichloro-3-nitropyridine (R-1, 401.2 mg, 2.1 mmol, 1.1 eq.) at 0° C. After stirring at room temperature for 2 hrs, the reaction was quenched by a saturated aqueous solution of NH₄Cl (20 mL) and extracted with EtOAc (20 mL×3). The combined organic phase was dried over anhydrous Na₂SO₄ and concentrated under reduced pressure. The residue was purified by flash column chromatography on silica gel (eluted with PE:EtOAc=0 to 5:1) to afford Int-47 (469.5 mg, 1.35 mmol, 71%) as a yellow solid. LCMS (ESI): m/z=349 [M+H]⁺.

Step 4: A mixture of Int-47 (469.5 mg, 1.35 mmol, 1.0 eq.), Fe powder (378.0 mg, 6.75 mmol, 5 eq.) and NH₄Cl (722.1 mg, 13.5 mmol, 10 eq.) in EtOH (20 mL) and H₂O (5 mL) was stirred at 80° C. for 2 hrs. After completion, the mixture was filtered through a pad of Celite and the filter cake was washed with MeOH (20 mL×3) and the combined filtrates were concentrated under reduced pressure. The residue was diluted with water (30 mL) and extracted with EtOAc (30 mL×3). The combined organic layer was dried over anhydrous Na₂SO₄ and concentrated under reduced pressure. The residue was purified by flash column chromatography through silica gel (eluted with PE:EtOAc=2:1 to 1:1) to afford Int-48 (304.9 mg, 0.96 mmol, 71%) as a white solid. LCMS (ESI): m/z=319 [M+H]⁺.

Step 5: To a stirred solution of Int-48 (304.9 mg, 0.96 mmol, 1.0 eq.) in concentrated HCl (40 mL) and glacial acetic acid (20 mL) was added aqueous solution of NaNO₂ (2.0 M, 1 mL, 2.0 eq.) at 0° C., slowly. After stirring for 30 min, to the mixture was added CuCl₂ (129.1 mg, 0.96 mmol, 1.0 eq.) and SO₂ was bubbled into the reaction mixture at 0° C. for 15 min. After completion, the mixture was poured into ice water (100 mL) and extracted with EtOAc (60 mL×3). The combined organic phase was washed with a saturated aqueous solution of NaHCO₃ (80 mL×3), dried over anhydrous Na₂SO₄ and concentrated under reduced pressure. The residue was purified by flash column chromatography through silica gel (eluted with PE:EtOAc=2:1 to 1:1) to afford Int-49 (163.6 mg, 0.41 mmol, 43%) as a yellow solid. LCMS (ESI): m/z=402 [M+H]⁺.

Step 6: To a stirred solution of Int-49 (163.6 mg, 0.41 mmol, 1.0 eq.) in DCM (10 mL) was added methylamine hydrochloride (55.4 mg, 0.82 mmol, 2.0 eq.) and TEA (0.5 mL) at 0° C. After stirring at room temperature for 30 min, the mixture was diluted with H₂O (10 mL) and extracted with EtOAc (10 mL×3). The organic layers were combined, dried over anhydrous Na₂SO₄ and concentrated under reduced pressure. The residue was purified by flash column chromatography through silica gel (eluted with PE:EtOAc=1:1 to 1:3) to afford Int-50 (109.1 mg, 0.276 mmol, 67%) as a white solid. LCMS (ESI): m/z=397 [M+H]⁺.

Step 7: A mixture of Int-50 (109.1 mg, 0.276 mmol, 1.0 eq.), (5-chloropyridin-3-yl)boronic acid (R-10, 45.0 mg, 0.304 mmol, 1.1 eq.), Pd(PPh₃)₄ (34.7 mg, 0.03 mmol, 0.11 eq.) and Na₂CO₃ (86.4 mg, 0.828 mmol, 3.0 eq.) in dioxane (10 mL) and water (2 mL) was stirred at 90° C. under N₂ atmosphere for 6 hrs. After completion, the reaction mixture was diluted with H₂O (20 mL) and extracted with EtOAc (20 mL×3). The organic layers were combined, dried over anhydrous Na₂SO₄ and concentrated under reduced pressure. The residue was purified by prep-HPLC (column: Welch XBridge C18 250×21 mm, gradient: 30-95% MeCN in H₂O [with 0.1% FA]) to give Compound I-167 (23.2 mg, 0.05 mmol, 18%).

Compound I-167: Retention time: 1.527 min; LCMS (ESI): m/z=465 [M+H]⁺; ¹H NMR (400 MHz, DMSO) δ 9.13-9.05 (m, 2H), 8.64 (d, J=1.8 Hz, 1H), 8.52-8.44 (m, 1H), 8.20-8.12 (m, 1H), 8.02 (d, J=0.5 Hz, 1H), 2.63 (s, 3H), 2.44 (d, J=5.1 Hz, 6H). ¹⁹F NMR (376 MHz, DMSO) δ −68.31 (s).

The following compounds were synthesized with the appropriate reagents using Method S: Compounds I-133, I-168, I-258, I-265, I-280, I-281, I-285, I-292, I-309, I-310, and I-315.

### Example 23: Method T: Synthesis of Compound I-220

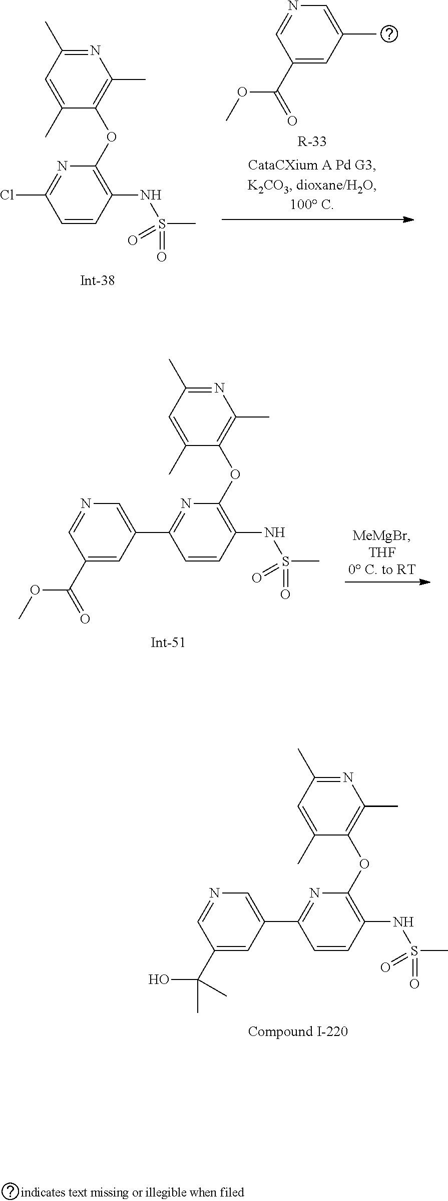

Step 1: A mixture of Int-38 (100 mg, 0.29 mmol, 1.0 eq.), (5-(methoxycarbonyl)pyridin-3-yl)boronic acid (R-33, 58.33 mg, 0.322 mmol, 1.1 eq.), cataCXium A Pd G3 (21.33 mg, 0.03 mmol, 0.1 eq.) and K₂CO₃ (121.3 mg, 0.879 mmol, 3.0 eq.) in dioxane (10 mL) and water (2 mL) was stirred under N₂ atmosphere at 100° C. for 6 hrs. After completion, the reaction mixture was diluted with H₂O (30 mL) and extracted with EtOAc (20 mL×3). The organic layers were combined, dried over anhydrous Na₂SO₄ and concentrated under reduced pressure. The residue was purified by flash column chromatography through silica gel (eluted with PE:EtOAc=1:1 to 1:3) to afford Int-51 (104.3 mg, 0.24 mmol, 83%) as a white solid. LCMS (ESI): m/z=443 [M+H]⁺.

Step 2: To a stirred solution of Int-51 (104.3 mg, 0.24 mmol, 1 eq) in THE (20 mL) was added a THF solution of MeMgBr (1 M, 0.84 mL, 0.84 mmol, 3.5 eq.) at 0° C. under N₂ atmosphere, slowly. After stirring at room temperature for 2 hrs, the reaction mixture was quenched with H₂O (30 mL) and extracted with EtOAc (12 mL×3). The organic layers were combined, dried over anhydrous Na₂SO₄ and concentrated under reduced pressure. The residue was purified by flash column chromatography through silica gel (eluted with PE:EtOAc=1:1 to 1: 4) to afford Compound I-220 (11.2 mg, 0.025 mmol, 10.7%).

Compound I-220: Retention time: 0.421 min; LCMS (ESI): m/z=443.2 [M+H]⁺. ¹H NMR (400 MHz, DMSO-d₆) δ 10.02 (s, 1H), 8.73 (dd, J=14.8, 2.1 Hz, 2H), 8.21 (t, J=2.1 Hz, 1H), 8.01 (d, J=8.1 Hz, 1H), 7.92 (d, J=8.1 Hz, 1H), 7.64 (s, 1H), 3.23 (s, 3H), 2.64 (s, 3H), 2.44 (s, 3H), 2.26 (s, 3H), 1.43 (s, 6H).

### Example 24: Method U: Synthesis of Compound I-212

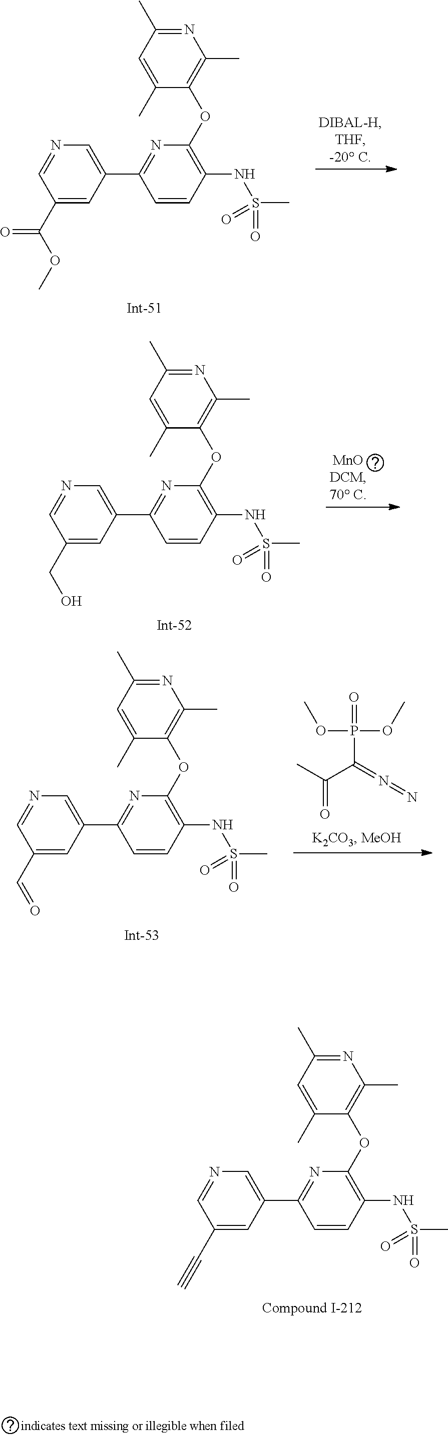

Step 1: To a stirred mixture of Int-51 (80 mg, 0.18 mmol, 1 eq.) in THE (1 mL) was added DIBAL-H (0.5 mL, 1.5 M, 0.75 mmol, 4.2 eq.) at −78° C. under N₂ atmosphere, dropwise. After stirring at −20° C. for 2 hrs, an aqueous solution of potassium sodium tartrate tetrahydrate (30 mL) was added. Then, the mixture was stirred at room temperature for another 30 min and extracted with EtOAc (20 mL×3). The combined organic layers were dried over anhydrous Na₂SO₄ and concentrated under reduced pressure. The residue was purified by silica column (eluted with PE:EtOAc=1:1) to afford Int-52 (66.3 mg, 0.16 mmol, 87%) as a yellow solid. LCMS (ESI): m/z=415 [M+H]⁺.

Step 2: To a stirred solution of Int-52 (65 mg, 0.16 mmol, 1 eq.) in THE (2 mL) was added MnO₂ (139 mg, 1.6 mmol, 10 eq.). After stirring at 70° C. overnight, the mixture reaction was cooled to room temperature and filtered through a pad of Celite and the filter cake was washed with DCM (20 mL×3). The combined filtrates were concentrated under reduced pressure to afford Int-53 (30 mg, crude) as a white solid, which was used for the next step directly. LCMS (ESI): m/z=413 [M+H]⁺.

Step 3: To a stirred mixture of Int-53 (30 mg, crude) in MeOH (2 mL) was added dimethyl (1-diazo-2-oxopropyl)phosphonate (27.95 mg, 0.15 mmol) and K₂CO₃ (25.13 mg, 0.18 mmol) under N₂ atmosphere at 0° C., slowly. After stirring at room temperature for 1 h, the mixture was diluted with water (20 mL) and extracted with EtOAc (10 mL×3). The combined organic phase was dried over anhydrous Na₂SO₄ and concentrated under reduced pressure. The residue was purified by prep-HPLC (column: YMC-Actus Triart C18 250×20 mm×5 um, gradient: 30%-95% MeCN in H₂O [with 0.1% TFA]) to afford Compound I-212 (5 mg, 0.012 mmol, 7.7% for two steps) as a white solid.

Compound I-212: Retention time: 1.178 min; LCMS (ESI): m/z=409.2 [M+H]⁺. ¹H NMR (400 MHz, DMSO) δ 9.88 (s, 1H), 8.81 (d, J=2.1 Hz, 1H), 8.60 (d, J=1.9 Hz, 1H), 8.11 (t, J=2.0 Hz, 1H), 7.91 (d, J=8.1 Hz, 1H), 7.83 (d, J=8.1 Hz, 1H), 7.10 (s, 1H), 4.48 (s, 1H), 3.18 (s, 3H), 2.44 (s, 3H), 2.23 (s, 3H), 2.07 (s, 3H).

### Example 25: Method V: Synthesis of Compound I-161

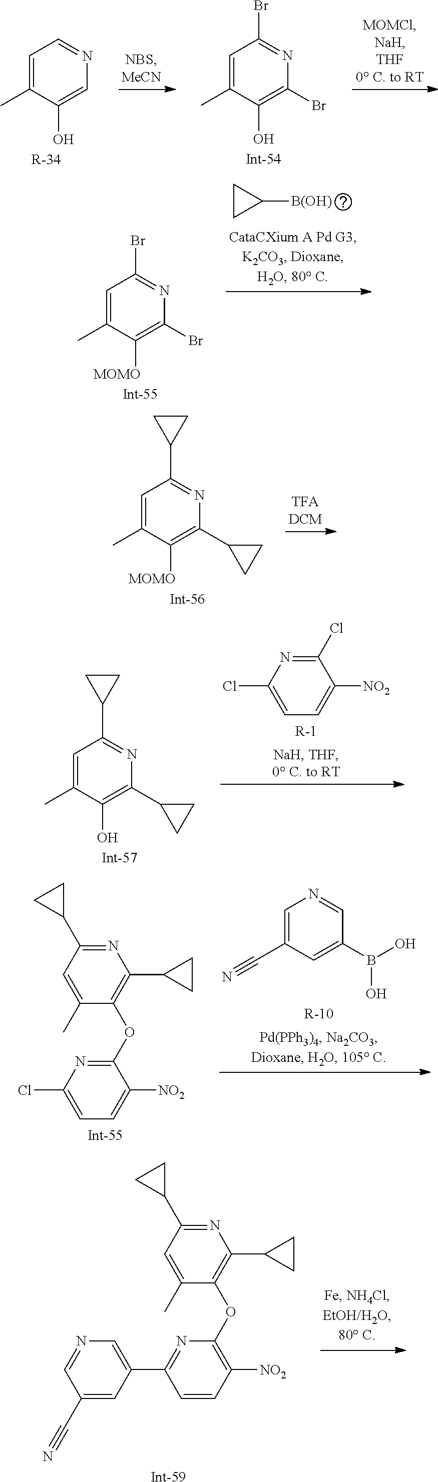

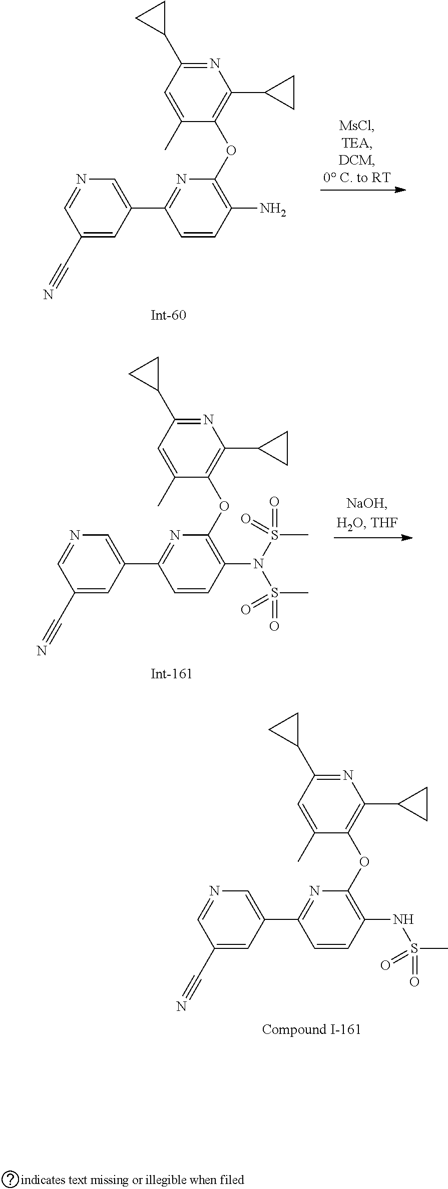

Step 1: To a stirred solution of 4-methylpyridin-3-ol (R-34, 1.0 g, 9.2 mmol, 1.0 eq.) in MeCN (50 mL) was added NBS (4.1 g, 23 mmol, 2.5 eq.), slowly. After stirring at r.t. for 2 hrs, the reaction was diluted with H₂O (80 mL) and extracted with EtOAc (50 mL×3). The combined organic phase was dried over anhydrous Na₂SO₄ and concentrated under reduced pressure. The residue was purified by flash column chromatography on silica gel (eluted with PE:EtOAc=0 to 1:3) to afford Int-54 (1.25 g, 4.7 mmol, 51.3%) as a white solid. LCMS (ESI): m/z=266 [M+H]⁺.

Step 2: To a stirred solution of Int-54 (1.25 g, 4.7 mmol, 1 e.) in THE (50 mL) was added NaH (282 mg, 7.05 mmol, 1.5 eq.) under N₂ atmosphere at 0° C. slowly. After stirring for 30 min, to the mixture was added MOMCl (454 mg, 5.6 mmol, 1.2 eq.), then the mixture was stirred at r.t. for another 2 hrs. After completion, the mixture was quenched with a saturated aqueous solution of NH₄Cl (50 mL) and extracted with EtOAc (40 mL×3). The combined organic phase was dried over anhydrous Na₂SO₄ and concentrated under reduced pressure. The residue was purified by flash column chromatography on silica gel (eluted with PE:EtOAc=4:1) to afford Int-55 (1.1 g, 3.6 mmol, 76.6%) as a white solid. LCMS (ESI): m/z=310 [M+H]⁺.

Step 3: A mixture of Int-55 (1.1 g, 3.6 mmol, 1 eq.), cyclopropylboronic acid (1.5 g, 18.0 mmol, 5.0 eq.), cataCXium A Pd G3 (524.4 mg, 0.72 mmol, 0.2 eq.) and K₂CO₃ (2.0 g, 14.4 mmol, 4.0 eq.) in dioxane (50 mL) and water (10 mL) was stirred at 80° C. under N₂ atmosphere overnight. After completion, the mixture was diluted with H₂O (70 mL) and extracted with EtOAc (50 mL×3). The combined organic phase was dried over anhydrous Na₂SO₄ and concentrated under reduced pressure. The residue was purified by flash column chromatography on silica gel (eluted with PE:EtOAc=4:1) to afford Int-56 (298.4 mg, 1.28 mmol, 35.6%) as a white solid. LCMS (ESI): m/z=234 [M+H]⁺.

Step 4 and 5: To a stirred solution of Int-56 (298.4 mg, 1.28 mmol, 1.0 eq.) in DCM (20 mL) was added TFA (8 mL). After stirring at r.t. for 30 min, the mixture was concentrated under reduced pressure to remove the solvent and TFA. The crude Int-57 was dissolved in anhydrous THE (20 mL), then to the resulting mixture was added NaH (128 mg, 3.2 mmol, 2.5 eq.) under N₂ atmosphere at 0° C. slowly. After stirring for 30 min, to the mixture was added 2,6-dichloro-3-nitropyridine (R-1, 290 mg, 1.5 mmol, 1.2 eq.). The resulting mixture was stirred at r.t. for another 2 hrs. After completion, the reaction was quenched with a saturated aqueous solution of NH₄Cl (30 mL) and extracted with EtOAc (20 mL×3). The combined organic phase was dried over anhydrous Na₂SO₄ and concentrated under reduced pressure. The residue was purified by flash column chromatography through silica gel (eluted with PE:EtOAc=3:1) to afford Int-58 (296.4 mg, 0.86 mmol, 67.1%) as a yellow solid. LCMS (ESI): m/z=346 [M+H]⁺.

Step 6: A mixture of Int-58 (296.4 mg, 0.86 mmol, 1.0 eq.), (5-cyanopyridin-3-yl)boronic acid (R-10, 148 mg, 1.0 mmol, 1.2 eq.), Pd(PPh₃)₄ (104 mg, 0.09 mmol, 0.1 eq.) and Na₂CO₃ (273.5 mg, 2.58 mmol, 3.0 eq.) in dioxane (40 mL) and water (8 mL) was stirred under N₂ atmosphere at 80° C. for 4 hrs. After completion, the reaction mixture was diluted with H₂O (50 mL) and extracted with EtOAc (40 mL×3). The organic layers were combined, dried over anhydrous Na₂SO₄ and concentrated under reduced pressure. The residue was purified by flash column chromatography on silica gel (eluted with PE:EtOAc=1: 4) to afford Int-59 (263 mg, 0.64 mmol, 74%) as a yellow solid. LCMS (ESI): m/z=414 [M+H]⁺.

Step 7: A mixture of Int-59 (263 mg, 0.64 mmol, 1.0 eq.), Fe powder (179.2 mg, 3.2 mmol, 5 eq.) and NH₄Cl (342.4 mg, 6.4 mmol, 10 eq.) in EtOH (50 mL) and H₂O (25 mL) was stirred at 80° C. for 2 hrs. After completion, the reaction mixture was filtered through a pad of Celite and the filter cake was washed with MeOH. The combined filtrates were concentrated, the residue was triturated with H₂O and filtered to afford Int-60 (210 mg, crude) as a brown solid, which was used for next step, directly. LCMS (ESI): m/z=384 [M+H]⁺.

Step 8 and 9: To a stirred solution of Int-60 (100 mg, crude) in DCM (20 mL) was added MsCl (0.5 mL) and TEA (1 mL) at 0° C. slowly. After stirring at r.t. for 10 min, the reaction mixture was diluted with H₂O (30 mL) and extracted with EtOAc (20 mL×3). The combined organic layers were concentrated under reduced pressure. The crude Int-61 was dissolved with THF (20 mL), then to the mixture was added an aqueous solution of NaOH (4 M, 2 mL). After stirring at r.t. for 30 min, the mixture was neutralized by HCl (1 M) and extracted with EtOAc (20 mL×3). The combined organic phase was dried over anhydrous Na₂SO₄ and concentrated under reduced pressure. The residue was purified by Prep-HPLC (column: XB—C18 5 um×21.2×250 mm, gradient: 15-95% MeCN in H₂O (with 0.1% TFA)) to afford Compound I-161 (18.4 mg, 0.04 mmol, 15.4% over 2 steps) as a white solid.

Compound I-161: Retention time: 3.861 min; LCMS (ESI): m/z=462.1 [M+H]⁺; ¹H NMR (400 MHz, DMSO-d₆) δ (ppm) 10.02 (s, 1H), 9.06 (d, J=2.2 Hz, 1H), 8.97 (d, J=1.9 Hz, 1H), 8.55 (t, J=2.1 Hz, 1H), 8.00-7.86 (m, 2H), 7.05 (s, 1H), 3.23 (s, 3H), 2.10-1.99 (m, 5H), 0.97-0.64 (m, 8H).

The following compounds were synthesized using Method V with the appropriate alcohols, boronic acids/boronates and sulfonyl chlorides: Compounds I-162 and I-171.

### Example 26: Method W: Synthesis of Compound I-137

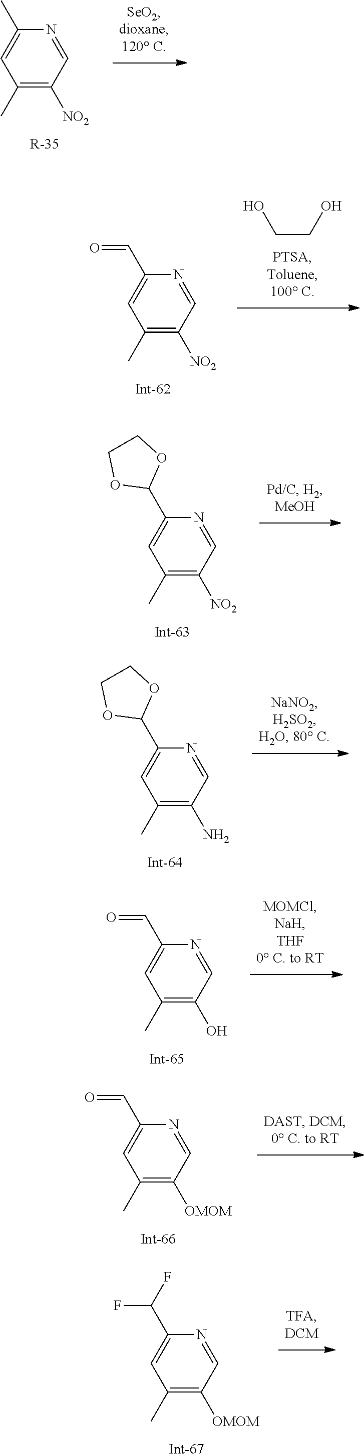

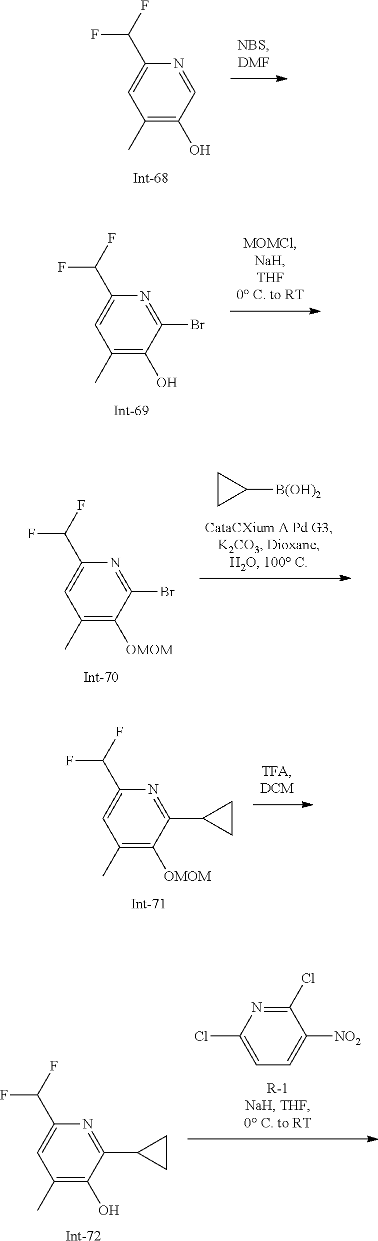

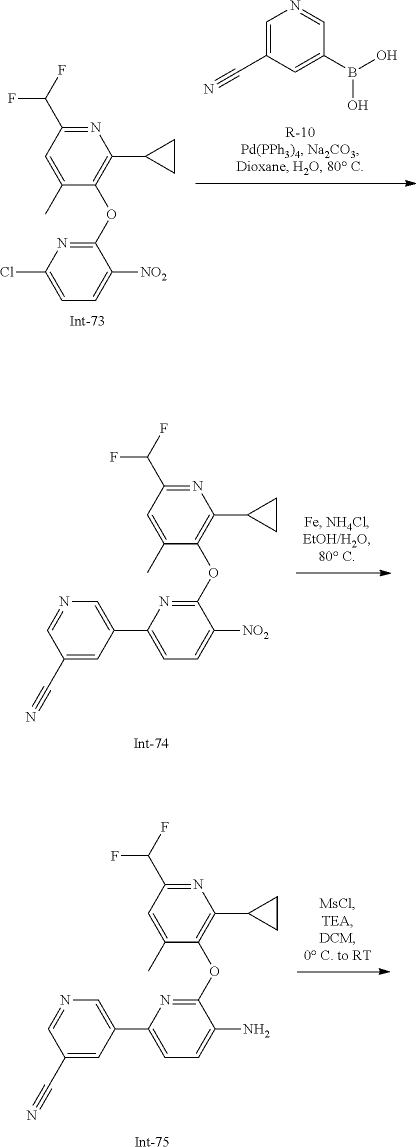

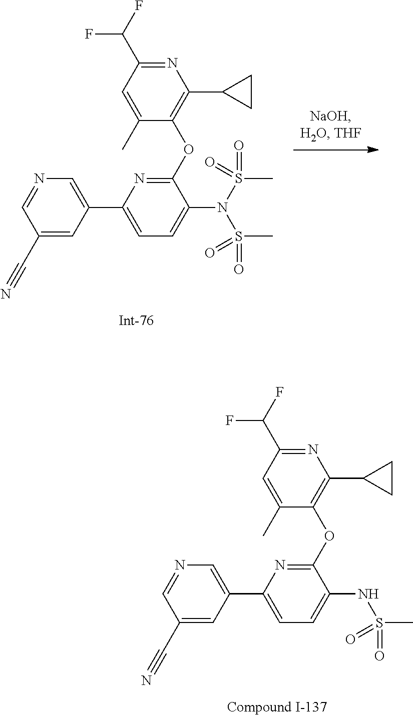 [SMILES: Cc1cc(C(F)F)nc(C2CC2)c1Oc1nc(-c2cncc(C#N)c2)ccc1N(S(C)(=O)=O)S(C)(=O)=O ; "Int-76" ; Cc1cc(C(F)F)nc(C2CC2)c1Oc1nc(-c2cncc(C#N)c2)ccc1NS(C)(=O)=O ; "Compound I-137"]

Step 1: To a stirred solution of 2, 4-dimethyl-5-nitropyridine (R-35, 10.0 g, 65.8 mmol, 1.0 eq.) in dioxane (100 mL) was added SeO₂ (18.4 g, 164.4 mmol, 2.5 eq.) slowly. After stirring 120° C. for 5 hrs, the reaction was diluted with H₂O (150 mL) and extracted with EtOAc (120 mL×3). The combined organic phase was dried over anhydrous Na₂SO₄ and concentrated under reduced pressure. The residue was purified by flash column chromatography on silica gel (eluted with PE:EtOAc=0 to 10:1) to afford Int-62 (4.5 g, 27.1 mmol, 41.2 0%) as a yellow solid. LCMS (ESI): m/z=167 [M+H]⁺.

Step 2: To a stirred solution of Int-62 (4.5 g, 27.1 mmol, 1.0 eq.), ethylene glycol (16.8 g, 271 mmol, 10.0 eq.) in Tol. (100 mL) was added PTSA (464.9 mg, 2.7 mmol, 0.1 eq.), slowly. After stirring at 100° C. for 5 hrs, the reaction was diluted with H₂O (160 mL) and extracted with EtOAc (120 mL×3). The combined organic phase was dried over anhydrous Na₂SO₄ and concentrated under reduced pressure. The residue was purified by flash column chromatography on silica gel (eluted with PE:EtOAc=0 to 10:1) to afford Int-63 (5.1 g, 24.2 mmol, 89.3%) as a yellow solid. LCMS (ESI): m/z=211 [M+H].

Step 3: To a mixture of Int-63 (5.1 g, 24.2 mmol, 1.0 eq.) in MeOH (30 mL) was added Pd/C (250 mg). The mixture was stirred under hydrogen atmosphere at r.t. for 5 hrs. After completion, the mixture was filtered, the filter cake was washed with MeOH (20 mL×3) and the combined filtrates were concentrated under reduced pressure. The residue was purified by flash column chromatography through silica gel (eluted with PE:EtOAc=0 to 3:1) to afford Int-64 (3.6 g, 20.2 mmol, 83.6 0%) as a white solid. LCMS (ESI): m/z=211 [M+H].

Step 4 and 5: To a stirred solution of Int-64 (3.6 g, 20.2 mmol, 1.0 eq.) in water (100 mL) was added concentrated sulfuric acid (20 mL) and aqueous solution of NaNO₂ (2 M, 15 mL, 30 mmol, 1.5 eq.) at 0° C. slowly. After stirring at 80° C. for 6 hrs, the mixture was poured into ice water (150 mL) and neutralized by an aqueous solution of NaOH (4 M). After completion, the resulting mixture was extracted with EtOAc (100 mL×3), and the organic phase was combined, dried over anhydrous Na₂SO₄ and concentrated under reduced pressure. The crude Int-65 was dissolved with anhydrous THE (100 mL), then to the resulting mixture was added NaH (1.2 g, 30 mmol, 1.5 eq.) under N₂ atmosphere at 0° C. slowly. After stirring for 30 min, to the mixture was added MOMCl (1.95 g, 24.2 mmol, 1.2 eq.). After stirring at r.t. for 2 hrs, the mixture was quenched with a saturated aqueous solution of NH₄Cl (50 mL) and extracted with EtOAc (50 mL×3). The combined organic phase was dried over anhydrous Na₂SO₄ and concentrated under reduced pressure. The residue was purified by flash column chromatography on silica gel (eluted with PE:EtOAc=4:1) to afford Int-66 (1.5 g, 8.3 mmol, 41.4% for two steps) as a white solid. LCMS (ESI): m/z=182 [M+H]⁺.

Step 6: To a stirred solution of Int-66 (1.5 g, 8.3 mmol, 1.0 eq.) in DCM (50 mL) was added DAST (5.4 g, 33.2 mmol, 4.0 eq.) at 0° C. After stirring at r.t. for 30 mins, the reaction mixture was diluted with H₂O (100 mL) and extracted with DCM (80 mL×3). The organic layers were combined, dried over anhydrous Na₂SO₄ and concentrated under reduced pressure. The residue was purified by flash column chromatography through silica gel (eluted with PE:EtOAc=4:1) to afford Int-67 (1.2 g, 5.8 mmol, 69.5%) as a white solid. LCMS (ESI): m/z=204 [M+H]⁺.

Step 7: To a stirred solution of Int-67 (1.2 g, 5.8 mmol, 1.0 eq.) in DCM (20 mL) was added TFA (5 mL). After stirring at r.t. for 30 min, the mixture was concentrated under reduced pressure to remove the solvent. The residue was purified by flash column chromatography through silica gel (eluted with EtOAc) to afford Int-68 (763.4 mg, 4.8 mmol, 82.8%) as a white solid. LCMS (ESI): m/z=160 [M+H]⁺.

Step 8 and 9: To a stirred solution of Int-68 (763.4 mg, 4.8 mmol, 1.0 eq.) in DMF (30 mL) was added NBS (1.0 g, 5.8 mmol, 1.2 eq.). After stirring at r.t. for 2 hrs, the mixture was diluted with H₂O (50 mL) and extracted with EtOAc (30 mL×3). The organic layers were combined, washed with brine (30 mL×3), dried over anhydrous Na₂SO₄ and concentrated under reduced pressure. The crude Int-69 was dissolved with anhydrous THE (40 mL), then to the resulting mixture was added NaH (230 mg, 7.2 mmol, 1.5 eq.) under N₂ atmosphere at 0° C., slowly. After stirring for 30 min, to the mixture was added MOMCl (580 mg, 7.2 mmol, 1.5 eq.). After stirring at r.t. for 2 hrs, the mixture was quenched with a saturated aqueous solution of NH₄Cl (50 mL) and extracted with EtOAc (40 mL×3). The combined organic phase was dried over anhydrous Na₂SO₄ and concentrated under reduced pressure. The residue was purified by flash column chromatography on silica gel (eluted with PE:EtOAc=4:1) to afford Int-70 (955.4 mg, 3.4 mmol, 70.3% for two steps) as a white solid. LCMS (ESI): m/z=282 [M+H]⁺.

Step 10: A mixture of Int-70 (955.4 mg, 3.4 mmol, 1.0 eq.), cyclopropylboronic acid (584 mg, 6.8 mmol, 2.0 eq.), cataCXium A Pd G3 (255 mg, 0.35 mmol, 0.1 eq.) and K₂CO₃ (1.4 g, 10.2 mmol, 3.0 eq.) in dioxane (30 mL) and H₂O (6 mL) was stirred at 100° C. under N₂ atmosphere for 3 hrs. After completion, the mixture was diluted with H₂O (50 mL) and extracted with EtOAc (30 mL×3), the combined organic phase was dried over anhydrous Na₂SO₄ and concentrated under reduced pressure. The residue was purified by flash column chromatography through silica gel (eluted with PE:EtOAc=4:1) to afford Int-71 (681 mg, 2.8 mmol, 82.3%) as a white solid. LCMS (ESI): m/z=244 [M+H]⁺.

Step 11 and 12: To a stirred solution of Int-71 (681 mg, 2.8 mmol, 1.0 eq.) in DCM (20 mL) was added TFA (8 mL). After stirring at r.t. for 30 min, the mixture was concentrated under reduced pressure to remove the solvent and TFA. The crude Int-72 was dissolved with anhydrous THE (40 mL), then to the resulting mixture was added NaH (280 mg, 7.0 mmol, 2.5 eq.) under N₂ atmosphere at 0° C., slowly. After stirring for 30 min, to the mixture was added 2, 6-dichloro-3-nitropyridine (R-1, 656.2 mg, 3.4 mmol, 1.2 eq.). The resulting mixture was stirred at r.t. for another 2 hrs. After completion, the reaction was quenched with a saturated aqueous solution of NH₄Cl (40 mL) and extracted with EtOAc (30 mL×3). The combined organic phase was dried over anhydrous Na₂SO₄ and concentrated under reduced pressure. The residue was purified by flash column chromatography through silica gel (eluted with PE:EtOAc=2:1) to afford Int-73 (609 mg, 1.7 mmol, 61.3% for two steps) as a yellow solid. LCMS (ESI): m/z=356 [M+H]⁺.

Step 13: A mixture of Int-73 (609 mg, 1.7 mmol, 1.0 eq.), (5-cyanopyridin-3-yl)boronic acid (R-10, 296 mg, 2.0 mmol, 1.2 eq.), Pd(PPh₃)₄ (196.5 mg, 0.17 mmol, 0.1 eq.) and Na₂CO₃ (530.6 mg, 5.1 mmol, 3.0 eq.) in dioxane (40 mL) and water (10 mL) was stirred under N₂ atmosphere at 80° C. for 4 hrs. After completion, the reaction mixture was diluted with H₂O (50 mL) and extracted with EtOAc (30 mL×3). The organic layers were combined, dried over anhydrous Na₂SO₄ and concentrated under reduced pressure. The residue was purified by flash column chromatography on silica gel (eluted with PE:EtOAc=1: 5) to afford Int-74 (517.8 mg, 1.2 mmol, 72%) as a yellow solid. LCMS (ESI): m/z=424 [M+H]⁺.

Step 14: A mixture of Int-74 (517.8 g, 1.2 mmol, 1.0 eq.), Fe powder (336 mg, 6.0 mmol, 5 eq.) and NH₄Cl (642 mg, 12 mmol, 10 eq.) in EtOH (50 mL) and H₂O (20 mL) was stirred at 80° C. for 3 hrs. After completion, the mixture was filtered through a pad of Celite, the filter cake was washed with MeOH, the combined filtrates were concentrated, the residue was triturated with H₂O and filtered to afford Int-75 (480 mg, crude product) as a brown solid, which was used for the next step directly. LCMS (ESI): m/z=394 [M+H]⁺.

Step 15 and 16: To a stirred solution of Int-75 (120 mg, crude) in DCM (10 mL) was added MsCl (1 mL) and TEA (2 mL) at 0° C. slowly. After stirring at r.t. for 10 min, the reaction mixture was diluted with H₂O (30 mL) and extracted with EtOAc (20 mL×3). The combined organic layers were concentrated under reduced pressure. The crude Int-76 was dissolved with THF (10 mL), then to the mixture was added an aqueous solution of NaOH (4 M, 1 mL). After stirring at r.t. for 30 min, the mixture was neutralized by HCl (1 M) and extracted with EtOAc (20 mL×3). The combined organic phase was dried over anhydrous Na₂SO₄ and concentrated under reduced pressure. The residue was purified by Prep-HPLC (column: YMC-Actus Triart C18 250×20 mm×5 um, gradient: 40-95% MeCN in H₂O (with 0.1% NH₄HCO₃)) to afford Compound I-137 (19.3 mg, 0.041 mmol, 13.7% for three steps) as a white solid.

Compound I-137: Retention time: 1.538 min; LCMS (ESI): m/z=472.1 [M+H]⁺; ¹H NMR (400 MHz, DMSO-d₆) δ (ppm) 10.10 (s, 1H), 9.00 (d, J=2.0 Hz, 1H), 8.96 (d, J=1.8 Hz, 1H), 8.56 (t, J=1.9 Hz, 1H), 8.01 (d, J=8.1 Hz, 1H), 7.93 (d, J=8.1 Hz, 1H), 7.51 (s, 1H), 6.87 (t, J=55.1 Hz, 1H), 3.25 (s, 3H), 2.20 (s, 3H), 2.18-2.12 (m, 1H), 1.01-0.87 (m, 2H), 0.86-0.68 (m, 2H); ¹⁹F NMR (376 MHz, DMSO-d₆) δ (ppm) −114.46 (d, J=12.7 Hz).

The following compound was synthesized using Method W with the appropriate alcohols, boronic acids/boronates and sulfonyl chlorides: Compound I-179.

### Example 27: Method X: Synthesis of Compound I-200

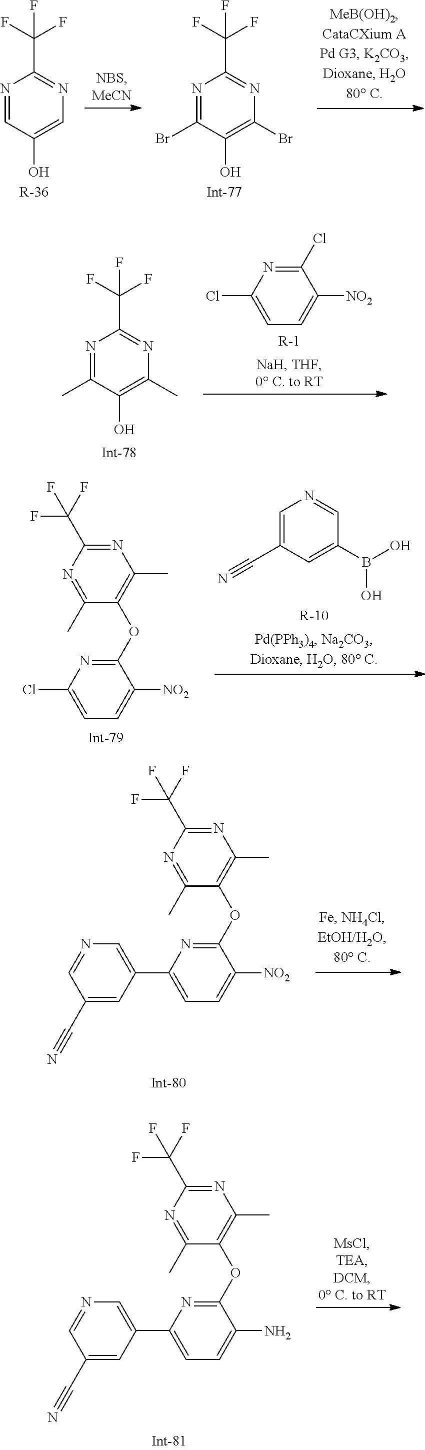

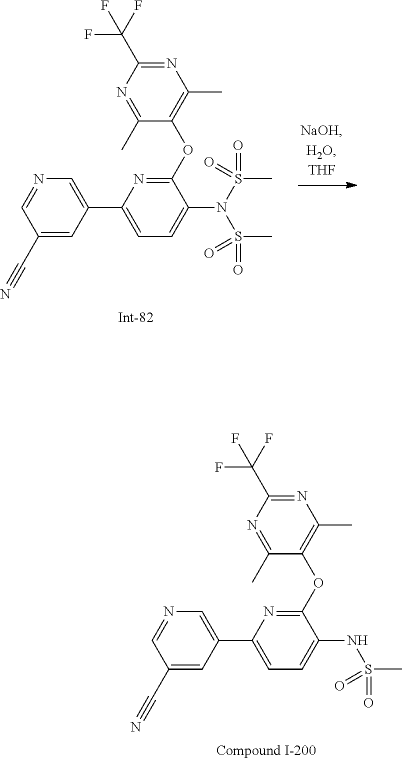 [SMILES: Cc1nc(C(F)(F)F)nc(C)c1Oc1nc(-c2cncc(C#N)c2)ccc1N(S(C)(=O)=O)S(C)(=O)=O ; Cc1nc(C(F)(F)F)nc(C)c1Oc1nc(-c2cncc(C#N)c2)ccc1NS(C)(=O)=O]

Step 1: To a stirred solution of 2-(trifluoromethyl)pyrimidin-5-ol (R-36, 1.0 g, 6.1 mmol, 1.0 eq.) in MeCN (30 mL) was added NBS (2.7 g, 15.3 mmol, 2.5 eq.), slowly. After stirring at r.t. for 2 hrs, the reaction was diluted with H₂O (70 mL) and extracted with EtOAc (30 mL×3). The combined organic phase was dried over anhydrous Na₂SO₄ and concentrated under reduced pressure. The residue was purified by flash column chromatography on silica gel (eluted with PE:EtOAc=0 to 1:1) to afford Int-77 (897.5 mg, 2.8 mmol, 45.9%) as a white solid. LCMS (ESI): m/z=321 [M+H]⁺.

Step 2: A mixture of Int-77 (897.5 mg, 2.8 mmol, 1.0 eq.), methylboronic acid (838.0 mg, 14.0 mmol, 5.0 eq.), cataCXium A Pd G3 (203.9 mg, 0.28 mmol, 0.1 eq.) and K₂CO₃ (1.55 g, 11.2 mmol, 4.0 eq.) in dioxane (30 mL) and water (6 mL) was stirred at 80° C. under N₂ atmosphere overnight. After completion, the mixture was diluted with H₂O (50 mL) and extracted with EtOAc (40 mL×3). The combined organic phase was dried over anhydrous Na₂SO₄ and concentrated under reduced pressure. The residue was purified by flash column chromatography on silica gel (eluted with PE:EtOAc=1:1) to afford Int-78 (371 mg, 1.9 mmol, 67.9%) as a yellow solid. LCMS (ESI): m/z=193 [M+H]⁺.

Step 3: To a stirred solution of Int-78 (371 mg, 1.9 mmol, 1.0 eq.) in THE (10 mL) was added NaH (92 mg, 2.3 mmol, 1.2 eq.) at 0° C. under N₂ atmosphere, slowly. After stirring at 0° C. for 30 min, to the mixture was added 2,6-dichloro-3-nitropyridine (R-1, 405.3 mg, 2.1 mmol, 1.1 eq.) at 0° C. After stirring at r.t. for 2 hrs, the reaction was quenched by saturated aqueous solution of NH₄Cl (50 mL) and extracted with EtOAc (20 mL×3). The combined organic phase was dried over anhydrous Na₂SO₄ and concentrated under reduced pressure. The residue was purified by flash column chromatography on silica gel (eluted with PE:EtOAc=0 to 5:1) to afford Int-79 (469.5 mg, 1.35 mmol, 71%) as a yellow solid. LCMS (ESI): m/z=349 [M+H]⁺.

Step 4: A mixture of Int-79 (142.7 mg, 0.41 mmol, 1.0 eq.), (5-cyanopyridin-3-yl)boronic acid (R-10, 72.5 mg, 0.49 mmol, 1.2 eq.), Pd(PPh₃)₄ (46.2 mg, 0.04 mmol, 0.1 eq.) and Na₂CO₃ (128 mg, 1.23 mmol, 3.0 eq.) in dioxane (20 mL) and water (4 mL) was stirred under nitrogen atmosphere at 80° C. for 2 hrs. After completion, the reaction mixture was diluted with H₂O (30 mL) and extracted with EtOAc (20 mL×3). The organic layers were combined, dried over anhydrous Na₂SO₄ and concentrated under reduced pressure. The residue was purified by flash column chromatography through silica gel (eluted with EA) to afford Int-80 (116.5 mg, 0.28 mmol, 68.3%) as a yellow solid. LCMS (ESI): m/z=417 [M+H]⁺.

Step 5: A mixture of Int-80 (116.5 mg, 0.28 mmol, 1.0 eq.), Fe powder (78 mg, 1.4 mmol, 5 eq.) and NH₄Cl (150 mg, 2.8 mmol, 10 eq.) in EtOH (20 mL) and H₂O (10 mL) was stirred at 80° C. for 2 hrs. After completion, the reaction mixture was filtered through a pad of Celite and the filter cake was washed with MeOH. The combined filtrates were concentrated, the residue was triturated with H₂O, then filtered to afford Int-81 (80 mg, crude product) as a brown solid. LCMS (ESI): m/z=387 [M+H]⁺.

Step 6 and 7: To a stirred solution of Int-81 (80 mg, crude) in DCM (10 mL) was added MsCl (0.5 mL) and TEA (1 mL) at 0° C., slowly. After stirring at r.t. for 10 min, the reaction mixture was diluted with H₂O (30 mL) and extracted with EtOAc (20 mL×3). The combined organic layers were concentrated under reduced pressure. The crude Int-82 was dissolved in THE (10 mL), then to the resulting mixture was added an aqueous solution of NaOH (4 M, 1 mL). After stirring at r.t. for 30 min, the mixture was neutralized by HCl (1 M) and extracted with EtOAc (15 mL×3). The combined organic phase was dried over anhydrous Na₂SO₄ and concentrated under reduced pressure. The residue was purified by Prep-HPLC (column: YMC-Actus Triart C18 250×20 mm×5 um, gradient: 30-95% MeCN in H₂O (with 0.1% TFA)) to afford Compound I-200 (14.7 mg, 0.032 mmol, 11.3% for three steps) as a white solid.

Compound I-200: Retention time: 1.358 min; LCMS (ESI): m/z=465.1 [M+H]⁺; ¹H NMR (400 MHz, DMSO-d₆) δ (ppm) 10.13 (s, 1H), 9.03 (d, J=1.9 Hz, 1H), 8.98 (d, J=1.4 Hz, 1H), 8.52 (s, 1H), 8.05 (d, J=8.1 Hz, 1H), 7.99 (d, J=8.1 Hz, 1H), 3.26 (s, 3H), 2.43 (s, 6H); 19F NMR (376 MHz, DMSO-d₆) δ (ppm) −68.29 (s).

The following compounds were synthesized using Method X with the appropriate alcohols, boronic acids/boronates and sulfonyl chlorides: Compounds I-131, I-138, I-145, I-151, and I-205.

### Example 28: Method Y: Synthesis of Compound I-155

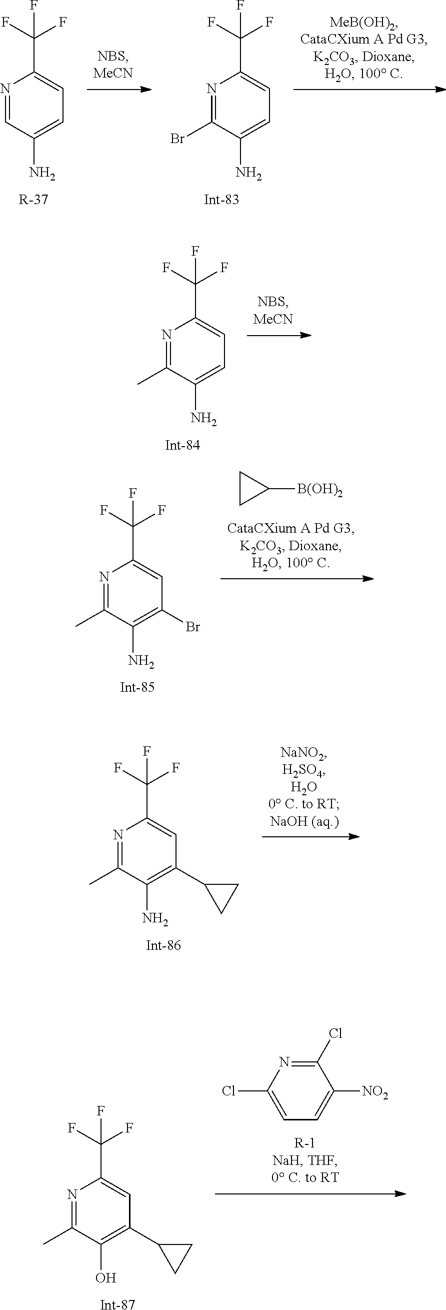

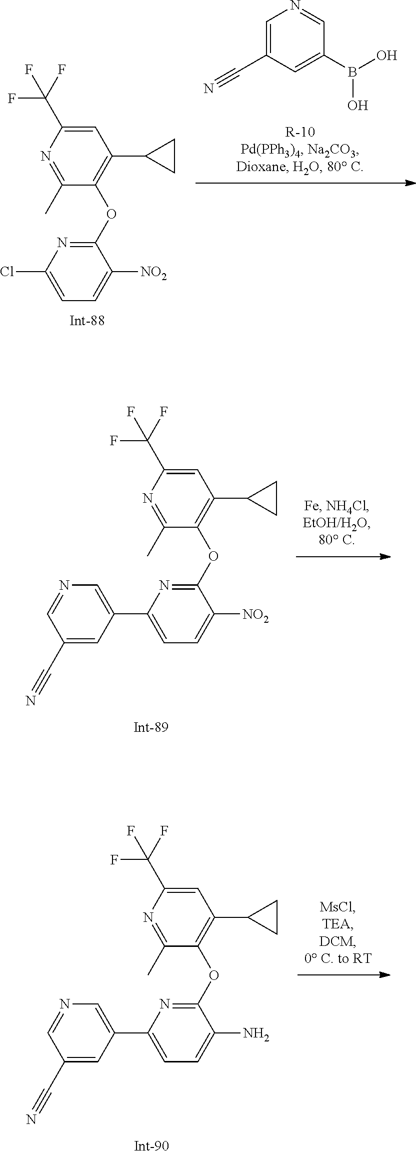

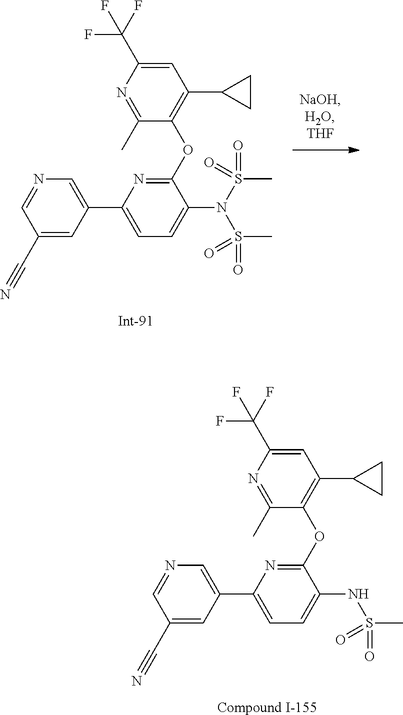 [SMILES: Cc1nc(C(F)(F)F)cc(C2CC2)c1Oc1nc(-c2cncc(C#N)c2)ccc1N(S(C)(=O)=O)S(C)(=O)=O ; "Int-91" ; Cc1nc(C(F)(F)F)cc(C2CC2)c1Oc1nc(-c2cncc(C#N)c2)ccc1NS(C)(=O)=O ; "Compound I-155"]

Step 1: To a stirred solution of 6-(trifluoromethyl)pyridin-3-amine (R-37, 1.6 g, 10.0 mmol, 1.0 eq.) in MeCN (50 mL) was added NBS (1.96 g, 11.0 mmol, 1.1 eq.). After stirring at r.t. for 2 hrs, the mixture was diluted with H₂O (90 mL) and extracted with EtOAc (30 mL×3). The organic layers were combined, dried over anhydrous Na₂SO₄ and concentrated under reduced pressure. The residue was purified by flash column chromatography through silica gel (eluted with PE:EtOAc=10:1) to afford Int-83 (2.0 g, 8.3 mmol, 83.0% o) as a colorless oil. LCMS (ESI): m/z=241 [M+H].

Step 2: A mixture of Int-83 (2.0 g, 8.3 mmol, 1.0 eq.), MeB(OH)₂ (2.0 g, 16.6 mmol, 2.0 eq.), cataCXium A Pd G3 (604.5 mg, 0.83 mmol, 0.1 eq.) and K₂CO₃ (3.4 g, 24.9 mmol, 3.0 eq.) in dioxane (50 mL) and H₂O (10 mL) was stirred at 100° C. under N₂ atmosphere for 3 hrs. After completion, the mixture was diluted with H₂O (60 mL) and extracted with EtOAc (40 mL×3), the combined organic phase was dried over anhydrous Na₂SO₄ and concentrated under reduced pressure. The residue was purified by flash column chromatography through silica gel (eluted with PE:EtOAc=5:1) to afford Int-84 (1.15 g, 6.56 mmol, 79.0% o) as a yellow solid. LCMS (ESI): m/z=177 [M+H]⁺.

Step 3: To a stirred solution of Int-84 (1.15 g, 6.56 mmol, 1.0 eq.) in MeCN (30 mL) was added NBS (1.4 g, 7.9 mmol, 1.2 eq.). After stirring at r.t. for 2 hrs, the mixture was diluted with H₂O (50 mL) and extracted with EtOAc (30 mL×3). The organic layers were combined, dried over anhydrous Na₂SO₄ and concentrated under reduced pressure. The residue was purified by flash column chromatography through silica gel (eluted with PE:EtOAc=5:1) to afford Int-85 (1.4 g, 5.58 mmol, 85%) as a yellow solid. LCMS (ESI): m/z=255 [M+H]⁺.

Step 4: A mixture of Int-85 (1.4 g, 5.58 mmol, 1.0 eq.), cyclopropylboronic acid (955.3 g, 11.2 mmol, 2.0 eq.), cataCXium A Pd G3 (407.8 mg, 0.56 mmol, 0.1 eq.) and K₂CO₃ (2.3 g, 16.7 mmol, 3.0 eq.) in dioxane (50 mL) and H₂O (10 mL) was stirred at 100° C. under N₂ atmosphere for 3 hrs. After completion, the mixture was diluted with H₂O (60 mL) and extracted with EtOAc (40 mL×3), the combined organic phase was dried over anhydrous Na₂SO₄ and concentrated under reduced pressure. The residue was purified by flash column chromatography through silica gel (eluted with PE:EtOAc=4:1) to afford Int-86 (990 mg, 4.58 mmol, 82.1%) as a yellow solid. LCMS (ESI): m/z=217 [M+H]⁺.

Steps 5 and 6: To a stirred solution of Int-86 (990 mg, 4.58 mmol, 1.0 eq.) in water (50 mL) was added concentrated sulfuric acid (10 mL) and aqueous solution of NaNO₂ (1 M, 7 mL, 7 mmol, 1.5 eq.) at 0° C., slowly. After stirring at r.t. for 6 hrs, the mixture was poured into ice water (50 mL) and neutralized by an aqueous solution of NaOH (4 M). After completion, the resulting mixture was extracted with EtOAc (30 mL×3), and the organic phase was combined, dried over anhydrous Na₂SO₄ and concentrated under reduced pressure. The crude residue Int-87 was dissolved in anhydrous THE (20 mL), then to the resulting mixture was added NaH (275 mg, 6.9 mmol, 1.5 eq.) under N₂ atmosphere at 0° C., slowly. After stirring at 0° C. for 30 min, to the mixture was added 2, 6-dichloro-3-nitropyridine (R-1, 1.06 g, 5.5 mmol, 1.2 eq.). After stirring at r.t. for 2 hrs, the mixture was quenched with a saturated aqueous solution of NH₄Cl (50 mL) and extracted with EtOAc (20 mL×3). The combined organic phase was dried over anhydrous Na₂SO₄ and concentrated under reduced pressure. The residue was purified by flash column chromatography on silica gel (eluted with PE:EtOAc=1: 5) to afford Int-88 (717.6 mg, 1.92 mmol, 42% for two steps) as a yellow solid. LCMS (ESI): m/z=374 [M+H]⁺.

Step 7: A mixture of Int-88 (717.6 mg, 1.92 mmol, 1.0 eq.), (5-cyanopyridin-3-yl)boronic acid (R-10, 341 mg, 2.3 mmol, 1.2 eq.), Pd(PPh₃)₄ (231 mg, 0.2 mmol, 0.1 eq.) and Na₂CO₃ (610.6 mg, 5.76 mmol, 3.0 eq.) in dioxane (100 mL) and water (20 mL) was stirred under N₂ atmosphere at 80° C. for 4 hrs. After completion, the reaction mixture was diluted with H₂O (50 mL) and extracted with EtOAc (30 mL×3). The organic layers were combined, dried over anhydrous Na₂SO₄ and concentrated under reduced pressure. The residue was purified by flash column chromatography on silica gel (eluted with PE:EtOAc=1: 9) to afford Int-89 (643.7 mg, 1.46 mmol, 76%) as a yellow solid. LCMS (ESI): m/z=442 [M+H]⁺.

Step 8: A mixture of Int-89 (643.7 mg, 1.46 mmol, 1.0 eq.), Fe powder (408.8 mg, 7.3 mmol, 5 eq.) and NH₄Cl (781 mg, 14.6 mmol, 10 eq.) in EtOH (50 mL) and H₂O (20 mL) was stirred at 80° C. for 3 hrs. After completion, the mixture was filtered through a pad of Celite and the filter cake was washed with MeOH (20 mL×3). The combined filtrate was concentrated under reduced pressure to afford Int-90 (621 mg, crude product) as a brown solid, which was used for the next step directly. LCMS (ESI): m/z=412 [M+H]⁺.

Steps 9 and 10: To a stirred solution of Int-90 (621 mg, crude) in DCM (20 mL) was added MsCl (85 mg, 0.74 mmoL) and TEA (2 mL) at 0° C., slowly. After stirring at r.t. for 10 min, the reaction mixture was diluted with H₂O (50 mL) and extracted with EtOAc (20 mL×3). The combined organic layers were concentrated under reduced pressure. The crude residue Int-91 was dissolved in dioxane (10 mL) and to the mixture was added an aqueous solution of NaOH (4 M, 1 mL). After stirring at r.t. for 30 min, the mixture was neutralized by HCl (1 M) and extracted with EtOAc (20 mL×3). The combined organic phase was dried over anhydrous Na₂SO₄ and concentrated under reduced pressure. The residue was purified by flash column chromatography through silica gel (eluted with PE:EtOAc=1:4) to afford Compound I-155 (136 mg, 0.28 mmol, 19.2% for three steps) as a white solid. LCMS (ESI): m/z=490 [M+H]⁺.

Compound I-155: Retention time: 1.558 min; LCMS (ESI): m/z=490 [M+H]⁺; ¹H NMR (400 MHz, DMSO) δ 10.13 (s, 1H), 9.01 (d, J=2.1 Hz, 1H), 8.97 (d, J=1.9 Hz, 1H), 8.53 (t, J=2.0 Hz, 1H), 8.03 (d, J=8.1 Hz, 1H), 7.96 (d, J=8.1 Hz, 1H), 7.35 (s, 1H), 3.26 (s, 3H), 2.36 (s, 3H), 2.08-1.99 (m, 1H), 0.93 (t, J=7.1 Hz, 4H).

The following compounds were synthesized using Method Y with the appropriate amines, boronic acids/boronates and sulfonyl chlorides: Compounds I-140, I-141, I-152, I-158, I-163, I-184, I-198, I-217, I-218, I-219, I-225, and I-240.

### Example 29: Method Z: Synthesis of Compound I-175

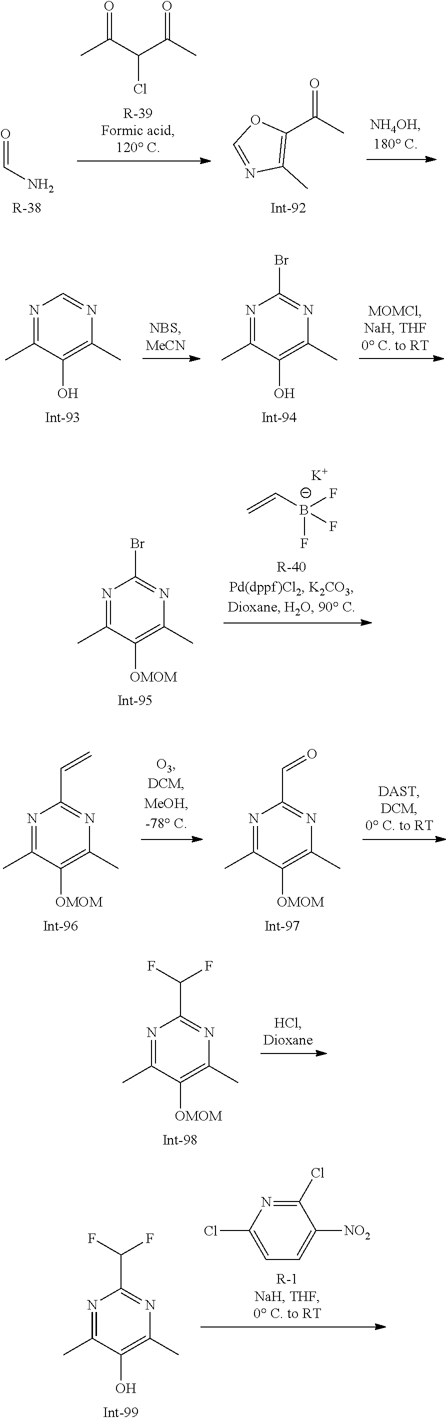

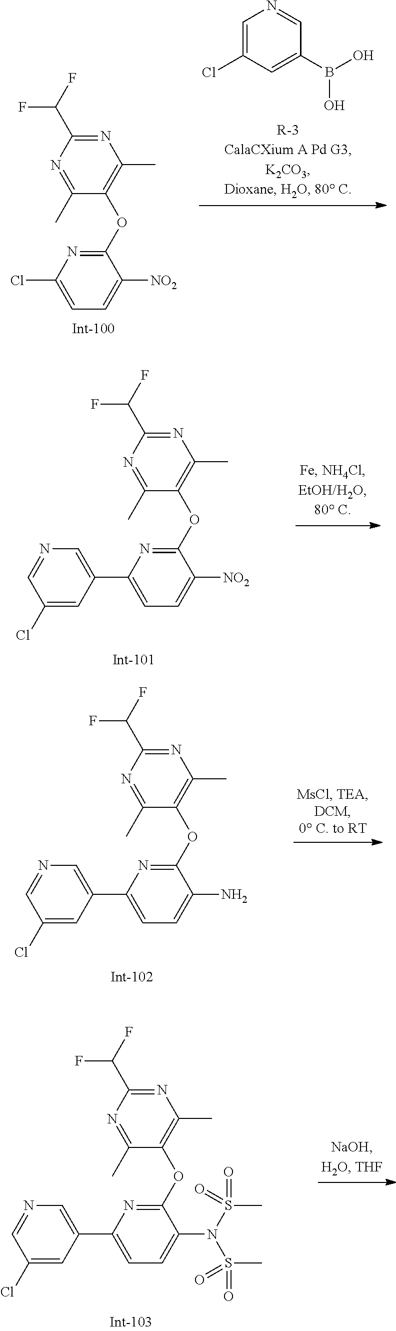 [SMILES: Cc1nc(C(F)F)nc(C)c1Oc1nc(Cl)ccc1[N+](=O)[O-] ; OB(O)c1cncc(Cl)c1 ; Cc1nc(C(F)F)nc(C)c1Oc1nc(-c2cncc(Cl)c2)ccc1[N+](=O)[O-] ; Cc1nc(C(F)F)nc(C)c1Oc1nc(-c2cncc(Cl)c2)ccc1N ; Cc1nc(C(F)F)nc(C)c1Oc1nc(-c2cncc(Cl)c2)ccc1N(S(C)(=O)=O)S(C)(=O)=O]

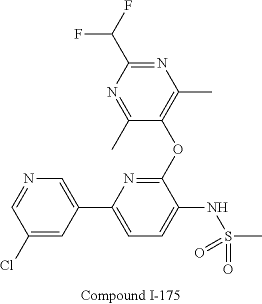 [SMILES: Cc1nc(C(F)F)nc(C)c1Oc1nc(-c2cncc(Cl)c2)ccc1NS(C)(=O)=O]

Step 1: A mixture of formamide (R-38, 50 g, 1.1 mol, 2.0 eq.) and 3-chloropentane-2, 4-dione (R-39, 74.4 g, 550 mmol, 1.0 eq.) in formic acid (30 mL) was stirred at 120° C. for 16 hrs. After completion, the reaction mixture was poured into ice water (500 mL), then neutralized with aqueous solution of NaOH (4 M) and extracted with EtOAc (120 mL×3). The combined organic phase was dried over anhydrous Na₂SO₄ and concentrated under reduced pressure. The residue was purified by flash column chromatography through silica gel (eluted with PE:EtOAc=0 to 5:1) to afford Int-92 (10.3 g, 82.4 mmol, 15% o) as a brown oil. LCMS (ESI): m/z=126 [M+H].

Step 2: A stirred mixture of Int-92 (10.3 g, 82.4 mmol, 1.0 eq.) inNHl₄OH (50 mL) was stirred at 180° C. for 8 hrs. After completion, the mixture was cooled to r.t. and extracted with EtOAc (100 mL×3). The combined organic phase was dried over anhydrous Na₂SO₄ and concentrated under reduced pressure. The residue was purified by flash column chromatography through silica gel (eluted with PE:EtOAc=100:1 to 1:100) to afford Int-93 (7.1 g, 57.2 mmol, 69.5% o) as a yellow solid. LCMS (ESI): m/z=125 [M+H].

Step 3: To a stirred solution of Int-93 (3.8 g, 30.4 mmol, 1.0 eq.) in MeCN (20 mL) was added NBS (6.5 g, 36.5 mmol, 1.2 eq.), slowly. After stirring at r.t. for 1 h, the reaction mixture was diluted with H₂O (100 mL) and extracted with EtOAc (50 mL×3). The combined organic phase was dried over anhydrous Na₂SO₄ and concentrated under reduced pressure. The residue was purified by flash column chromatography through silica gel (eluted with PE:EtOAc=0 to 2:1) to afford Int-94 (2.0 g, 9.9 mmol, 32.6%) as a brown solid. LCMS (ESI): m/z=203 [M+H]⁺.

Step 4: To a stirred solution of Int-94 (1 g, 4.95 mmol, 1.0 eq.) in THF (10 mL) was added NaH (237.6 mg, 5.94 mmol, 1.2 eq.) under N₂ atmosphere at 0° C. After stirring for 30 min, to the mixture was added MOMCl (597.8 mg, 7.43 mmol, 1.5 eq.), slowly. The resulting mixture was stirred at r.t. for 1 h, then the reaction was quenched with a saturated aqueous solution of NH₄Cl (50 mL) and extracted with EtOAc (20 mL×3). The combined organic phase was dried over anhydrous Na₂SO₄ and concentrated under reduced pressure. The residue was purified by flash column chromatography through silica gel (eluted with PE:EtOAc=0 to 5:1) to afford Int-95 (876 mg, 3.56 mmol, 72%) as a yellow solid. LCMS (ESI): m/z=247 [M+H]⁺.

Step 5: A mixture of Int-95 (476 mg, 1.93 mmol, 1.0 eq.), potassium vinyltrifluoroborate (R-40, 388.9 mg, 2.90 mmol, 1.5 eq.), Pd(dppf)Cl₂ (141.4 mg, 0.19 mmol, 0.1 eq.) and K₂CO₃ (801.1 mg, 5.80 mmol, 3.0 eq.) in dioxane (50 mL) and water (10 mL) was stirred under N₂ atmosphere at 90° C. overnight. After completion, the mixture was diluted with H₂O (50 mL) and extracted with EtOAc (30 mL×3). The combined organic phase was dried over anhydrous Na₂SO₄ and concentrated under reduced pressure. The residue was purified by flash column chromatography through silica gel (eluted with PE:EtOAc=4:1) to afford Int-96 (321 mg, 1.65 mmol, 85.5%) as a white solid. LCMS (ESI): m/z=195 [M+H]⁺.

Step 6: To a stirred solution of Int-96 (321 mg, 1.65 mmol, 1.0 eq.) in a mixed solvent of DCM (6 mL) and MeOH (2 mL) was bubbled O₃ at −78° C. for 30 mins. Then, N₂ was bubbled through the mixture for another 1 h to remove the remaining O₃. After completion, the mixture was concentrated under reduced pressure and the residue was purified by flash column chromatography through silica gel (eluted with PE:EtOAc=3:1) to afford Int-97 (220 mg, 1.12 mmol, 67.8%) as a white solid. LCMS (ESI): m/z=197 [M+H]⁺.

Step 7: To a stirred solution of Int-97 (220 mg, 1.12 mmol, 1.0 eq.) in DCM (5 mL) was added DAST (722.8 mg, 4.5 mmol, 4.0 eq.) at 0° C. After stirring at r.t. for 30 mins, the reaction mixture was diluted with H₂O (40 mL) and extracted with DCM (20 mL×3). The organic layers were combined, dried over anhydrous Na₂SO₄ and concentrated under reduced pressure. The residue was purified by flash column chromatography through silica gel (eluted with PE:EtOAc=10:1 to 3:1) to afford Int-98 (151 mg, 0.69 mmol, 61.7%) as a white solid. LCMS (ESI): m/z=219 [M+H]⁺.

Step 8: To a stirred solution of Int-98 (151 mg, 0.69 mmol, 1.0 eq.) in dioxane (5 mL) was added HCl/dioxane (5 mL). After stirring at r.t. for 30 min, the mixture was concentrated under reduced pressure to give Int-99 (150 mg, crude) as a white solid. LCMS (ESI): m/z=175 [M+H]⁺.

Step 9 through 13: Conducted using analogous procedures used in Method Y (Step 6-10) to provide Compound I-175, which was purified by flash column chromatography through silica gel (eluted with PE:EtOAc=1:100) to afford Compound I-175 (3.0 mg, 0.0065 mmol, 50.2%) as white solid.

Compound I-175: Retention time: 1.288 min; LCMS (ESI): m/z=456 [M+H]⁺; ¹H NMR (400 MHz, DMSO) δ 10.07 (s, 1H), 8.77 (d, J=1.8 Hz, 1H), 8.60 (d, J=2.3 Hz, 1H), 8.09 (t, J=2.0 Hz, 1H), 7.98 (dd, J=21.5, 8.1 Hz, 2H), 6.99 (t, J=54.2 Hz, 1H), 3.24 (s, 3H), 2.39 (s, 6H); ¹⁹F NMR (376 MHz, DMSO) δ −117.42 (s).

The following compounds were synthesized using Method Z with the appropriate alcohols, boronic acids/boronates and sulfonyl chlorides: Compounds I-144 and I-173.

### Example 30: Method ZA: Synthesis of Compound I-204

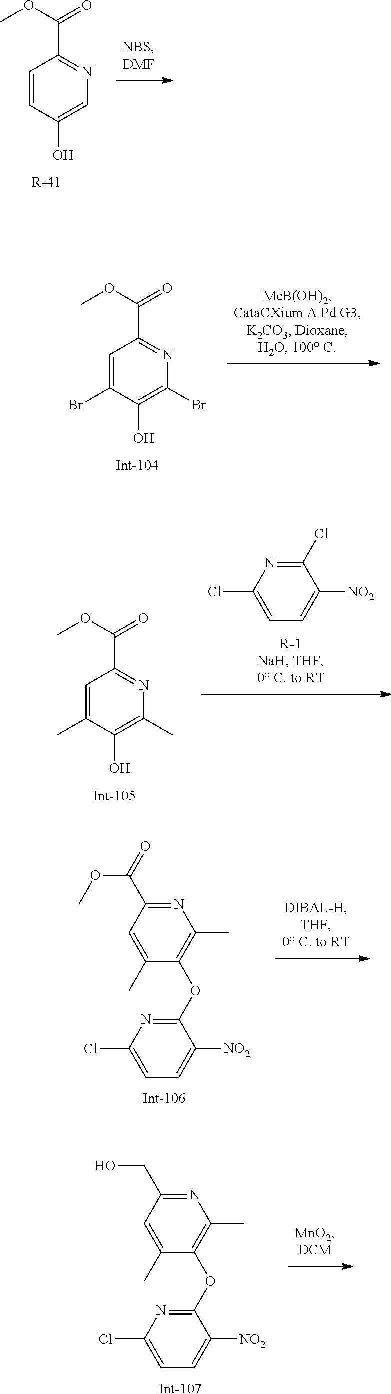

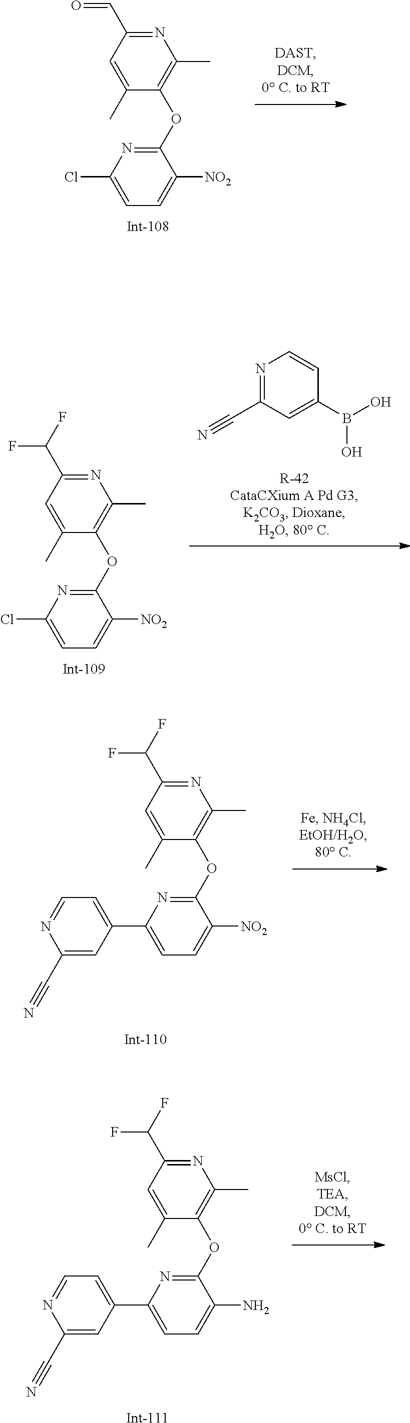

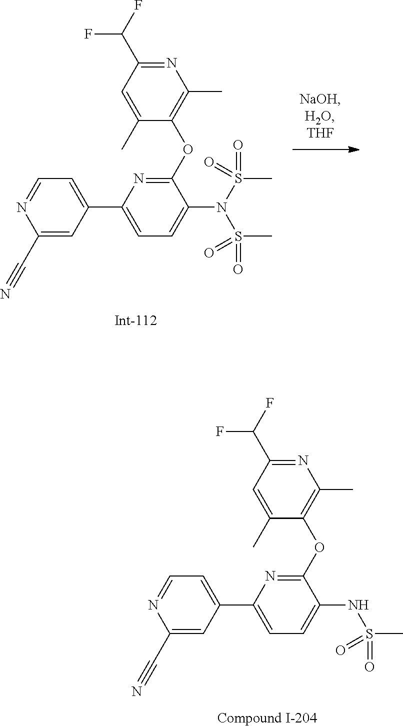 [SMILES: Cc1cc(C(F)F)nc(C)c1Oc1nc(-c2ccnc(C#N)c2)ccc1N(S(C)(=O)=O)S(C)(=O)=O ; "Int-112" ; Cc1cc(C(F)F)nc(C)c1Oc1nc(-c2ccnc(C#N)c2)ccc1NS(C)(=O)=O ; "Compound I-204"]

Step 1: To a stirred solution of methyl-5-hydroxypicolinate (R-41, 10 g, 65.4 mmol, 1.0 eq.) in DMF (100 mL) was added NBS (28.9 g, 163.4 mmol, 2.5 eq.), slowly. After stirring at r.t. for 1 h, the reaction was diluted with H₂O (200 mL) and extracted with EtOAc (100 mL×3). The combined organic phase was dried over anhydrous Na₂SO₄ and concentrated under reduced pressure. The residue was purified by flash column chromatography on silica gel (eluted with PE:EtOAc=0 to 1: 99) to afford Int-104 (17.1 g, 55.4 mmol, 84.70%) as a yellow solid. LCMS (ESI): m/z=310 [M+H]⁺.

Step 2: A mixture of Int-104 (2.0 g, 6.5 mmol, 1.0 eq.), methylboronic acid (1.9 g, 32.4 mmol, 5.0 eq.), cataCXium A Pd G3 (469.95 mg, 0.65 mmol, 0.1 eq.) and K₂CO₃ (2.7 g, 19.4 mmol, 3.0 eq.) in dioxane (60 mL) and water (10 mL) was stirred under N₂ atmosphere at 100° C. overnight. After completion, the mixture was diluted with H₂O (100 mL) and extracted with EtOAc (60 mL×3). The combined organic phase was dried over anhydrous Na₂SO₄ and concentrated under reduced pressure to afford Int-105 (824 mg, crude) as a yellow solid. LCMS (ESI): m/z=182 [M+H]⁺.

Step 3: To a stirred solution of Int-105 (824 mg, crude) in THF (5 mL) was added NaH (218.5 mg, 5.46 mmol) under N₂ atmosphere at 0° C., slowly. After stirring for 30 mins, to the mixture was added 2, 6-dichloro-3-nitropyridine (R-1, 961.5 mg, 5.0 mmol), the resulting mixture was stirred at r.t. for another 1 h. After completion, the reaction was quenched with a saturated aqueous solution of NH₄Cl (50 mL) and extracted with EtOAc (20 mL×3). The combined organic phase was dried over anhydrous Na₂SO₄ and concentrated under reduced pressure. The residue was purified by flash column chromatography through silica gel (eluted with PE:EtOAc=0 to 5:1) to afford Int-106 (302 mg, 0.89 mmol, 13.8% for two steps) as a yellow solid. LCMS (ESI): m/z=338 [M+H]⁺.

Step 4: To a stirred solution of Int-106 (302 mg, 0.89 mmol, 1.0 eq.) in THE (50 mL) was added a THE solution of DIBAL-H (1M, 2.7 mL, 2.7 mmol, 3.0 eq.) under N₂ atmosphere at 0° C. After stirring at r.t. for 2 hrs, to the mixture was added aqueous solution of potassium sodium tartrate tetrahydrate (50 mL). The mixture was stirred at r.t. for 30 mins and extracted with EtOAc (30 mL×3). The combined organic phase was dried over anhydrous Na₂SO₄ and concentrated under reduced pressure. The residue was purified by flash column chromatography through silica gel (eluted with PE:EtOAc=10:1 to 2:1) to afford Int-107 (178 mg, 0.57 mmol, 64.2%) as a white solid. LCMS (ESI): m/z=310 [M+H]⁺.

Step 5: To a stirred solution of Int-107 (178 mg, 0.57 mmol, 1.0 eq.) in DCM (10 mL) was added MnO₂ (251 mg, 2.88 mmol, 5.0 eq.). After stirring at r.t. for 30 mins, the reaction mixture was filtered through a pad of Celite and the filter cake was washed with DCM (20 mL×3). The combined filtrates were concentrated under reduced pressure to give Int-108 (152 mg, 0.49 mmol, 85.9%) as a white solid. LCMS (ESI): m/z=308 [M+H]⁺.

Step 6: To a stirred solution of Int-108 (152 mg, 0.49 mmol, 1.0 eq.) in DCM (10 mL) was added DAST (318.8 mg, 1.98 mmol, 4.0 eq.) at 0° C. After stirring at r.t. for 30 mins, the reaction mixture was diluted with H₂O (40 mL) and extracted with DCM (20 mL×3). The organic layers were combined, dried over anhydrous Na₂SO₄ and concentrated under reduced pressure. The residue was purified by flash column chromatography through silica gel (eluted with PE:EtOAc=10:1 to 3:1) to afford Int-109 (93 mg, 0.28 mmol, 57.1%) as a white solid. LCMS (ESI): m/z=330 [M+H]⁺.

Step 7 through 10: Conducted using analogous procedures used in Method Y (Step 7-10) to provide Compound I-204, which was purified by prep-HPLC (column: YMC-Actus Triart C18 250×20 mm×5 um, gradient: 30%-95% MeCN in H₂O [with 0.1% TFA]) to afford Compound I-204 (14.1 mg, 0.032 mmol, 75.4%) as white solid.

Compound 1-204: Retention time: 1.359 min; LCMS (EST): m/z=446.4 [M+H]J; ¹H NMR (400 MHz, DMSO) δ 10.16 (s, 1H), 8.73 (d, J=4.7 Hz, 1H), 8.24 (s, 1H), 8.04 (s, 2H), 7.83 (d, J=2.9 Hz, 1H), 7.64 (s, 1H), 6.96 (t, J=55.0 Hz, 1H), 3.27 (s, 3H), 2.34 (s, 3H), 2.20 (s, 3H); ¹⁹F NMR (376 MHz, DMSO) δ −114.42 (d).

The following compounds were synthesized using Method ZA with the appropriate alcohols, boronic acids/boronates and sulfonyl chlorides: Compounds I-197, I-199, I-202, I-203, and I-230.

### Example 31: Method ZB: Synthesis of Compound I-196

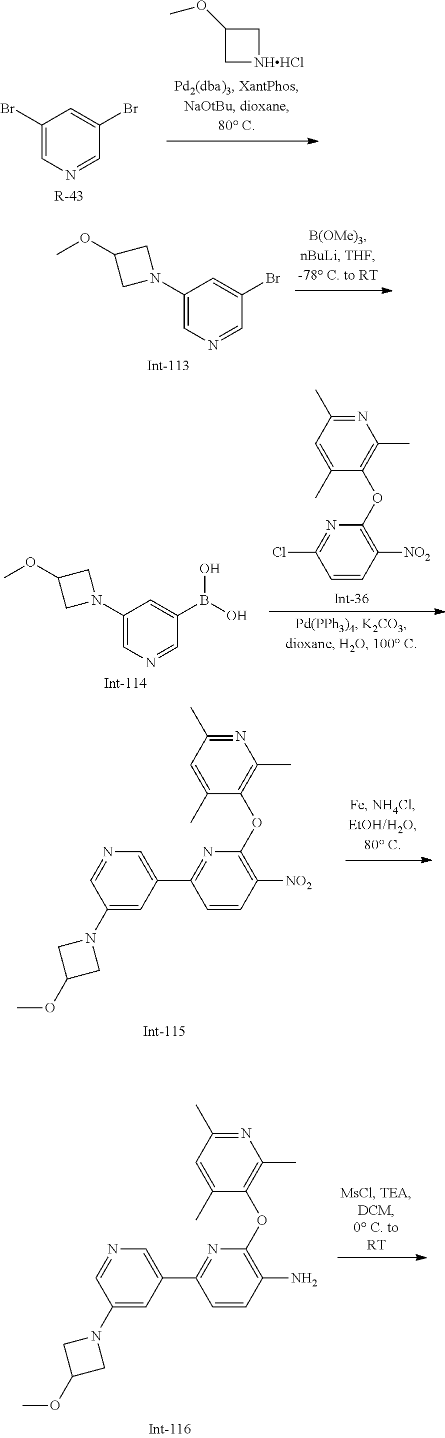

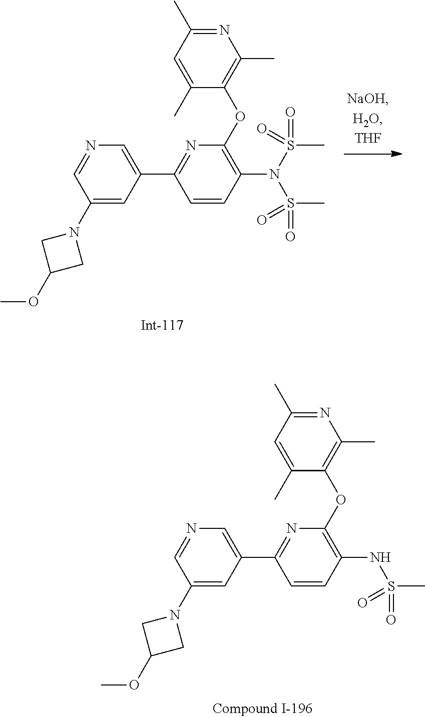

Step 1: A mixture of 3, 5-dibromopyridine (R-43, 1.0 g, 4.3 mmol, 1.0 eq.), 3-methoxyazetidine hydrochloride (584.5 mg, 4.73 mmol, 1.1 eq.), Pd₂(dba)₃ (366.3 mg, 0.4 mmol, 0.1 eq), XantPhos (372 mg, 0.65 mmol, 0.15 eq.) and NaO^{t}Bu (1.2 g, 13 mmol, 3 eq.) in dioxane (40 mL) was stirred under N₂ atmosphere at 80° C. for 2 hrs. After completion, the mixture was diluted with H₂O (40 mL) and extracted with EtOAc (20 mL×3). The combined organic phase was dried over anhydrous Na₂SO₄ and concentrated under reduced pressure. The residue was purified by flash column chromatography on silica gel (eluted with PE:EtOAc=0 to 5:1) to afford Int-113 (822.1 mg, 3.4 mmol, 79.0%) as a colorless oil. LCMS (ESI): m/z=243 [M+H]⁺.

Steps 2 and 3: To a stirred solution of Int-113 (822.1 mg, 3.4 mmol, 1.0 eq.) in anhydrous THE (20 mL) was added the THF solution of ⁿBuLi (2.5 M, 1.6 mL, 4.0 mmol, 1.2 eq.) under N₂ atmosphere at −78° C. After stirring for 30 min, to the mixture was added B(OMe)₃ (530 mg, 5.1 mmol, 1.5 eq.), the resulting mixture was stirred at r.t. for another 30 min. After completion, the reaction was quenched by MeOH (5 mL) and concentrated under reduced pressure to provide crude Int-114. The crude residue Int-114 was dissolved in dioxane (20 mL) and H₂O (4 mL), then, to the resulting solution was added Int-36 (996 mg, 3.4 mmol, 1.0 eq.), Pd(PPh₃)₄ (392 mg, 0.34 mmol, 0.1 eq.) and K₂CO₃ (1.38 g, 10 mmol, 3 eq.). After being stirred under N₂ atmosphere at 100° C. for 3 hrs, the mixture was diluted with H₂O (20 mL) and extracted with EtOAc (20 mL×3). The combined organic phase was dried over anhydrous Na₂SO₄ and concentrated under reduced pressure. The residue was purified by flash column chromatography on silica gel (eluted with PE:EtOAc=0 to 2:1) to afford Int-115 (343.5 mg, 0.82 mmol, 21% for two steps) as a yellow solid. LCMS (ESI): m/z=422 [M+H]⁺.

Step 4: A mixture of Int-115 (343.5 mg, 0.82 mmol, 1.0 eq.), Fe powder (230 mg, 4.1 mmol, 5 eq.) and NH₄Cl (438.7 mg, 8.2 mmol, 10 eq.) in EtOH (10 mL) and H₂O (5 mL) was stirred at 80° C. for 2 hrs. After completion, the mixture was filtered through a pad of Celite and the filter cake was washed with MeOH (20 mL×3). The filtrates were combined and concentrated under reduced pressure afford Int-116 (250 mg, crude) as a brown solid. LCMS (ESI): m/z=392 [M+H]⁺.

Steps 5 and 6: To a stirred solution of Int-116 (100 mg, crude) in DCM (5 mL) was added MsCl (0.5 mL) and TEA (1 mL) at 0° C., slowly. After stirring at r.t. for 10 min, the reaction mixture was diluted with H₂O (10 mL) and extracted with EtOAc (15 mL×3). The combined organic layers were concentrated under reduced pressure. The crude residue Int-117 was dissolved in dioxane (10 mL), then to the resulting mixture was added an aqueous solution of NaOH (4 M, 1 mL). After stirring at r.t. for 30 min, the mixture was neutralized by TFA (1 M) and extracted with EtOAc (20 mL×3). The combined organic phase was dried over anhydrous Na₂SO₄ and concentrated under reduced pressure. The residue was purified by Prep-HPLC (column: YMC-Actus Triart C8 250×20 mm, gradient: 30-95% MeCN in aqueous solution of NH₄HCO₃ (10 mM)) to afford Compound I-196 (12.1 mg, 0.026 mmol, 7.9% for three steps) as a white solid.

Compound I-196: Retention time: 0.885 min; LCMS (ESI): m/z=470.1 [M+H]f; ¹H NMR (400 MHz, DMSO) δ 9.83 (s, 1H), 8.25 (d, J=1.6 Hz, 1H), 7.86 (d, J=8.0 Hz, 1H), 7.77 (d, J=2.6 Hz, 1H), 7.72 (d, J=8.1 Hz, 1H), 7.10 (s, 1H), 6.98-6.94 (m, 1H), 4.39-4.30 (m, 1H), 4.06-3.98 (m, 2H), 3.58 (dd, J=8.3, 4.1 Hz, 2H), 3.25 (s, 3H), 3.15 (s, 3H), 2.43 (s, 3H), 2.21 (s, 3H), 2.06 (s, 3H).

The following compounds were synthesized using Method ZB with the appropriate amines, boronic acids/boronates and sulfonyl chlorides: Compounds I-185, I-190, I-194, I-195, I-201, I-263, and I-278.

### Example 32: Method ZC: Synthesis of Compound I-326

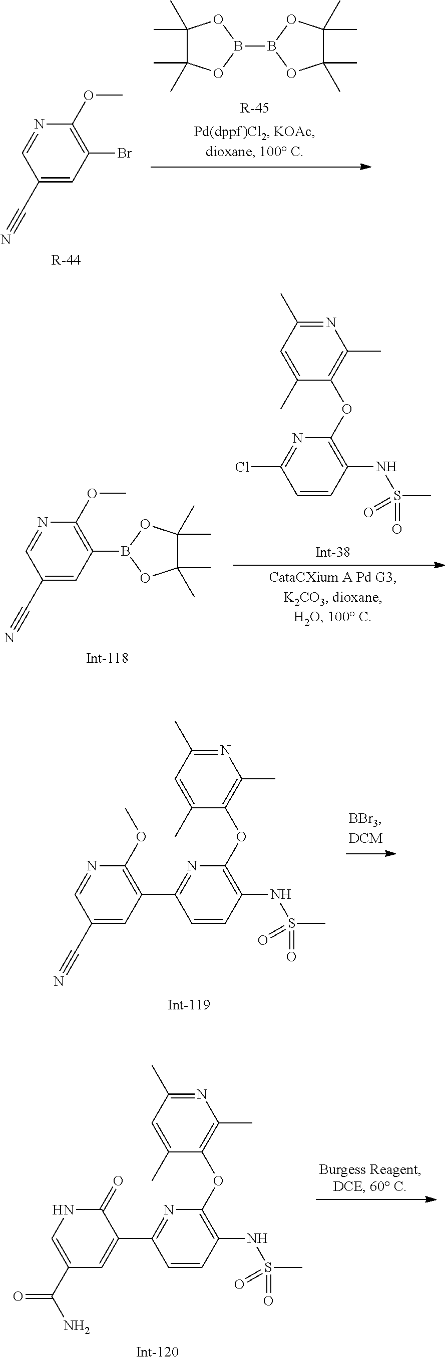

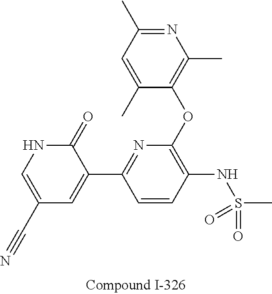 [SMILES: Cc1cc(C)c(Oc2nc(-c3cc(C#N)c[nH]c3=O)ccc2NS(C)(=O)=O)c(C)n1]

Step 1: A mixture of 5-bromo-6-methoxynicotinonitrile (R-44, 1.0 g, 4.7 mmol, 1.0 eq.), 4, 4, 4′, 4′, 5, 5, 5′, 5′-octamethyl-2, 2′-bi (1, 3, 2-dioxaborolane) (R-45, 1.4 g, 5.6 mmol, 1.2 eq.), Pd(dppf)Cl₂ (37 mg, 0.05 mmol, 0.1 eq.) and KOAc (1.4 g, 14.1 mmol, 3.0 eq.) in dioxane (50 mL) was stirred at 100° C. under N₂ atmosphere for 3 hrs. After completion, the mixture was diluted with H₂O (50 mL) and extracted with EtOAc (30 mL×3), the combined organic phase was dried over anhydrous Na₂SO₄ and concentrated under reduced pressure. The residue was purified by flash column chromatography through silica gel (eluted with PE:EtOAc=4:1) to afford Int-118 (892.5 mg, 3.4 mmol, 72.3%) as a white solid. LCMS (ESI): m/z=261 [M+H]⁺.

Step 2: To a mixture of Int-38 (340 mg, 1.0 mmol, 1 eq.), Int-118 (260 mg, 1.0 mmol, 1.0 eq.) and K₂CO₃ (415 mg, 3.0 mmol, 3.0 eq.) in dioxane (50 mL) and water (10 mL) was added cataCXium A Pd G3 (73 mg, 0.1 mmol, 0.1 eq.). The resulting mixture was stirred at 100° C. under N₂ atmosphere overnight. After completion, the reaction mixture was diluted with H₂O (30 mL) and extracted with EtOAc (30 mL×3). The combined organic phase was dried over anhydrous Na₂SO₄ and concentrated under reduced pressure. The residue was purified by flash column chromatography on silica gel (eluted with PE:EtOAc=1:1) to afford Int-119 (250 mg, 0.57 mmol, 57%) as a white solid. LCMS (ESI): m/z=440 [M+H]⁺.

Step 3: To a stirred solution of Int-119 (100 mg, 0.23 mmol) in DCM (20 mL) was added BBr₃ (2 mL). After stirring at r.t. for 2 hrs, the mixture was quenched with MeOH (20 mL) and concentrated under reduced pressure to remove the solvent. The residue was diluted with H₂O (20 mL) and extracted with EtOAc (30 mL×3). The combined organic phase was dried over anhydrous Na₂SO₄ and concentrated under reduced pressure. The residue was purified by flash column chromatography on silica gel (eluted with DCM:MeOH=5:1) to afford Int-120 (44 mg, 0.1 mmol, 42%) as a white solid. LCMS (ESI): m/z=444 [M+H]⁺.

Step 4: To a stirred solution of Int-120 (44 mg, 0.1 mmol, 1 eq.) in DCE (10 mL) was added Burgess reagent (120 mg, 0.5 mmol, 5 eq.). After stirring at 60° C. for 2 hrs, the residue was diluted with H₂O (20 mL) and extracted with EtOAc (30 mL×3). The combined organic phase was dried over anhydrous Na₂SO₄ and concentrated under reduced pressure. The residue was purified by pre-HPLC (column: YMC-Actus Triart C18 250×20 mm x Sum, gradient: 15-95% MeCN in H₂O (with 0.1% of FA)) to afford Compound I-326 (8.5 mg, 0.02 mmol, 20%) as a white solid.

Compound I-326: Retention time: 0.439 min; LC-MS (ESI) m/z 426.1 [M+H]⁺; ¹H NMR (400 MHz, DMSO) δ 12.84 (s, 1H), 9.92 (s, 1H), 8.31 (s, 1H), 8.27 (d, J=8.2 Hz, 1H), 7.92 (d, J=8.2 Hz, 1H), 7.63 (d, J=2.5 Hz, 1H), 7.50 (s, 1H), 3.21 (s, 4H), 2.58 (s, 3H), 2.37 (s, 3H), 2.20 (s, 3H).

The following compounds were synthesized using Method ZC with the appropriate aryl halides, boronic acids/boronates and sulfonyl chlorides: Compounds I-307 and I-324.

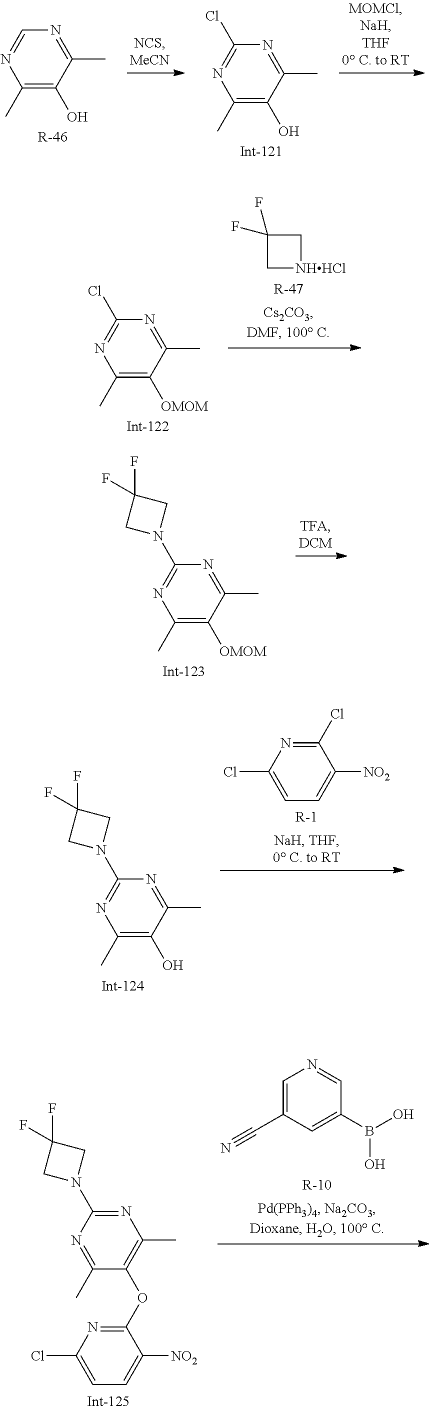

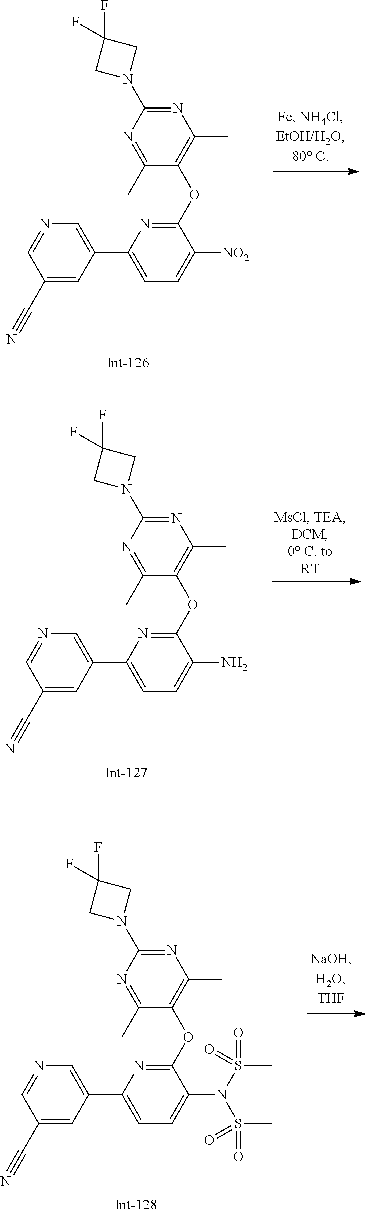 [SMILES: Cc1nc(N2CC(F)(F)C2)nc(C)c1Oc1nc(-c2cncc(C#N)c2)ccc1[N+](=O)[O-] ; Cc1nc(N2CC(F)(F)C2)nc(C)c1Oc1nc(-c2cncc(C#N)c2)ccc1N ; Cc1nc(N2CC(F)(F)C2)nc(C)c1Oc1nc(-c2cncc(C#N)c2)ccc1N(S(C)(=O)=O)S(C)(=O)=O]

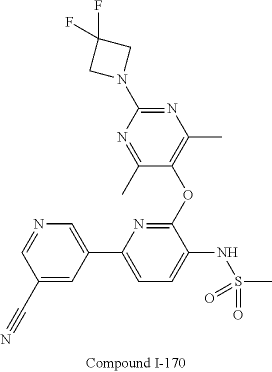 [SMILES: Cc1nc(N2CC(F)(F)C2)nc(C)c1Oc1nc(-c2cncc(C#N)c2)ccc1NS(C)(=O)=O]

### Example 33: Method ZD: Synthesis of Compound I-170

Step 1: To a stirred solution of 4, 6-dimethylpyrimidin-5-ol (R-46, 2.0 g, 16.1 mmol, 1.0 eq.) in MeCN (60 mL) was added NCS (2.56 g, 19.2 mmol, 1.2 eq.) slowly. After stirring at r.t. for 1 h, the reaction mixture was diluted with H₂O (150 mL) and extracted with EtOAc (80 mL×3). The combined organic phase was dried over anhydrous Na₂SO₄ and concentrated under reduced pressure. The residue was purified by flash column chromatography through silica gel (eluted with PE:EtOAc=0 to 2:1) to afford Int-121 (1.68 g, 10.6 mmol, 66.0%) as a white solid. LCMS (ESI): m/z=159 [M+H]⁺.

Step 2: To a stirred solution of Int-121 (1.68 g, 10.6 mmol, 1.0 eq.) in THF (60 mL) was added NaH (80 mg, 60% dispersed in mineral oil 2.0 mmol, 1.2 eq.) under N₂ atmosphere at 0° C. After stirring for 30 min, added MOMCl (1.27 g, 16 mmol, 1.5 eq.) slowly. The resulting mixture was stirred at r.t. for 1 h, then the reaction was quenched with ice H₂O (80 mL) and extracted with EtOAc (50 mL×3). The combined organic phase was dried over anhydrous Na₂SO₄ and concentrated under reduced pressure. The residue was purified by flash column chromatography through silica gel (eluted with PE:EtOAc=0 to 6: 1) to afford Int-122 (1.86 g, 9.2 mmol, 86.9%) as a yellow solid. LCMS (ESI): m/z=203 [M+H]⁺.

Step 3: A mixture of Int-122 (80 mg, 0.4 mmol, 1 eq.), 3,3-difluoroazetidine hydrochloride (R-47, 103.6 mg, 0.8 mmol, 2 eq.) and Cs₂CO₃ (391 mg, 1.2 mmol, 3 eq.) in DMF (5 mL) was stirred at 100° C. for 16 hrs. After completion, the resulting solution was diluted with H₂O (70 mL) and extract with EtOAc (30 mL×3). The organic phase was combined, washed with brine (30 mL×3) dried over anhydrous Na₂SO₄ and concentrated under reduced pressure. The residue was purified by column chromatography through silica gel (eluted with PE:EtOAc=5:1) to afford Int-123 (40 mg, 0.15 mmol, 39%) as a white solid. LCMS (ESI): m/z=260 [M+H]⁺.

Step 4: To a solution of Int-123 (20 mg, 0.08 mmol) in DCM (2 mL) was added TFA (0.5 mL). After stirring at r.t. for 1 h, the mixture was concentrated in vacuum to afford Int-124 (30 mg, crude) as a yellow oil, which used for the next step directly. LCMS (ESI): m/z=216 [M+H]⁺.

Step 5: To a stirred solution of Int-124 (30 mg, 0.14 mmol, 1.0 eq.) in THE (5 mL) was added NaH (10 mg, 60% dispersed in mineral oil, 0.21 mmol, 1.5 eq.) at 0° C. under N₂ atmosphere slowly. After stirring at 0° C. for 30 min, to the mixture was added 2, 6-dichloro-3-nitropyridine (R-1, 30.0 mg, 0.16 mmol, 1.1 eq.) at 0° C. After stirring at r.t. for 2 hrs, the reaction was quenched by ice water (30 mL) and extracted with EtOAc (20 mL×3). The combined organic phase was dried over anhydrous Na₂SO₄ and concentrated under reduced pressure. The residue was purified by flash column chromatography on silica gel (eluted with PE:EtOAc=0 to 4:1) to afford Int-125 (20 mg, 0.054 mmol, 38.5%) as a yellow solid. LCMS (ESI): m/z=372 [M+H]⁺.

Step 6: A mixture of Int-125 (20.0 mg, 0.05 mmol, 1.0 eq.), (5-cyanopyridin-3-yl)boronic acid (R-10, 9.0 mg, 0.06 mmol, 1.2 eq.), Pd(PPh₃)₄ (6.0 mg, 0.005 mmol, 0.1 eq.) and K₂CO₃ (21 mg, 0.15 mmol, 3.0 eq.) in dioxane (20 mL) and water (4 mL) was stirred under nitrogen atmosphere at 100° C. for 4 hrs. After completion, the reaction mixture was diluted with H₂O (20 mL) and extracted with EtOAc (15 mL×3). The organic layers were combined, dried over anhydrous Na₂SO₄ and concentrated under reduced pressure. The residue was purified by flash column chromatography through silica gel (eluted with EA) to afford Int-126 (30 mg, 80% purity, 0.05 mmol, 99%) as a yellow solid. LCMS (ESI): m/z=440 [M+H]⁺.

Step 7: A mixture of Int-126 (24 mg, 0.05 mmol, 1.0 eq.), Fe powder (14 mg, 0.25 mmol, 5 eq.) and NH₄Cl (27 mg, 0.5 mmol, 10 eq.) in EtOH (30 mL) and H₂O (15 mL) was stirred at 80° C. for 2 hrs. After completion, the reaction mixture was filtered through a pad of Celite and the filter cake was washed with MeOH. The combined filtrates were concentrated, the residue was triturated with H₂O, then filtered to afford Int-127 (30 mg, crude) as a brown solid. LCMS (ESI): m/z=410 [M+H]⁺.

Steps 8 and 9: To a stirred solution of Int-127 (30 mg, crude) in DCM (10 mL) was added MsCl (0.5 mL) and TEA (1 mL) at 0° C. slowly. After stirring at r.t. for 10 min, the reaction mixture was diluted with H₂O (30 mL) and extracted with EtOAc (20 mL×3). The combined organic layers were concentrated under reduced pressure to provide Int-128. The crude Int-128 (35 mg) was dissolved in THE (10 mL), then to the resulting mixture was added an aqueous solution of NaOH (4 M, 1 mL). After stirring at r.t. for 30 min, the mixture was neutralized by HCl (1 M) and extracted with EtOAc (15 mL×3). The combined organic phase was dried over anhydrous Na₂SO₄ and concentrated under reduced pressure. The residue was purified through Prep-HPLC (column: Welch XBridge C18 250×21 mm, gradient: 20-95% MeCN in H₂O (with 0.1% TFA)) to afford Compound I-170 (8.5 mg, 0.017 mmol, 34.9% over 3 steps) as a white solid.

Compound I-170: Retention time: 1.522 min; LCMS (ESI): m/z=488.1 [M+H]f; 1H NMR (400 MHz, DMSO-d₆) δ (ppm) 9.96 (br, 1H), 9.06 (d, J=2.2 Hz, 1H), 8.98 (d, J=1.9 Hz, 1H), 8.60 (t, J=2.1 Hz, 1H), 7.96 (d, J=8.1 Hz, 1H), 7.90 (d, J=8.1 Hz, 1H), 4.48 (t, J=12.5 Hz, 4H), 3.20 (s, 3H), 2.18 (s, 6H); ¹⁹F NMR (377 MHz, DMSO-d₆) δ (ppm)−99.06 (s).

The following compounds were synthesized using Method ZD with the appropriate alcohols, amines, boronic acids/boronates and sulfonyl chlorides: Compounds I-159, I-160, and I-169.

### Example 34: Method ZE: Synthesis of Compound I-174

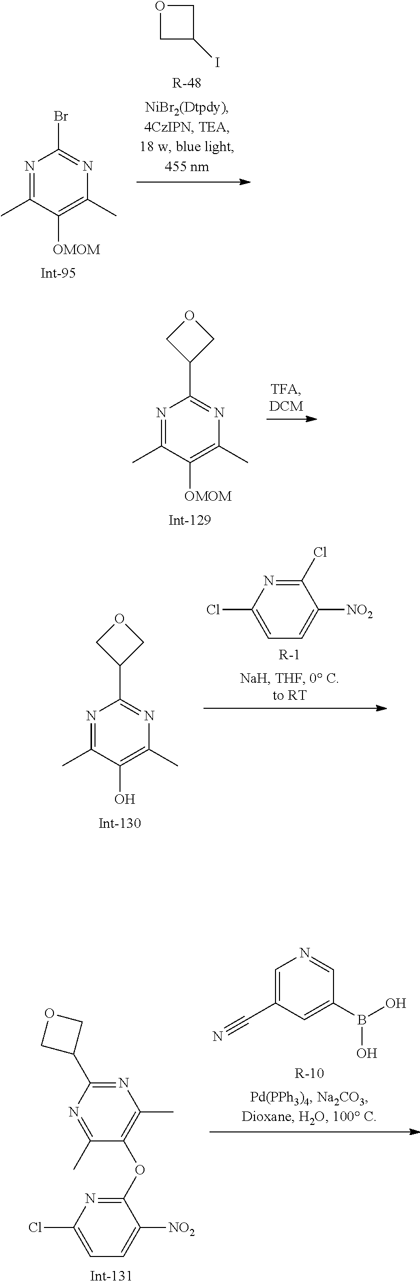

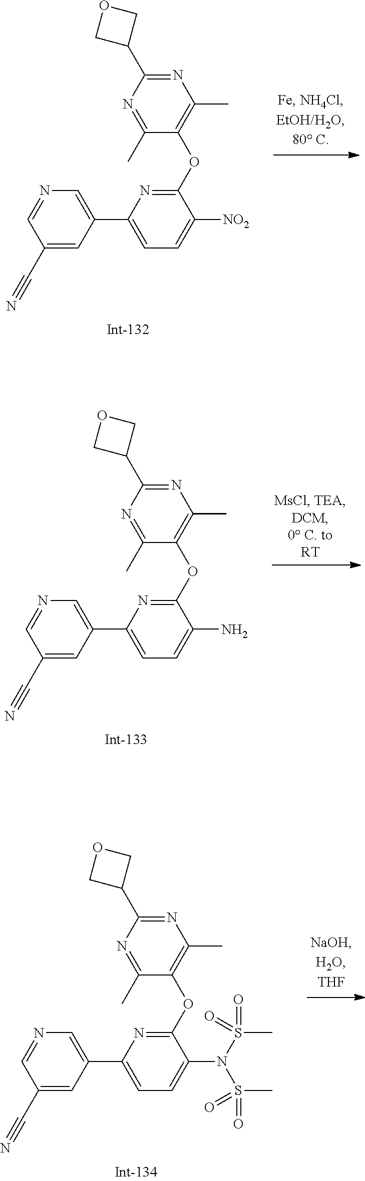 [SMILES: Cc1nc(C2COC2)nc(C)c1Oc1nc(-c2cncc(C#N)c2)ccc1[N+](=O)[O-] ; Cc1nc(C2COC2)nc(C)c1Oc1nc(-c2cncc(C#N)c2)ccc1N ; Cc1nc(C2COC2)nc(C)c1Oc1nc(-c2cncc(C#N)c2)ccc1N(S(C)(=O)=O)S(C)(=O)=O]

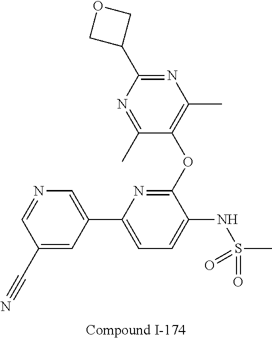 [SMILES: Cc1nc(C2COC2)nc(C)c1Oc1nc(-c2cncc(C#N)c2)ccc1NS(C)(=O)=O]

Step 1: To a stirred solution of Int-95 (100 mg, 0.40 mmol, 1 eq.) in dioxane (3 mL) was added 3-iodooxetane (R-48, 90 mg, 0.40 mmol, 1 eq.), NiBr₂(Dtpdy) (20 mg, 0.04 mmol, 0.1 eq.), 4CzIPN (64 mg, 0.08 mmol, 0.2 eq.) and TEA (0.16 mL, 1.20 mmol, 3 eq.). The resulting mixture was stirred under the irradiation of blue light (455 nm) at 25° C. for 2 hrs. After completion, the reaction mixture was diluted with H₂O (50 mL) and extracted with EtOAc (20 mL×3). The combined organic phase was dried over anhydrous Na₂SO₄ and concentrated under reduced pressure. The residue was purified by flash column chromatography through silica gel (eluted with PE:EtOAc=0 to 1:1) to Int-129 (30 mg, 0.13 mmol, 33%) as a yellow oil. (ESI): m/z=225 [M+H]⁺.

Step 2: To a stirred solution of Int-129 (30 mg, 0.13 mmol) in DCM (2 mL) was added TFA (0.5 mL). After stirring at r.t. for 1 h, the solution was concentrated under reduced pressure to afford Int-130 (30 mg, crude) as a yellow oil, which used for the next step directly. LCMS (ESI): m/z=181 [M+H]⁺.

Steps 3 through 7: Conducted using analogous procedures used in Method ZD (Step 5-9) to provide Compound I-174, which was purified through Prep-HPLC (column: Welch XBridge C18 250×21 mm, gradient: 20-95% MeCN in H₂O (with 0.1% TFA)) to afford Compound I-174 (2.0 mg, 0.0044 mmol) as white solid.

Compound I-174: Retention time: 0.944 min; LCMS (ESI): m/z=453.3 [M+H]⁺; ¹H NMR (400 MHz, DMSO) δ 8.98 (d, J=2.2 Hz, 1H), 8.86 (d, J=1.7 Hz, 1H), 8.40 (s, 1H), 7.80 (s, 2H), 4.95-4.85 (m, 4H), 4.53-4.42 (m, 1H), 2.99 (s, 3H), 2.30 (s, 6H).

The following compound was synthesized using Method ZE with the appropriate alcohols, amines, boronic acids/boronates and sulfonyl chlorides: Compound I-143.

### Example 35: Method ZF: Synthesis of Compound I-165

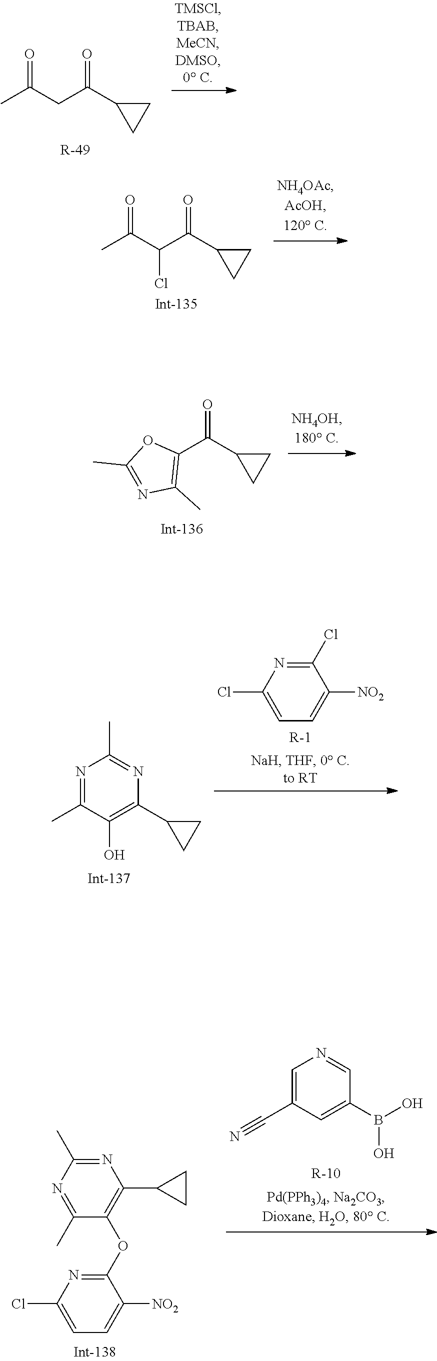

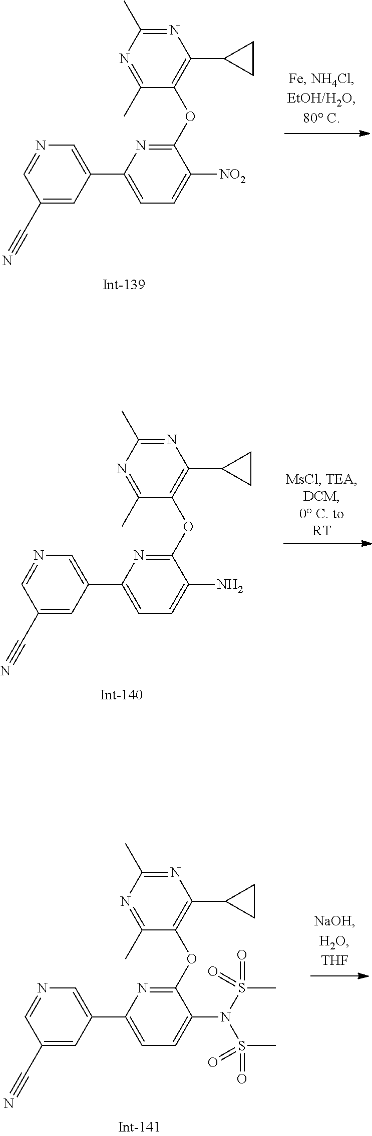 [SMILES: Cc1nc(C)c(Oc2nc(-c3cncc(C#N)c3)ccc2[N+](=O)[O-])c(C2CC2)n1 ; Cc1nc(C)c(Oc2nc(-c3cncc(C#N)c3)ccc2N)c(C2CC2)n1 ; Cc1nc(C)c(Oc2nc(-c3cncc(C#N)c3)ccc2N(S(C)(=O)=O)S(C)(=O)=O)c(C2CC2)n1]

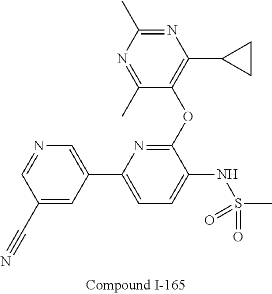 [SMILES: Cc1nc(C)c(Oc2nc(-c3cncc(C#N)c3)ccc2NS(C)(=O)=O)c(C2CC2)n1]

Step 1: To a stirred solution of tert-butylammonium bromide (13.9 g, 90 mmol, 1.5 eq.) and TMSCl (9 mL, 180 mmol, 3.0 eq.) in anhydrous acetonitrile (50 mL) was added the acetonitrile solution of 1-cyclopropylbutane-1, 3-dione (R-49, 58 mmol, 5 M, 11.6 mL, 1 eq.), followed by the addition of anhydrous DMSO (14 mL), dropwise. After stirring at 0° C. for 1.5 hrs, the mixture was diluted with water (500 mL) and extracted with EtOAc (200 mL×3). The combined organic phase was dried over anhydrous Na₂SO₄ and concentrated under reduced pressure. The residue was purified by flash column chromatography on silica gel (eluted with PE:EtOAc=0 to 9: 1) to afford Int-135 (5.76 g, 36 mmol, 62.2%) as a colorless oil. LCMS (ESI): m/z=161 [M+H]⁺.

Step 2: A mixture of Int-135 (5.0 g, 31.3 mol, 1.0 eq.) and NH₄OAc (7.2 g, 93.8 mol, 3.0 eq.) in AcOH (40 mL) was stirred at 120° C. for 16 hrs. After cooling to r.t., the mixture was diluted with H₂O (200 mL), neutralized by an aqueous solution of NaOH (4 M) and extracted with EtOAc (100 mL×3). The combined organic phase was dried over anhydrous Na₂SO₄ and concentrated under reduced pressure. The residue was purified by flash column chromatography on silica gel (eluted with PE:EtOAc=0 to 5:1) to afford Int-136 (2.2 g, 13.3 mmol, 43%) as a brown oil. LCMS (ESI): m/z=166 [M+H]⁺.

Step 3: A mixture of Int-136 (2.2 g, 13.3 mmol, 1.0 eq.) in NH₄OH (20 mL) was stirred at 180° C. for 8 hrs. After completion, the mixture was concentrated under reduced pressure to remove the solvent. The residue was purified by flash column chromatography on silica gel (eluted with PE:EtOAc=100:1 to 1:100) to afford Int-137 (1.3 g, 7.9 mmol, 59.6%) as a yellow solid. LCMS (ESI): m/z=165 [M+H]⁺.

Step 4 through 8: Conducted using analogous procedures used in Method ZD (Step 5-9) to provide Compound I-165, which was purified through Prep-HPLC (column: YMC-Actus Triart C18 250×20 mm x Sum, gradient: 10-95% MeCN in H₂O (with 0.1% FA)) to afford Compound I-165 (5.2 mg, 0.012 mmol) as a white solid.

Compound I-165: Retention time: 1.159 min; LCMS (ESI): m/z=437 [M+H]f; ¹HNMR (400 MHz, DMSO) δ 10.09 (s, 1H), 9.06 (d, J=2.2 Hz, 1H), 8.99 (d, J=1.9 Hz, 1H), 8.60 (t, J=2.0 Hz, 1H), 8.01 (d, J=8.1 Hz, 1H), 7.94 (d, J=8.1 Hz, 1H), 3.25 (s, 3H), 2.54 (s, 3H), 2.25 (s, 3H), 2.10 (ddd, J=13.1, 8.1, 4.8 Hz, 1H), 0.97 (d, J=30.8 Hz, 4H).

The following compounds were synthesized using Method ZF with the appropriate reagents, including boronic acids/boronates and sulfonyl chlorides: Compounds I-150, I-164, I-181, I-183, I-191, I-192, I-193, I-234, I-235, and I-236.

### Example 36: Method ZG: Synthesis of Compound I-177

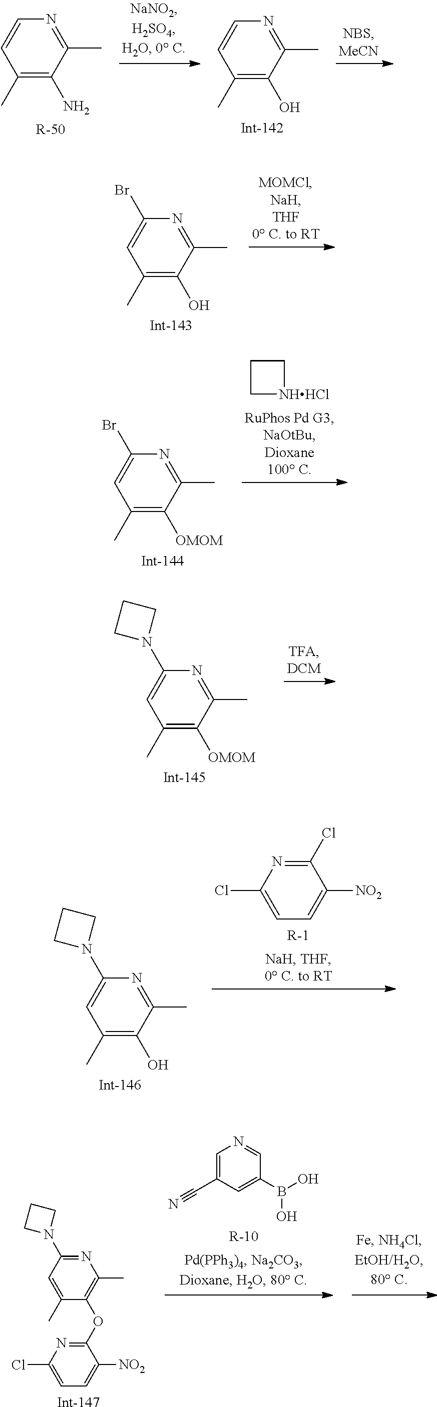

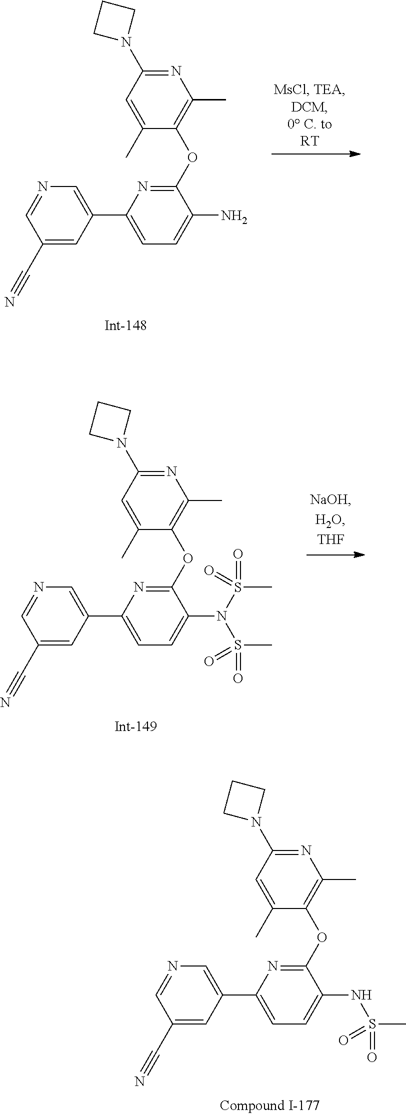 [SMILES: Cc1cc(N2CCC2)nc(C)c1Oc1nc(-c2cncc(C#N)c2)ccc1N ; Cc1cc(N2CCC2)nc(C)c1Oc1nc(-c2cncc(C#N)c2)ccc1N(S(C)(=O)=O)S(C)(=O)=O ; Cc1cc(N2CCC2)nc(C)c1Oc1nc(-c2cncc(C#N)c2)ccc1NS(C)(=O)=O]

Step 1: To a stirred solution of R-50 (400 mg, 3.28 mmol, 1.0 eq.) in H₂O (20 mL) was added concentrated sulfuric acid (4 mL) and aqueous solution of NaNO₂ (3.3 mL, 2.0 M, 6.6 mmol, 2.0 eq.) at 0° C., slowly. After stirring for 30 mins, the mixture was poured into ice water (50 mL) and neutralized by aqueous solution of NaOH (1 M). The resulting mixture was extracted with EtOAc (30 mL×3). The combined organic phase was dried over anhydrous Na₂SO₄ and concentrated under reduced pressure. The residue was purified by flash column chromatography through silica gel (eluted with PE:EtOAc=2:1 to 1:1) to afford Int-142 (221 mg, 1.8 mmol, 54.8%) as yellow solid. LCMS (ESI): m/z=124 [M+H]⁺.

Step 2: To a stirred solution of Int-142 (221 mg, 1.8 mmol, 1.0 eq.) in MeCN (10 mL) was added NBS (384.5 mg, 2.16 mmol, 1.2 eq.), slowly. After stirring at r.t. for 30 mins, the reaction mixture was diluted with H₂O (30 mL) and extracted with EtOAc (20 mL×3). The combined organic phase was dried over anhydrous Na₂SO₄ and concentrated under reduced pressure. The residue was purified by flash column chromatography on silica gel (eluted with PE:EtOAc=10:1 to 4:1) to afford Int-143 (230 mg, 1.14 mmol, 63.6%) as a white solid. LCMS (ESI): m/z=202 [M+H]⁺.

Step 3: To a stirred solution of Int-143 (230 mg, 1.14 mmol, 1.0 eq.) in THE (10 mL) was added NaH (57.5 mg, 1.37 mmol, 1.2 eq.) at 0° C. under N₂ atmosphere, slowly. After stirring at 0° C. for 30 mins, to the mixture was added chloro(methoxy)methane (100.3 mg, 1.25 mmol, 1.1 eq.), dropwise. After stirring at r.t. for 2 hrs, the reaction was quenched by saturated aqueous solution of NH₄Cl (40 mL) and extracted by EtOAc (20 mL×3). The combined organic phase was dried over anhydrous Na₂SO₄ and concentrated under reduced pressure. The residue was purified by flash column chromatography on silica gel (eluted with PE:EtOAc=0 to 5:1) to afford Int-144 (255 mg, 1.04 mmol, 91%) as a white solid. LCMS (ESI): m/z=246 [M+H]⁺.

Step 4: A mixture of Int-144 (100 mg, 0.69 mmol, 1.0 eq.), azetidine hydrochloride (71.1 mg, 0.76 mmol, 1.1 eq.), RuPhos Pd G3 (56 mg, 0.07 mmol, 0.1 eq.) and NaO^{t}Bu (198.9 mg, 2.07 mmol, 3.0 eq.) in dioxane (20 mL) was stirred under N₂ atmosphere at 100° C. for 6 hrs. After completion, the reaction mixture was diluted with H₂O (40 mL) and extracted with EtOAc (20 mL×3). The organic layers were combined, dried over anhydrous Na₂SO₄ and concentrated under reduced pressure. The residue was purified by flash column chromatography on silica gel (eluted with PE:EtOAc=0 to 2:1) to afford Int-145 (55.5 mg, 0.25 mmol, 36%) as a white solid. LCMS (ESI): m/z=223 [M+H]⁺.

Step 5: To a stirred solution of Int-145 (55.5 mg, 0.25 mmol, 1.0 eq.) in DCM (5 mL) were added TFA (1 mL). After stirring at r.t. for 20 min, the reaction mixture was concentrated under reduced pressure to afford Int-146 (50 mg, crude) as a white solid, which was used for next step, directly. LCMS (ESI): m/z=215 [M+H]⁺.

Steps 6 through 10: Conducted using analogous procedures used in Method ZD (Step 5-9) to provide Compound I-177, which was purified by Prep-HPLC (column: YMC-Actus Triart C8 250×20 mm, gradient: 30-95% MeCN in aqueous solution of NH₄HCO₃ (10 mM)) to afford Compound-177 (4.2 mg, 0.0093 mmol) as a white solid.

Compound I-177: Retention time: 2.665 min; LCMS (ESI): m/z=451 [M+H]⁺; ¹H NMR (400 MHz, DMSO-d₆) 1H NMR (400 MHz, DMSO-d₆) δ 9.90 (s, 1H), 9.05 (d, J=2.2 Hz, 1H), 8.97 (d, J=1.9 Hz, 1H), 8.57 (t, J=2.1 Hz, 1H), 7.94-7.83 (m, 2H), 6.18 (s, 1H), 3.92 (t, J=7.3 Hz, 4H), 3.19 (s, 3H), 2.33-2.27 (m, 2H), 2.11 (s, 3H), 2.01 (s, 3H).

The following compound was synthesized using Method ZG with the appropriate amines, boronic acids/boronates and sulfonyl chlorides: Compound I-180.

### Example 37: Method ZH: Synthesis of Compound I-176

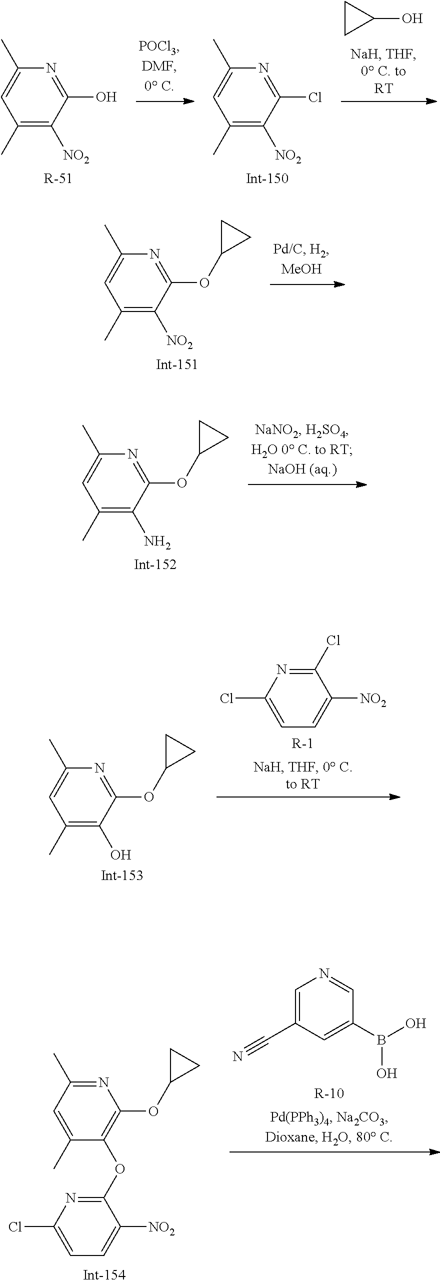

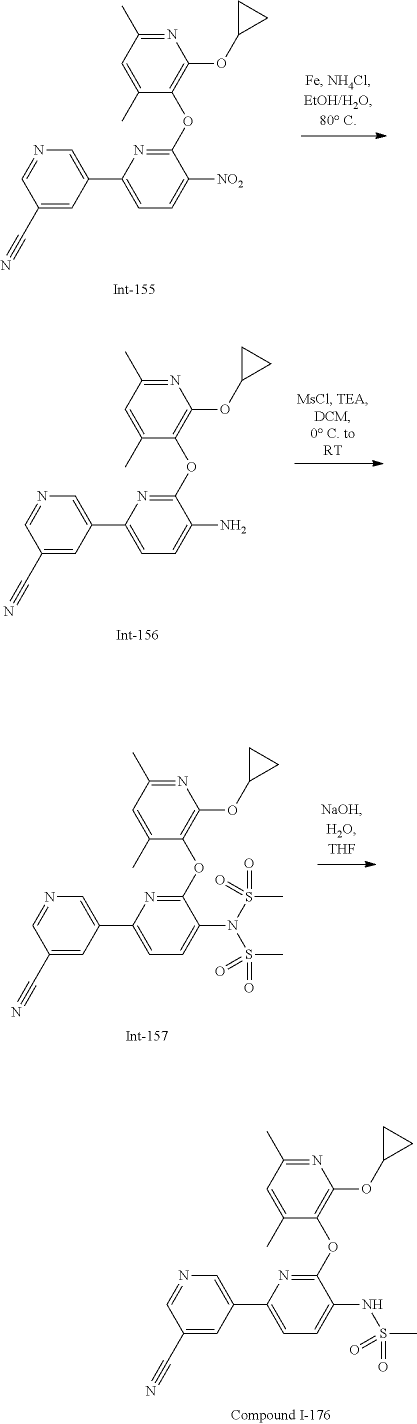 [SMILES: Cc1cc(C)c(Oc2nc(-c3cncc(C#N)c3)ccc2[N+](=O)[O-])c(OC2CC2)n1 ; Cc1cc(C)c(Oc2nc(-c3cncc(C#N)c3)ccc2N)c(OC2CC2)n1 ; Cc1cc(C)c(Oc2nc(-c3cncc(C#N)c3)ccc2N(S(C)(=O)=O)S(C)(=O)=O)c(OC2CC2)n1 ; Cc1cc(C)c(Oc2nc(-c3cncc(C#N)c3)ccc2NS(C)(=O)=O)c(OC2CC2)n1]

Step 1: To a stirred solution of 4, 6-dimethyl-3-nitropyridin-2-ol (R-51, 300 mg, 1.76 mmol, 1.0 eq.) in DMF (10 mL) was added POCl₃ (819.6 mg, 5.28 mmol, 3 eq.) at 0° C., slowly. After stirring at 90° C. for 3 hrs, the reaction mixture was quenched with H₂O (40 mL) and extracted with EtOAc (20 mL×3). The combined organic phase was dried over anhydrous Na₂SO₄ and concentrated under reduced pressure. The residue was purified by flash column chromatography on silica gel (eluted with PE:EtOAc=5:1 to 2:1) to afford Int-150 (272 mg, 1.46 mmol, 83%) as a white solid. LCMS (ESI): m/z=187 [M+H]⁺.

Step 2: To a stirred solution of cyclopropanol (34 mg, 0.59 mmol, 1 eq.) in THE (10 mL) was added NaH (30 mg, 0.75 mmol, 1.2 eq.) under N₂ atmosphere at 0° C., slowly. After stirring for 30 min, to the mixture was added Int-150 (100 mg, 0.54 mmol, 0.9 eq.) and the mixture was stirred at r.t. for another 2 hrs. After completion, the reaction was quenched by saturated aqueous solution of NH₄Cl (30 mL) and extracted with EtOAc (20 mL×3). The combined organic phase was dried over anhydrous Na₂SO₄ and concentrated under reduced pressure. The residue was purified by flash column chromatography on silica gel (eluted with PE:EtOAc=5:1 to 2:1) to afford Int-151 (91 mg, 0.44 mmol, 81%) as a white solid. LCMS (ESI): m/z=209 [M+H]⁺.

Step 3: To a stirred solution of Int-151 (91 mg, 0.44 mmol) in MeOH (10 mL) was added Pd/C (94 mg). The resulting mixture was stirred under H₂ atmosphere at r.t. overnight. After completion, the mixture was filtered, and the resulting filter cake was washed with MeOH (10 mL×3). The combined organic phase was concentrated under reduced pressure. The residue was purified by flash column chromatography on silica gel (eluted with PE:EtOAc=0 to 3:1) to afford Int-152 (74 mg, 0.41 mmol, 93.2%) as a white solid. LCMS (ESI): m/z=179 [M+H]⁺.

Steps 4 through 9: Conducted using analogous procedures used in Method Y (Step 5-10) to provide Compound I-176, which was purified by Prep-HPLC (column: YMC-Actus Triart C8 250×20 mm, gradient: 30-95% MeCN in aqueous solution of NH₄HCO₃ (10 mM)) to afford Compound I-176 (5.1 mg, 0.011 mmol) as a white solid.

Compound I-176: Retention time: 1.425 min; LCMS (ESI): m/z=452 [M+H]⁺; ¹H NMR (400 MHz, DMSO-d₆) δ 9.86 (s, 1H), 9.03 (d, J=2.2 Hz, 1H), 8.98 (d, J=1.9 Hz, 1H), 8.59 (t, J=2.1 Hz, 1H), 7.94-7.86 (m, 2H), 6.90 (s, 1H), 4.24 (tt, J=6.1, 3.0 Hz, 1H), 3.14 (s, 3H), 2.42 (s, 3H), 2.15 (s, 3H), 0.59-0.52 (m, 2H), 0.23-0.15 (m, 2H).

The following compound was synthesized using Method ZH with the appropriate alcohols, boronic acids/boronates and sulfonyl chlorides: Compound I-209.

### Example 38: Method ZI: Synthesis of Compound I-222

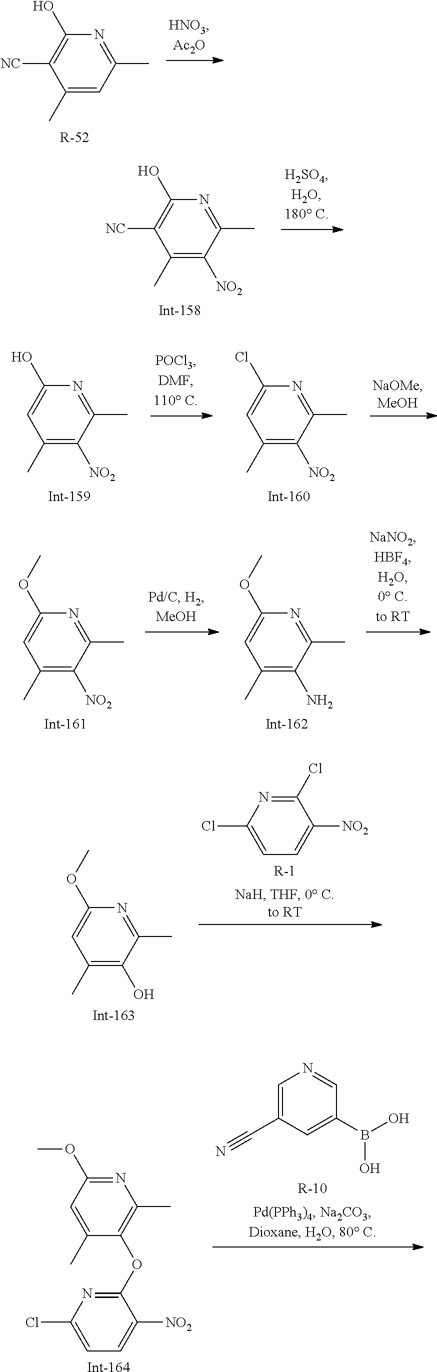

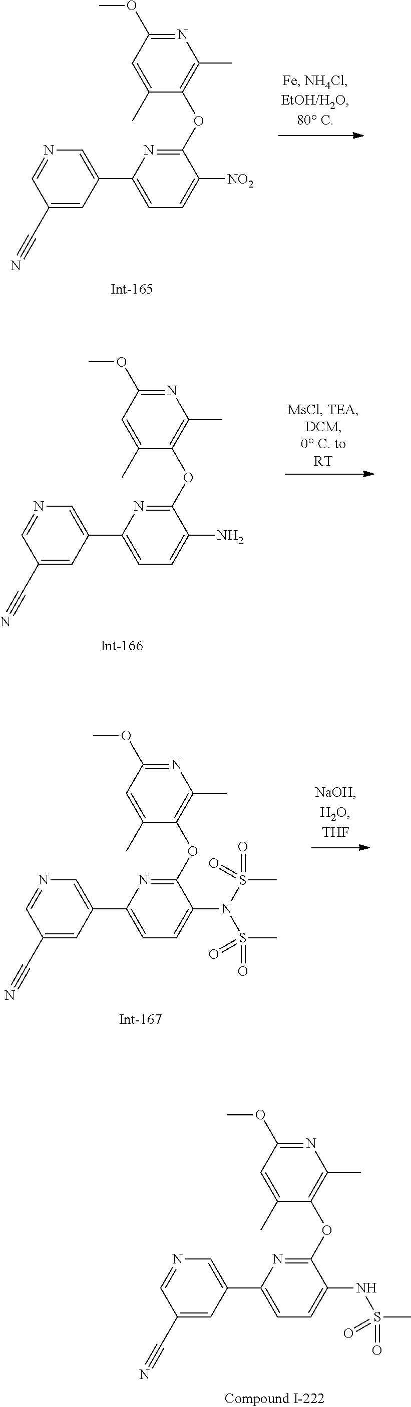 [SMILES: COc1cc(C)c(Oc2nc(-c3cncc(C#N)c3)ccc2[N+](=O)[O-])c(C)n1 ; COc1cc(C)c(Oc2nc(-c3cncc(C#N)c3)ccc2N)c(C)n1 ; COc1cc(C)c(Oc2nc(-c3cncc(C#N)c3)ccc2N(S(C)(=O)=O)S(C)(=O)=O)c(C)n1 ; COc1cc(C)c(Oc2nc(-c3cncc(C#N)c3)ccc2NS(C)(=O)=O)c(C)n1]

Step 1: To a stirred solution of 2-hydroxy-4, 6-dimethylnicotinonitrile (R-52, 14.8 g, 100 mmol) in HNO₃ (200 mL) was added Ac₂O (70 mL). After stirring at r.t. for 10 hrs, to the mixture was added a mixed solvent of PE and EtOAc (1:1, 200 mL) to give a suspension. The mixture was filtrated, the cake was washed with PE (50 mL×3) and dried in vacuum to afford Int-158 (9.2 g, 47.7 mmol, 47.7%) as a white solid. LCMS (ESI): m/z=194 [M+H]⁺.

Step 2: To a stirred solution of Int-158 (7 g, 36.3 mmol, 1.0 eq.) in water (200 mL) was added sulfuric acid (35 mL), slowly. The resulting mixture was stirred at 180° C. under N₂ atmosphere for 2 hrs. After completion, the reaction mixture was cooled to r.t. and extracted with EtOAc (100 mL×5). The combined organic phase was dried over anhydrous Na₂SO₄ and concentrated under reduced pressure. The residue was purified by flash column chromatography on silica gel (eluted with PE:EtOAc=1: 5) to afford Int-159 (4.0 g, 23.8 mmol, 65.6%) as a yellow solid. LCMS (ESI): m/z=169 [M+H]⁺.

Step 3: To a stirred solution of Int-159 (2.4 g, 14.3 mmol, 1.0 eq.) in POCl₃ (30 mL) was added DMF (1 mL), slowly. After stirring at 110° C. for 16 hrs, the reaction mixture was concentrated under reduced pressure. The residue was diluted with H₂O (150 mL) and the resulting solution was extracted with EtOAc (100 mL×3). Then the combined organic phase was dried over anhydrous Na₂SO₄ and concentrated under reduced pressure. The residue was purified by flash column chromatography on silica gel (eluted with PE:EtOAc=0 to 1:1) to afford Int-160 (1.2 g, 6.45 mmol, 45.1%) as a white solid. LCMS (ESI): m/z=187 [M+H]⁺.

Step 4: To a stirred solution of Int-160 (350 mg, 1.9 mmol, 1.0 eq.) in MeOH (10 mL) was added NaOMe (513 mg, 9.5 mmol, 5 eq.), slowly. After stirring at r.t. for 2 hrs, the reaction mixture was diluted with H₂O (40 mL) and extracted with EtOAc (30 mL×3). The combined organic phase was dried over anhydrous Na₂SO₄ and concentrated under reduced pressure. The residue was purified by flash column chromatography on silica gel (eluted with PE:EtOAc=0 to 5:1) to afford Int-161 (300 mg, 1.65 mmol, 86.8%) as a white solid. LCMS (ESI): m/z=183 [M+H]⁺.

Step 5: A mixture of Int-161 (300 mg, 1.65 mmol) and Pd/C (80 mg) in MeOH (20 mL) was stirred under H₂ atmosphere at r.t. for 2 hrs. After completion, the reaction mixture was filtered through a pad of Celite and the filter cake was washed with MeOH (30 mL×3). The combined filtrates were concentrated under reduced pressure. The residue was purified by flash column chromatography on silica gel (eluted with PE:EtOAc=0 to 1:10) to afford Int-162 (240 mg, 1.58 mmol, 95.6%) as a white solid. LCMS (ESI): m/z=153 [M+H]⁺.

Step 6: To a stirred solution of Int-162 (240 mg, 1.58 mmol, 1 eq.) in water (7 mL) was added HBF₄ (7 mL) and aqueous solution of NaNO₂ (1.25 mL, 2 M, 2.5 mmol, 1.6 eq.) at 0° C., slowly. After stirring at r.t. for 1 h, the mixture was stirred at 100° C. for 5 hrs. After completion, the mixture was poured into ice water (50 mL), neutralized by aqueous solution of NaOH (4 M) and extracted with EtOAc (50 mL×3). The organic phase was combined, dried over anhydrous Na₂SO₄ and concentrated under reduced pressure. The residue was purified by flash column chromatography on silica gel (eluted with EtOAc) to afford Int-163 (91 mg, 0.59 mmol, 37.6%) as a white solid. LCMS (ESI): m/z=154[M+H]⁺.

Steps 7 through 11: Conducted using analogous procedures used in Method Y (Steps 6-10) to provide Compound I-222, which was purified by Prep-HPLC (column: welch xbridge xb 5 μm 21.2×250 mm, gradient: 25-95% MeCN in H₂O (with 0.1% TFA) to afford Compound I-222 (12.1 mg, 0.028 mmol) as a white solid.

Compound I-222: Retention time: 1.317 min; LCMS (ESI): m/z=426.4 [M+H]⁺; 1H NMR (400 MHz, DMSO-d₆) δ 9.97 (s, 1H), 9.03 (d, J=2.2 Hz, 1H), 8.97 (d, J=1.9 Hz, 1H), 8.57 (t, J=2.2 Hz, 1H), 7.96 (d, J=8.1 Hz, 1H), 7.89 (d, J=8.1 Hz, 1H), 6.69 (s, 1H), 3.85 (s, 3H), 3.22 (s, 3H), 2.21 (s, 3H), 2.07 (s, 3H).

The following compounds were synthesized using Method ZI with the appropriate alcohols, boronic acids/boronates and sulfonyl chlorides: Compound I-132 and I-178.

### Example 39: Method ZJ: Synthesis of Compound I-182

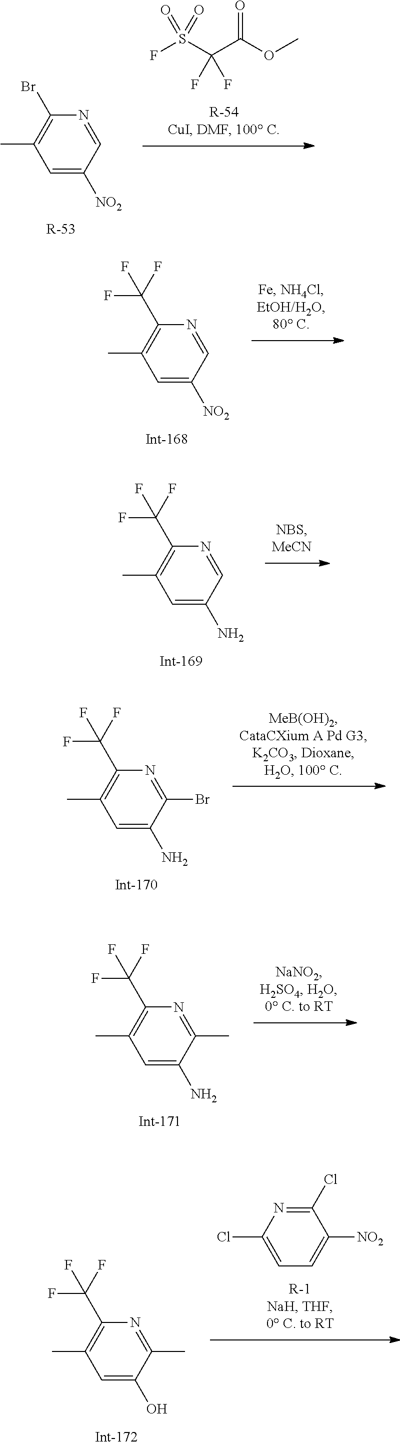

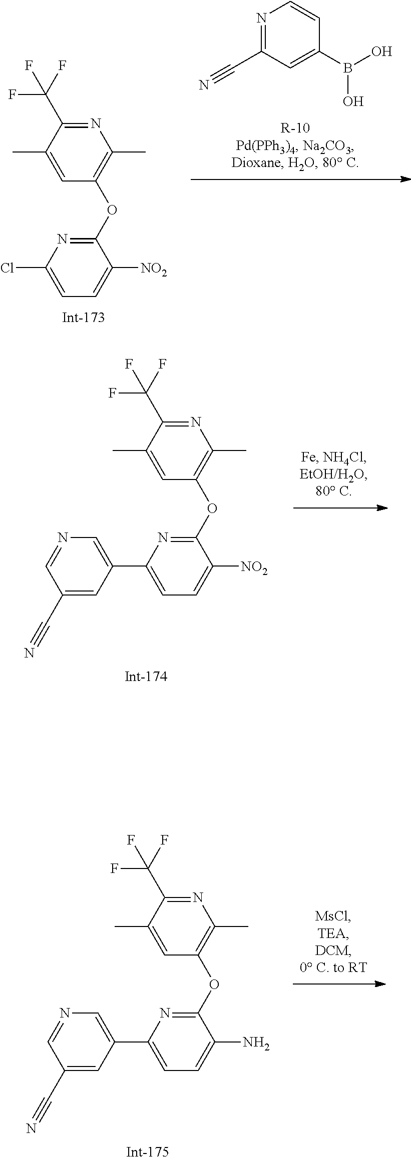

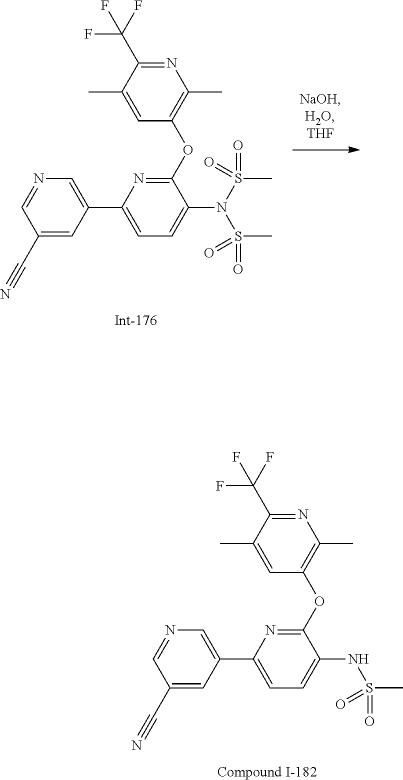 [SMILES: Cc1cc(Oc2nc(-c3cncc(C#N)c3)ccc2N(S(C)(=O)=O)S(C)(=O)=O)c(C)nc1C(F)(F)F ; "Int-176" ; Cc1cc(Oc2nc(-c3cncc(C#N)c3)ccc2NS(C)(=O)=O)c(C)nc1C(F)(F)F ; "Compound I-182"]

Step 1: A mixture of 2-bromo-3-methyl-5-nitropyridine (R-53, 200 mg, 0.92 mmol, 1.0 eq.), methyl 2, 2-difluoro-2-(fluorosulfonyl)acetate (R-54, 350 mg, 1.84 mmol, 2.0 eq.) and CuI (350 mg, 1.84 mmol, 2.0 eq.) in DMF (6 mL) was stirred under N₂ atmosphere at 100° C. for 2 hrs. After completion, the reaction mixture was diluted with water (50 mL) and extracted with EtOAc (50 mL×3). The combined organic layers were washed with brine (50 mL×3), dried over anhydrous Na₂SO₄ and concentrated under reduced pressure. The residue was purified by flash column chromatography through silica gel (eluted with PE:EtOAc=5:1) to afford Int-168 (100 mg, 0.49 mmol, 53.3%) as a white solid. LCMS (ESI): m/z=207 [M+H]⁺.

Step 2: A mixture of Int-168 (100 mg, 0.49 mmol, 1.0 eq.), NH₄Cl (262 mg, 4.9 mmol, 10.0 eq.) and Fe (271 mg, 4.9 mmol, 10.0 eq.) in EtOH (30 mL) and H₂O (10 mL) was stirred at 80° C. for 1 h. After completion, the reaction mixture was filtered through a pad of Celite®, the filter cake was washed with MeOH (20 mL×3). The combined organic layers were dried over anhydrous Na₂SO₄ and concentrated under reduced pressure. The residue was purified by flash column chromatography through silica gel (eluted with PE:EtOAc=4:1 to 3:1) to afford Int-169 (60 mg, 0.34 mmol, 69.40) as a white solid. LCMS (ESI): m/z=177 [M+H]⁺.

Steps 3 through 10: Conducted using analogous procedures used in Method Y (Steps 3-10) to provide Compound I-182, which was purified by Prep-HPLC (column: YMC-Actus Triart C18 250×20 mm×5 um, gradient: 10-95% MeCN in H₂O (with 0.100 FA)) to afford Compound 1-182 (3.1 mg, 0.0065 mmol) as a white solid.

Compound 1-182: Retention time: 1.457 min; LCMS (EST): m/z=464 [M+H]⁺; ¹H NMR (400 MHz, DMSO) δ 10.02 (s, 1H1), 9.12 (d, J=2.1 Hz, 1H1), 9.00 (d, J=1.9 Hz, 1H), 8.60 (t, J=2.1 Hz, 1H), 8.00 (q, J=8.1 Hz, 2H), 7.87 (s, 1H), 3.22 (s, 3H), 2.45 (s, 3H), 2.43 (s, 3H).

### Example 40: Method ZK: Synthesis of Compound I-251

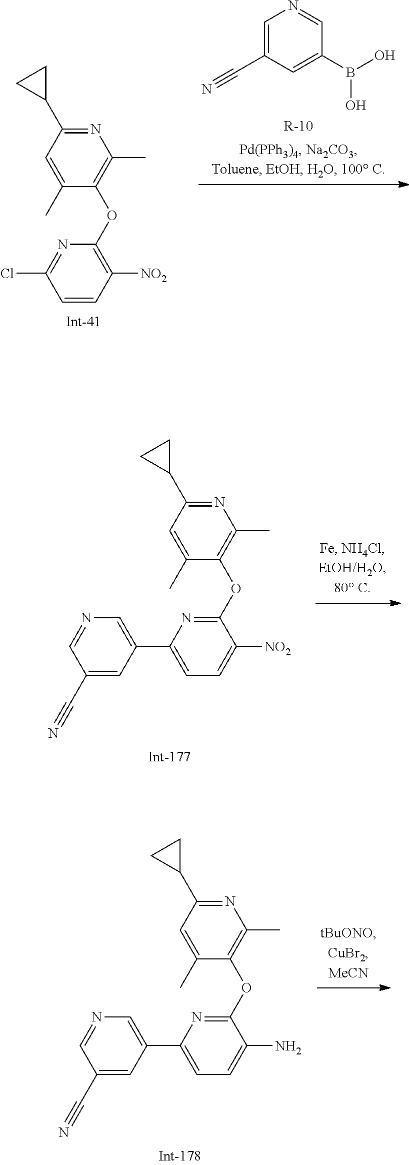 [SMILES: Cc1cc(C2CC2)nc(C)c1Oc1nc(Cl)ccc1[N+](=O)[O-] ; N#Cc1cncc(B(O)O)c1 ; "R-10" ; Cc1cc(C2CC2)nc(C)c1Oc1nc(-c2cncc(C#N)c2)ccc1[N+](=O)[O-] ; Cc1cc(C2CC2)nc(C)c1Oc1nc(-c2cncc(C#N)c2)ccc1N]

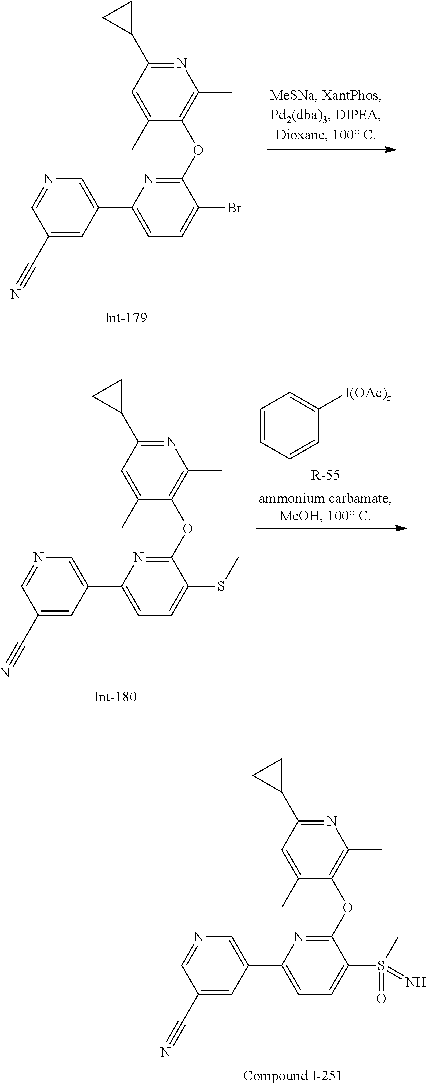

Step 1: To a solution of Int-41 (50 mg, 0.16 mmol, 1.0 eq., prepared as in Method R) in toluene (6 mL), EtOH (6 mL) and H₂O (2 mL) were added Na₂CO₃ (68 mg, 0.63 mmol, 4.0 eq.), (5-cyanopyridin-3-yl) boronic acid (R-10, 36 mg, 0.24 mmol, 1.5 eq.) and Pd(PPh₃) 4 (18 mg, 16 μmol, 0.1 eq.). The reaction mixture was stirred at 100° C. for 2 hrs under N₂. After completion, the reaction mixture was poured into water (30 mL) and then extracted with EtOAc (15 mL×3). The organic layers were combined and washed with brine (20 mL), dried over anhydrous Na₂SO₄, and concentrated under reduced pressure. The residue was purified by flash column chromatography on silica gel (eluted with PE:EtOAc=3:1 to 2:1) to afford Int-177 (50 mg, 0.13 mmol, 79%) as a white solid. LC-MS (ESI) m/z=388 [M+H]⁺.

Step 2: A mixture of Int-177 (50 mg, 0.13 mmol, 1.0 eq.), Fe powder (36 mg, 0.65 mmol, 5 eq.) and NH₄Cl (70 mg, 1.3 mmol, 10 eq.) in EtOH (20 mL) and H₂O (10 mL) was stirred at 80° C. for 2 hrs. After completion, the reaction mixture was filtered through a pad of Celite and the filter cake was washed with MeOH. The combined filtrates were concentrated, the residue was triturated with H₂O, then filtered to afford Int-178 (44.2 mg, crude) as a brown solid. LCMS (ESI): m/z=358 [M+H]⁺.

Step 3: To a stirred solution of Int-178 (380 mg, 1.06 mmol, 1.0 eq.) in MeCN (30 mL) was added ^{t}BuONO (164.6 mg, 1.6 mmol, 1.5 eq.) and CuBr₂ (356.2 mg, 1.6 mmol, 1.5 eq.) at 0° C. slowly. After stirring at r.t. for 2 hrs, the mixture was diluted with H₂O (50 mL) and extracted with EtOAc (30 mL×3). The combined organic phase was dried over anhydrous Na₂SO₄ and concentrated under reduced pressure. The residue was purified by flash column chromatography on silica gel (eluted with PE:EtOAc=3:1) to afford Int-179 (200 mg, 0.48 mmol, 45%) as a yellow oil. LCMS (ESI): m/z=421 [M+H]⁺.

Step 4: A mixture of Int-179 (200 mg, 0.48 mmol, 1.0 eq.), MeSNa (50.5 mg, 0.72 mmol, 1.5 eq.), Pd₂(dba)₃ (86.9 mg, 0.095 mmol, 0.2 eq.), XantPhos (82.4 mg, 0.142 mmol, 0.3 eq.), and DIEA (368.2 mg, 2.85 mmol, 6.0 eq.) in dioxane (20 mL) was stirred at 100° C. under N₂ atmosphere for 16 hrs. After completion, the mixture was diluted with H₂O (50 mL) and extracted with EtOAc (30 mL×3). The combined organic phase was dried over anhydrous Na₂SO₄ and concentrated under reduced pressure. The residue was purified by flash column chromatography on silica gel (eluted with PE:EtOAc=3:1) to afford Int-180 (90 mg, 0.23 mmol, 48.3%) as a white solid. LCMS (ESI): m/z=389 [M+H]⁺.

Step 5: To a stirred mixture of Int-180 (90 mg, 0.23 mmol, 1.0 eq.) in MeOH (10 mL) was added (diacetoxyiodo)benzene (R-55, 222.2 mg, 0.69 mmol, 3.0 eq.) and ammonium carbamate (72.34 mg, 0.93 mmol, 4.0 eq.). After stirring at 100° C. for 3 h, the reaction mixture was diluted with H₂O (60 mL) and extracted with EtOAc (30 mL×3). The organic layers were combined, dried over anhydrous Na₂SO₄ and concentrated under reduced pressure. The residue was purified by prep-HPLC (column: YMC-Actus Triart C18 250×20 mm×5 um, gradient: 5%-95% MeCN in H₂O [with 0.1% TFA]) to afford Compound I-251 (28.2 mg, 0.067 mmol, 29%) as a white solid.

Compound I-251: Retention time: 1.313 min; LCMS (ESI): m/z=420.3 [M+H]⁺; ¹H NMR (400 MHz, DMSO-d₆) δ 9.10 (d, J=2.1 Hz, 1H), 9.08 (d, J=1.8 Hz, 1H), 8.68 (t, J=2.0 Hz, 1H), 8.53 (d, J=7.9 Hz, 1H), 8.16 (d, J=7.9 Hz, 1H), 7.36 (s, 1H), 3.49 (s, 3H), 2.38 (d, J=9.3 Hz, 3H), 2.29-2.22 (m, 1H), 2.20 (d, J=9.4 Hz, 3H), 1.17 (d, J=7.8 Hz, 2H), 1.09-1.01 (m, 2H).

The following compounds were synthesized using Method ZK with the appropriate alcohols, thiols, and boronic acids/boronates: Compounds I-116, I-245, I-248, I-249, I-279, I-287, I-295, I-296, I-297, I-301, I-302, I-303, I-304, I-316, I-317, and I-319.

### Example 41: Method ZL: Synthesis of Compound I-115

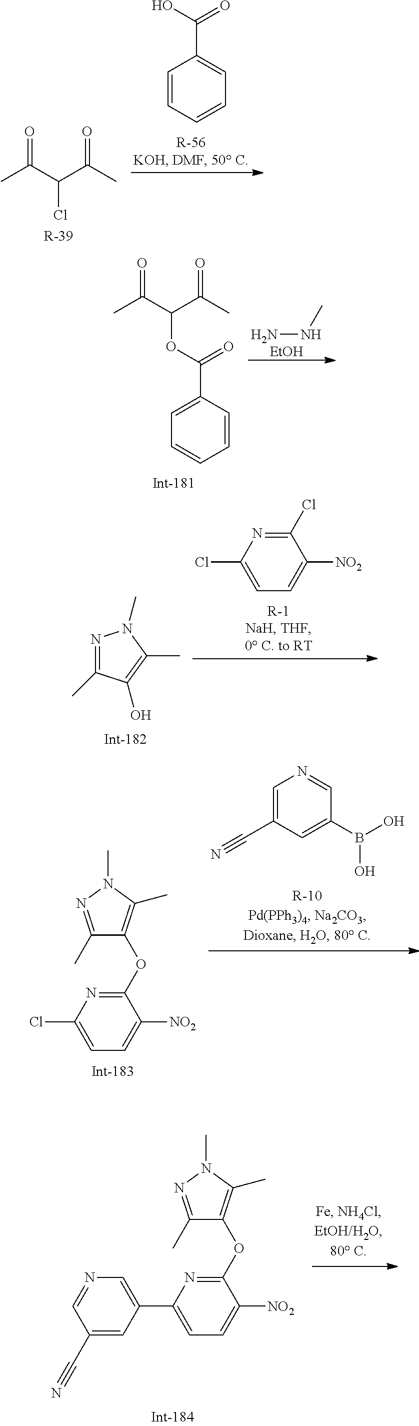

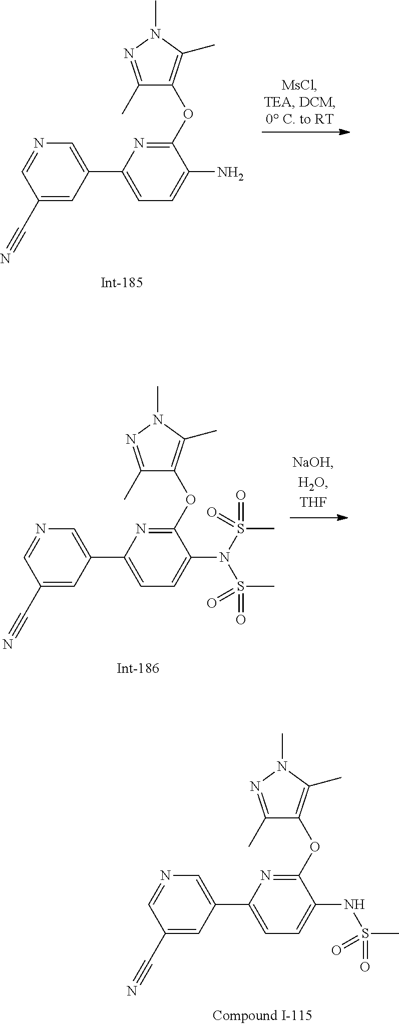 [SMILES: Cc1nn(C)c(C)c1Oc1nc(-c2cncc(C#N)c2)ccc1N ; Cc1nn(C)c(C)c1Oc1nc(-c2cncc(C#N)c2)ccc1N(S(C)(=O)=O)S(C)(=O)=O ; Cc1nn(C)c(C)c1Oc1nc(-c2cncc(C#N)c2)ccc1NS(C)(=O)=O]

Step 1: To a stirred solution of benzoic acid (R-56, 1.82 g 149 mmol, 1.0 eq.) in DMF (20 mL) was added KOH (920 mg, 16.4 mmol, 1.1 eq.) and 3-chloropentane-2, 4-dione (R-39, 2 g, 14.9 mmol, 1.0 eq.). The resulting mixture was stirred at 50° C. for 13 hrs. After completion, the reaction mixture was partitioned by EtOAc (100 mL) and water (100 mL). The organic phase was washed with brine (100 mL×3), dried over Na₂SO₄, and concentrated under reduced pressure to afford Int-181 (4.1 g, crude) as a yellow oil, which was used in the next step directly.

Step 2: A mixture of Int-181 (1.0 g, crude) and methylhydrazine (5 mL) in EtOH (20 mL) was stirred at r.t. overnight. After completion, the mixture was diluted with H₂O (40 mL) and extracted with EtOAc (40 mL×3). The combined organic phase was dried over anhydrous Na₂SO₄ and concentrated under reduced pressure. The residue was purified by flash column chromatography on silica gel (eluted with PE:EtOAc=4:1) to afford Int-182 (316 mg, 2.5 mmol, 68.700 for two steps) as a yellow oil. LCMS (ESI): m/z=127 [M+H]⁺.

Steps 3 through 7: Conducted using analogous procedures used in Method Y (Steps 6-10) to provide Compound I-115, which was purified by Prep-HPLC (column: YMC-Actus Triart C8 250×20 mm×5 um, gradient: 5-95% MeCN in aqueous solution of TFA (10 mM)) to afford Compound I-115 (3.1 mg, 0.0078 mmol) as a white solid.

Compound I-115: Retention time: 1.00 min; LCMS (ESI): m/z=399 [M+H]⁺; ¹H NMR (400 MHz, DMSO-d₆) δ 9.87 (s, 1H), 9.19 (d, J=1.7 Hz, 1H), 9.00 (s, 1H), 8.65 (s, 1H), 7.89 (s, 2H), 3.70 (s, 3H), 3.18 (s, 3H), 2.11 (s, 3H), 1.98 (s, 3H).

The following compounds were synthesized using Method ZL with the appropriate alcohols, boronic acids/boronates and sulfonyl chlorides: Compound I-107, I-113, and I-206.

### Example 42: Method ZM: Synthesis of Compound I-216

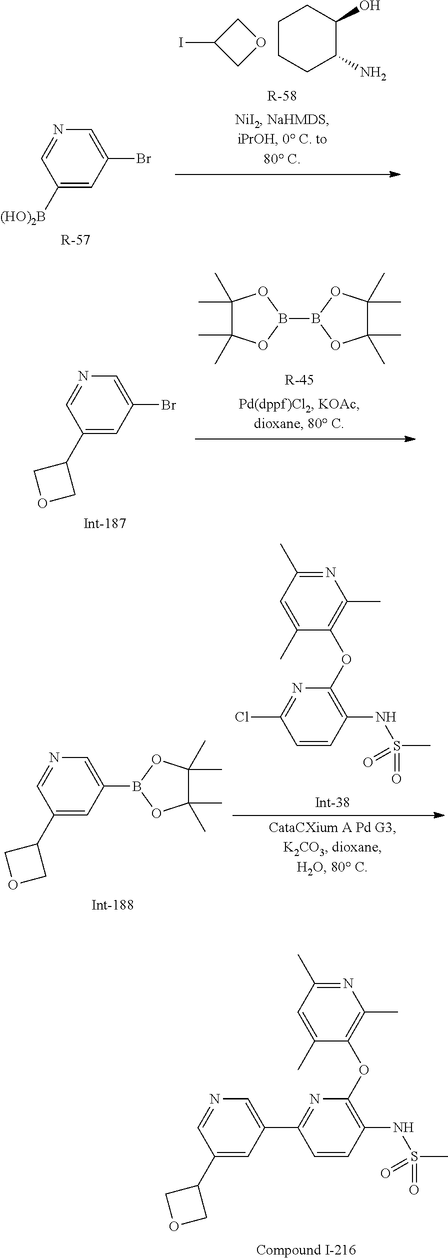

Step 1: To a stirred solution of (5-bromopyridin-3-yl)boronic acid (R-57, 500 mg, 2.5 mmol, 1 eq), NiI₂ (23.4 mg, 0.075 mmol, 0.03 eq.) and (1R, 2R)-2-aminocyclohexan-1-ol (R-58, 23.0 mg, 0.2 mmol, 0.08 eq.) in isopropanol (2 mL) was added NaHMDS (1.239 mL, 2.5 mmol, 1 eq.) at 0° C., dropwise. The resulting mixture was stirred under N₂ atmosphere at r.t. for 10 min. Then, to the mixture was added 3-iodooxetane (459.9 mg, 2.5 mmol, 1.0 eq.) and the resulting mixture was stirred at 80° C. for 1.5 h. After completion, the resulting solution was diluted with H₂O (70 mL) and extract with EtOAc (50 mL×3). The combined organic phase was dried over anhydrous Na₂SO₄ and concentrated under reduced pressure. The residue was purified by silica column (eluting with EtOAc:PE=1:2) to afford Int-187 (100 mg, 0.467 mmol, 18.9%) as a white solid. LCMS (ESI): m/z=214 [M+H]⁺.

Steps 2 and 3: To a stirred solution of Int-187 (100 mg, 0.47 mmol, 1.0 eq.), 4,4,4′,4′,5,5,5′,5′-octamethyl-2,2′-bi(1,3,2-dioxaborolane) (R-45, 238.7 mg, 0.94 mmol, 2.0 eq.) and KOAc (138 mg, 1.40 mmol, 3.0 eq.) in dioxane (3 mL) was added Pd(dppf)Cl₂ (81.7 mg, 0.1 mmol, 0.2 eq.). The resulting mixture was stirred under N₂ atmosphere at 80° C. overnight to provide Int-188. After cooling to r.t., to the mixture was added N-(6-chloro-2-((2, 4, 6-trimethylpyridin-3-yl)oxy)pyridin-3-yl)methanesulfonamide (Int-38, 160.3 mg, 0.47 mmol, 1.0 eq.), Cs₂CO₃ (456.1 mg, 1.4 mmol, 3.0 eq.) and cataCXium A Pd G3 (36.4 mg, 0.05 mmol, 0.1 eq.). The resulting mixture was stirred at 100° C. under N₂ atmosphere for 5 hrs, then the mixture was diluted with H₂O (40 mL) and extracted with EtOAc (20 mL×3). The combined organic phase was dried over anhydrous Na₂SO₄ and concentrated under reduced pressure. The residue was purified by prep-HPLC (column: Welch XBridge C18 250×21 mm, gradient: 20-95% MeCN in H₂O (with 0.1% FA)) to afford Compound I-216 (24.4 mg, 0.055 mmol, 11.8% for two steps) as a white solid.

Compound I-216: Retention time: 0.91 min; LCMS (ESI): m/z=441.3 [M+H]f; ¹H NMR (400 MHz, DMSO) δ 9.89 (s, 1H), 8.73 (d, J=2.0 Hz, 1H), 8.48 (d, J=2.0 Hz, 1H), 8.13 (t, J=2.0 Hz, 1H), 7.93 (d, J=8.1 Hz, 1H), 7.84 (d, J=8.1 Hz, 1H), 7.09 (s, 1H), 4.92 (dd, J=8.4, 6.0 Hz, 2H), 4.57 (t, J=6.4 Hz, 2H), 4.35-4.25 (m, 1H), 3.20 (s, 3H), 2.43 (s, 3H), 2.23 (s, 3H), 2.08 (s, 3H).

### Example 43: Method ZN: Synthesis of Compound I-289

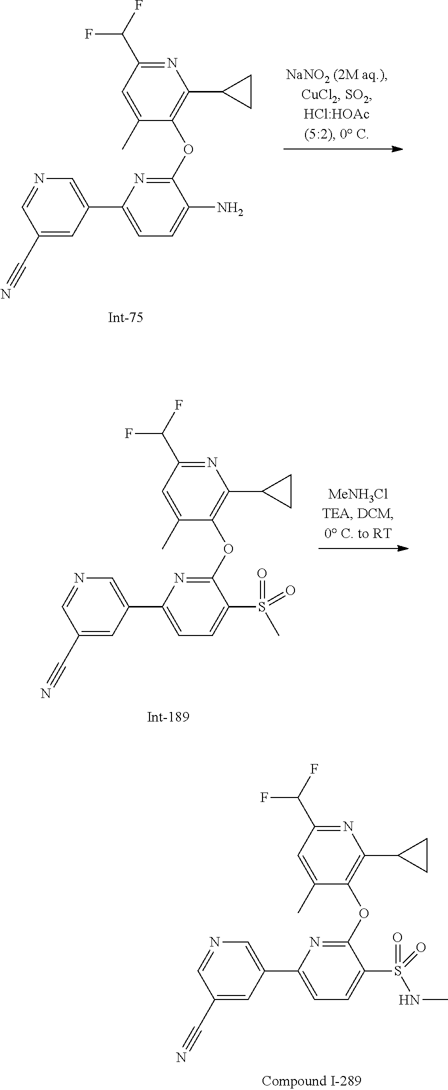 [SMILES: Cc1cc(C(F)F)nc(C2CC2)c1Oc1nc(-c2cncc(C#N)c2)ccc1N ; Cc1cc(C(F)F)nc(C2CC2)c1Oc1nc(-c2cncc(C#N)c2)ccc1S(C)(=O)=O ; CNS(=O)(=O)c1ccc(-c2cncc(C#N)c2)nc1Oc1c(C)cc(C(F)F)nc1C1CC1]

Steps 1 and 2: To a stirred solution ofTInt-75 (40 mg, 0.1 mmol, 1.0 eq. synthesized as in Method W) in concentrated HCl (5 mL) and glacial acetic acid (2 mL) was added aqueous solution of NaNO₂ (2.0 M, 0.1 mL, 2.0 eq.) at 0° C., slowly. After stirring for 30 min, to the mixture was added CuCl₂ (27 mg, 0.2 mmol, 1.0 eq.), then, SO₂ was bubbled through the reaction mixture at 0° C. for 15 min. After completion, the mixture was poured into ice water (10 mL) and extracted with EtOAc (10 mL×3). The combined organic phase was washed with a saturated aqueous solution of NaHCO₃ (10 mL×3), dried over anhydrous Na₂SO₄ and concentrated under reduced pressure. The crude product Int-189 was dissolved in DCM (10 mL), then to the resulting mixture was added methylamine hydrochloride (33.5 mg, 0.5 mmol, 5.0 eq.) and TEA (0.5 mL) at 0° C. After stirring at r.t. for 30 min, the mixture was diluted with H₂O (10 mL) and extracted with EtOAc (10 mL×3). The organic layers were combined, dried over anhydrous Na₂SO₄ and concentrated under reduced pressure. The residue was purified by Prep-HPLC (column: YMC-Actus Triart C18 250×20 mm×5 um, gradient: 30-95% MeCN in H₂O (with 0.1% TFA)) to afford Compound I-289 (18.3 mg, 0.039 mmol, 39%) as a white solid.

Compound I-289: Retention time: 1.563 min; LCMS (ESI): m/z=472.1 [M+H]⁺; 1H NMR (400 MHz, DMSO) δ 9.05 (dd, J=5.9, 2.0 Hz, 2H), 8.66 (t, J=2.0 Hz, 1H), 8.44 (d, J=7.9 Hz, 1H), 8.10 (d, J=7.9 Hz, 1H), 8.02 (q, J=4.7 Hz, 1H), 7.55 (s, 1H), 6.89 (t, J=55.1 Hz, 1H), 2.63 (d, J=4.8 Hz, 3H), 2.22 (s, 3H), 2.15-2.04 (m, 1H), 1.12-1.01 (m, 1H), 0.96-0.86 (m, 1H), 0.77 (dd, J=15.2, 8.3 Hz, 2H); ¹⁹F NMR (376 MHz, DMSO) −114.48 (d, J=8.3 Hz).

The following compounds were synthesized using Method ZN with the appropriate alcohols, boronic acids/boronates and amines: Compounds I-288, I-308, I-312, I-320, and I-321.

### Example 44: Method ZO: Synthesis of Compound I-293

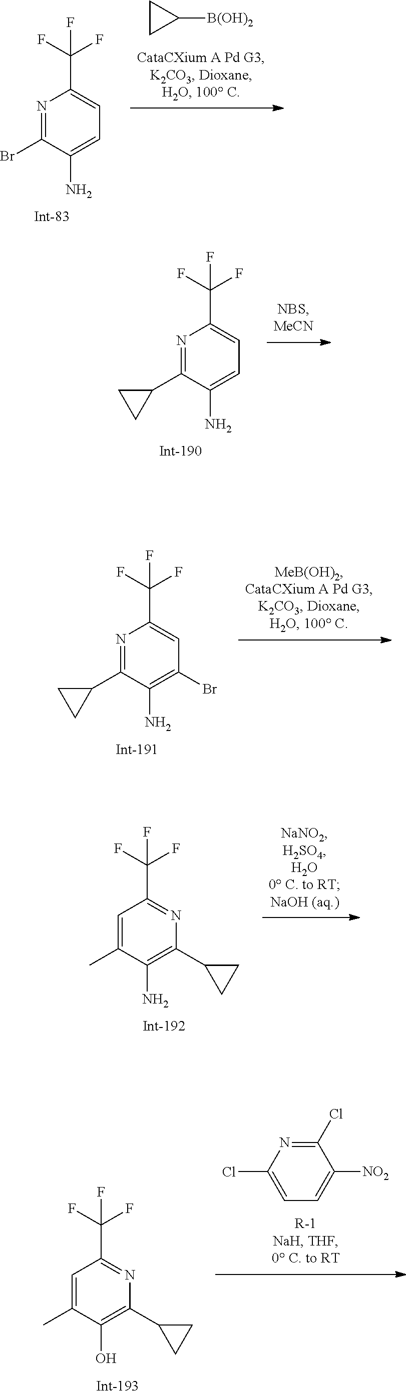

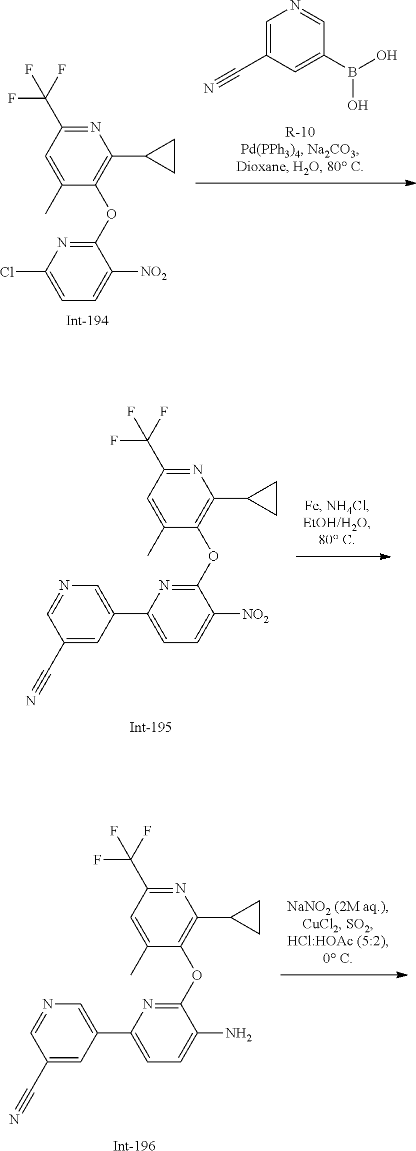

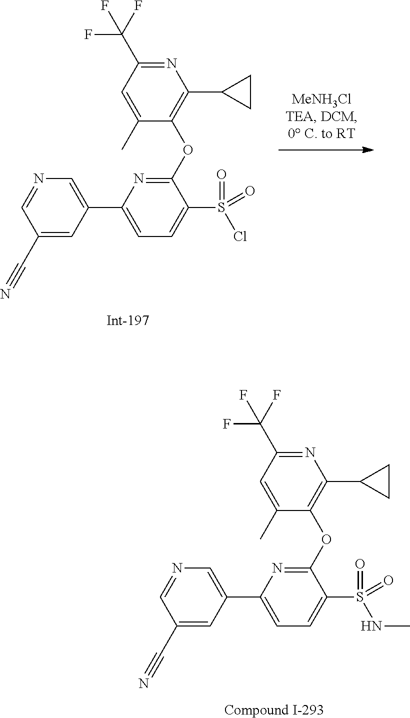 [SMILES: Cc1cc(C(F)(F)F)nc(C2CC2)c1Oc1nc(-c2cncc(C#N)c2)ccc1S(=O)(=O)Cl ; "Int-197" ; CNS(=O)(=O)c1ccc(-c2cncc(C#N)c2)nc1Oc1c(C)cc(C(F)(F)F)nc1C1CC1 ; "Compound I-293"]

Step 1: A mixture of Int-83 (2.0 g, 8.3 mmol, 1.0 eq.), cyclopropylboronic acid (1.4 g, 16.6 mmol, 2.0 eq.), cataCXium A Pd G3 (604.5 mg, 0.83 mmol, 0.1 eq.) and K₂CO₃ (3.4 g, 24.9 mmol, 3.0 eq.) in dioxane (100 mL) and H₂O (20 mL) was stirred at 100° C. under N₂ atmosphere for 3 hrs. After completion, the mixture was diluted with H₂O (60 mL) and extracted with EtOAc (40 mL×3), the combined organic phase was dried over anhydrous Na₂SO₄ and concentrated under reduced pressure. The residue was purified by flash column chromatography through silica gel (eluted with PE:EtOAc=5:1) to afford Int-190 (1.3 g, 6.4 mmol, 77.1%) as a yellow solid. LCMS (ESI): m/z=203 [M+H]⁺.

Step 2: To a stirred solution of Int-190 (1.3 g, 6.4 mmol, 1.0 eq.) in MeCN (50 mL) was added NBS (1.4 g, 7.9 mmol, 1.2 eq.). After stirring at r.t. for 2 hrs, the mixture was diluted with H₂O (50 mL) and extracted with EtOAc (40 mL×3). The organic layers were combined, dried over anhydrous Na₂SO₄ and concentrated under reduced pressure. The residue was purified by flash column chromatography through silica gel (eluted with PE:EtOAc=5:1) to afford Int-191 (1.56 g, 5.57 mmol, 87%) as a yellow solid. LCMS (ESI): m/z=281 [M+H]⁺.

Step 3: A mixture of Int-191 (1.56 g, 5.57 mmol, 1.0 eq.), methylboronic acid (668 mg, 11.2 mmol, 2.0 eq.), cataCXium A Pd G3 (407.8 mg, 0.56 mmol, 0.1 eq.) and K₂CO₃ (2.3 g, 16.7 mmol, 3.0 eq.) in dioxane (50 mL) and H₂O (10 mL) was stirred at 100° C. under N₂ atmosphere for 3 hrs. After completion, the mixture was diluted with H₂O (60 mL) and extracted with EtOAc (40 mL×3), the combined organic phase was dried over anhydrous Na₂SO₄ and concentrated under reduced pressure. The residue was purified by flash column chromatography through silica gel (eluted with PE:EtOAc=4:1) to afford Int-192 (990 mg, 4.58 mmol, 82.1%) as a yellow solid. LCMS (ESI): m/z=217 [M+H]⁺.

Steps 4 and 5: To a stirred solution of Int-192 (990 mg, 4.58 mmol, 1.0 eq.) in water (50 mL) was added concentrated sulfuric acid (10 mL) and aqueous solution of NaNO₂ (1 M, 7 mL, 7 mmol, 1.5 eq.) at 0° C., slowly. After stirring at r.t. for 6 hrs, the mixture was poured into ice water (50 mL) and neutralized with an aqueous solution of NaOH (4 M). After completion, the resulting mixture was extracted with EtOAc (30 mL×3), and the organic phase was combined, dried over anhydrous Na₂SO₄ and concentrated under reduced pressure. The crude product Int-193 was dissolved in anhydrous THE (20 mL), then to the resulting mixture was added NaH (275 mg, 6.9 mmol, 1.5 eq.) under N₂ atmosphere at 0° C., slowly. After stirring at 0° C. for 30 min, to the mixture was added 2,6-dichloro-3-nitropyridine (R-1, 1.06 g, 5.5 mmol, 1.2 eq.). After stirring at r.t. for 2 hrs, the mixture was quenched with saturated aqueous solution of NH₄Cl (50 mL) and extracted with EtOAc (20 mL×3). The combined organic phase were concentrated under reduced pressure, the residue was diluted with H₂O (30 mL) and extracted with EtOAc (15 mL×3). The combined organic layers were dried over anhydrous Na₂SO₄ and concentrated under reduced pressure. The residue was purified by flash column chromatography on silica gel (eluted with PE:EtOAc=1:2) to afford Int-194 (717.6 mg, 1.92 mmol, 42% for two steps) as a yellow solid. LCMS (ESI): m/z=374 [M+H]⁺.

Step 6: A mixture of Int-194 (717.6 mg, 1.92 mmol, 1.0 eq.), (5-cyanopyridin-3-yl)boronic acid (R-10, 341 mg, 2.3 mmol, 1.2 eq.), Pd(PPh₃)₄ (231 mg, 0.2 mmol, 0.1 eq.) and Na₂CO₃ (610.6 mg, 5.76 mmol, 3.0 eq.) in dioxane (100 mL) and water (20 mL) was stirred under N₂ atmosphere at 80° C. for 4 hrs. After completion, the reaction mixture was diluted with H₂O (50 mL) and extracted with EtOAc (30 mL×3). The organic layers were combined, dried over anhydrous Na₂SO₄ and concentrated under reduced pressure. The residue was purified by flash column chromatography on silica gel (eluted with PE:EtOAc=1: 9) to afford Int-195 (643.7 mg, 1.46 mmol, 76%) as a yellow solid. LCMS (ESI): m/z=442 [M+H]⁺.

Step 7: A mixture of Int-195 (643.7 mg, 1.46 mmol, 1.0 eq.), Fe powder (408.8 mg, 7.3 mmol, 5 eq.) and NH₄Cl (781 mg, 14.6 mmol, 10 eq.) in EtOH (50 mL) and H₂O (20 mL) was stirred at 80° C. for 3 hrs. After completion, the mixture was filtered through a pad of Celite and the filter cake was washed with MeOH (20 mL×3). The combined organic layers were concentrated under reduced pressure, the residue was diluted with H₂O (30 mL) and extracted with EtOAc (15 mL×3). The organic layers were combined, dried over anhydrous Na₂SO₄ and concentrated under reduced pressure. The residue was purified by flash column chromatography through silica gel (eluted with DCM:MeOH=5:1) to afford to afford Int-196 (410 mg, 1.0 mmol, 68.5%) as a white solid. LCMS (ESI): m/z=412 [M+H]⁺.

Steps 8 and 9: To a stirred solution of Int-196 (41 mg, 0.1 mmol, 1.0 eq.) in concentrated HCl (5 mL) and glacial acetic acid (2 mL) was added an aqueous solution of NaNO₂ (2.0 M, 0.1 mL, 2.0 eq.) at 0° C., slowly. After stirring for 30 min, to the mixture was added CuCl₂ (27 mg, 0.2 mmol, 1.0 eq.), then, SO₂ was bubbled through the mixture at 0° C. for 15 min. After completion, the mixture was poured into ice water (10 mL) and extracted with EtOAc (10 mL×3). The combined organic phases were washed with a saturated aqueous solution of NaHCO₃ (10 mL×3), dried over anhydrous Na₂SO₄ and concentrated under reduced pressure. The crude product Int-197 was dissolved in DCM (10 mL), then to the resulting mixture was added methylamine hydrochloride (33.5 mg, 0.5 mmol, 5.0 eq.) and TEA (0.5 mL) at 0° C. After stirring at r.t. for 30 min, the mixture was diluted with H₂O (10 mL) and extracted with EtOAc (10 mL×3). The organic layers were combined and concentrated under reduced pressure. The residue was purified by Prep-HPLC (column: YMC-Actus Triart C18 250×20 mm×5 um, gradient: 10-95% MeCN in H₂O (with 0.1% FA)) to afford Compound I-293 (16.1 mg, 0.033 mmol, 33% for two steps) as a white solid.

Compound I-293: Retention time: 1.936 min; LCMS (ESI): m/z=490.1 [M+H]⁺; ¹H NMR (400 MHz, DMSO) δ 9.06 (t, J=1.7 Hz, 2H), 8.63 (t, J=2.1 Hz, 1H), 8.45 (d, J=7.9 Hz, 1H), 8.12 (d, J=7.9 Hz, 1H), 8.03 (s, 1H), 7.78 (s, 1H), 2.62 (s, 3H), 2.24 (s, 3H), 2.13 (td, J=8.2, 4.1 Hz, 1H), 0.96 (dd, J=81.0, 36.0 Hz, 4H); 1⁹F NMR (376 MHz, DMSO) δ −66.13 (s).

The following compounds were synthesized using Method ZO with the appropriate alcohols, boronic acids/boronates and amines: Compounds I-290, I-291, I-294, I-298, I-305, I-306, I-322, and I-323.

### Example 45: Method ZP: Synthesis of Compound I-299

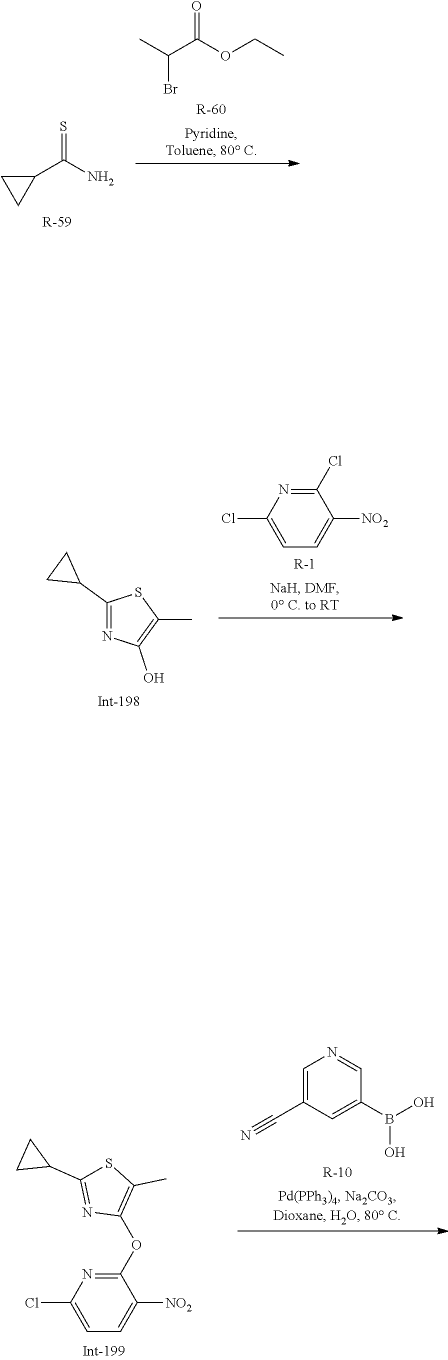

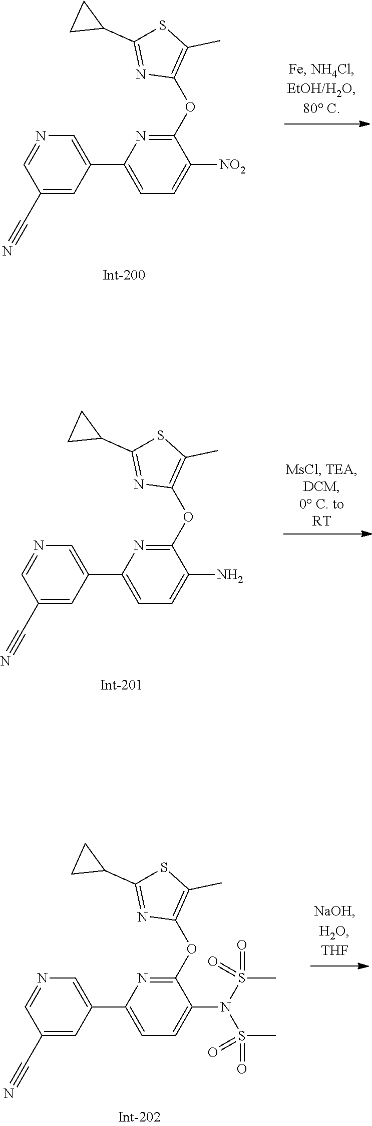 [SMILES: Cc1sc(C2CC2)nc1Oc1nc(-c2cncc(C#N)c2)ccc1[N+](=O)[O-] ; Cc1sc(C2CC2)nc1Oc1nc(-c2cncc(C#N)c2)ccc1N ; Cc1sc(C2CC2)nc1Oc1nc(-c2cncc(C#N)c2)ccc1N(S(C)(=O)=O)S(C)(=O)=O]

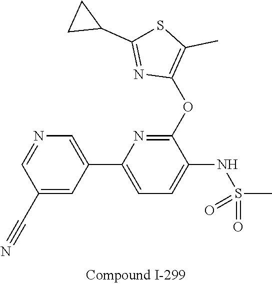 [SMILES: Cc1sc(C2CC2)nc1Oc1nc(-c2cncc(C#N)c2)ccc1NS(C)(=O)=O]

Step 1: To a stirred solution of cyclopropanecarbothioamide (R-59, 7.0 g, 69.3 mmol, 1.0 eq.) in toluene (100 mL) was added ethyl 2-bromopropanoate (R-60, 18.7 g, 104 mmol, 1.5 eq.) and pyridine (11 g, 138.6 mmol, 2.0 eq.). The resulting mixture was stirred at 80° C. for 10 hrs. After completion, the reaction mixture was diluted with H₂O (40 mL) and extracted with EtOAc (100 mL×3). The organic phase was washed with brine (100 mL×3), dried over anhydrous Na₂SO₄, and concentrated under reduced pressure to afford Int-198 (500 mg, crude) as a yellow oil, which was used to next step directly. LCMS (ESI): m/z=156 [M+H]⁺.

Step 2: To a stirred solution of Int-198 (100 mg, crude) in anhydrous DMF (20 mL) was added NaH (40 mg, 1.0 mmol) under N₂ atmosphere at 0° C., slowly. After stirring for 30 min, to the mixture was added 2, 6-dichloro-3-nitropyridine (R-1, 193 mg, 1.0 mmol). The resulting mixture was stirred at r.t. for another 2 hrs. After completion, the reaction was quenched with saturated aqueous solution of NH₄Cl (40 mL) and extracted with EtOAc (20 mL×3). The combined organic phase was washed with water 3 times, dried over anhydrous Na₂SO₄ and concentrated under reduced pressure. The residue was purified by flash column chromatography through silica gel (eluted with PE:EtOAc=2:1) to afford Int-199 (590 mg, 1.9 mmol, 13.7% for two steps) as a yellow solid. LCMS (ESI): m/z=312 [M+H]⁺.

Steps 3 through 6: Conducted using analogous procedures used in Method Y (Steps 7-10) to provide Compound I-299, which was purified by Prep-HPLC (column: YMC-Actus Triart C8 250×20 mm×5 um, gradient: 5-95% MeCN in aqueous solution of TFA (10 mM)) to afford Compound I-299 (5.3 mg, 0.012 mmol) as a white solid.

Compound I-299: Retention time: 1.496 min; LCMS (ESI): m/z=428.1 [M+H]f; 1H NMR (400 MHz, MeOD) δ 9.19 (d, J=2.1 Hz, 1H), 8.84 (d, J=1.7 Hz, 1H), 8.52 (t, J=1.9 Hz, 1H), 8.03 (d, J=8.3 Hz, 1H), 7.80 (d, J=8.1 Hz, 1H), 3.14 (s, 3H), 2.29 (td, J=8.3, 4.2 Hz, 1H), 2.25 (s, 3H), 1.15 (td, J=6.9, 4.3 Hz, 2H), 1.01-0.95 (m, 2H).

The following compound was synthesized using Method ZP with the appropriate reagents: Compound I-300.

### Example 46: Method ZQ: Synthesis of Compound I-313

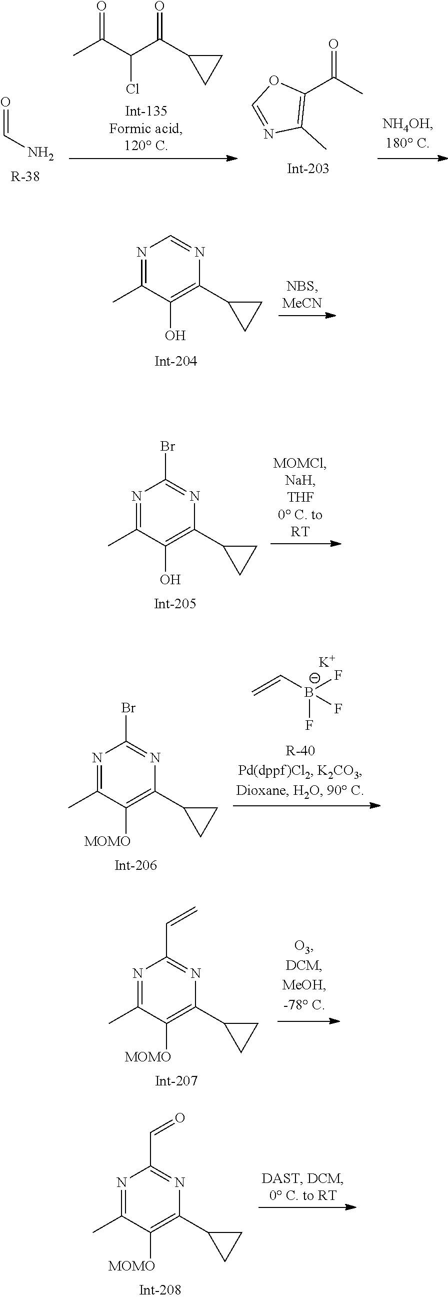

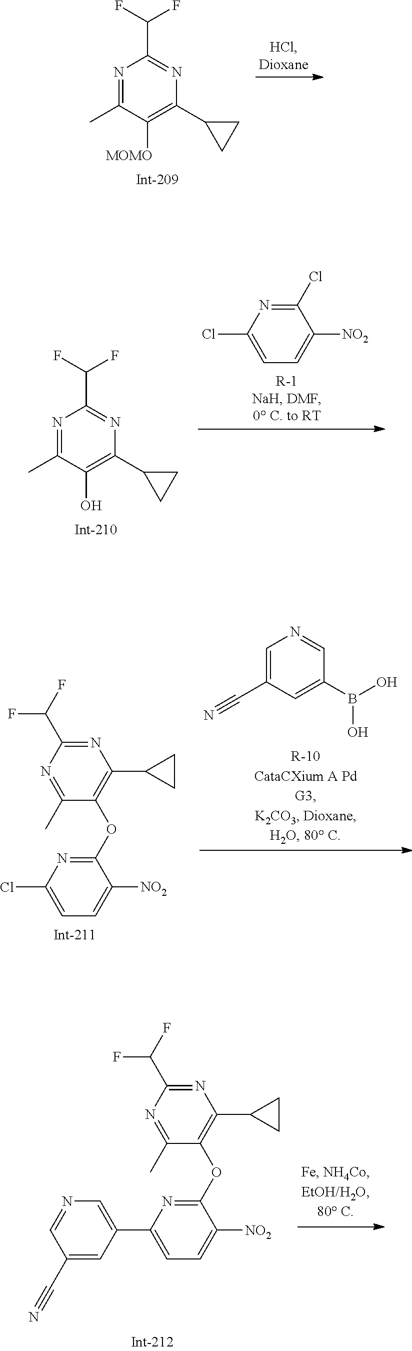

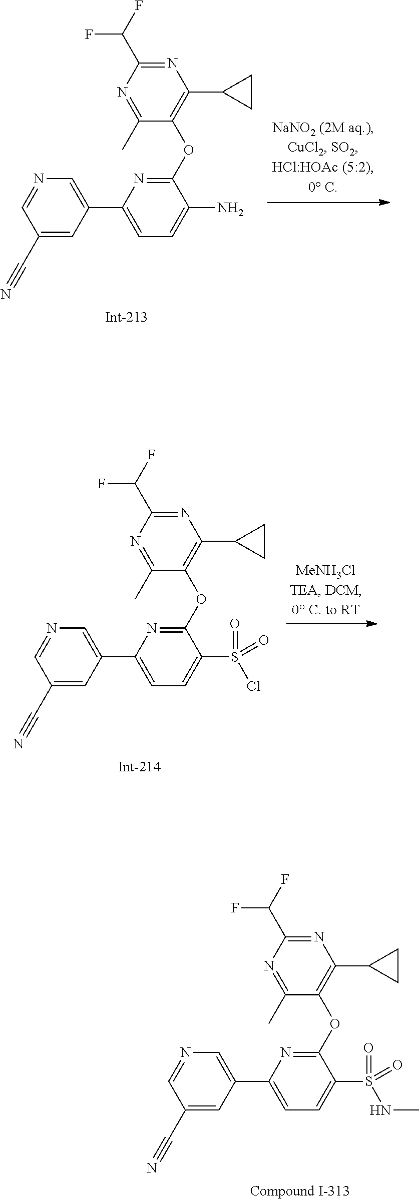 [SMILES: Cc1nc(C(F)F)nc(C2CC2)c1Oc1nc(-c2cncc(C#N)c2)ccc1N ; Cc1nc(C(F)F)nc(C2CC2)c1Oc1nc(-c2cncc(C#N)c2)ccc1S(=O)(=O)Cl ; CNS(=O)(=O)c1ccc(-c2cncc(C#N)c2)nc1Oc1c(C)nc(C(F)F)nc1C1CC1]

Step 1: A mixture of formamide (R-38, 50 g, 1.1 mol, 2.0 eq.), 2-chloro-1-cyclopropylbutane-1, 3-dione (Int-135, 88 g, 550 mmol, 1.0 eq.) in formic acid (500 mL) was stirred at 120° C. for 16 hrs. After completion, the reaction mixture was poured into ice water (500 mL), then neutralized with aqueous solution of NaOH (4 M) and extracted with EtOAc (200 mL×3). The combined organic phase was dried over anhydrous Na₂SO₄ and concentrated under reduced pressure. The residue was purified by flash column chromatography through silica gel (eluted with PE:EtOAc=0 to 5:1) to afford Int-203 (12.5 g, 82.7 mmol, 15%) as a brown oil. LCMS (ESI): m/z=152 [M+H]⁺.

Step 2: A stirred mixture of Int-203 (12.5 g, 82.7 mmol, 1.0 eq.) in NH₄OH (50 mL) was stirred at 180° C. for 8 hrs. After completion, the mixture was cooled to r.t. and extracted with EtOAc (100 mL×3). The combined organic phase was dried over anhydrous Na₂SO₄ and concentrated under reduced pressure. The residue was purified by flash column chromatography through silica gel (eluted with PE:EtOAc=1:10) to afford Int-204 (8.6 g, 57.2 mmol, 69.5%) as a yellow solid. LCMS (ESI): m/z=151 [M+H]⁺.

Step 3: To a stirred solution of Int-204 (4.6 g, 30.4 mmol, 1.0 eq.) in MeCN (20 mL) was added NBS (6.5 g, 36.5 mmol, 1.2 eq.), slowly. After stirring at r.t. for 1 h, the reaction mixture was diluted with H₂O (100 mL) and extracted with EtOAc (50 mL×3). The combined organic phase was dried over anhydrous Na₂SO₄ and concentrated under reduced pressure. The residue was purified by flash column chromatography through silica gel (eluted with PE:EtOAc=1:10) to afford Int-205 (2.3 g, 10.0 mmol, 33.2%) as a brown solid. LCMS (ESI): m/z=229 [M+H]⁺.

Step 4: To a stirred solution of Int-205 (1.1 g, 4.95 mmol, 1.0 eq.) in THF (10 mL) was added NaH (237.6 mg, 5.94 mmol, 1.2 eq.) under N₂ atmosphere at 0° C. After stirring for 30 min, to the mixture was added MOMCl (597.8 mg, 7.43 mmol, 1.5 eq.), slowly. The resulting mixture was stirred at r.t. for 1 h, then the reaction was quenched with saturated aqueous solution of NH₄Cl (50 mL) and extracted with EtOAc (20 mL×3). The combined organic phase was dried over anhydrous Na₂SO₄ and concentrated under reduced pressure. The residue was purified by flash column chromatography through silica gel (eluted with PE:EtOAc=5:1) to afford Int-206 (968 mg, 3.56 mmol, 72%) as a yellow solid. LCMS (ESI): m/z=273 [M+H]⁺.

Step 5: A mixture of Int-206 (525 mg, 1.93 mmol, 1.0 eq.), potassium vinyltrifluoroborate (R-40, 388.9 mg, 2.90 mmol, 1.5 eq.), Pd(dppf)Cl₂ (141.4 mg, 0.19 mmol, 0.1 eq.) and K₂CO₃ (801.1 mg, 5.80 mmol, 3.0 eq.) in dioxane (50 mL) and water (10 mL) was stirred at 90° C. under N₂ atmosphere overnight. After completion, the mixture was diluted with H₂O (50 mL) and extracted with EtOAc (30 mL×3). The combined organic phase was dried over anhydrous Na₂SO₄ and concentrated under reduced pressure. The residue was purified by flash column chromatography through silica gel (eluted with PE:EtOAc=4:1) to afford Int-207 (363 mg, 1.65 mmol, 85.5%) as a white solid. LCMS (ESI): m/z=221 [M+H]⁺.

Step 6: To a stirred solution of Int-207 (363 mg, 1.65 mmol, 1.0 eq.) in a mixed solvent of DCM (6 mL) and MeOH (2 mL) was bubbled O₃ for 30 mins at −78° C. Then, to the mixture was bubbled N₂ for another 1 h to remove the remaining O₃. After completion the mixture was concentrated under reduced pressure and the residue was purified by flash column chromatography through silica gel (eluted with PE:EtOAc=3:1) to afford Int-208 (250 mg, 1.12 mmol, 67.8%) as a white solid. LCMS (ESI): m/z=223 [M+H]⁺.

Step 7: To a stirred solution of Int-208 (250 mg, 1.12 mmol, 1.0 eq.) in DCM (5 mL) was added DAST (722.8 mg, 4.5 mmol, 4.0 eq.) at 0° C. After stirring at r.t. for 30 min, the reaction mixture was diluted with H₂O (40 mL) and extracted with DCM (20 mL×3). The organic layers were combined, dried over anhydrous Na₂SO₄ and concentrated under reduced pressure. The residue was purified by flash column chromatography through silica gel (eluted with PE:EtOAc=10:1 to 3:1) to afford Int-209 (168 mg, 0.69 mmol, 61.7%) as a white solid. LCMS (ESI): m/z=245 [M+H]⁺.

Step 8: To a stirred solution of Int-209 (168 mg, 0.69 mmol, 1.0 eq.) in dioxane (5 mL) was added HCl/dioxane (5 mL). After stirring at r.t. for 30 min, the mixture was concentrated under reduced pressure to give Int-210 (150 mg, crude) as a white solid. LCMS (ESI): m/z=201 [M+H]⁺.

Step 9 through 13: Conducted using analogous procedures used in Method ZO (Steps 5-9) to provide Compound I-313, which was purified by Prep-HPLC (column: YMC-Actus Triart C18 250×20 mm x Sum, gradient: 30-95% MeCN in H₂O (with 0.1% TFA)) to afford Compound I-313 (13.6 mg, 0.029 mmol, 29%) as a white solid.

Compound I-313: Retention time: 1.399 min; LCMS (ESI): m/z=473.1 [M+H]⁺; 1H NMR (400 MHz, DMSO) δ 9.08 (d, J=2.0 Hz, 2H), 8.69 (t, J=2.1 Hz, 1H), 8.47 (d, J=7.9 Hz, 1H), 8.15 (d, J=7.9 Hz, 1H), 8.05 (q, J=4.7 Hz, 1H), 6.92 (t, J=54.2 Hz, 1H), 2.64 (d, J=4.8 Hz, 3H), 2.39 (s, 3H), 2.15 (td, J=7.9, 3.9 Hz, 1H), 1.25-0.93 (m, 4H); 1⁹F NMR (376 MHz, DMSO) δ −117.62 (s).

The following compound was synthesized using Method ZQ with the appropriate reagents: Compound I-314.

### Example 47: Method ZR: Synthesis of Compound I-327

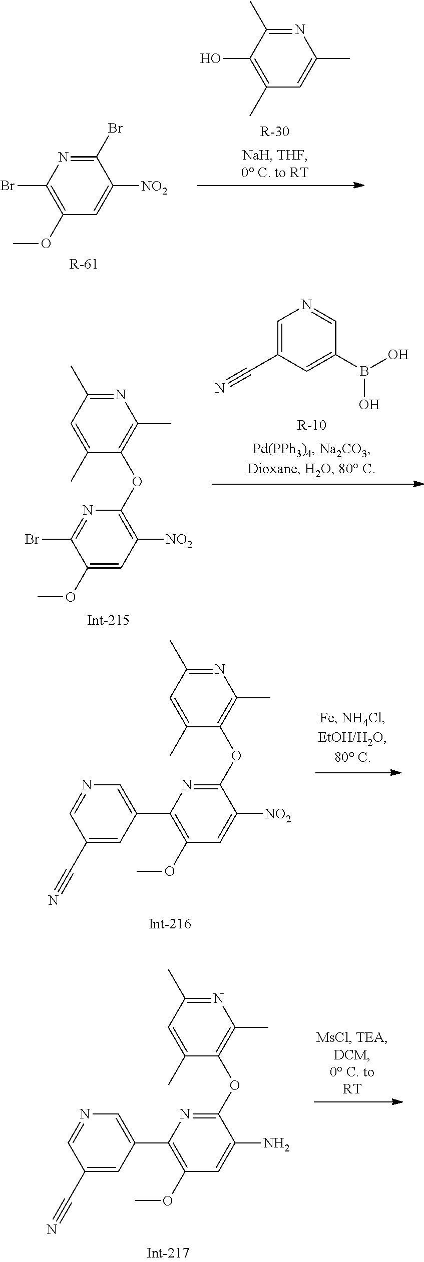

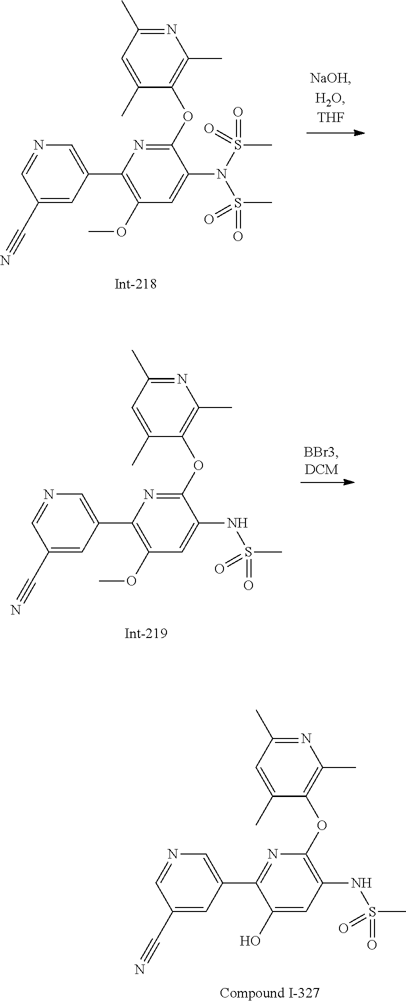 [SMILES: COc1cc(N(S(C)(=O)=O)S(C)(=O)=O)c(Oc2c(C)cc(C)nc2C)nc1-c1cncc(C#N)c1 ; COc1cc(NS(C)(=O)=O)c(Oc2c(C)cc(C)nc2C)nc1-c1cncc(C#N)c1 ; Cc1cc(C)c(Oc2nc(-c3cncc(C#N)c3)c(O)cc2NS(C)(=O)=O)c(C)n1]

Step 1: To a stirred solution of 2, 4, 6-trimethylpyridin-3-ol (R-61, 526 mg, 3.8 mmol, 1.2 eq.) in THF (30 mL) was added NaH (192 mg, 4.8 mmol, 1.3 eq.) under N₂ atmosphere at 0° C., slowly. After stirring at 0° C. for 30 min, to the mixture was added 2, 6-dibromo-3-methoxy-5-nitropyridine (R-30, 1.0 g, 3.2 mmol, 1.0 eq.). After stirring at r.t. for 2 hrs, the mixture was quenched with saturated aqueous solution of NH₄Cl (50 mL) and extracted with EtOAc (30 mL×3). The combined organic phase was dried over anhydrous Na₂SO₄ and concentrated under reduced pressure. The residue was purified by flash column chromatography on silica gel (eluted with PE:EtOAc=5:1) to afford Int-215 (844 mg, 2.3 mmol, 72% o) as a yellow solid. LCMS (ESI): m/z=368 [M+H]⁺.

Step 2: A mixture of Int-215 (844 mg, 2.3 mmol, 1.0 eq.), (5-cyanopyridin-3-yl)boronic acid (R-10, 408 mg, 2.8 mmol, 1.2 eq.), Pd(PPh₃)₄ (266 mg, 0.23 mmol, 0.1 eq.) and Na₂CO₃ (718 mg, 6.9 mmol, 3.0 eq.) in dioxane (100 mL) and water (20 mL) was stirred under N₂ atmosphere at 80° C. for 4 hrs. After completion, the reaction mixture was diluted with H₂O (50 mL) and extracted with EtOAc (30 mL×3). The organic layers were combined, dried over anhydrous Na₂SO₄ and concentrated under reduced pressure. The residue was purified by flash column chromatography on silica gel (eluted with PE:EtOAc=1: 9) to afford Int-216 (647 mg, 1.65 mmol, 72%) as a yellow solid. LCMS (ESI): m/z=392 [M+H]⁺.

Step 3: A mixture of Int-216 (647 mg, 1.65 mmol, 1.0 eq.), Fe powder (465 mg, 8.3 mmol, 5.0 eq.) and NH₄Cl (905 mg, 16.6 mmol, 10.0 eq.) in EtOH (50 mL) and H₂O (20 mL) was stirred at 80° C. for 3 hrs. After completion, the mixture was filtered through a pad of Celite and the filter cake was washed with MeOH (20 mL×3). The combined organic layers were concentrated under reduced pressure, the residue was diluted with H₂O (30 mL) and extracted with EtOAc (15 mL×3). The organic layers were combined, dried over anhydrous Na₂SO₄ and concentrated under reduced pressure to afford Int-217 (605 mg, crude product) as a brown solid, which was used for next step, directly. LCMS (ESI): m/z=362 [M+H]⁺.

Steps 4 and 5: To a stirred solution of Int-217 (605 mg, crude) in DCM (50 mL) was added MsCl (390 mg, 3.4 mmoL) and TEA (5 mL) at 0° C., slowly. After stirring at r.t. for 10 min, the reaction mixture was diluted with H₂O (50 mL) and extracted with EtOAc (50 mL×3). The combined organic layers were concentrated under reduced pressure. The residue was dissolved with THE (50 mL) and to the mixture was added an aqueous solution of NaOH (4 M, 2 mL). After stirring at r.t. for 30 min, the mixture was neutralized by HCl (1 M) and extracted with EtOAc (50 mL×3). The combined organic phase was dried over anhydrous Na₂SO₄ and concentrated under reduced pressure. The residue was purified by flash column chromatography through silica gel (eluted with DCM:MeOH=10:1) to afford Int-219 (343 mg, 0.78 mmol, 47% for three steps) as a white solid. LCMS (ESI): m/z=440 [M+H]⁺.

Step 6: To a stirred solution of Int-219 (30 mg, 0.068 mmol) in DCM (10 mL) was added BBr₃ (0.5 mL). After stirring at r.t. for 30 min, the mixture was quenched with MeOH (5 mL) and concentrated under reduced pressure to remove the solvent. The residue was purified by pre-HPLC (column: YMC-Actus Triart C18 250×20 mm×5 um, gradient: 15-95% MeCN in H₂O (with 0.1% of FA)) to afford Compound I-327 (6.5 mg, 0.015 mmol, 22%) as a white solid.

Compound I-327: Retention time: 0.48 min; LC-MS (ESI) m/z 426.2 [M+H]⁺; ¹H NMR (400 MHz, DMSO) δ 10.69 (s, 1H), 8.99 (d, J=2.1 Hz, 1H), 8.86 (d, J=2.0 Hz, 1H), 8.48 (t, J=2.1 Hz, 1H), 7.66 (s, 1H), 7.07 (s, 1H), 3.20 (s, 3H), 2.41 (s, 3H), 2.23 (s, 3H), 2.07 (s, 3H).

The following compound was synthesized using Method ZR with the appropriate reagents: Compound I-325.

### Example 48: Method ZS: Synthesis of Compound I-311

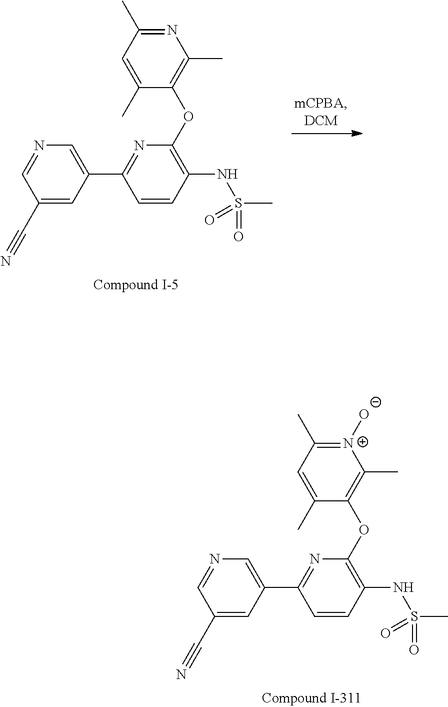 [SMILES: Cc1cc(C)c(Oc2nc(-c3cncc(C#N)c3)ccc2NS(C)(=O)=O)c(C)n1 ; Cc1cc(C)[n+]([O-])c(C)c1Oc1nc(-c2cncc(C#N)c2)ccc1NS(C)(=O)=O]

Step 1: To a stirred solution of Compound I-5 (80 mg, 0.2 mmol, 1.0 eq.) in DCM (5 mL) was added m-CPBA (42 mg, 0.24 mmol, 1.2 eq.). After stirring at r.t. for 2 hrs, the reaction mixture was diluted with H₂O (40 mL) and extracted with EtOAc (20 mL×3). The organic layers were combined and concentrated under reduced pressure. The residue was purified by Prep-HPLC (column: YMC-Actus Triart C18 250×20 mm×5 um C18 250×21 mm, gradient: 15-95% MeCN in H₂O (with 0.1% FA)) to afford Compound I-311 (31 mg, 0.073 mmol, 36%) as a white solid.

Compound I-311: Retention time: 0.48 min; LC-MS (ESI) m/z 426.2 [M+H]⁺; ¹H NMR (400 MHz, DMSO) δ 10.06 (s, 1H), 9.00 (d, J=2.1 Hz, 1H), 8.97 (d, J=1.8 Hz, 1H), 8.59 (t, J=1.9 Hz, 1H), 8.00 (d, J=8.1 Hz, 1H), 7.92 (d, J=8.1 Hz, 1H), 7.40 (s, 1H), 3.23 (s, 3H), 2.41 (s, 3H), 2.23 (s, 3H), 2.06 (s, 3H).

### Example 49: Method ZT: Synthesis of Compound I-286

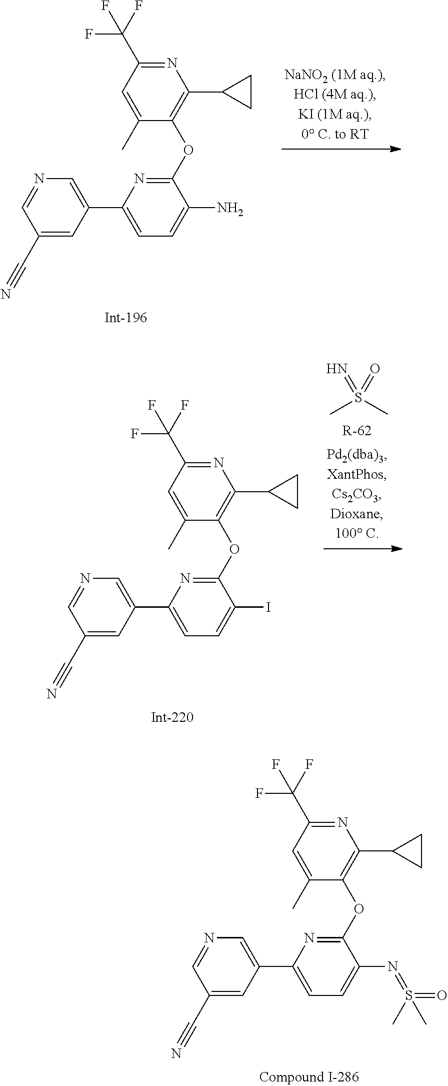

Step 1: To a stirred mixture of Int-196 (580 mg, 1.4 mmol, 1.0 eq.) in an aqueous solution of HCl (4M, 20 mL) was added an aqueous solution of NaNO₂ (1M, 3.0 mL, 3.0 mmol, 2 eq.) at 0° C., slowly. After stirring for 30 min, to the mixture was added an aqueous solution of KI (1M, 3.0 mL, 3.0 mmol, 2 eq.) at 0° C., slowly. After stirring at r.t. for 2 hrs, the mixture was neutralized with an aqueous solution of NaOH (1M) and extracted with EtOAc (30 mL×3). The combined organic phases were dried over anhydrous Na₂SO₄ and concentrated under reduced pressure. The residue was purified by flash column chromatography on silica gel (eluted with PE:EtOAc=1: 5) to afford Int-220 (510 mg, 0.98 mmol, 68%) as a yellow solid. LCMS (ESI): m/z=523 [M+H]⁺.

Step 2: A mixture of Int-220 (50 mg, 0.1 mmol, 1.0 eq.), S,S-dimethyl sulfoximine (R-62, 14 mg, 0.15 mmol, 1.5 eq.), Pd₂(dba)₃ (18.3 mg, 0.02 mmol, 0.2 eq.), XantPhos (17.4 mg, 0.03 mmol, 0.3 eq.) and Cs₂CO₃ (196 mg, 0.6 mmol, 6.0 eq.) in dioxane (20 mL) was stirred at 100° C. under N₂ atmosphere for 10 hrs. After completion, the mixture was diluted with H₂O (50 mL) and extracted with EtOAc (30 mL×3). The combined organic phases were dried over anhydrous Na₂SO₄ and concentrated under reduced pressure. The residue was purified by Prep-HPLC (column: YMC-Actus Triart C18 250×20 mm×5 um C18 250×21 mm, gradient: 5-95% MeCN in H₂O (with 0.1% FA)) to afford Compound I-286 (13 mg, 0.027 mmol, 27%) as a white solid.

Compound I-286: Retention time: 1.616 min; LC-MS (ESI) m/z 488.1 [M+H]f; ¹H NMR (400 MHz, DMSO-d₆) δ 8.98 (d, J=2.2 Hz, 1H), 8.88 (d, J=1.9 Hz, 1H), 8.43 (t, J=2.1 Hz, 1H), 7.80 (d, J=8.0 Hz, 1H), 7.71 (s, 1H), 7.65 (d, J=8.0 Hz, 1H), 3.41 (s, 6H), 2.20 (s, 3H), 2.16-2.11 (m, 1H), 0.93-0.84 (m, 4H); ¹⁹F NMR (376 MHz, DMSO) δ −66.02 (s).

### Example 50: ABCC6 WT Assay

To assess activity against ABCC6, compounds were screened in an assay measuring protein expression, using cells transfected with cDNA encoding HiBit-tagged ABCC6 wild-type.

Described here is an assay for measuring the expression of the wild type of human ABCC6 (henceforth referred to as ABCC6 WT) and the use of this assay to measure the efficacy of compounds to increase the expression of ABCC6 WT. The assay was designed to characterize the efficacy and potency of compounds to increase the expression of ABCC6 WT by calculating an Eₘₐₓ and EC₅₀ value. HEK-293 cells transiently expressing ABCC6 WT (HEK-293/ACC6 WT) were used for this assay and were cultured at 37° C. (5% CO₂, humidified) in Dulbecco's Modified Eagle Medium (DMEM, Gibco product #11965-092) supplemented with 10% fetal bovine serum. For the assay, HEK-293 cells were transfected with 0.24 mg/10 cm plate of cDNA encoding HiBit-tagged ABCC6 wild-type using Lipofectamine 2000 (Thermo Fisher, product #11668019). Twenty-four hours after transfection, the HEK-293/ACC6 WT cells were seeded in white/opaque 384-well plates (PerkinElmer, cat. No. 6007688) at 10,000 cells/well in 20 μl growth medium and allowed to settle for 4 hours at 37° C. (5% CO₂, humidified). Compound serial dilutions are prepared in growth medium as ten times (10×) the final concentration and using a dilution factor of 2. After the four-hour incubation period, 2.2 μL of the 10× dilutions are transferred to the assay plate.

Dose response curves include ten doses with the top final concentration at 20 μM in 0.15% DMSO. Liquid handling is performed with an Integra mini96-tip head (INTEGRA). After addition of compound treatment, cells are incubated for 24 hours at 37° C. (5% CO₂, humidified). After the 24-hour treatment, plates are allowed to equilibrate for 15 minutes at room temperature. Using Integra mini-96, 20 μl of HiBit lytic assay reagent (Promega, product #N3040), which is prepared according to manufacturer's instructions, is added to each well. The plates are rotated at room temperature for 10 minutes and luminescent signal is acquired using an EnVision Multimode Plate Reader (PerkinElmer).

Results are presented below in Table 4 for ABCC6 WT. Compounds having an activity designated as “A” provided an EC₅₀ less<1 μM, Eₘₐₓ≥1.2; compounds having an activity designated as “B” provided an EC₅₀≥1 μM and <10 μM, Eₘₐₓ≥1.2; compounds having an activity designated as “C” provided an EC₅₀≥10 μM and <25 μM, Eₘₐₓ≥1.2; compounds having an activity designated as “D” provided an EC₅₀≥25 μM, Eₘₐₓ≤1.2; compounds having an activity designated as “E” provided an EC₅₀≥25 μM, Eₘₐₓ≥1.2; and compounds having an activity designated as “F” provided an EC₅₀ less than 25 μM, Eₘₐₓ≤1.2.

TABLE 4
Efficacy and Potency of Compounds In
Increasing the Expression of ABCC6 WT

| | ABCC6 WT |
|---|---|
| Compound No. | EC₅₀, Eₘₐₓ |
| I-1 | A |
| I-2 | C |
| I-3 | A |
| I-4 | A |
| I-5 | A |
| I-6 | A |
| I-7 | A |
| I-8 | A |
| I-9 | A |
| I-10 | A |
| I-11 | B |
| I-12 | A |
| I-13 | A |
| I-14 | B |
| I-15 | B |
| I-16 | B |
| I-17 | B |
| I-18 | B |
| I-19 | D |
| I-20 | B |
| I-21 | B |
| I-22 | B |
| I-23 | C |
| I-24 | A |
| I-25 | B |
| I-26 | E |
| I-27 | C |
| I-28 | E |
| I-29 | A |
| I-30 | B |
| I-31 | B |
| I-32 | B |
| I-33 | B |
| I-34 | B |
| I-35 | D |
| I-36 | A |
| I-37 | A |
| I-38 | D |
| I-39 | A |
| I-40 | A |
| I-41 | A |
| I-42 | B |
| I-43 | D |
| I-44 | B |
| I-45 | A |
| I-46 | A |
| I-47 | B |
| I-48 | A |
| I-49 | A |
| I-50 | F |
| I-51 | A |
| I-52 | A |
| I-53 | B |
| I-54 | A |
| I-55 | A |
| I-56 | A |
| I-57 | A |
| I-58 | F |
| I-59 | D |
| I-60 | B |
| I-61 | B |
| I-62 | E |
| I-63 | A |
| I-64 | F |
| I-65 | D |
| I-66 | B |
| I-67 | B |
| I-68 | D |
| I-69 | A |
| I-70 | A |
| I-71 | A |
| I-72 | B |
| I-73 | B |
| I-74 | B |
| I-75 | F |
| I-76 | D |
| I-77 | A |
| I-78 | A |
| I-79 | B |
| I-80 | E |
| I-81 | B |
| I-82 | N/A |
| I-83 | B |
| I-84 | E |
| I-85 | D |
| I-86 | B |
| I-87 | B |
| I-88 | B |
| I-89 | F |
| I-90 | A |
| I-92 | B |
| I-93 | F |
| I-94 | B |
| I-95 | A |
| I-96 | B |
| I-97 | A |
| I-98 | A |
| I-99 | B |
| I-100 | A |
| I-101 | B |
| I-102 | B |
| I-103 | A |
| I-104 | B |
| I-305 | A |
| I-306 | A |
| I-307 | B |
| I-308 | B |
| I-309 | B |
| I-310 | A |
| I-311 | B |
| I-312 | F |
| I-313 | B |
| I-314 | E |
| I-315 | B |
| I-316 | B |
| I-295 | A |
| I-296 | B |
| I-297 | B |
| I-298 | B |
| I-299 | A |
| I-300 | A |
| I-301 | B |
| I-302 | C |
| I-303 | B |
| I-304 | B |
| I-286 | A |
| I-287 | E |
| I-288 | A |
| I-289 | A |
| I-290 | E |
| I-291 | B |
| I-292 | A |
| I-293 | B |
| I-294 | A |
| I-133 | A |
| I-134 | B |
| I-135 | B |
| I-136 | B |
| I-137 | A |
| I-138 | A |
| I-139 | A |
| I-140 | A |
| I-141 | A |
| I-142 | E |
| I-143 | A |
| I-144 | A |
| I-145 | A |
| I-146 | E |
| I-147 | A |
| I-148 | A |
| I-149 | F |
| I-150 | A |
| I-151 | A |
| I-152 | A |
| I-153 | B |
| I-154 | F |
| I-155 | A |
| I-156 | E |
| I-157 | E |
| I-158 | A |
| I-159 | A |
| I-160 | B |
| I-161 | A |
| I-162 | A |
| I-163 | A |
| I-164 | F |
| I-165 | A |
| I-166 | B |
| I-167 | B |
| I-168 | B |
| I-169 | B |
| I-170 | A |
| I-171 | A |
| I-172 | B |
| I-173 | A |
| I-174 | A |
| I-175 | A |
| I-176 | A |
| I-177 | A |
| I-178 | A |
| I-179 | A |
| I-180 | A |
| I-181 | A |
| I-182 | A |
| I-183 | A |
| I-184 | A |
| I-185 | A |
| I-186 | F |
| I-187 | D |
| I-188 | A |
| I-131 | A |
| I-190 | A |
| I-191 | B |
| I-192 | A |
| I-193 | A |
| I-194 | D |
| I-195 | A |
| I-196 | A |
| I-197 | A |
| I-198 | E |
| I-199 | B |
| I-200 | A |
| I-201 | B |
| I-202 | A |
| I-203 | A |
| I-204 | A |
| I-205 | A |
| I-206 | B |
| I-113 | B |
| I-208 | A |
| I-209 | A |
| I-132 | A |
| I-211 | A |
| I-212 | A |
| I-213 | A |
| I-107 | A |
| I-115 | A |
| I-216 | A |
| I-217 | A |
| I-218 | A |
| I-219 | A |
| I-220 | D |
| I-221 | B |
| I-222 | A |
| I-120 | A |
| I-127 | A |
| I-225 | A |
| I-226 | A |
| I-227 | A |
| I-228 | A |
| I-229 | B |
| I-230 | A |
| I-231 | A |
| I-232 | A |
| I-116 | D |
| I-234 | A |
| I-235 | A |
| I-236 | A |
| I-237 | A |
| I-238 | A |
| I-239 | A |
| I-240 | A |
| I-241 | C |
| I-242 | A |
| I-243 | A |
| I-244 | B |
| I-245 | A |
| I-246 | A |
| I-247 | A |
| I-248 | B |
| I-249 | E |
| I-250 | E |
| I-251 | C |
| I-252 | B |
| I-253 | B |
| I-254 | A |
| I-255 | B |
| I-256 | A |
| I-257 | B |
| I-258 | C |
| I-259 | B |
| I-260 | B |
| I-261 | B |
| I-262 | B |
| I-263 | B |
| I-264 | E |
| I-265 | B |
| I-266 | A |
| I-267 | A |
| I-268 | A |
| I-269 | B |
| I-270 | B |
| I-271 | A |
| I-272 | B |
| I-273 | A |
| I-274 | A |
| I-275 | A |
| I-276 | A |
| I-277 | A |
| I-278 | B |
| I-279 | A |
| I-280 | A |
| I-281 | A |
| I-282 | B |
| I-283 | A |
| I-284 | A |
| I-285 | B |
| I-317 | A |
| I-319 | A |
| I-320 | B |
| I-321 | B |
| I-322 | B |
| I-325 | E |
| I-326 | B |
| I-327 | A |

### Example 51: ABCB4 WT Assay

To assess activity against ABCB4, compounds were screened in an assay measuring protein expression, using cells transfected with cDNA encoding HiBit-tagged ABCB4 wild-type.

Described here is an assay for measuring the expression of the wild type of human ABCB4 (henceforth referred to as ABCB4 WT) and the use of this assay to measure the efficacy of compounds to increase the expression of ABCB4 WT. The assay was designed to characterize the efficacy and potency of compounds to increase the expression of ABCB4 WT by calculating an Eₘₐₓ and EC₅₀ value. HEK-293 cells stably expressing ABCB4 WT (HEK-293/ABCB4 WT) were used for this assay and were maintained at 37° C. (5% CO₂, humidified) in Dulbecco's Modified Eagle Medium (DMEM, Gibco product #12100) supplemented with 10% fetal bovine serum (Biosera product #FB-1058/500), 1% PenStrep (HDB product #15140-122), and 0.5 mg/mL puromycin (Gibco product #A11138-03). For the assay, the HEK-293/ABCB4 WT cells were seeded in white/opaque 384-well plates (Corning product #3570) at 10,000 cells/well in 20 μl growth medium and allowed to settle for 4 hours at 37° C. (5% CO₂, humidified). Compound serial dilutions are prepared in DMSO as three hundred times (333×) the final concentration and using a dilution factor of 3 using Bravo (Agilent). After the four-hour incubation period, 60 nL of DMSO solutions are transferred to the assay plate.

Dose response curves include eleven doses with the top final concentration at 30 μM in 0.3% DMSO. Liquid handling is performed with ECHO 550 Acoustic Liquid Handler (Labcyte). After addition of compound treatment, cells are incubated for 24 hours at 37° C. (5% CO₂, humidified). After the 24-hour treatment, plates are allowed to equilibrate to room temperature for 10 minutes. Using Dragonfly Automated Liquid Handler (TTP LabTech), 20 μl of HiBit lytic assay reagent (Promega, product #N3040), which is prepared according to manufacturer's instructions, is added to each well. The plates are rotated at room temperature for 2 minutes and incubated for 1 hour at 25° C. before luminescent signal is acquired using an EnVision Multimode Plate Reader (PerkinElmer).

Results are presented below in Table 5 for ABCB4 WT. Compounds having an activity designated as “A” provided an EC₅₀<1 μM, Eₘₐₓ≥1.2; compounds having an activity designated as “B” provided an EC₅₀≥1 and <10 μM, Eₘₐₓ≥1.2; compounds having an activity designated as “C” provided an EC₅₀≥10 and <25 μM, Eₘₐₓ≥1.2; compounds having an activity designated as “D” provided an EC₅₀≥25 μM, Eₘₐₓ≤1.2; compounds having an activity designated as “E” provided an EC₅₀≥25 μM, Eₘₐₓ≥1.2; and compounds having an activity designated as “F” provided an EC₅₀<25 μM, Eₘₐₓ<1.2.

TABLE 5
Efficacy and Potency of Compounds In
Increasing the Expression of ABCB4 WT

| Compound No. | ABCB4 WT EC₅₀, Eₘₐₓ |
|---|---|
| I-1 | E |
| I-2 | E |
| I-3 | A |
| I-4 | A |
| I-5 | A |
| I-6 | D |
| I-7 | A |
| I-8 | A |
| I-9 | D |
| I-10 | D |
| I-11 | D |
| I-12 | F |
| I-13 | B |
| I-14 | E |
| I-15 | B |
| I-16 | E |
| I-17 | E |
| I-18 | E |
| I-19 | E |
| I-20 | E |
| I-21 | F |
| I-22 | B |
| I-23 | C |
| I-24 | A |
| I-25 | B |
| I-26 | B |
| I-27 | C |
| I-28 | B |
| I-29 | D |
| I-30 | B |
| I-31 | E |
| I-32 | D |
| I-33 | C |
| I-34 | B |
| I-35 | D |
| I-36 | D |
| I-37 | A |
| I-38 | E |
| I-39 | A |
| I-40 | A |
| I-41 | D |
| I-42 | D |
| I-43 | D |
| I-44 | E |
| I-45 | D |
| I-46 | F |
| I-47 | A |
| I-48 | A |
| I-49 | A |
| I-50 | B |
| I-51 | A |
| I-52 | B |
| I-53 | E |
| I-54 | D |
| I-55 | D |
| I-56 | D |
| I-57 | F |
| I-58 | D |
| I-59 | D |
| I-60 | D |
| I-61 | D |
| I-62 | D |
| I-63 | D |
| I-64 | F |
| I-65 | D |
| I-66 | D |
| I-67 | B |
| I-68 | D |
| I-69 | F |
| I-70 | E |
| I-71 | D |
| I-72 | E |
| I-73 | A |
| I-74 | B |
| I-75 | B |
| I-76 | B |
| I-77 | B |
| I-78 | F |
| I-79 | D |
| I-80 | D |
| I-81 | F |
| I-82 | N/A |
| I-83 | D |
| I-84 | D |
| I-85 | F |
| I-86 | B |
| I-87 | B |
| I-88 | F |
| I-89 | B |
| I-90 | B |
| I-92 | E |
| I-93 | D |
| I-94 | B |
| I-95 | A |
| I-96 | B |
| I-97 | B |
| I-98 | F |
| I-99 | D |
| I-100 | D |
| I-101 | D |
| I-102 | D |
| I-103 | A |
| I-104 | B |
| I-305 | D |
| I-306 | D |
| I-307 | D |
| I-308 | F |
| I-309 | A |
| I-310 | D |
| I-311 | B |
| I-312 | D |
| I-313 | B |
| I-314 | B |
| I-315 | E |
| I-316 | B |
| I-295 | D |
| I-296 | A |
| I-297 | B |
| I-298 | E |
| I-299 | A |
| I-300 | A |
| I-301 | D |
| I-302 | C |
| I-303 | D |
| I-304 | B |
| I-286 | D |
| I-287 | F |
| I-288 | B |
| I-289 | C |
| I-290 | E |
| I-291 | A |
| I-292 | B |
| I-293 | A |
| I-294 | C |
| I-133 | B |
| I-134 | A |
| I-135 | A |
| I-136 | A |
| I-137 | A |
| I-138 | A |
| I-139 | A |
| I-140 | A |
| I-141 | A |
| I-142 | D |
| I-143 | A |
| I-144 | A |
| I-145 | A |
| I-146 | D |
| I-147 | F |
| I-148 | A |
| I-149 | D |
| I-150 | A |
| I-151 | A |
| I-152 | A |
| I-153 | D |
| I-154 | F |
| I-155 | A |
| I-156 | E |
| I-157 | F |
| I-158 | A |
| I-159 | A |
| I-160 | F |
| I-161 | A |
| I-162 | E |
| I-163 | A |
| I-164 | B |
| I-165 | B |
| I-166 | B |
| I-167 | B |
| I-168 | B |
| I-169 | B |
| I-170 | A |
| I-171 | A |
| I-172 | A |
| I-173 | A |
| I-174 | E |
| I-175 | A |
| I-176 | A |
| I-177 | A |
| I-178 | A |
| I-179 | A |
| I-180 | A |
| I-181 | A |
| I-182 | A |
| I-183 | A |
| I-184 | A |
| I-185 | A |
| I-186 | E |
| I-187 | D |
| I-188 | E |
| I-131 | A |
| I-190 | A |
| I-191 | A |
| I-192 | F |
| I-193 | A |
| I-194 | D |
| I-195 | A |
| I-196 | E |
| I-197 | A |
| I-198 | A |
| I-199 | A |
| I-200 | A |
| I-201 | D |
| I-202 | F |
| I-203 | F |
| I-204 | A |
| I-205 | D |
| I-206 | F |
| I-113 | F |
| I-208 | A |
| I-209 | A |
| I-132 | A |
| I-211 | A |
| I-212 | A |
| I-213 | A |
| I-107 | D |
| I-115 | A |
| I-216 | A |
| I-217 | A |
| I-218 | A |
| I-219 | A |
| I-220 | D |
| I-221 | E |
| I-222 | A |
| I-120 | F |
| I-127 | A |
| I-225 | F |
| I-226 | A |
| I-227 | F |
| I-228 | F |
| I-229 | F |
| I-230 | A |
| I-231 | B |
| I-232 | F |
| I-116 | D |
| I-234 | B |
| I-235 | F |
| I-236 | F |
| I-237 | F |
| I-238 | F |
| I-239 | F |
| I-240 | A |
| I-241 | C |
| I-242 | B |
| I-243 | A |
| I-244 | E |
| I-245 | B |
| I-246 | F |
| I-247 | A |
| I-248 | B |
| I-249 | F |
| I-250 | E |
| I-251 | D |
| I-252 | E |
| I-253 | B |
| I-254 | F |
| I-255 | D |
| I-256 | A |
| I-257 | D |
| I-258 | F |
| I-259 | D |
| I-260 | D |
| I-261 | B |
| I-262 | B |
| I-263 | B |
| I-264 | A |
| I-265 | A |
| I-266 | A |
| I-267 | A |
| I-268 | A |
| I-269 | A |
| I-270 | F |
| I-271 | B |
| I-272 | F |
| I-273 | F |
| I-274 | F |
| I-275 | D |
| I-276 | A |
| I-277 | D |
| I-278 | D |
| I-279 | A |
| I-280 | A |
| I-281 | A |
| I-282 | D |
| I-283 | A |
| I-284 | A |
| I-285 | E |
| I-317 | A |
| I-319 | B |
| I-320 | F |
| I-321 | B |
| I-322 | F |
| I-325 | F |
| I-326 | F |
| I-327 | A |

### Example 52: Biological Activity Assay

Exemplary compounds were evaluated in an A4 correction dose-response assay. Experimental procedures and results are provided below.

### Part I—Experimental Procedure

To assess activity against ABCA4 P1380L, the compounds were screened in an assay measuring protein expression, using cells transfected with cDNA encoding HiBit-tagged ABCA4 P1380L. Here we describe an assay for measuring the expression of the P1380L variant of human ABCA4 (henceforth referred to as ABCA4 P1380L) and the use of this assay to measure the efficacy of compounds to increase the expression of ABCA4 P1380L. The assay was designed to characterize the efficacy and potency of compounds to increase the expression of ABCA4 P1380L by calculating an Eₘₐₓ and EC₅₀ value. HEK-293 cells stably expressing ABCA4 P1380L (HEK-293/ABCA4 P1380L) were used for this assay and were maintained at 37° C. (5% CO₂, humidified) in Dulbecco's Modified Eagle Medium (DMEM, Gibco product #12100) supplemented with 10% fetal bovine serum (Biosera product #FB-1058/500), 1% PenStrep (HDB product #15140-122), and 0.5 mg/mL puromycin (Gibco product #A11138-03). For the assay, the HEK-293/ABCA4 P1380L cells were seeded in white/opaque 384-well plates (Corning product #3570) at 10,000 cells/well in 20 μl growth medium and allowed to settle for 4 hours at 37° C. (5% CO₂, humidified). Compound serial dilutions are prepared in DMSO as three hundred times (333×) the final concentration and using a dilution factor of 3 using Bravo (Agilent). After the four-hour incubation period, 60 nL of DMSO solutions are transferred to the assay plate.

Dose response curves include eleven doses with the top final concentration at 30 μM in 0.3% DMSO. Liquid handling is performed with ECHO 550 Acoustic Liquid Handler (Labcyte). After addition of compound treatment, cells are incubated for 24 hours at 37° C. (5% CO₂, humidified). After the 24-hour treatment, plates are allowed to equilibrate to room temperature for 10 minutes. Using Dragonfly Automated Liquid Handler (TTP LabTech), 20 μl of HiBit lytic assay reagent (Promega, product #N3040), which is prepared according to manufacturer's instructions, is added to each well. The plates are rotated at room temperature for 2 minutes and incubated for 1 hour at 25° C. before luminescent signal is acquired using an EnVision Multimode Plate Reader (PerkinElmer).

### Part II—Results

Results are shown in the table below. Compounds having an activity designated as “A” provided an EC_{50≤1} μM, Mean MaxAct (fold)≥1.2; compounds having an activity designated as “B” provided an EC₅₀≥1 and <10 μM, Mean MaxAct (fold)≥1.2; compounds having an activity designated as “C” provided an EC₅₀≥10 and <25 μM, Mean MaxAct (fold)≥1.2; compounds having an activity designated as “D” provided an EC₅₀≥25 μM, Mean MaxAct (fold)≤1.2; compounds having an activity designated as “E” provided an EC₅₀≥25 μM, Mean MaxAct (fold)≥1.2; and compounds having an activity designated as “F” provided an EC₅₀<25 μM, Mean MaxAct (fold)<1.2.

| Compound No. | ABCA4 P1380L EC₅₀, Eₘₐₓ |
|---|---|
| I-1 | A |
| I-2 | E |
| I-3 | A |
| I-4 | A |
| I-5 | A |
| I-6 | C |
| I-7 | A |
| I-8 | A |
| I-9 | E |
| I-10 | A |
| I-11 | E |
| I-12 | E |
| I-13 | B |
| I-14 | E |
| I-15 | B |
| I-16 | E |
| I-17 | E |
| I-18 | C |
| I-19 | D |
| I-20 | E |
| I-21 | E |
| I-22 | B |
| I-23 | C |
| I-24 | B |
| I-25 | E |
| I-26 | E |
| I-27 | B |
| I-28 | B |
| I-29 | D |
| I-30 | A |
| I-31 | E |
| I-32 | B |
| I-33 | E |
| I-34 | C |
| I-35 | D |
| I-36 | A |
| I-37 | A |
| I-38 | E |
| I-39 | A |
| I-40 | A |
| I-41 | A |
| I-42 | B |
| I-43 | E |
| I-44 | C |
| I-45 | A |
| I-46 | A |
| I-47 | E |
| I-48 | D |
| I-49 | D |
| I-50 | E |
| I-51 | A |
| I-52 | B |
| I-53 | B |
| I-54 | F |
| I-55 | D |
| I-56 | D |
| I-57 | B |
| I-58 | D |
| I-59 | B |
| I-60 | F |
| I-61 | D |
| I-62 | E |
| I-63 | C |
| I-64 | B |
| I-65 | D |
| I-66 | E |
| I-67 | E |
| I-68 | D |
| I-69 | B |
| I-70 | D |
| I-71 | D |
| I-72 | D |
| I-73 | A |
| I-74 | B |
| I-75 | B |
| I-76 | D |
| I-77 | F |
| I-78 | B |
| I-79 | F |
| I-80 | D |
| I-81 | B |
| I-82 | N/A |
| I-83 | B |
| I-84 | F |
| I-85 | D |
| I-86 | F |
| I-87 | E |
| I-88 | B |
| I-89 | E |
| I-90 | B |
| I-92 | E |
| I-93 | B |
| I-94 | B |
| I-95 | A |
| I-96 | E |
| I-97 | D |
| I-98 | D |
| I-99 | E |
| I-100 | C |
| I-101 | B |
| I-102 | A |
| I-103 | B |
| I-104 | B |
| I-305 | D |
| I-306 | F |
| I-307 | D |
| I-308 | D |
| I-309 | D |
| I-310 | D |
| I-311 | B |
| I-312 | D |
| I-313 | C |
| I-314 | E |
| I-315 | E |
| I-316 | B |
| I-295 | F |
| I-296 | A |
| I-297 | B |
| I-298 | E |
| I-299 | A |
| I-300 | F |
| I-301 | F |
| I-302 | D |
| I-303 | E |
| I-304 | B |
| I-286 | D |
| I-287 | D |
| I-288 | B |
| I-289 | E |
| I-290 | D |
| I-291 | F |
| I-292 | B |
| I-293 | F |
| I-294 | F |
| I-133 | A |
| I-134 | D |
| I-135 | D |
| I-136 | B |
| I-137 | A |
| I-138 | A |
| I-139 | D |
| I-140 | A |
| I-141 | A |
| I-142 | D |
| I-143 | A |
| I-144 | A |
| I-145 | A |
| I-146 | B |
| I-147 | A |
| I-148 | A |
| I-149 | D |
| I-150 | A |
| I-151 | A |
| I-152 | A |
| I-153 | D |
| I-154 | F |
| I-155 | A |
| I-156 | F |
| I-157 | D |
| I-158 | A |
| I-159 | F |
| I-160 | E |
| I-161 | A |
| I-162 | A |
| I-163 | F |
| I-164 | B |
| I-165 | B |
| I-166 | B |
| I-167 | B |
| I-168 | B |
| I-169 | B |
| I-170 | A |
| I-171 | A |
| I-172 | A |
| I-173 | A |
| I-174 | B |
| I-175 | A |
| I-176 | A |
| I-177 | A |
| I-178 | E |
| I-179 | E |
| I-180 | E |
| I-181 | A |
| I-182 | A |
| I-183 | A |
| I-184 | A |
| I-185 | A |
| I-186 | D |
| I-187 | D |
| I-188 | B |
| I-131 | A |
| I-190 | A |
| I-191 | A |
| I-192 | B |
| I-193 | A |
| I-194 | C |
| I-195 | E |
| I-196 | E |
| I-197 | B |
| I-198 | E |
| I-199 | A |
| I-200 | A |
| I-201 | B |
| I-202 | A |
| I-203 | A |
| I-204 | A |
| I-205 | A |
| I-206 | F |
| I-113 | F |
| I-208 | A |
| I-209 | A |
| I-132 | A |
| I-211 | C |
| I-212 | A |
| I-213 | A |
| I-107 | D |
| I-115 | F |
| I-216 | E |
| I-217 | A |
| I-218 | A |
| I-219 | A |
| I-220 | E |
| I-221 | B |
| I-222 | A |
| I-120 | A |
| I-127 | B |
| I-225 | D |
| I-226 | E |
| I-227 | B |
| I-228 | A |
| I-229 | B |
| I-230 | A |
| I-231 | B |
| I-232 | F |
| I-116 | D |
| I-234 | A |
| I-235 | F |
| I-236 | F |
| I-237 | A |
| I-238 | B |
| I-239 | A |
| I-240 | F |
| I-241 | E |
| I-242 | B |
| I-243 | A |
| I-244 | C |
| I-245 | B |
| I-246 | A |
| I-247 | A |
| I-248 | B |
| I-249 | D |
| I-250 | D |
| I-251 | D |
| I-252 | A |
| I-253 | F |
| I-254 | A |
| I-255 | E |
| I-256 | E |
| I-257 | F |
| I-258 | B |
| I-259 | F |
| I-260 | D |
| I-261 | C |
| I-262 | B |
| I-263 | B |
| I-264 | A |
| I-265 | B |
| I-266 | A |
| I-267 | F |
| I-268 | A |
| I-269 | B |
| I-270 | F |
| I-271 | A |
| I-272 | B |
| I-273 | F |
| I-274 | A |
| I-275 | F |
| I-276 | A |
| I-277 | F |
| I-278 | B |
| I-279 | A |
| I-280 | A |
| I-281 | A |
| I-282 | E |
| I-283 | A |
| I-284 | F |
| I-285 | E |
| I-317 | E |
| I-319 | E |
| I-320 | F |
| I-321 | F |
| I-322 | B |
| I-325 | B |
| I-326 | A |
| I-327 | A |

### Example 53: Biological Activity Assay

Exemplary compounds were evaluated in a D2 correction dose-response assay. Experimental procedures and results are provided below.

### Part I—Experimental Procedure

To assess activity against ABCD2, the compounds were screened in an assay measuring protein expression, using cells transfected with cDNA encoding HiBit-tagged ABCD2 wild-type. Here we describe an assay for measuring the expression of the wild type of human ABCD2 (henceforth referred to as ABCD2 WT) and the use of this assay to measure the efficacy of compounds to increase the expression of ABCD2 WT. The assay was designed to characterize the efficacy and potency of compounds to increase the expression of ABCD2 WT by calculating an Eₘₐₓ and EC₅₀ value. HEK-293 cells stably expressing ABCD2 WT (HEK-293/ABCD2 WT) were used for this assay and were maintained at 37° C. (5% CO₂, humidified) in Dulbecco's Modified Eagle Medium (DMEM, Gibco product #12100) supplemented with 10% fetal bovine serum (Biosera product #FB-1058/500), 1% PenStrep (HDB product #15140-122), and 0.5 mg/mL puromycin (Gibco product #A11138-03). For the assay, the HEK-293/ABCD2 WT cells were seeded in white/opaque 384-well plates (Corning product #3570) at 10,000 cells/well in 20 μl growth medium and allowed to settle for 4 hours at 37° C. (5% CO₂, humidified). Compound serial dilutions are prepared in DMSO as three hundred times (333×) the final concentration and using a dilution factor of 3 using Bravo (Agilent). After the four-hour incubation period, 60 nL of DMSO solutions are transferred to the assay plate.

Dose response curves include eleven doses with the top final concentration at 30 μM in 0.3% DMSO. Liquid handling is performed with ECHO 550 Acoustic Liquid Handler (Labcyte). After addition of compound treatment, cells are incubated for 24 hours at 37° C. (5% CO₂, humidified). After the 24-hour treatment, plates are allowed to equilibrate to room temperature for 10 minutes. Using Dragonfly Automated Liquid Handler (TTP LabTech), 20 μl of HiBit lytic assay reagent (Promega, product #N3040), which is prepared according to manufacturer's instructions, is added to each well. The plates are rotated at room temperature for 2 minutes and incubated for 1 hour at 25° C. before luminescent signal is acquired using an EnVision Multimode Plate Reader (PerkinElmer).

### Part II—Results

Results are shown in the table below. Compounds having an activity designated as “A” provided an EC_{50<1} μM, Mean MaxAct (fold)≥1.2; compounds having an activity designated as “B” provided an EC₅₀≥1 and <10 μM, Mean MaxAct (fold)≥1.2; compounds having an activity designated as “C” provided an EC₅₀≥10 and <25 μM, Mean MaxAct (fold)≥1.2; compounds having an activity designated as “D” provided an EC₅₀≥25 μM, Mean MaxAct (fold)<1.2; compounds having an activity designated as “E” provided an EC₅₀≥25 μM, Mean MaxAct (fold)≥1.2; and compounds having an activity designated as “F” provided an EC₅₀<25 μM, Mean MaxAct (fold)<1.2.

| Compound No. | ABCD2 WT EC₅₀, Eₘₐₓ |
|---|---|
| I-1 | B |
| I-2 | C |
| I-3 | B |
| I-4 | A |
| I-5 | A |
| I-6 | A |
| I-7 | A |
| I-8 | A |
| I-9 | A |
| I-10 | A |
| I-11 | B |
| I-12 | A |
| I-13 | E |
| I-14 | B |
| I-15 | E |
| I-16 | C |
| I-17 | B |
| I-18 | E |
| I-19 | E |
| I-20 | C |
| I-21 | E |
| I-22 | B |
| I-23 | C |
| I-24 | B |
| I-25 | B |
| I-26 | B |
| I-27 | E |
| I-28 | E |
| I-29 | D |
| I-30 | B |
| I-31 | E |
| I-32 | B |
| I-33 | C |
| I-34 | E |
| I-35 | E |
| I-36 | A |
| I-37 | A |
| I-38 | E |
| I-39 | A |
| I-40 | A |
| I-41 | A |
| I-42 | B |
| I-43 | E |
| I-44 | C |
| I-45 | A |
| I-46 | A |
| I-47 | B |
| I-48 | A |
| I-49 | D |
| I-50 | E |
| I-51 | D |
| I-52 | A |
| I-53 | D |
| I-54 | B |
| I-55 | A |
| I-56 | B |
| I-57 | A |
| I-58 | D |
| I-59 | B |
| I-60 | B |
| I-61 | B |
| I-62 | A |
| I-63 | E |
| I-64 | B |
| I-65 | D |
| I-66 | C |
| I-67 | B |
| I-68 | D |
| I-69 | D |
| I-70 | A |
| I-71 | F |
| I-72 | A |
| I-73 | B |
| I-74 | F |
| I-75 | F |
| I-76 | B |
| I-77 | A |
| I-78 | B |
| I-79 | B |
| I-80 | B |
| I-81 | B |
| I-82 | N/A |
| I-83 | B |
| I-84 | C |
| I-85 | B |
| I-86 | B |
| I-87 | E |
| I-88 | B |
| I-89 | B |
| I-90 | B |
| I-92 | E |
| I-93 | B |
| I-94 | B |
| I-95 | A |
| I-96 | B |
| I-97 | B |
| I-98 | A |
| I-99 | E |
| I-100 | A |
| I-101 | B |
| I-102 | E |
| I-103 | B |
| I-104 | E |
| I-305 | B |
| I-306 | B |
| I-307 | B |
| I-308 | E |
| I-309 | B |
| I-310 | B |
| I-311 | B |
| I-312 | E |
| I-313 | E |
| I-314 | E |
| I-315 | C |
| I-316 | C |
| I-295 | C |
| I-296 | A |
| I-297 | B |
| I-298 | B |
| I-299 | A |
| I-300 | A |
| I-301 | C |
| I-302 | C |
| I-303 | E |
| I-304 | A |
| I-286 | A |
| I-287 | E |
| I-288 | B |
| I-289 | B |
| I-290 | E |
| I-291 | B |
| I-292 | B |
| I-293 | B |
| I-294 | A |
| I-133 | A |
| I-134 | B |
| I-135 | B |
| I-136 | B |
| I-137 | A |
| I-138 | A |
| I-139 | B |
| I-140 | A |
| I-141 | A |
| I-142 | B |
| I-143 | A |
| I-144 | E |
| I-145 | A |
| I-146 | C |
| I-147 | A |
| I-148 | A |
| I-149 | E |
| I-150 | A |
| I-151 | A |
| I-152 | A |
| I-153 | B |
| I-154 | E |
| I-155 | E |
| I-156 | A |
| I-157 | C |
| I-158 | A |
| I-159 | A |
| I-160 | E |
| I-161 | E |
| I-162 | E |
| I-163 | A |
| I-164 | B |
| I-165 | B |
| I-166 | B |
| I-167 | B |
| I-168 | B |
| I-169 | C |
| I-170 | B |
| I-171 | A |
| I-172 | E |
| I-173 | A |
| I-174 | B |
| I-175 | E |
| I-176 | A |
| I-177 | E |
| I-178 | B |
| I-179 | A |
| I-180 | E |
| I-181 | E |
| I-182 | A |
| I-183 | A |
| I-184 | A |
| I-185 | A |
| I-186 | D |
| I-187 | D |
| I-188 | B |
| I-131 | A |
| I-190 | A |
| I-191 | C |
| I-192 | E |
| I-193 | A |
| I-194 | D |
| I-195 | E |
| I-196 | A |
| I-197 | B |
| I-198 | E |
| I-199 | B |
| I-200 | A |
| I-201 | B |
| I-202 | E |
| I-203 | A |
| I-204 | A |
| I-205 | A |
| I-206 | E |
| I-113 | F |
| I-208 | E |
| I-209 | A |
| I-132 | A |
| I-211 | A |
| I-212 | A |
| I-213 | E |
| I-107 | A |
| I-115 | A |
| I-216 | A |
| I-217 | A |
| I-218 | C |
| I-219 | A |
| I-220 | D |
| I-221 | E |
| I-222 | A |
| I-120 | B |
| I-127 | E |
| I-225 | A |
| I-226 | A |
| I-227 | B |
| I-228 | B |
| I-229 | B |
| I-230 | A |
| I-231 | A |
| I-232 | A |
| I-116 | D |
| I-234 | A |
| I-235 | A |
| I-236 | A |
| I-237 | A |
| I-238 | B |
| I-239 | A |
| I-240 | A |
| I-241 | C |
| I-242 | E |
| I-243 | C |
| I-244 | C |
| I-245 | A |
| I-246 | A |
| I-247 | E |
| I-248 | E |
| I-249 | B |
| I-250 | E |
| I-251 | E |
| I-252 | B |
| I-253 | B |
| I-254 | A |
| I-255 | B |
| I-256 | B |
| I-257 | B |
| I-258 | C |
| I-259 | B |
| I-260 | B |
| I-261 | B |
| I-262 | B |
| I-263 | B |
| I-264 | E |
| I-265 | B |
| I-266 | E |
| I-267 | A |
| I-268 | E |
| I-269 | B |
| I-270 | B |
| I-271 | E |
| I-272 | E |
| I-273 | A |
| I-274 | B |
| I-275 | A |
| I-276 | E |
| I-277 | A |
| I-278 | B |
| I-279 | C |
| I-280 | A |
| I-281 | A |
| I-282 | B |
| I-283 | B |
| I-284 | A |
| I-285 | C |
| I-317 | B |
| I-319 | B |
| I-320 | E |
| I-321 | B |
| I-322 | E |
| I-325 | C |
| I-326 | E |
| I-327 | A |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### REFERENCES

1. Vauthier, V. et al. (2017) Targeted pharmacotherapies for defective ABC transporters. Biochem Pharmacol 136, 1-11.10.1016/j.bcp.2017.02.020
2. Dean, M. et al. (2022) The human ATP-binding cassette (ABC) transporter superfamily. Hum Mutat 43, 1162-1182.10.1002/humu.24418
3. Liu, X. (2019) ABC Family Transporters. Adv Exp Med Biol 1141, 13-100. 10.1007/978-981-13-7647-4_2
4. Molday, R. S. et al. (2022) Structure and function of ABCA4 and its role in the visual cycle and Stargardt macular degeneration. Prog Retin Eye Res 89, 101036. 10.1016/j.preteyeres.2021.101036
5. Kroll, T. et al. (2021) Structure and Function of Hepatobiliary ATP Binding Cassette Transporters. Chem Rev 121, 5240-5288. 10.1021/acs.chemrev.0c00659
6. Wanders, R. J. et al. (2007) The peroxisomal ABC transporter family. Pflugers Arch 453, 719-734. 10.1007/s00424-006-0142-x
7. Shaughnessy, C. A. et al. (2021) Elexacaftor is a CFTR potentiator and acts synergistically with ivacaftor during acute and chronic treatment. Sci Rep 11, 19810. 10.1038/s41598-021-99184-1
8. Thomas, C. and Tampe, R. (2020) Structural and Mechanistic Principles of ABC Transporters. Annu Rev Biochem 89, 605-636. 10.1146/annurev-biochem-011520-105201
9. Byrne, J. A. et al. (2009) Missense mutations and single nucleotide polymorphisms in ABCB11 impair bile salt export pump processing and function or disrupt pre-messenger RNA splicing. Hepatology 49, 553-567. 10.1002/hep.22683
10. Gunaydin, M. and Bozkurter Cil, A. T. (2018) Progressive familial intrahepatic cholestasis: diagnosis, management, and treatment. Hepat Med 10, 95-104. 10.2147/HMER.S137209
11. Bosma, P. J. et al. (2020) Gene Therapy for Progressive Familial Intrahepatic Cholestasis: Current Progress and Future Prospects. Int J Mol Sci 22. 10.3390/ijms22010273
12. Boyer, J. L. (2013) Bile formation and secretion. Compr Physiol 3, 1035-1078. 10.1002/cphy.c120027
13. Gerloff, T. et al. (1998) The sister of P-glycoprotein represents the canalicular bile salt export pump of mammalian liver. J Biol Chem 273, 10046-10050. 10.1074/jbc.273.16.10046
14. van der Woerd, W. L. et al. (2017) Current and future therapies for inherited cholestatic liver diseases. World J Gastroenterol 23, 763-775. 10.3748/wjg.v23.i5.763
15. Wang, L. et al. (2020) Cryo-EM structure of human bile salts exporter ABCB11. Cell Res 30, 623-625. 10.1038/s41422-020-0302-0
16. Boyer, J. L. and Soroka, C. J. (2021) Bile formation and secretion: An update. J Hepatol 75, 190-201. 10.1016/j.jhep.2021.02.011
17. Al-Dury, S. and Marschall, H. U. (2018) Ileal Bile Acid Transporter Inhibition for the Treatment of Chronic Constipation, Cholestatic Pruritus, and NASH. Front Pharmacol 9, 931. 10.3389/fphar.2018.00931
18. van Wessel, D. B. E. et al. (2020) Genotype correlates with the natural history of severe bile salt export pump deficiency. J Hepatol 73, 84-93. 10.1016/j.jhep.2020.02.007
19. Stattermayer, A. F. et al. (2020) Variants in ABCB4 (MDR3) across the spectrum of cholestatic liver diseases in adults. J Hepatol 73, 651-663. 10.1016/j.jhep.2020.04.036
20. Nayagam, J. S. et al. (2022) Clinical phenotype of adult-onset liver disease in patients with variants in ABCB4, ABCB11, and ATP8B1. Hepatol Commun. 10.1002/hep4.2051
21. Stapelbroek, J. M. et al. (2010) Liver disease associated with canalicular transport defects: current and future therapies. J Hepatol 52, 258-271. 10.1016/j.jhep.2009.11.012
22. Thompson, R. J. et al. (2022) Odevixibat treatment in progressive familial intrahepatic cholestasis: a randomised, placebo-controlled, phase 3 trial. Lancet Gastroenterol Hepatol 7, 830-842. 10.1016/S2468-1253 (22)00093-0
23. Le Saux, O. et al. (2000) Mutations in a gene encoding an ABC transporter cause pseudoxanthoma elasticum. Nat Genet 25, 223-227. 10.1038/76102
24. Iwanaga, A. and Murota, H. (2022) Author reply to comment on “Clinical practice guidelines for pseudoxanthoma elasticum (2017)”: The importance of mutation analysis. J Dermatol 49, e250. 10.1111/1346-8138.16379
25. Borst, P. et al. (2019) PXE, a Mysterious Inborn Error Clarified. Trends Biochem Sci 44, 125-140. 10.1016/j.tibs.2018.10.005
26. Nitschke, Y. and Rutsch, F. (2012) Generalized arterial calcification of infancy and pseudoxanthoma elasticum: two sides of the same coin. Front Genet 3, 302. 10.3389/fgene.2012.00302
27. Ferreira, C. R. et al. (2021) Ectopic Calcification and Hypophosphatemic Rickets: Natural History of ENPP1 and ABCC6 Deficiencies. J Bone Miner Res 36, 2193-2202. 10.1002/jbmr.4418
28. Bartstra, J. W. et al. (2021) Genotype-phenotype correlation in pseudoxanthoma elasticum. Atherosclerosis 324, 18-26. 10.1016/j.atherosclerosis.2021.03.012
29. Matsuzaki, Y. et al. (2005) Tissue-specific expression of the ABCC6 gene. J Invest Dermatol 125, 900-905. 10.1111/j.0022-202X.2005.23897.x
30. Scheffer, G. L. et al. (2002) MRP6 (ABCC6) detection in normal human tissues and tumors. Lab Invest 82, 515-518. 10.1038/labinvest.3780444
31. Jansen, R. S. et al. (2014) ABCC6-mediated ATP secretion by the liver is the main source of the mineralization inhibitor inorganic pyrophosphate in the systemic circulation-brief report. Arterioscler Thromb Vasc Biol 34, 1985-1989. 10.1161/ATVBAHA.114.304017
32. Jansen, R. S. et al. (2013) ABCC6 prevents ectopic mineralization seen in pseudoxanthoma elasticum by inducing cellular nucleotide release. Proc Natl Acad Sci USA 110, 20206-20211. 10.1073/pnas.1319582110
33. Iwanaga, A. et al. (2022) Clinical practice guidelines for pseudoxanthoma elasticum (2017): Clinical Practice Guidelines for Pseudoxanthoma Elasticum Drafting Committee. J Dermatol 49, e91-e98. 10.1111/1346-8138.16301
34. Fong, V. and Patel, S. B. (2021) Recent advances in ABCG5 and ABCG8 variants. Curr Opin Lipidol 32, 117-122. 10.1097/MOL.0000000000000734
35. Hooper, A. J. et al. (2017) Clinical utility gene card for: Sitosterolaemia. Eur J Hum Genet 25. 10.1038/ejhg.2016.187
36. Bhattacharyya, A. K. and Connor, W. E. (1974) Beta-sitosterolemia and xanthomatosis. A newly described lipid storage disease in two sisters. J Clin Invest 53, 1033-1043. 10.1172/JCI107640
37. Patel, S. B. et al. (1998) Sitosterolemia: exclusion of genes involved in reduced cholesterol biosynthesis. J Lipid Res 39, 1055-1061
38. Williams, K. et al. (2021) Sitosterolemia: Twenty Years of Discovery of the Function of ABCG5ABCG8. Int J Mol Sci 22. 10.3390/ijms22052641
39. Graf, G. A. et al. (2002) Coexpression of ATP-binding cassette proteins ABCG5 and ABCG8 permits their transport to the apical surface. J Clin Invest 110, 659-669. 10.1172/JCI16000
40. Jakulj, L. et al. (2016) Transintestinal Cholesterol Transport Is Active in Mice and Humans and Controls Ezetimibe-Induced Fecal Neutral Sterol Excretion. Cell Metab 24, 783-794. 10.1016/j.cmet.2016.10.001
41. Zein, A. A. et al. (2019) ABCG5/G8: a structural view to pathophysiology of the hepatobiliary cholesterol secretion. Biochem Soc Trans 47, 1259-1268. 10.1042/BST20190130
42. Chen, Z. C. et al. (2008) Significant association of ABCG8:D19H gene polymorphism with hypercholesterolemia and insulin resistance. J Hum Genet 53, 757-763. 10.1007/s10038-008-0310-2
43. Rust, S. et al. (1999) Tangier disease is caused by mutations in the gene encoding ATP-binding cassette transporter 1. Nat Genet 22, 352-355. 10.1038/11921
44. Schaefer, E. J. et al. (2016) Diagnosis and treatment of high density lipoprotein deficiency. Prog Cardiovasc Dis 59, 97-106. 10.1016/j.pcad.2016.08.006
45. Maranghi, M. et al. (2019) A novel splicing mutation in the ABCA1 gene, causing Tangier disease and familial HDL deficiency in a large family. Biochem Biophys Res Commun 508, 487-493. 10.1016/j.bbrc.2018.11.064
46. Koseki, M. et al. (2021) Current Diagnosis and Management of Tangier Disease. J Atheroscler Thromb 28, 802-810. 10.5551/jat.RV17053
47. Langmann, T. et al. (1999) Molecular cloning of the human ATP-binding cassette transporter 1 (hABC1): evidence for sterol-dependent regulation in macrophages. Biochem Biophys Res Commun 257, 29-33. 10.1006/bbrc.1999.0406
48. Hoekstra, M. et al. (2003) Specific gene expression of ATP-binding cassette transporters and nuclear hormone receptors in rat liver parenchymal, endothelial, and Kupffer cells. J Biol Chem 278, 25448-25453. 10.1074/jbc. M301189200
49. Wang, X. et al. (2007) Macrophage ABCA1 and ABCG1, but not SR—BI, promote macrophage reverse cholesterol transport in vivo. J Clin Invest 117, 2216-2224. 10.1172/JCI32057
50. Oram, J. F. and Lawn, R. M. (2001) ABCA1. The gatekeeper for eliminating excess tissue cholesterol. J Lipid Res 42, 1173-1179
51. Oram, J. F. et al. (2000) ABCA1 is the cAMP-inducible apolipoprotein receptor that mediates cholesterol secretion from macrophages. J Biol Chem 275, 34508-34511. 10.1074/jbc. M006738200
52. Brunham, L. R. et al. (2007) Beta-cell ABCA1 influences insulin secretion, glucose homeostasis and response to thiazolidinedione treatment. Nat Med 13, 340-347. 10.1038/nm1546
53. Wang, N. et al. (2000) Specific binding of ApoA-I, enhanced cholesterol efflux, and altered plasma membrane morphology in cells expressing ABC1. J Biol Chem 275, 33053-33058. 10.1074/jbc. M005438200
54. Jacobo-Albavera, L. et al. (2021) The Role of the ATP-Binding Cassette A1 (ABCA1) in Human Disease. Int J Mol Sci 22. 10.3390/ijms22041593
55. Al-Khuzaei, S. et al. (2021) An Overview of the Genetics of ABCA4 Retinopathies, an Evolving Story. Genes (Basel) 12. 10.3390/genes12081241
56. Tsang, S. and Sharma, T. (2018) Stargardt disease. In Atlas of Inherited Retinal Diseases (Tsang, S. and Sharma, T., eds), pp. 140-151, Springer International Publishing AG
57. Tanna, P. et al. (2017) Stargardt disease: clinical features, molecular genetics, animal models and therapeutic options. Br J Ophthalmol 101, 25-30. 10.1136/bjophthalmol-2016-308823
58. Kawaguchi, K. and Morita, M. (2016) ABC Transporter Subfamily D: Distinct Differences in Behavior between ABCD1-3 and ABCD4 in Subcellular Localization, Function, and Human Disease. Biomed Res Int 2016, 6786245. 10.1155/2016/6786245
59. Turk, B. R. et al. (2020) X-linked adrenoleukodystrophy: Pathology, pathophysiology, diagnostic testing, newborn screening and therapies. Int J Dev Neurosci 80, 52-72. 10.1002/jdn.10003
60. Barthel, A. et al. (2019) An Update on Addison's Disease. Exp Clin Endocrinol Diabetes 127, 165-175. 10.1055/a-0804-2715
61. Kawaguchi, K. et al. (2016) Translocation of the ABC transporter ABCD4 from the endoplasmic reticulum to lysosomes requires the escort protein LMBD1. Sci Rep 6, 30183. 10.1038/srep30183
62. Weinhofer. I. et al. (2018) Impaired plasticity of macrophages in X-linked adrenoleukodystrophy. Brain 141, 2329-2342. 10.1093/brain/awy127
63. Beers, M. F. and Mulugeta, S. (2017) The biology of the ABCA3 lipid transporter in lung health and disease. Cell Tissue Res 367, 481-493. 10.1007/s00441-016-2554-z
64. Flamein, F. et al. (2012) Molecular and cellular characteristics of ABCA3 mutations associated with diffuse parenchymal lung diseases in children. Hum Mol Genet 21, 765-775. 10.1093/hmg/ddr508
65. Hammel, M. et al. (2007) Targeted inactivation of the murine Abca3 gene leads to respiratory failure in newborns with defective lamellar bodies. Biochem Biophys Res Commun 359, 947-951. 10.1016/j.bbrc.2007.05.219
66. Wambach, J. A. et al. (2022) First Steps toward Personalized Therapies for ABCA3 Deficiency. Am J Respir Cell Mol Biol 66, 349-350. 10.1165/rcmb.2021-0405ED
67. Sanders, M. et al. (2021) Genomic, transcriptomic, and protein landscape profile of CFTR and cystic fibrosis. Hum Genet 140, 423-439. 10.1007/s00439-020-02211-w
68. Amaral, M. D. (2015) Novel personalized therapies for cystic fibrosis: treating the basic defect in all patients. J Intern Med 277, 155-166. 10.1111/joim.12314
69. Breijyeh, Z. and Karaman, R. (2020) Comprehensive Review on Alzheimer's Disease: Causes and Treatment. Molecules 25. 10.3390/molecules25245789
70. Anstey, K. J. et al. (2017) Updating the Evidence on the Association between Serum Cholesterol and Risk of Late-Life Dementia: Review and Meta-Analysis. J Alzheimers Dis 56, 215-228. 10.3233/JAD-160826
71. Torrandell-Haro, G. et al. (2020) Statin therapy and risk of Alzheimer's and age-related neurodegenerative diseases. Alzheimers Dement (NY) 6, e12108. 10.1002/trc2.12108
72. Bossaerts, L. et al. (2022) The role of ATP-binding cassette subfamily A in the etiology of Alzheimer's disease. Mol Neurodegener 17, 31. 10.1186/si3024-022-00536-w
73. Koldamova, R. P. et al. (2005) The liver X receptor ligand T0901317 decreases amyloid beta production in vitro and in a mouse model of Alzheimer's disease. J Biol Chem 280, 4079-4088. 10.1074/jbc. M411420200
74. Koldamova, R. et al. (2005) Lack of ABCA1 considerably decreases brain ApoE level and increases amyloid deposition in APP23 mice. J Biol Chem 280, 43224-43235. 10.1074/jbc. M504513200
75. Zhu, X. et al. (2010) Macrophage ABCA1 reduces MyD88-dependent Toll-like receptor trafficking to lipid rafts by reduction of lipid raft cholesterol. J Lipid Res 51, 3196-3206. 10.1194/jlr. M006486
76. Sviridov, D. et al. (2020) Lipid rafts as a therapeutic target. J Lipid Res 61, 687-695. 10.1194/jlr.TR120000658
77. Ehehalt, R. et al. (2003) Amyloidogenic processing of the Alzheimer beta-amyloid precursor protein depends on lipid rafts. J Cell Biol 160, 113-123. 10.1083/jcb.200207113
78. Choy, R. W. et al. (2012) Amyloid precursor protein (APP) traffics from the cell surface via endosomes for amyloid beta (Abeta) production in the trans-Golgi network. Proc Natl Acad Sci USA 109, E2077-2082. 10.1073/pnas.1208635109
79. Marquer, C. et al. (2011) Local cholesterol increase triggers amyloid precursor protein-Bacel clustering in lipid rafts and rapid endocytosis. FASEB J 25, 1295-1305. 10.1096/fj.10-168633
80. Wahrle, S. E. et al. (2005) Deletion of Abca1 increases Abeta deposition in the PDAPP transgenic mouse model of Alzheimer disease. J Biol Chem 280, 43236-43242. 10.1074/jbc. M508780200
81. Lefterov. I. et al. (2009) Memory deficits in APP23/Abcal+/− mice correlate with the level of Abeta oligomers. ASN Neuro 1. 10.1042/AN20090015
82. Jiang, Q. et al. (2008) ApoE promotes the proteolytic degradation of Abeta. Neuron 58, 681-693. 10.1016/j.neuron.2008.04.010
83. Leissring, M. A. et al. (2003) Enhanced proteolysis of beta-amyloid in APP transgenic mice prevents plaque formation, secondary pathology, and premature death. Neuron 40, 1087-1093. 10.1016/s0896-6273 (03)00787-6
84. Husain, M. A. et al. (2021) APOE and Alzheimer's Disease: From Lipid Transport to Physiopathology and Therapeutics. Front Neurosci 15, 630502. 10.3389/fnins.2021.630502
85. Wahrle, S. E. et al. (2008) Overexpression of ABCA1 reduces amyloid deposition in the PDAPP mouse model of Alzheimer disease. J Clin Invest 118, 671-682. 10.1172/JCI33622
86. Boehm-Cagan, A. et al. (2016) ABCA1 Agonist Reverses the ApoE4-Driven Cognitive and Brain Pathologies. J Alzheimers Dis 54, 1219-1233. 10.3233/JAD-160467
87. Noveir, S. D. et al. (2022) Effect of the ABCA1 agonist CS-6253 on amyloid-beta and lipoprotein metabolism in cynomolgus monkeys. Alzheimers Res Ther 14, 87. 10.1186/s13195-022-01028-1
88. Reitz, C. et al. (2013) Variants in the ATP-binding cassette transporter (ABCA7), apolipoprotein E 4, and the risk of late-onset Alzheimer disease in African Americans. JAMA 309, 1483-1492. 10.1001/jama.2013.2973
89. Cukier, H. N. et al. (2016) ABCA7 frameshift deletion associated with Alzheimer disease in African Americans. Neurol Genet 2, e79. 10.1212/NXG.0000000000000079
90. Bellenguez, C. et al. (2017) Contribution to Alzheimer's disease risk of rare variants in TREM2, SORL1, and ABCA7 in 1779 cases and 1273 controls. Neurobiol Aging 59, 220 e221-220 e229. 10.1016/j.neurobiolaging.2017.07.001
91. De Roeck, A. et al. (2017) Deleterious ABCA7 mutations and transcript rescue mechanisms in early onset Alzheimer's disease. Acta Neuropathol 134, 475-487. 10.1007/s00401-017-1714-x
92. Apostolova, L. G. et al. (2018) Associations of the Top 20 Alzheimer Disease Risk Variants With Brain Amyloidosis. JAMA Neurol 75, 328-341. 10.1001/jamaneurol.2017.4198
93. Stage, E. et al. (2016) The effect of the top 20 Alzheimer disease risk genes on gray-matter density and FDG PET brain metabolism. Alzheimers Dement (Amst) 5, 53-66. 10.1016/j.dadm.2016.12.003
94. Lyssenko, N. N. and Pratico, D. (2021) ABCA7 and the altered lipidostasis hypothesis of Alzheimer's disease. Alzheimers Dement 17, 164-174. 10.1002/alz.12220
95. Lamartiniere, Y. et al. (2018) ABCA7 Downregulation Modifies Cellular Cholesterol Homeostasis and Decreases Amyloid-beta Peptide Efflux in an in vitro Model of the Blood-Brain Barrier. J Alzheimers Dis 64, 1195-1211. 10.3233/JAD-170883
96. Tanaka, N. et al. (2010) Helical apolipoproteins of high-density lipoprotein enhance phagocytosis by stabilizing ATP-binding cassette transporter A7. J Lipid Res 51, 2591-2599. 10.1194/jlr. M006049
97. Jehle, A. W. et al. (2006) ATP-binding cassette transporter A7 enhances phagocytosis of apoptotic cells and associated ERK signaling in macrophages. J Cell Biol 174, 547-556. 10.1083/jcb.200601030
98. Aikawa, T. et al. (2019) ABCA7 haplodeficiency disturbs microglial immune responses in the mouse brain. Proc Natl Acad Sci USA 116, 23790-23796. 10.1073/pnas.1908529116
99. Satoh, K. et al. (2015) ATP-binding cassette transporter A7 (ABCA7) loss of function alters Alzheimer amyloid processing. J Biol Chem 290, 24152-24165. 10.1074/jbc. M115.655076
100. Sakae, N. et al. (2016) ABCA7 Deficiency Accelerates Amyloid-beta Generation and Alzheimer's Neuronal Pathology. J Neurosci 36, 3848-3859. 10.1523/JNEUROSCI.3757-15.2016
101. Kim, W. S. et al. (2013) Deletion of Abca7 increases cerebral amyloid-beta accumulation in the J20 mouse model of Alzheimer's disease. J Neurosci 33, 4387-4394.

1. A compound represented by Formula I: 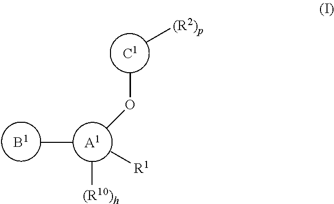
or a pharmaceutically acceptable salt thereof, wherein:
Ring A¹ is (i) a 6-membered heteroarylene having 1 or 2 heteroatoms selected from nitrogen; (ii) phenylene; or (iii) a 5-7 membered saturated or partially unsaturated monocyclic oxo-heterocyclyl having 1-2 heteroatoms independently selected from nitrogen, oxygen, and sulfur;
Ring B¹ is a 5-6 membered monocyclic heteroaryl having 1-3 heteroatoms independently selected from nitrogen, oxygen, and sulfur, a 3-7 membered monocyclic heterocyclyl having 1-2 heteroatoms independently selected from nitrogen, oxygen, and sulfur, a 3-7 membered saturated or partially unsaturated monocyclic oxo-heterocyclyl having 1-2 heteroatoms independently selected from nitrogen, oxygen, and sulfur, a 7-12 membered bridged or spirocyclic heterocyclyl having 1-2 heteroatoms independently selected from nitrogen, oxygen, and sulfur, or a partially aromatic 9-10 membered bicyclic ring containing 2, 3, or 4 heteroatoms independently selected from nitrogen, oxygen, and sulfur, wherein each ring is substituted with j instances of R³; or Ring B¹ is phenyl substituted with k instances of R³;
Ring C¹ is phenylene, a 5- or 6-membered heteroarylene containing 1 or 2 heteroatoms selected from nitrogen, oxygen, and sulfur, or pyridinylene-N-oxide;
R¹ is —N(R⁹)—SO₂—R⁶, —SO₂—N(R⁹)—R⁶, —SO₂—N(R⁷)₂, —S(NR⁹)(O)—R⁶, —N═S(O)(R⁹)₂, or 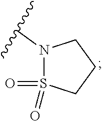 [SMILES: *N1CCCS1(=O)=O]
R² represents independently for each occurrence hydroxyl, C₁₋₃ alkyl, halo, C₁₋₃ haloalkyl, C₁₋₃ hydroxyalkyl, cyano, C₃₋₄ cycloalkyl, —O—R¹¹, a 3-5 membered heterocyclyl having 1-2 heteroatoms independently selected from nitrogen, oxygen, and sulfur, or a 7-12 membered bridged or spirocyclic heterocyclyl having 1-2 heteroatoms independently selected from nitrogen, oxygen, and sulfur, wherein each cyclokalkyl and heterocyclyl is substituted with q instances of R¹²; or two vicinal occurrences of R² are taken together with the atoms to which they are attached to form a 3-6 membered ring having 0, 1, or 2 heteroatoms independently selected from nitrogen, oxygen, and sulfur;
R³ represents independently for each occurrence halo, cyano, C₁₋₆ alkyl, C₂₋₃ alkynyl, C₁₋₆ haloalkyl, hydroxyl, (C₀₋₂ alkylene)-C₁₋₆ alkoxyl, C₁₋₃ hydroxyalkyl, —CON(R⁴)₂, —C(O)—R⁵, —C(O)—OR⁵, —S(O)₂—R⁵, —N(R⁴)—C(O)—R⁵, —S(NR⁴)(O)—R⁵, C₁₋₆ haloalkoxyl, C₃₋₄ cycloalkyl, or a 3-6 membered heterocyclyl having 1-2 heteroatoms independently selected from nitrogen, oxygen, and sulfur, wherein each cycloalkyl and heterocyclyl is substituted with r instances of R¹³;
R⁴ represents independently for each occurrence hydrogen, C₁₋₄ alkyl, or cyclopropyl;
R⁵ and R⁶ each represent independently for each occurrence C₁₋₄ alkyl, C₃₋₅ cycloalkyl, —(C₀₋₂ alkylene)-phenyl, —(C₀₋₂ alkylene)-(3-6 membered monocyclic heterocyclyl having 1-2 heteroatoms independently selected from nitrogen, oxygen, and sulfur), or —(C₀₋₂ alkylene)-(5-10 membered heteroaryl having 1-3 heteroatoms independently selected from nitrogen, oxygen, and sulfur), wherein the cycloalkyl, phenyl, heterocyclyl, and heteroaryl are substituted with m instances of R⁸, and wherein each alkylene is substituted with n instances of R⁸;
R⁷ is hydrogen, or two R⁷ groups attached to the same nitrogen atom are taken together with the nitrogen to which they are attached to form a 3-6 membered saturated monocyclic ring having in addition to said nitrogen atom 0, 1, or 2 heteroatoms independently selected from nitrogen, oxygen, and sulfur;
R⁸ represents independently for each occurrence C₁₋₄ alkyl, halo, C₁₋₃ haloalkyl, hydroxyl, C₁₋₄ alkoxy, cyano, or cyclopropyl;
R⁹ represents independently for each occurrence hydrogen, C₁₋₃ alkyl, or cyclopropyl;
R¹⁰ is C₁₋₃ alkyl, C₁₋₃ haloalkyl, halo, hydroxyl, or cyclopropyl;
R¹¹ is C₁₋₄ alkyl, C₁₋₄ haloalkyl, cyclopropyl, or a 3-5 membered saturated heterocyclyl having 1-2 heteroatoms independently selected from nitrogen, oxygen, and sulfur;
R¹² is C₁₋₃ alkyl, halo, cyano, or —SO₂—(C₁₋₃ alkyl);
R¹³ is halo, C₁₋₃ alkyl, or C₁₋₃ alkoxyl;
h is 0, 1, or 2;
j is 0, 1, or 2;
k is 1 or 2;
m is 0, 1, or 2;
n is 0, 1, 2, or 3;
p is 1, 2, 3, or 4;
q is 0, 1, or 2; and
r is 0, 1, or 2.

2. The compound of claim 1, wherein the compound is a compound of Formula I.

3. (canceled)

4. The compound of claim 1, wherein Ring C¹ is pyridinylene.

5. The compound of claim 1, wherein Ring C¹ is pyrimidinylene.

6-9. (canceled)

10. The compound of claim 1, wherein Ring A¹ is a 6-membered monocyclic heteroaromatic ring containing 1 or 2 heteroatoms selected from nitrogen.

11. The compound of claim 1, wherein Ring A¹ is pyridinylene.

12. The compound of claim 1, wherein Ring A¹ is pyrimidinylene.

13. The compound of claim 1, wherein R¹ is —N(R⁹)—SO₂—R⁶.

14-20. (canceled)

21. The compound of claim 1, wherein the compound is a compound of Formula I-F, or a pharmaceutically acceptable salt thereof:

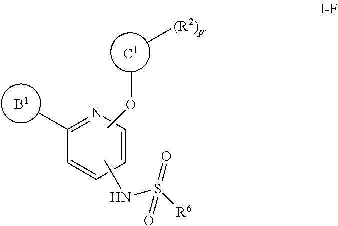

22. The compound of claim 1, wherein the compound is a compound of Formula I-G, I-H, I-J, I-K, or I-L, or a pharmaceutically acceptable salt thereof:

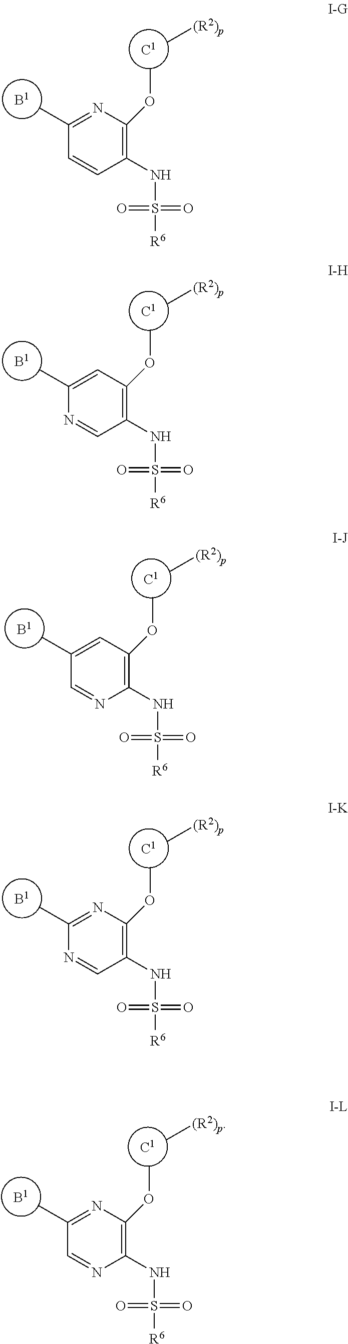

23. (canceled)

24. The compound of claim 21, wherein Ring C¹ is a 6-membered heteroarylene containing 1 or 2 heteroatoms selected from nitrogen, oxygen, and sulfur.

25. (canceled)

26. The compound of claim 21, wherein Ring C¹ is pyridinylene.

27. The compound of claim 21, wherein Ring C¹ is pyrimidinylene.

28. (canceled)

29. (canceled)

30. (canceled)

31. The compound of claim 1, wherein Ring B¹ is pyridinyl substituted with j instances of R³.

32. (canceled)

33. The compound of claim 1, wherein the compound is a compound of Formula I-W or a pharmaceutically acceptable salt thereof:

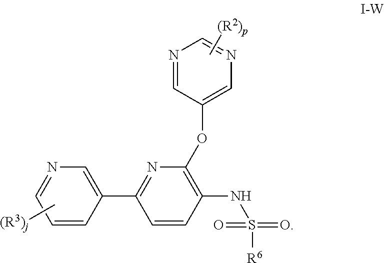

34. The compound of claim 1, wherein the compound is a compound of Formula I-X or a pharmaceutically acceptable salt thereof:

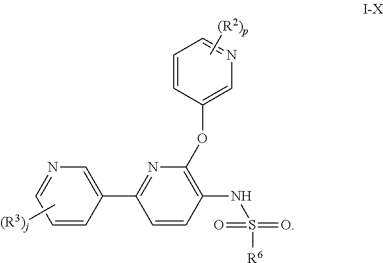

35-41. (canceled)

42. The compound of claim 1, wherein R³ represents independently for each occurrence halo, cyano, C₁₋₆ alkyl, C₁₋₆ haloalkyl, hydroxyl, or C₁₋₆ alkoxyl.

43. (canceled)

44. The compound of claim 1, wherein R² represents independently for each occurrence C₁₋₃ alkyl, C₁₋₃ haloalkyl, —O—(C₁₋₄ haloalkyl), or C₃₋₄ cycloalkyl.

45. (canceled)

46. (canceled)

47. (canceled)

48. The compound of claim 1, wherein R⁶ represents independently for each occurrence C₁₋₄ alkyl or C₃₋₅ cycloalkyl.

49. (canceled)

50. A compound in Table 1 or a pharmaceutically acceptable salt thereof.

51. The compound of claim 50, wherein the compound is selected from: 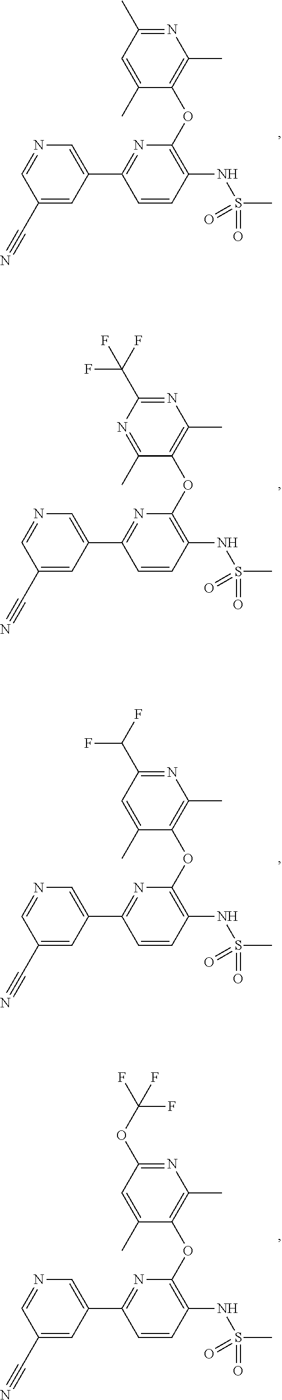 [SMILES: Cc1cc(C)c(Oc2nc(-c3cncc(C#N)c3)ccc2NS(C)(=O)=O)c(C)n1 ; Cc1nc(C(F)(F)F)nc(C)c1Oc1nc(-c2cncc(C#N)c2)ccc1NS(C)(=O)=O ; Cc1cc(C(F)F)nc(C)c1Oc1nc(-c2cncc(C#N)c2)ccc1NS(C)(=O)=O ; Cc1cc(OC(F)(F)F)nc(C)c1Oc1nc(-c2cncc(C#N)c2)ccc1NS(C)(=O)=O] 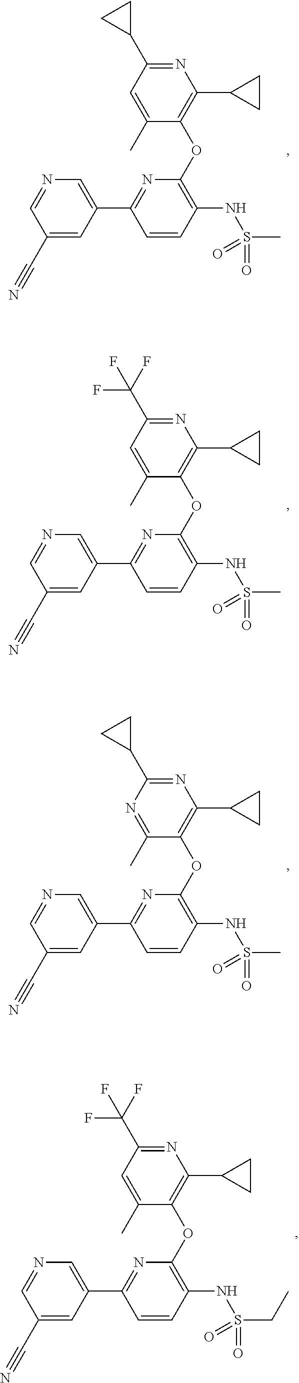 [SMILES: Cc1cc(C2CC2)nc(C2CC2)c1Oc1nc(-c2cncc(C#N)c2)ccc1NS(C)(=O)=O ; Cc1cc(C(F)(F)F)nc(C2CC2)c1Oc1nc(-c2cncc(C#N)c2)ccc1NS(C)(=O)=O ; Cc1nc(C2CC2)nc(C2CC2)c1Oc1nc(-c2cncc(C#N)c2)ccc1NS(C)(=O)=O ; CCS(=O)(=O)Nc1ccc(-c2cncc(C#N)c2)nc1Oc1c(C)cc(C(F)(F)F)nc1C1CC1] 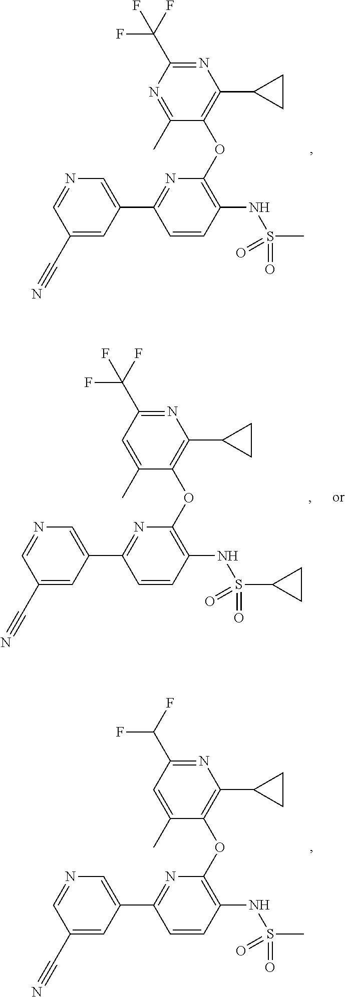 [SMILES: Cc1nc(C(F)(F)F)nc(C2CC2)c1Oc1nc(-c2cncc(C#N)c2)ccc1NS(C)(=O)=O ; Cc1cc(C(F)(F)F)nc(C2CC2)c1Oc1nc(-c2cncc(C#N)c2)ccc1NS(=O)(=O)C1CC1 ; Cc1cc(C(F)F)nc(C2CC2)c1Oc1nc(-c2cncc(C#N)c2)ccc1NS(C)(=O)=O]
or a pharmaceutically acceptable salt thereof.

52. A pharmaceutical composition comprising a compound of claim 1 and a pharmaceutically acceptable carrier.

53. A method of treating a disorder associated with ABC transporter dysfunction, comprising administering to a subject in need thereof a therapeutically effective amount of a compound of claim 1 to treat the disorder, wherein the disorder associated with ABC transporter dysfunction is characterized by dysfunction in a transporter selected from one or more of ABCA1, ABCA2, ABCA3, ABCA4, ABCA5, ABCA7, ABCA12, ABCB2, ABCB3, ABCB4, ABCB6, ABCB7, ABCB10, ABCB11, ABCC1, ABCC2, ABCC4, ABCC5, ABCC6 ABCC7, ABCC8, ABCC9, ABCC12, ABCD1, ABCD2, ABCD3, ABCD4, ABCG5, ABCG8, ABCG1, and ABCG4.

54. (canceled)

55. A method of treating or preventing a disorder, comprising administering to a subject in need thereof a therapeutically effective amount of a compound of claim 1 to treat or prevent the disorder, wherein the disorder is selected from Tangier disease, Surfactant metabolism dysfunction pulmonary 3, autosomal recessive Ichthyosis congenital 4A (ARCI), Bare lymphocyte syndrome type I, Bare lymphocyte syndrome type I due to TAP2 deficiency, Dyschromatosis universalis hereditaria 3, X-linked sideroblastic anemia with ataxia, Dubin-Johnson Syndrome, Cystic fibrosis (CF), Familial Hyperinsulinemic Hypoglycemia 1, Intellectual disability Myopathy Syndrome, congenital bile acid synthesis defect 5, Methylmalonic aciduria and homocystinuria cblJ type, Sitostrolemia, Stargardt disease, PFIC3, PFIC2, Pseudoxanthoma Elasticum, X-linked adrenoleukodystrophy (ALD), Cholestasis, Hyperbilirubinemia, Intrahepatic cholestasis of pregnancy, Biliary atresia, Alagille syndrome, primary biliary cholangitis, primary sclerosing cholangitis, NAFLD/NASH, Alzheimer's disease, Huntington's disease, Multiple sclerosis, Parkinson's disease, Hirschsprung disease, Zellweger syndrome, Type 2 diabetes, Obesity, Type 1 diabetes, Atherosclerosis, Dyslipidemia, generalized arterial calcification of infancy, calciphylaxis, Autosomal recessive cone-rod dystrophy, Gout, PFIC1, Myo5B deficiency cholestasis, PFIC4, low phospholipid associated cholelithiasis, chronic kidney disease, Progeria (Hutchinson-Gilford progeria syndrome), hemodialysis, end stage renal disease, aortic stenosis, peripheral arterial disease, or ischemic stroke.

56. The method of claim 55, wherein the disorder is selected from calciphylaxis, pseudoxanthoma elasticum, generalized arterial calcification of infancy, chronic kidney disease, Progeria (Hutchinson-Gilford progeria syndrome), hemodialysis, end stage renal disease, aortic stenosis, peripheral arterial disease, or ischemic stroke.

57. The method of claim 56, wherein the compound is a compound of claim 51.

58. A method of modulating the function of an ABC transporter in a subject, comprising administering to the subject an effective amount of a compound of claim 1 to thereby modulate the function of the ABC transporter in the subject, wherein the ABC transporter is selected from one or more of ABCA1, ABCA2, ABCA3, ABCA4, ABCA5, ABCA7, ABCA12, ABCB2, ABCB3, ABCB4, ABCB6, ABCB7, ABCB10, ABCB11, ABCC1, ABCC2, ABCC4, ABCC5, ABCC6 ABCC7, ABCC8, ABCC9, ABCC12, ABCD1, ABCD2, ABCD3, ABCD4, ABCG5, ABCG8, ABCG1, and ABCG4.

59. (canceled)

60. The method of claim 55 wherein the subject is a human.

61. The compound of claim 50, wherein the compound is 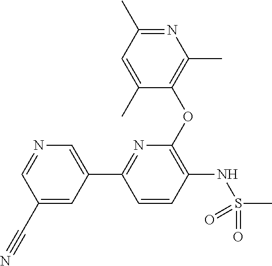 [SMILES: Cc1cc(C)c(Oc2nc(-c3cncc(C#N)c3)ccc2NS(C)(=O)=O)c(C)n1]
or a pharmaceutically acceptable salt thereof.

62. The compound of claim 50, wherein the compound is

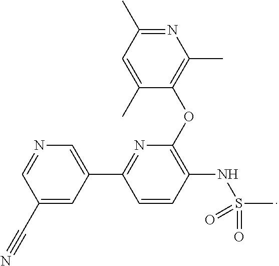 [SMILES: Cc1cc(C)c(Oc2nc(-c3cncc(C#N)c3)ccc2NS(C)(=O)=O)c(C)n1]

63. The compound of claim 50, wherein the compound is 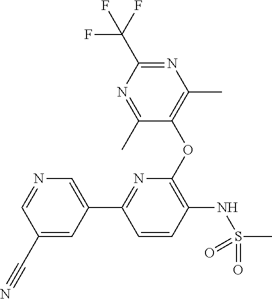 [SMILES: Cc1nc(C(F)(F)F)nc(C)c1Oc1nc(-c2cncc(C#N)c2)ccc1NS(C)(=O)=O]
or a pharmaceutically acceptable salt thereof.

64. The compound of claim 50, wherein the compound is

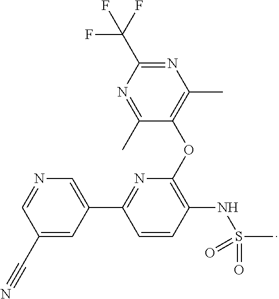 [SMILES: Cc1nc(C(F)(F)F)nc(C)c1Oc1nc(-c2cncc(C#N)c2)ccc1NS(C)(=O)=O]

65. A pharmaceutical composition comprising a compound of claim 61 and a pharmaceutically acceptable carrier.

66. A pharmaceutical composition comprising a compound of claim 62 and a pharmaceutically acceptable carrier.

67. A pharmaceutical composition comprising a compound of claim 63 and a pharmaceutically acceptable carrier.

68. A pharmaceutical composition comprising a compound of claim 64 and a pharmaceutically acceptable carrier.
